(19) United States
(12) Patent Application Publication
(10) Pub. No.: US 2025/0034112 A1
(43) Pub. Date: Jan. 30, 2025

(54) IMPROVED SELECTIVE JAK2 INHIBITORS AND METHODS OF USE

(71) Applicant: Yale University, New Haven, CT (US)

(72) Inventor: William L. Jorgensen, New Haven, CT (US)

(21) Appl. No.: 18/701,027

(22) PCT Filed: Oct. 13, 2022
(86) PCT No.: PCT/US2022/046554

§ 371 Date: Apr. 12, 2024

Related U.S. Application Data
(60) Provisional application No. 63/255,262, filed on Oct. 13, 2021.

(51) Int. Cl. C07D 401/12; A61K 31/4196; A61K 31/422; A61K 31/4439; A61K 31/519; A61K 31/5377; A61K 45/06; C07D 249/14; C07D 413/12; C07D 487/04

(52) U.S. Cl. CPC C07D 401/12; A61K 31/4196; A61K 31/422; A61K 31/4439; A61K 31/519; A61K 31/5377; A61K 45/06; C07D 249/14; C07D 413/12; C07D 487/04

(57) ABSTRACT

The compounds of Formula I described herein regulate activity of JAK2 by specifically binding to the JAK2 pseudokinase domain, JH2, and are useful as therapeutic agents in the treatment or amelioration of myeloproliferative disorders. Also provided herein are methods of treating myeloproliferative disorders, and methods of making compounds of Formula I.

### CROSS-REFERENCE TO RELATED APPLICATION

This application claims priority to U.S. Provisional Patent Application No. 63/255,262 entitled “SELECTIVE JAK2 INHIBITORS AND METHODS OF USE,” filed Oct. 13, 2021, the disclosure of which is incorporated herein by reference in its entirety.

### STATEMENT REGARDING FEDERALLY SPONSORED RESEARCH

This invention was made with government support under GM124165, OD021527, OD018007, OD001800, GM032136, and GM007324 awarded by National Institutes of Health and under DE-AC02-06CH11357 awarded by the Department of Energy. The government has certain rights in the invention.

### BACKGROUND

Janus kinases (JAKs) are a family of non-receptor tyrosine kinases that are essential components of the JAK-STAT signaling pathway. Aberrant signaling in this cascade is responsible for numerous diseases, including disorders of the immune system and many forms of cancer. Specifically, the Val617Phe mutation in JH2 stimulates the activity of the adjacent kinase domain (JH1) resulting in myeloproliferative disorders.

There is an ever-present need to develop new therapies to treat myeloproliferative disorders, such as chronic myelogenous leukemia (CML), polycythemia vera, primary myelofibrosis (also called chronic idiopathic myelofibrosis), essential thrombocythemia, chronic neutrophilic leukemia, and chronic eosinophilic leukemia. The present invention addresses and meets this need.

### BRIEF SUMMARY

In various aspects, a compound of Formula I, or a pharmaceutically acceptable salt, tautomer, or enantiomer thereof is provided:

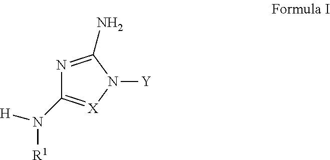

wherein,
Y is selected from the group consisting of —CH₂—R², —C(═O)NR²R³, optionally substituted C₅₋₁₀ heteroaryl, and optionally substituted C₅₋₆ heterocycloalkyl;
wherein the optional substitution is at least one substituent selected from the group consisting of C₁₋₅ alkyl, C₁₋₅ alkoxy, C₁₋₅ thioalkyl, C₁₋₅ aminoalkyl, C₅₋₁₀ aryl, C₅₋₁₀ heteroaryl, ═O (oxo), F, Cl, Br, I, C(═O)OR, NHC(═O)R, and OH;
R¹ and R² are each independently selected from the group consisting of C₂₋₆ alkenyl, C₃₋₇ cycloalkyl, C₆₋₁₀ aryl, 5-10 membered heteroaryl, 4-10 membered heterocycloalkyl, C₆₋₁₀-5-6 membered heterobiaryl, 5-6 membered-C₆₋₁₀ heterobiaryl, and C₆₋₁₀-C₆₋₁₀ biaryl,
   each optionally substituted by at least one group of formula (LL)_{zz}-GG, wherein
      LL is at each occurrence independently selected from the group consisting of —O—, —OR, —S—, —S(═O)—, —S(═O)₂—, —NR², —CH═, —C≡, —CH₂—, —CHR—, —CR₂—, —CH₃, —C(═O)—, —C(═NR)—;
      zz is an integer from 1 to 30;
      GG is a substituent selected from the group consisting of F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, and C(═NH)N(R)₂,
   wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
R³ is H or C₁₋₄ hydrocarbyl;
X is N or C—R⁴, wherein R⁴ is H or C₁₋₄ hydrocarbyl optionally substituted by 1 to 3 substituents selected from the group consisting of OR′, NHR′, and NR′₂,
   wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl; or wherein two R′ groups bonded to a nitrogen atom or to adjacent nitrogen atoms can together with the nitrogen atom or atoms form a heterocyclyl.

In various aspects, a compound of Formula II, or a pharmaceutically acceptable salt, tautomer, or enantiomer thereof is provided:

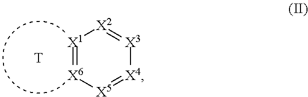

wherein
T is an optional 5 or 6 membered heterocyclic fused ring that is optionally substituted by at least one -(LL)_{zz}-GG;
each of X¹-X⁶ is independently N or C—Y;
each occurrence of Y is independently absent, H, -(Q)ₙ-(C₃-C₁₂)cycloalkyl, -(Q)ₙ-(C₃-C₁₈)heterocycloalkyl, -(Q)ₙ-(C₆-C₁₈)aryl, or -(Q)ₙ-(C₅-C₁₈)heteroaryl;
Q is absent, or independently selected at each occurrence from the group consisting of O, CH₂, NH, and N—C₁₋₄ alkyl;
n is an integer from 1 to 10;
each cycloalkyl, heterocycloalkyl, aryl, and heteroaryl in Y is optionally substituted by at least one -(LL)_{zz}-GG,
at each occurrence LL is independently selected from the group consisting of —O—, —OR, —S—, —S(═O)—, —S(═O)₂—, —NR—, —NR², —CH═, —C═, —CH₂—, —CHR—, —CR₂—, —CH₃, —C(═O)—, —C(═NR)—;
zz is an integer from 1 to 30;
GG is a substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl;
wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
wherein each alkyl, alkenyl, alkynyl, cycloalkyl, heterocycloalkyl, aryl, and heteroaryl in GG is optionally substituted aby at least one substituent selected from the group consisting of H, F, Cl, Br, I, OR′, OC(═O)N(R′)₂, CN, NO₂, CF₃, OCF₃, R′, N(′R)₂, SR′, SOR′, SO₂R′, SO₂N(R′)₂, SO₃R′, C(═O) R′, C(═O)OR′, OC(═O) R′, O(CH₂)₀₋₂C(═O)OR′, C(═O)N(R′)₂, OC(═O)N(R′)₂, (CH₂)₀₋₂N(R′)C(═O) R′, N(R′)SO₂R′, N(R′)C(═O)OR′, N(R′)C(═O) R′, N(R′)C(═O)N(R′)₂, and C(═NH)N(R′)₂;
wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; with the proviso that at least two of X¹-X⁶ is N and at least one of X¹-X⁶ is C—NH₂.

Compounds of Formula I and Formula II are useful in the treatment, amelioration, and/or prevention of myeloproliferative neoplasms, such as chronic myelogenous leukemia (CML), polycythemia vera, primary myelofibrosis, essential thrombocythemia, chronic neutrophilic leukemia, and chronic eosinophilic leukemia.

### BRIEF DESCRIPTION OF THE FIGURES

The drawings illustrate generally, by way of example, but not by way of limitation, various embodiments of the present application.

FIGS. 1A-1B show rendered views of the JAK2 ATP binding site. FIG. 1A is a rendering from the 1.86-Å crystal structure of 22 with wild-type JAK2 JH2 (PDB ID 7JYQ). Orientation of 22, with hydrogen bonds in the hinge area dashed. FIG. 1B shows the desired direction of growth in the ATP-binding pocket with the red arrow.

FIGS. 2A-2B show renderings of the JAK2 ATP binding site. Renderings from the (FIG. 2A) 2.16-Å crystal structure of 33b (PDB ID 7JYO) and (FIG. 2B) 2.02-Å crystal structure of 33m (PDB ID 6XJK) with wild type JAK2 JH2.

FIG. 3 shows the computed binding mode, using MCPRO, for 33n with WT JAK2 JH2 illustrating the expected eastward extension towards Arg715 (WT=wild type).

FIGS. 4A-4C (FIG. 1 in paper) shows representative structures of JAK2 JH2 binders discovered previously: (FIG. 4A) indoloxytriazines with single-digit μM affinity for JH2 and up to 14-fold selectivity over JH1, (FIG. 4B) diaminotriazoles with low triple-digit nM affinity for JH2 and up to 19-fold selectivity over JH1, and (FIG. 4C) pyrrolopyrimidines with double-digit nM affinity for JH2 and up to 360-fold selectivity over JH1.

FIG. 5 shows read optimization studies on diaminotriazole ligands. Compound 1 is the known pan-CDK and pan-JAK kinase inhibitor JNJ7706621. Compound 2 is a previously reported nM and selective JAK2 JH2 binder from optimization studies on 1. Compounds 3-14 are new JAK2 JH2-binding molecules reported in this work. Lead optimization stages: Ring and substituent optimization with eventual appending of the OBn moiety (phenyl-benzyloxy analogs), and permeability optimization.

FIGS. 6A-6B show the crystal structure of 2 in complex with WTJAK2 JH2. Shown is the biaryl-end of the ligand, bound to the polar ATP-binding region in the JH2 site (PDB ID: 60CC). Lead optimization strategies were (FIG. 6A) to improve the hydrogen-bonding network and interactions with T555, T557 and R715, and (FIG. 6B) grow towards W718.

FIG. 7 shows a comparison of 2 (crystal-structure pose in JAK2JH2, PDB ID: 60CC) with BOMB-generated poses for 6-membered heterocycle analogs 3, 5, and 6.

FIG. 8 shows a crystal structure of 6 bound to WTJAK2 JH2 (PDB ID: 7SZW), showing an extensive hydrogen-bonding network in the east side of the JH2 binding site. The pyridine N is hydrogen-bonded to a water molecule that also contacts the carboxylate group, N673, and R715.

FIG. 9 shows a superposition of the crystal structures of 6 (cyan, PDB ID: 7SZW) and a pyrrolopyrimidine analog (shown in FIG. 4C, X═CH2) bound to WT JAK2 JH2 (magenta, PDB ID: 7T1T). The phenethyl group projects from the 4-position of the pyridine ring of 6.

FIG. 10 shows a crystal structure of 11 bound to WT JAK2 JH2 (PDB ID: 7TOP). There are hydrogen bonds for the carboxylate group with T557, T555, and four water molecules. The benzyloxy phenyl ring makes an aryl-aryl interaction with W718 and a cation-π interaction with R715.

FIGS. 11A-11B show a crystal structure alignment showing the distortion caused in the αC helix, the β3-αC and β4-β5 loops of JAK2 JH2 upon binding of 11. WT JH2 bound to 11 (green, PDB ID: 7TOP), WT JAK2 JH2 bound to ATP (magenta, PDB ID: 4FVQ), and V617F JAK2JH2 bound to ATP (purple, PDB ID: 4FVR). Binding of 11 causes F594 and F595 to adopt a mutant-like conformation (FIG. 11A), as observed in the conformation of β3-αC and β4-β5 loops (FIG. 11B). In addition, the carboxylic acid of 11 pushes the backbone of residues Q553-G554-T555 (β1 region) upwards.

FIGS. 12A-12B show (FIG. 12A) Western blot analysis measuring the levels of STAT5 and phosphorylated-STAT5 (P-STAT5) in lysates from HEL cells after treatment with compounds 11 and 13 for 3 hours at the indicated concentrations. (FIG. 12B) Western blot analysis measuring the levels of STAT5 and phosphorylated-STAT5 (P-STAT5) in lysates from HEL and TF-1 cells after incubation with compound 13 at the indicated concentrations for 1 hour. The compound has similar activity in both cell lines, exhibiting complete inhibition at 20 μM. Original blots have been cropped for clarity.

FIG. 13 illustrates structural elements that promote binding to the JAK2 JH2 domain. Shown are a donor-acceptor-donor motif (magenta), an aromatic pharmacophore (green) attached to a terminal carboxylate (blue) through a variable linker (blue sphere), and a solvent-exposed aryl nitrile or sulfonamide moiety (red). These structural elements were adopted from our previously reported triazine and triazole series and used to reconfigure the false virtual screen hit JAK198 with further inspiration from several JAK2 drugs.

FIGS. 14A-14D show pyrrolopyrimidine (FIG. 14A) D- and (FIG. 14B) L-phenylalanine or (FIG. 14C) D- and (FIG. 14D) L-homophenylalanine analogues modeled via BOMB in the WT JAK2 JH2 domain starting from the JAK2 JH2/JAK67 crystal structure (PDB ID: 6XJK26). The D-isomers tended toward Phe594, whereas the L-isomers tended toward Trp718.

FIGS. 15A-15B show crystal structure of pyrrolopyrimidine 11 bound to the WT JAK2 JH2 domain (PDB ID: 7T1T, 2.08 Å resolution). Shown are (FIG. 15A) the entire ligand bound to the JAK2 JH2 domain, including interactions with key residues, and (FIG. 15B) the L-homophenylalanine urea moiety, with its orientation and interactions with binding-site residues.

### DETAILED DESCRIPTION

The disclosure provides certain compounds that are, in various embodiments, selective inhibitors of the JAK2 JH2 domain (a pseudokinase domain). JH2 domains do have a regulatory function for the JH1 kinase activity, such that mutations in JH2 can cause hyperactivation leading to numerous diseases and cancer.

In certain non-limiting embodiments of the disclosure, the compounds contemplated herein bind to the JAK2 JH2 ATP binding site with selectivity over the corresponding JAK2 JH1 ATP binding site. In certain embodiments, the compounds contemplated herein selectively reverse the activating effect of certain proliferative mutations (such as, but not limited to, V617F) in JAK2 JH2.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The substitution can be direct substitution, whereby the hydrogen atom is replaced by a functional group or substituent, or an indirect substitution, whereby an intervening linker group replaces the hydrogen atom, and the substituent or functional group is bonded to the intervening linker group. A non-limiting example of direct substitution is: RR—H→RR—Cl, wherein RR is an organic moiety/fragment/molecule. A non-limiting example of indirect substitution is: RR—H→RR-(LL)_{zz}-Cl, wherein RR is an organic moiety/fragment/molecule, LL is an intervening linker group, and ‘zz’ is an integer from 0 to 100 inclusive. When zz is 0, LL is absent, and direct substitution results. The intervening linker group LL is at each occurrence independently selected from the group consisting of —H, —O—, —S—, —S(═O)—, —S(—O)₂—, —NR—, —NR², —CH═, —C═, —CH₂—, —CHR—, —CR₂—, —CH₃, —C(═O)—, —C(═NR)—, and combinations thereof. LL can be linear, branched, cyclic, acyclic, and combinations thereof.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)—CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “heterocycloalkyl” as used herein refers to a cycloalkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10, 11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The term “aminoalkyl” as used herein refers to amine connected to an alkyl group, as defined herein. The amine group can appear at any suitable position in the alkyl chain, such as at the terminus of the alkyl chain or anywhere within the alkyl chain.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl) propyl, 3-(3,4-epoxycylohexyl) propyl, 2-(3,4-epoxycyclohexyl) ethyl, 2-(2,3-epoxycylopentyl) ethyl, 2-(4-methyl-3,4-epoxycyclohexyl) propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, (C₁-C₄) hydrocarbyl means the hydrocarbyl group can be methyl (C₁), ethyl (C₂), propyl (C₃), or butyl (C₄), and (C₀-C_{b}) hydrocarbyl means in certain embodiments there is no hydrocarbyl group. In certain embodiments, the hydrocarbyl is an alkyl group.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include

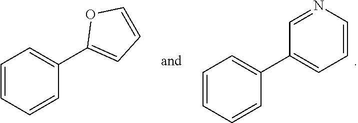 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Preparation of Compounds

Compounds of Formula I-IV or otherwise described herein can be prepared by the general schemes described herein, using the synthetic method known by those skilled in the art. The following examples illustrate non-limiting embodiments of the compound(s) described herein and their preparation.

### Selective Triazole-Based JAK2 Inhibitors

Janus Kinase 2 (JAK2) is one of four members of the JAK family of nonreceptor tyrosine kinases [JAK1, JAK2, JAK3 and Tyrosine Kinase 2 (TYK2)]. JAKs are associated with the cytoplasmic tails of cytokine receptors, and have an important role in signal transduction for the regulation of hematopoiesis and immune response. Cytokine binding to the corresponding receptor leads to a cascade of phosphorylation events that results in JAK activation, and subsequent binding of Signal Transducer and Activator of Transcription (STAT). STATs are thereby phosphorylated, dimerize, and the dimers translocate to the nucleus to bind DNA and regulate gene expression (JAK-STAT pathway).

Mutations in JAKs have been linked to hematological diseases. V617F, the most frequently-occurring mutation in JAK2, has been associated with the pathogenesis of myeloproliferative neoplasms (MPNs), like polycythemia vera (PV), essential thrombocythemia (ET), and primary myelofibrosis (MF). Structurally, JAK2 is comprised of seven Janus Homology (JH) domains, including a C-terminal kinase domain (JH1) responsible for the catalytic activity and an adjacent pseudokinase domain (JH2). The pseudokinase domain adopts a prototypical protein-kinase fold and can bind ATP, but it lacks critical residues for significant phosphorylation catalysis. Its primary role is to regulate the function of the JH1 domain. Recent insights shed light into the mechanism of cytokine receptor activation, revealing a critical role of the pseudokinase domain in receptor dimerization. The insights suggest that activating mutations like V617F drive stronger interactions between the pseudokinase domains, stabilizing the JAK2-receptor dimer and resulting in cytokine independent activation.

Despite the fact that occurrence of V617F mutation in the JH2 domain results in hyperactivation of the kinase activity, so far only the kinase domain of JAK2 has been utilized for therapeutic targeting by type-I ATP-competitive inhibitors. Current therapies for MPNs, like the FDA approved drug ruxolitinib (JAK1/JAK2 inhibitor) for the treatment of myelofibrosis, are non-selective and cause hematopoietic toxicities. Evidence from mutagenesis studies suggest that the hyperactivation caused by the V617F mutant could be attenuated by displacing ATP from the binding site of the JAK2 JH2 domain. Moreover, the studies indicate that the displacement event itself could attenuate the hyperactive V617F variant without affecting wild-type JAK2.

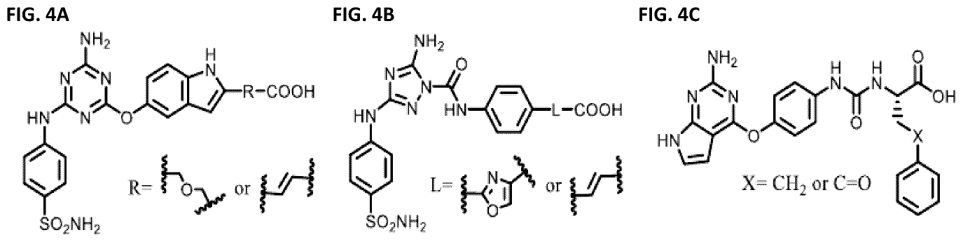

Despite these auspicious insights, the potential value of the pseudokinase domain of JAK2 as a pharmacological target has yet to be demonstrated by small molecules. Previously reported potent JAK2 JH2 ligands were devoid of selectivity for the pseudokinase over the kinase domain, which prohibited accurate evaluation of the JH2 domain targetability. Along these lines, we endeavored to identify small molecules that could selectively bind the JH2 over the JH1 domain and aim to use them as chemical probes to test this hypothesis. These efforts have led to the development of several series of novel JAK2 JH2 binders: indoloxytriazines, diaminotriazoles, and pyrrolopyrimidines (FIGS. 4A-4C).

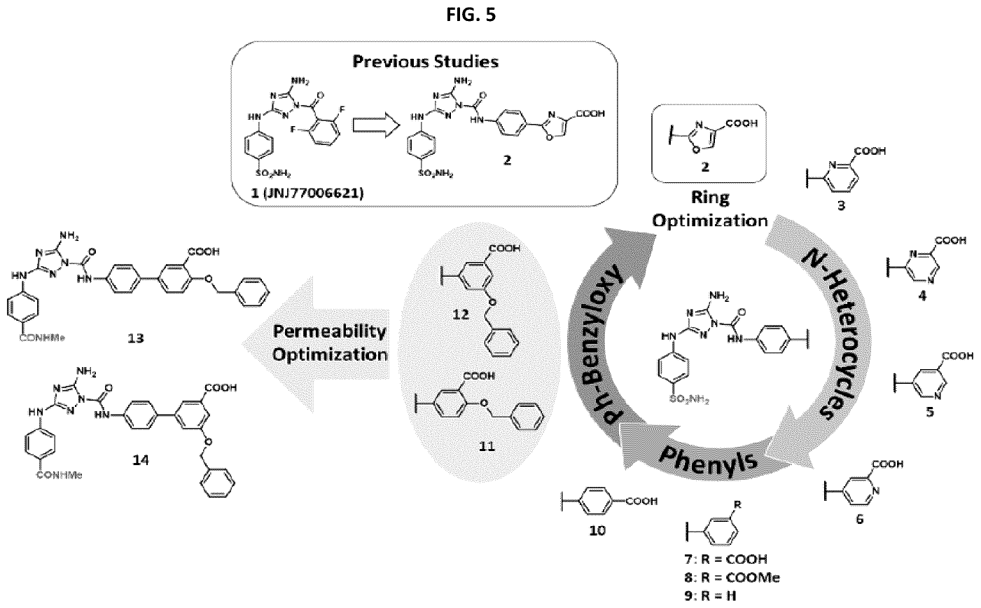

An initial step in these studies was the identification of JNJ7706621, a known Aurora A/B kinase and pan-CDK inhibitor, as a non-selective JAK2 JH2 ligand (K_{d}=0.67±0.18 UM for JH1 and 0.46±0.12 μM for JH2) through a high-throughput fluorescence polarization (FP) screen (FIG. 5, compound 1). We then developed a FP assay with a fluorescein-conjugate of JNJ7706621 as the tracer, which facilitated rapid binding affinity measurements with a lower detection limit. Leveraging structural data and computer-aided design, we previously optimized JNJ7706621 into a series of novel diaminotriazole ureas, which provided potent and selective JAK2 JH2 ligands. However, the affinity and properties of the ligands did not sufficient for testing in cellular assays. Newly discovered insights on essential pharmacophore elements of JH2 leads prompted us to further optimize the diaminotriazole urea series, aiming to achieve higher potency, selectivity, and enable cell testing. Provided herein are affinity and permeability optimizations that describe the development of potent, selective, and cell-permeable therapeutic agents (FIG. 5).

In various embodiments, biaryl and aryl-heteroaryl triazole JAK2 inhibitors were prepared according to the general approach illustrated with the synthetic route for Compound 12 in Scheme 1.

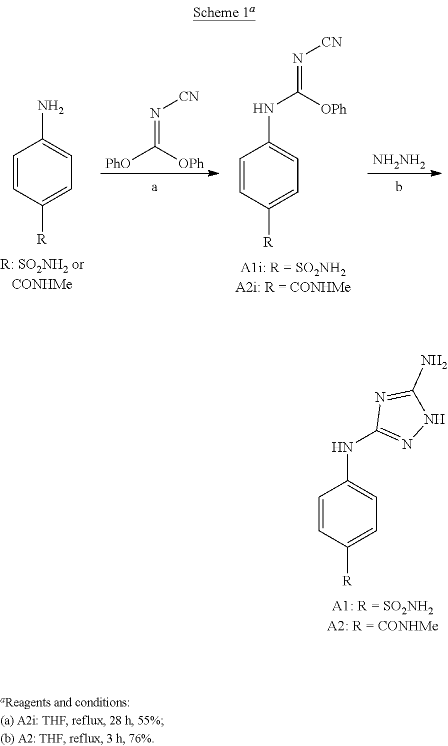

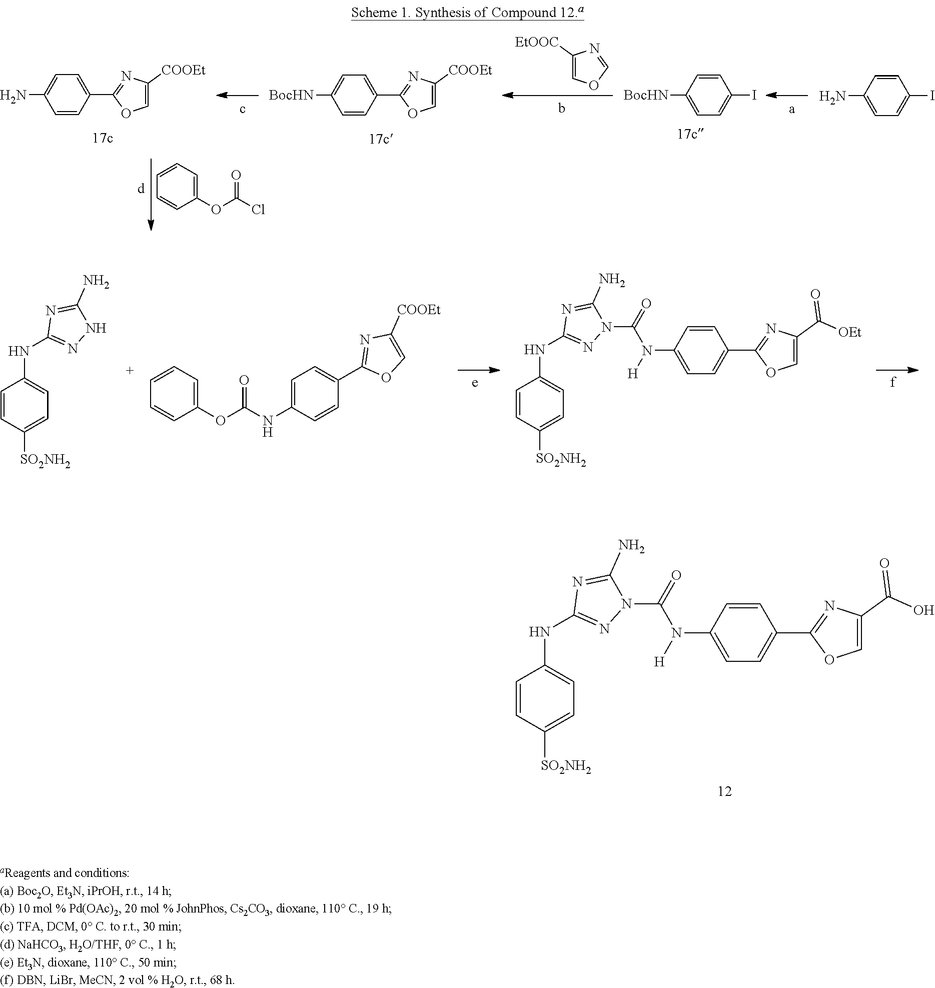

One non-limiting coupling step in this synthetic scheme is the regioselective acylation of a 1H-[1,2,4]triazole-3,5-diamine with a phenylcarbamate. One challenge observed in the synthesis of compounds of Formula I appeared to be associated with the poor solubility in organic solvents for the polar diaminotriazole precursor and the presence of its tautomeric 2-H form, which leads to the undesirable (and difficult-to-separate) regioisomeric 2-H byproduct. In certain embodiments, a phenyl carbamate is coupled to a diaminotriazole to provide compounds of Formula I. This approach is effective in generating the desired products when dioxane was used as the solvent, but the yields were not optimal since the problems with the diaminotriazole precursor remained. Better results were obtained by increasing the reaction temperature from 80 to 110° C., extending the reaction time, and diluting the mixture from 1.0 to 0.5 M to improve dissolution of the diaminotriazole.

In various embodiments, the reaction temperature for step ‘e’ in Scheme 1 is equal to, at least, or greater than about 85° C., 90° C., 95° C., 100° C., 105° C., 110° C., or 115° C. In various embodiments, the reaction temperature for step ‘e’ in Scheme 1 is about 85° C. to 115° C., 90° C. to 115° C., 95° C. to 115° C., or about 100° C. to 115° C. In some embodiments, the concentration of the diaminotriazole precursor in the reaction mixture prior to reaction with the phenyl carbamate is about 0.25 M, 0.3 M, 0.35 M, 0.4 M, 0.45 M, 0.5 M, 0.55 M, 0.6 M, 0.65 M, 0.7 M, or about 0.75 M. In various embodiments, the concentration of the diaminotriazole precursor in the reaction mixture prior to reaction with the phenyl carbamate is about 0.25 M to 0.75 M, 0.35 M to about 0.7 M, or 0.4 M to about 0.6 M.

In certain embodiments, a compound of Formula I, or a pharmaceutically acceptable salt, tautomer, or enantiomer thereof, is provided:

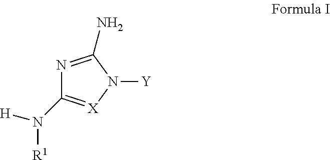

wherein,
Y is selected from the group consisting of —CH₂-R², —C(═O)NR²R³, optionally substituted C₅₋₁₀ heteroaryl, and optionally substituted C₅₋₆ heterocycloalkyl;
wherein the optional substitution is at least one substituent selected from the group consisting of C₁₋₅ alkyl, C₁₋₅ alkoxy, C₁₋₅ thioalkyl, C₁₋₅ aminoalkyl, C₅₋₁₀ aryl, C₅₋₁₀ heteroaryl, —O (oxo), F, Cl, Br, I, C(═O)OR, NHC(═O)R, and OH;
R¹ and R² are each independently selected from the group consisting of C₂₋₆ alkenyl, C₃₋₇ cycloalkyl, C₆₋₁₀ aryl, 5-10 membered heteroaryl, 4-10 membered heterocycloalkyl, C₆₋₁₀-5-6 membered heterobiaryl, 5-6 membered-C₆₋₁₀ heterobiaryl, and C₆₋₁₀-C₆₋₁₀ biaryl,
   each optionally substituted by at least one group of formula (LL)_{zz}-GG, wherein
      LL is at each occurrence independently selected from the group consisting of —O—, —OR, —S—, —S(═O)—, —S(═O)₂—, —NR², —CH═, —C═, —CH₂—, —CHR—, —CR₂—, —CH₃, —C(═O)—, —C(═NR)—;
      zz is an integer from 1 to 30;
      GG is a substituent selected from the group consisting of F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, and C(═NH)N(R)₂,
   wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
R³ is H or C₁₋₄ hydrocarbyl;
X is N or C—R⁴, wherein R⁴ is H or C₁₋₄ hydrocarbyl optionally substituted by 1 to 3 substituents selected from the group consisting of OR′, NHR′, and NR′₂,
   wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl; or wherein two R′ groups bonded to a nitrogen atom or to adjacent nitrogen atoms can together with the nitrogen atom or atoms form a heterocyclyl.

In various embodiments, (LL)_{zz}-GG does not comprise O—O. In various embodiments, (LL)_{zz}-GG does not comprise S—S. In various embodiments, (LL)_{zz}-GG does not comprise S—S—S.

In various embodiments, R¹ is optionally substituted C₆₋₁₀ aryl. In various embodiments, R² is C₆₋₁₀-5-6 membered heterobiaryl, 5-6 membered-C₆₋₁₀ heterobiaryl, or C₆₋₁₀-C₆₋₁₀ biaryl, each of which is at least disubstituted on a terminal ring.

In various embodiments, X is N. In various embodiments, R³ is H.

In various embodiments, R¹ has the structure:

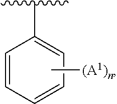

wherein:
each occurrence of A¹ is (LL)_{zz}-GG as defined herein. In various embodiments, A¹ independently selected from the group consisting of CN, OCF₃, SO₂R, SO₂N(R)₂, C(═O)R, and C(═O)OR, C(═O) (CH₂)ₘOCH₃, O(CH₂)ₘOCH₃, (C₅-C₁₀)heteroaryl, and (C₃-C₁₈)heterocycloalkyl, wherein the heteroaryl and heterocycloalkyl are each optionally substituted, and wherein each occurrence of R is independently selected from the group consisting of hydrogen, (C₁-C₆) hydrocarbyl, (C₅-C₁₀)heteroaryl, and (C₃-C₁₈)heterocycloalkyl;
m is an integer from 0 to 10; and
n is an integer from 0 to 5.

In various embodiments, the compound is of Formula Ia:

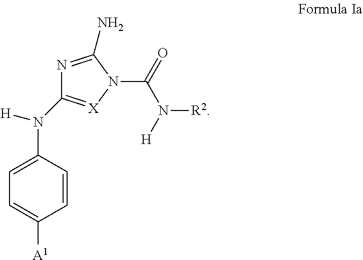

In various embodiments, A¹ is SO₂NH₂, CN, C(═O)NH—C₁₋₆ alkyl, C(═O) (CH₂)₂OCH₃, O(CH₂)₂OCH₃, OCF₃, (C₅-C₆)heteroaryl, or C(—O)—C₆ heterocycloalkyl, wherein each heteroaryl and heterocycloalkyl is optionally substituted.

In various embodiments, A¹ is

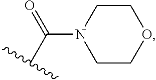 [SMILES: *C(=O)N1CCOCC1]

SO₂NH₂, C(═O)NHMe, or

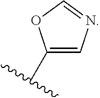 [SMILES: *c1cnco1]

In various embodiments, the compound is of Formula Ib, Formula Ic, or Formula Id:

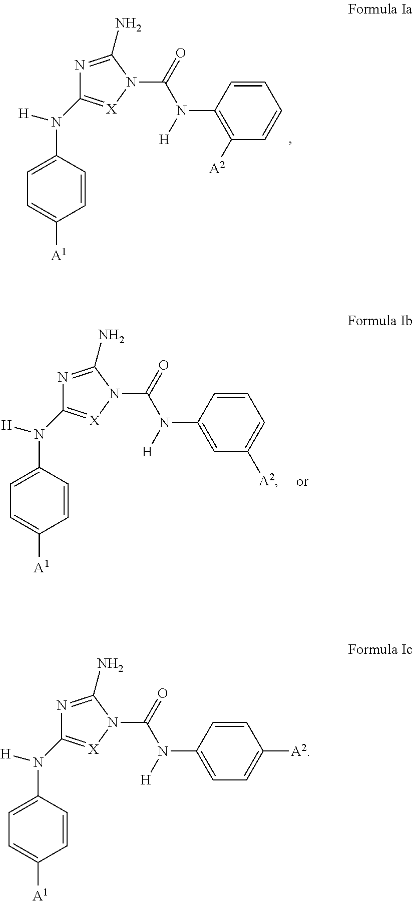

In various embodiments, A² is selected from the group consisting of:

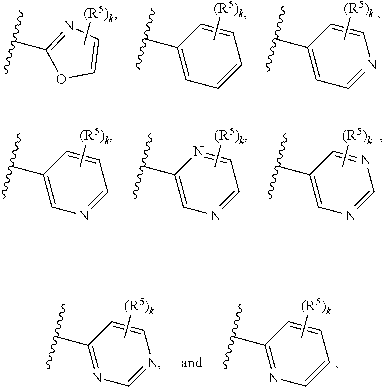

wherein k is 2 or 3.

In various embodiments, each R⁵ is independently (LL)_{zz}-GG,
LL is selected from group consisting of —O—, —CH₂—, and —CHR—;
zz is 0, 1, 2, or 3; and
GG is selected from the group consisting of C(—O)OH, C(—O)O—C₁₋₄ alkyl, (C₆-C₁₀)aryl, and (C₅-C₁₀)heteroaryl.

In various embodiments, (LL)_{zz}-GG is —OCH₂Ph.

In various embodiments, A² is selected from the group consisting of:

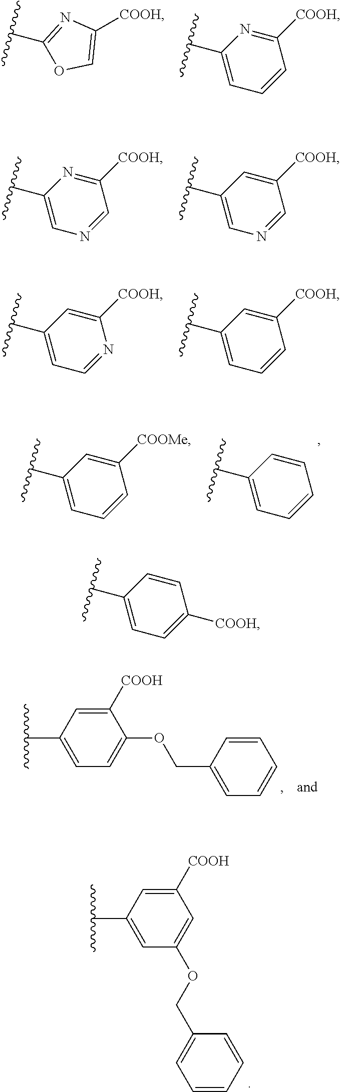

In various embodiments, the compound of Formula I is selected from the group consisting of:

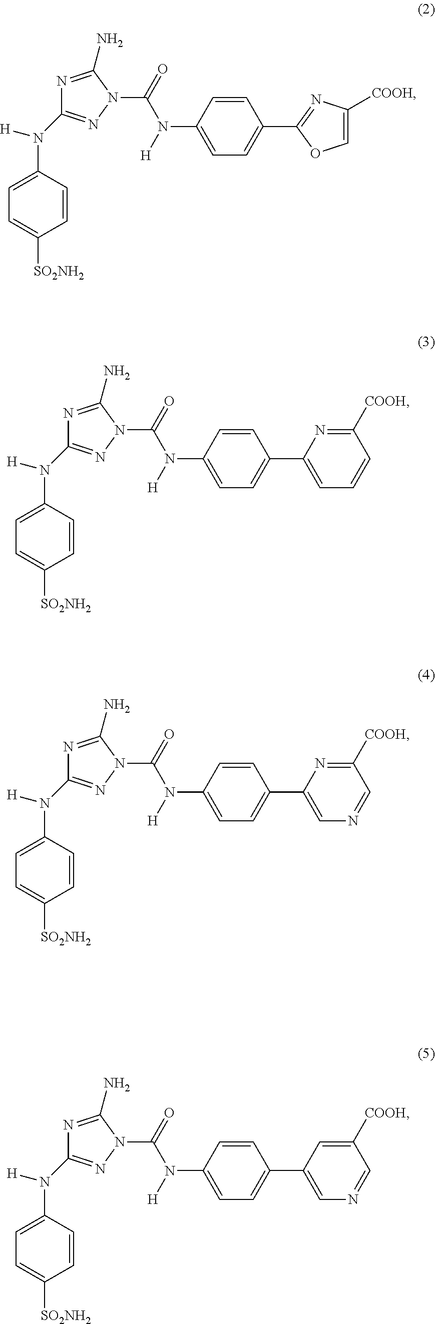 [SMILES: Nc1nc(Nc2ccc(S(N)(=O)=O)cc2)nn1C(=O)Nc1ccc(-c2nc(C(=O)O)co2)cc1 ; Nc1nc(Nc2ccc(S(N)(=O)=O)cc2)nn1C(=O)Nc1ccc(-c2cccc(C(=O)O)n2)cc1 ; Nc1nc(Nc2ccc(S(N)(=O)=O)cc2)nn1C(=O)Nc1ccc(-c2cncc(C(=O)O)n2)cc1 ; Nc1nc(Nc2ccc(S(N)(=O)=O)cc2)nn1C(=O)Nc1ccc(-c2cncc(C(=O)O)c2)cc1]

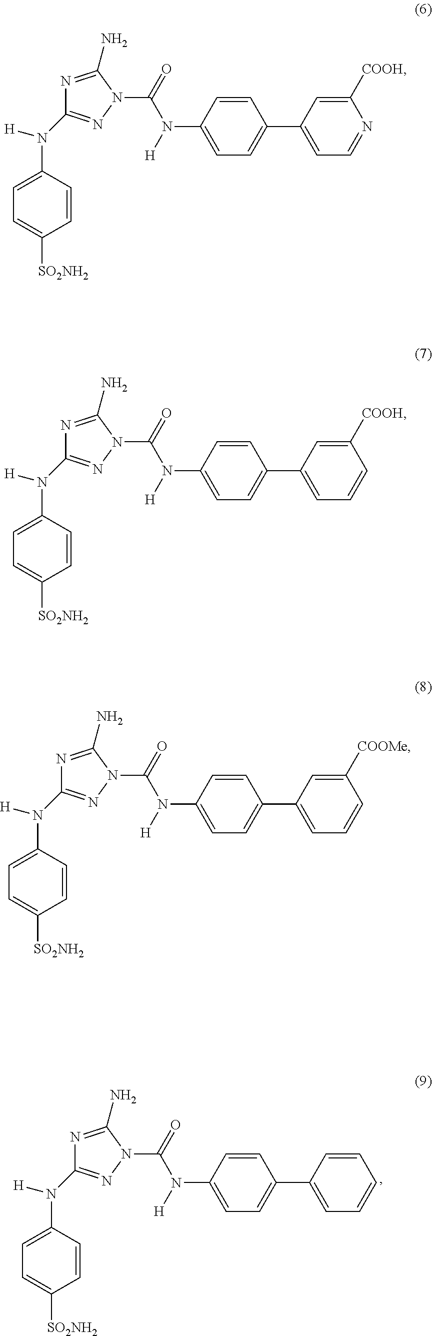 [SMILES: Nc1nc(Nc2ccc(S(N)(=O)=O)cc2)nn1C(=O)Nc1ccc(-c2ccnc(C(=O)O)c2)cc1 ; Nc1nc(Nc2ccc(S(N)(=O)=O)cc2)nn1C(=O)Nc1ccc(-c2cccc(C(=O)O)c2)cc1 ; COC(=O)c1cccc(-c2ccc(NC(=O)n3nc(Nc4ccc(S(N)(=O)=O)cc4)nc3N)cc2)c1 ; Nc1nc(Nc2ccc(S(N)(=O)=O)cc2)nn1C(=O)Nc1ccc(-c2ccccc2)cc1]

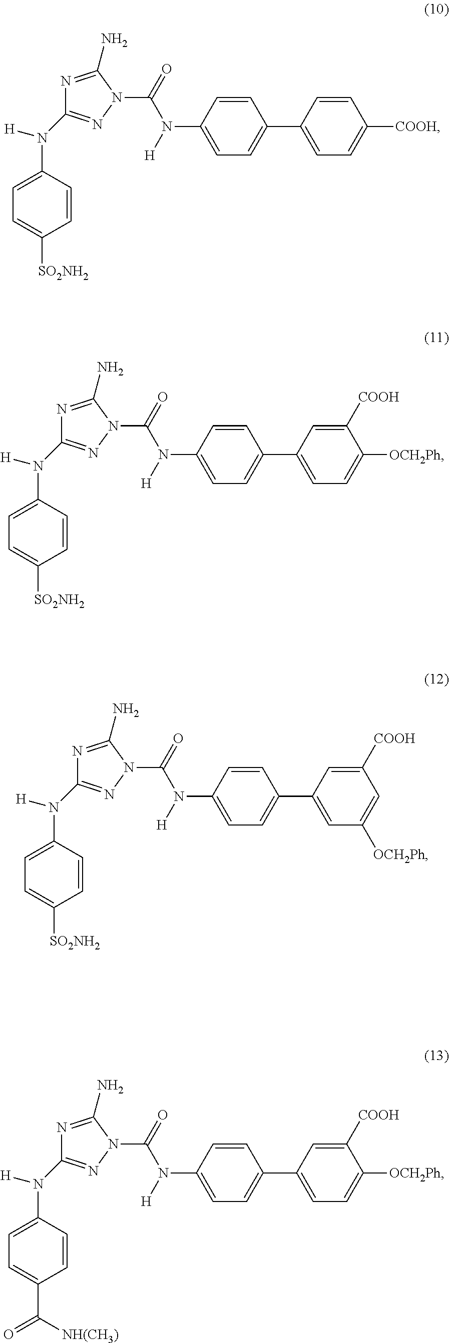

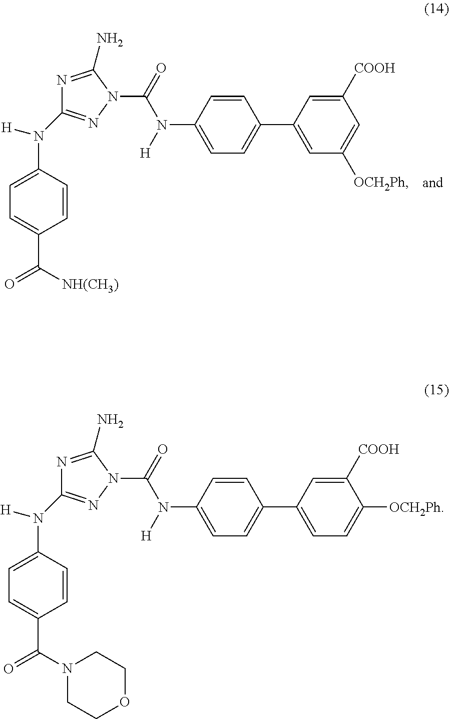

TABLE 1
Measured Binding Affinity of Compounds of Formula I (Kd, μM) with
JAK2 JH2^{a}

| Compound | K_{d} (μM) |
|---|---|
| 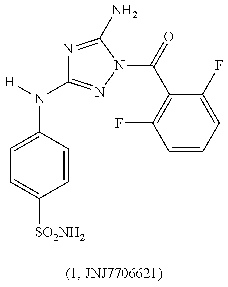 [SMILES: Nc1nc(Nc2ccc(S(N)(=O)=O)cc2)nn1C(=O)c1c(F)cccc1F] | 0.456 ± 0.124 |
| 2 | 0.346 ± 0.034 |
| 3 | 0.934 ± 0.256 |
| 4 | 1.4 ± 0.1 |
| 5 | 0.652 ± 0.045 |
| 6 | 0.394 ± 0.044 |
| 7 | 0.129 ± 0.002 |
| 8 | 27% @ 50 μM |
| 9 | 30.5% @ 50 μM |
| 10 | 0.155 ± 0.013 |
| 11 | 0.03710 ± 0.0005 |
| 12 | 0.075 ± 0.015 |
| 13 | 0.0334 ± 0.0033 |
| 14 | 0.044 ± 0.003 |

^{a})For each K_{d} measurement, 2-3 independent experiments were carried out in quadruplicate using the competitive fluorescence polarization (FP) assay.

In certain embodiments, a compound of Formula II, or a pharmaceutically acceptable salt, tautomer, or enantiomer thereof is provided:

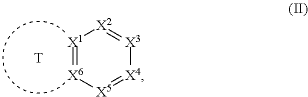

wherein
T is an optional 5 or 6 membered heterocyclic fused ring that is optionally substituted by at least one -(LL)_{zz}-GG;
each of X¹-X⁶ is independently N or C—Y;
each occurrence of Y is independently absent, H, -(Q)ₙ-(C₃-C₁₂)cycloalkyl, -(Q)-(C₃-C₁₈)heterocycloalkyl, -(Q)ₙ-(C₆-C₁₈)aryl, or -(Q) n-(C₅-C₁₈)heteroaryl;
Q is absent, or independently selected at each occurrence from the group consisting of O, CH₂, NH, and N—C₁₋₄ alkyl;
n is an integer from 1 to 10;
each cycloalkyl, heterocycloalkyl, aryl, and heteroaryl in Y is optionally substituted by at least one -(LL)_{zz}-GG,
at each occurrence LL is independently selected from the group consisting of —O—, —OR, —S—, —S(═O)—, —S(═O)₂—, —NR—, —NR², —CH═, —C═, —CH₂—, —CHR—, —CR₂—, —CH₃, —C(═O)—, —C(═NR)—;
zz is an integer from 1 to 30;
GG is a substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl;
wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
wherein each alkyl, alkenyl, alkynyl, cycloalkyl, heterocycloalkyl, aryl, and heteroaryl in GG is optionally substituted aby at least one substituent selected from the group consisting of H, F, Cl, Br, I, OR′, OC(═O)N(R′)₂, CN, NO₂, CF₃, OCF₃, R′, N(′R)₂, SR′, SOR′, SO₂R′, SO₂N(R′)₂, SO₃R′, C(═O) R′, C(═O)OR′, OC(═O) R′, O(CH₂)₀₋₂C(═O)OR′, C(═O)N(R′)₂, OC(═O)N(R′)₂, (CH₂)₀₋₂N(R′)C(═O) R′, N(R′)SO₂R′, N(R′)C(═O)OR′, N(R′)C(═O) R′, N(R′)C(═O)N(R′)₂, and C(═NH)N(R′)₂;
wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; with the proviso that at least two of X¹-X⁶ is N and at least one of X¹-X⁶ is C—NH₂.

In various embodiments, the compound of Formula II can be:

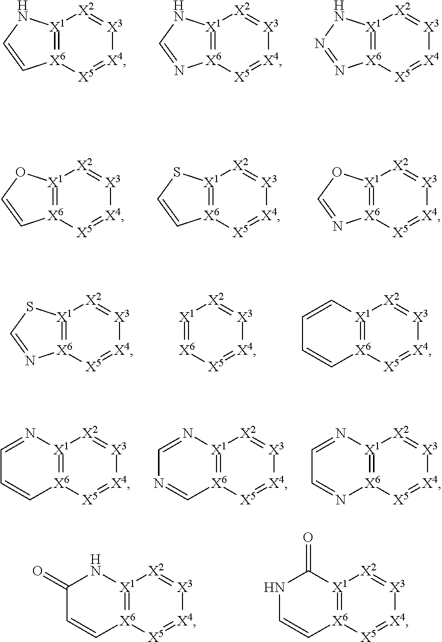

and the like. Although the various “T-rings” are depicted as unsubstituted, each may be substituted at any open valence, including the N—H, with at least one -(LL)_{zz}-GG as defined herein.

### Selective Triazine-Based JAK2 Inhibitors

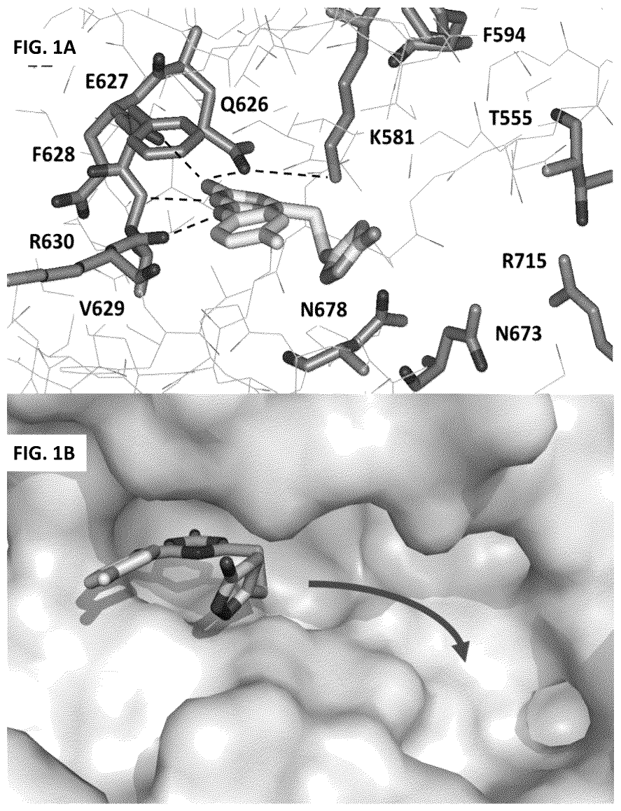

In some embodiments, triazine-containing inhibitors of JAK2 are also provided herein, which were identified based on identifying compound 22 in an in silico screen. Compound 22 shows good selectivity with no binding to JAK2 JH1 and K_{d} values of 149 and 65 μM with WT and V617F JAK2 JH2, respectively. A crystal structure for the WT complex was obtained (FIG. 1A) and confirmed the four expected hydrogen bonds with Gln626, Glu627, and Val629. The thiadiazole-containing appendage, however, bends outward towards the solvent and the electron density for the terminal cyclopentylmethyl group could not be resolved. In order to achieve both selectivity and stronger binding, modifications to grow the ligand to the right towards Thr555 and Arg715 were desired (FIG. 1B).

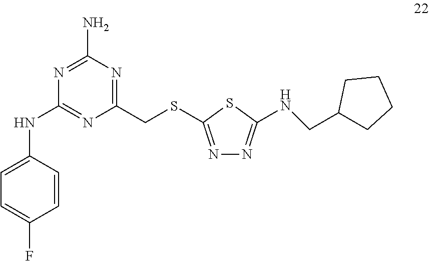 [SMILES: Nc1nc(CSc2nnc(NCC3CCCC3)s2)nc(Nc2ccc(F)cc2)n1]

Thus, alkylamino, phenoxy analogues 33a-m of 22 were prepared, as summarized in Scheme A¹. The syntheses featured sequential SNAr introduction of the anilinyl and aryloxy groups starting from cyanuric chloride or the aminodichlorotriazine. The new compounds reported here are summarized in Table A¹. Binding affinities were determined using the updated FP assay with a tracer derived from 1. The three protein domains, WT JAK2 JH2, V617F JAK2 JH2, and WT JAK2 JH1, were expressed and purified as previously described.

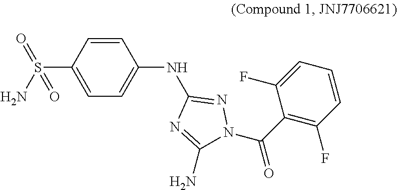 [SMILES: Nc1nc(Nc2ccc(S(N)(=O)=O)cc2)nn1C(=O)c1c(F)cccc1F]

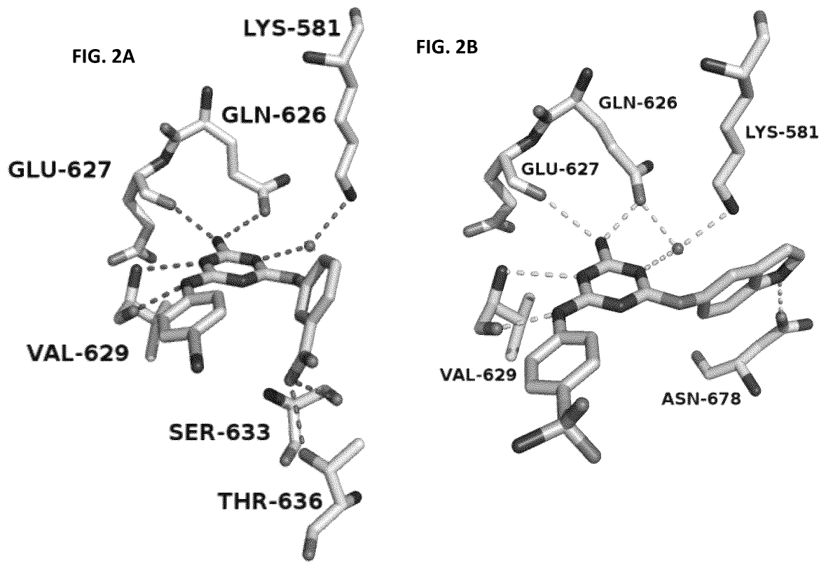

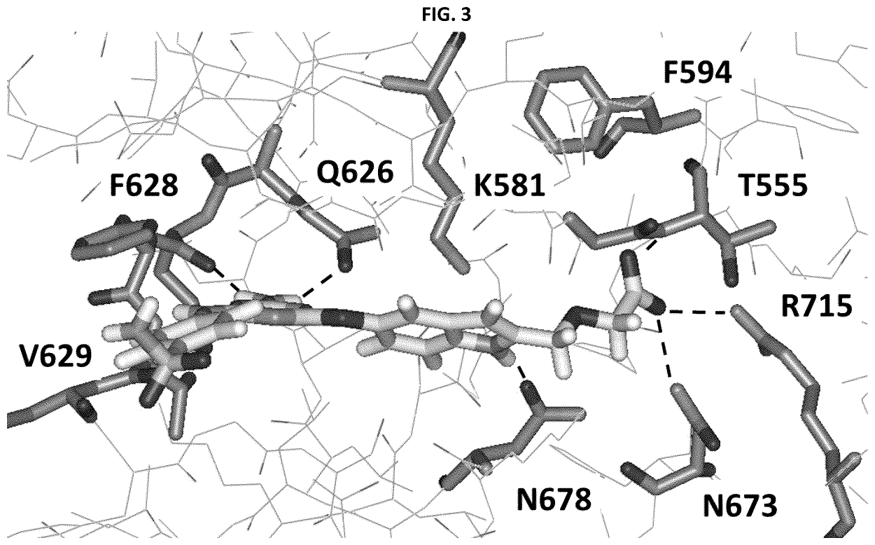

The SAR was initially explored for the p-cyanoanilinyl analogues 33a-f. The parent phenoxy compound 33a showed improved binding to WT JAK2 JH2 with a K_{d} of 73 μM, and brought a two-fold benefit to ca. 40 μM (33b-d, Table A¹). Fortunately, it was possible to obtain a crystal structure for the complex of 33b with WT JAK2 JH2 (FIG. 2A); however, it revealed that the phenoxy substituent is again directed outward with one oxygen atom of the m-carboxylate group engaged in three hydrogen bonds with the backbone nitrogen (2.79 Å) and hydroxyl oxygen (2.84 Å) of Ser633 and with the hydroxyl oxygen (2.95 Å) of Thr636. This suggested modification of 33d to increase the acidity of the hydroxyl group by addition of two ortho fluorine atoms yielding 33f. A lower K_{d} of 10.7 UM did result, though the expectation was that the substituent remained directed outward, as in FIG. 3A.

Modeling then considered bicyclic aryloxy possibilities, which led to the notion that a 5-indoloxy substituent might be directed inward to form a hydrogen bond between the indole NH and the sidechain carbonyl group of Asn678. Thus, the cyano and sulfamyl alternatives 33h and 33m were synthesized and yielded K_{d} values of 47 and 18 μM, respectively. Importantly, the structural prediction was confirmed by a crystal structure for 33m (FIG. 2B). The isomeric indole 33k and benzimidazole 331 are less potent, though they are also expected to benefit from cation-π interactions with Lys581.

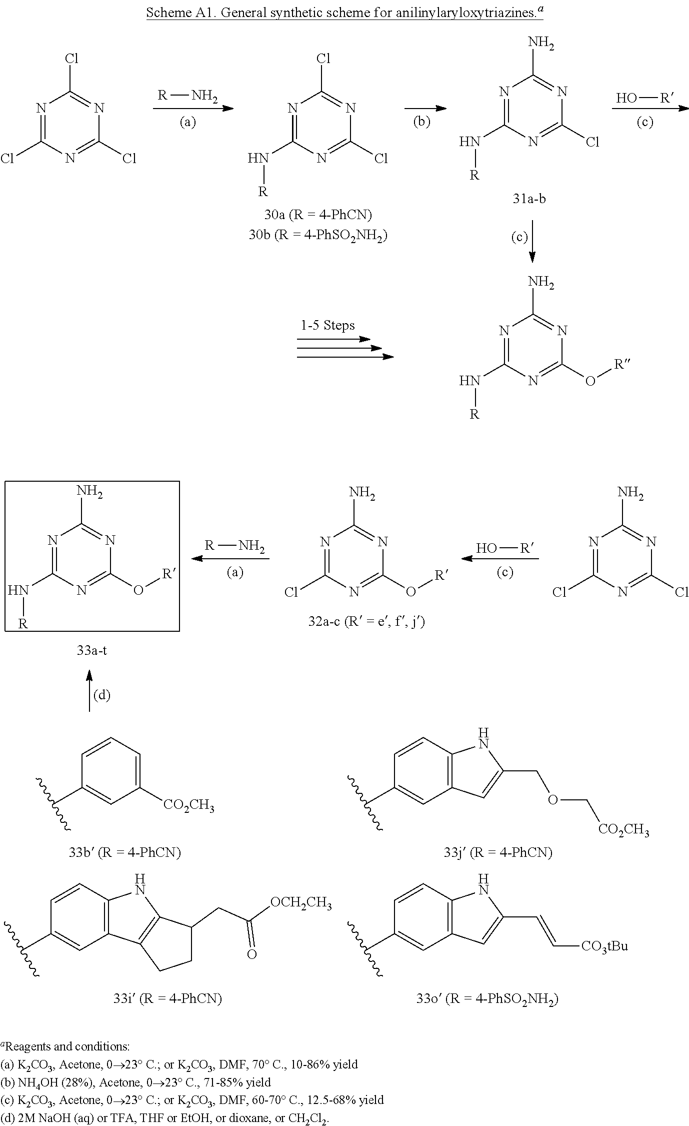

TABLE A1
Compounds (33) and their experimental binding affinities (Kd) using the
fluorescence polarization (FP) assay.

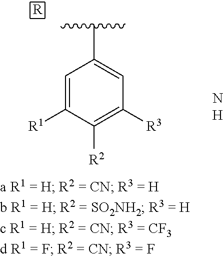

[symbol]

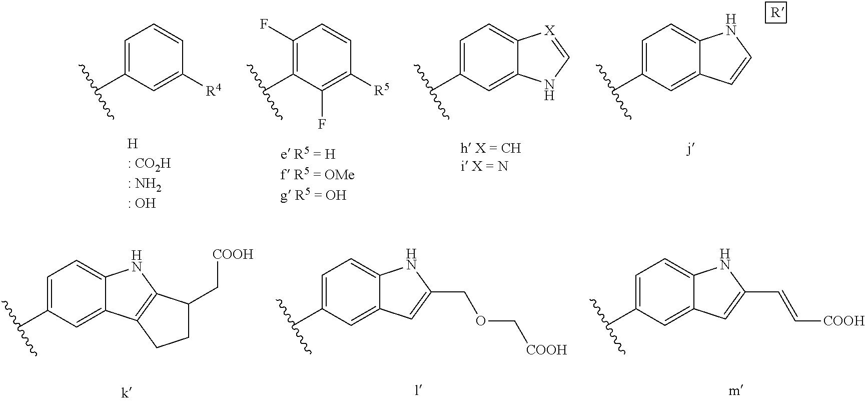

| | | | K_{d} (μM) | | |
|---|---|---|---|---|---|
| Compound | R | R′ | JAK2 JH1 | JAK2 JH2 WT | JAK2 JH2 V617F |
| 33a | a | a′ | | 73.4 ± 20.3 | |
| 33b | a | b′ | | 39.6 ± 3.2 | |
| 33c | a | c′ | | 40.7 ± 4.9 | |
| 33d | a | d′ | | 43.8 ± 12.2 | |
| 33e | a | f′ | | 17.4 ± 9.1 | |
| 33f | a | g′ | | 10.7 ± 1.9 | |
| 33g | a | h′ | | 34.9 ± 1.5 | |
| 33h | a | j′ | | 47.4 ± 6.9 | |
| 33i | a | k′ | | 14.0 ± 1.0 | |
| 33j | a | l′ | ND (15% @ 50 μM) | 5.9 ± 0.7 | 4.3 ± 0.1 |
| 33k | b | h′ | | 20.9 ± 9.2 | |
| 33l | b | i′ | | 31.6 ± 5.7 | |
| 33m | b | j′ | ND (16% @ 50 μM) | 18.3 ± 3.3 | 8.0 ± 0.8 |
| 33n | b | l′ | 41.1 ± 7.8 | 2.6 ± 0.1 | 2.4 ± 0.4 |
| 33o | b | m′ | 18.0 ± 2.7 | 3.3 ± 1.2 | 2.0 ± 0.1 |
| 33p | c | j′ | | 53.5 ± 17.9 | |
| 33q | d | j′ | | ND (15% @ 50 μM) | |
| 33r | f | e′ | | 122.7 ± 22.0 | |
| 33s | f | f′ | | 54.7 ± 12.2 | |
| 33t | f | j′ | | 23.2 ± 1.4 | |
| JNJ7706621 (1) | | | 0.671 ± 0.175 | 0.456 ± 0.124 | 0.599 ± 0.087 |

The next step was to further extend the indoles by attachments at C2 to terminate in a carboxylic acid group as in 2 (FIG. 3). This was realized with 33i, 33j, 33n, and 330, which did yield the strongest binding molecules at 2-3 μM for WT and V617F JAK2 JH2. The 9- and 17-fold selectivities of 33n and 330 for binding the V617F JH2 domain over WT JH1 were also gratifying (Table 1). The right-hand-sides of 33n and 330 were synthesized from the common intermediate 37 (Scheme A²), which arose from DIBAL-reduction of the C2-ethyl ester after TBS-protection of the C5-hydroxyl group. Rh (II)-catalyzed etherification of 37 followed by TBAF-deprotection of the silyl group yielded ester 34, while ester 35 was prepared by oxidation of 37 to the aldehyde, Wittig reaction, and deprotection. The two hydroxyesters underwent SNAr coupling with anilinylchlorotriazines to yield 33n directly (the ester hydrolyzed during coupling) and the ester of 330, which upon hydrolysis provided the corresponding acrylic acid.

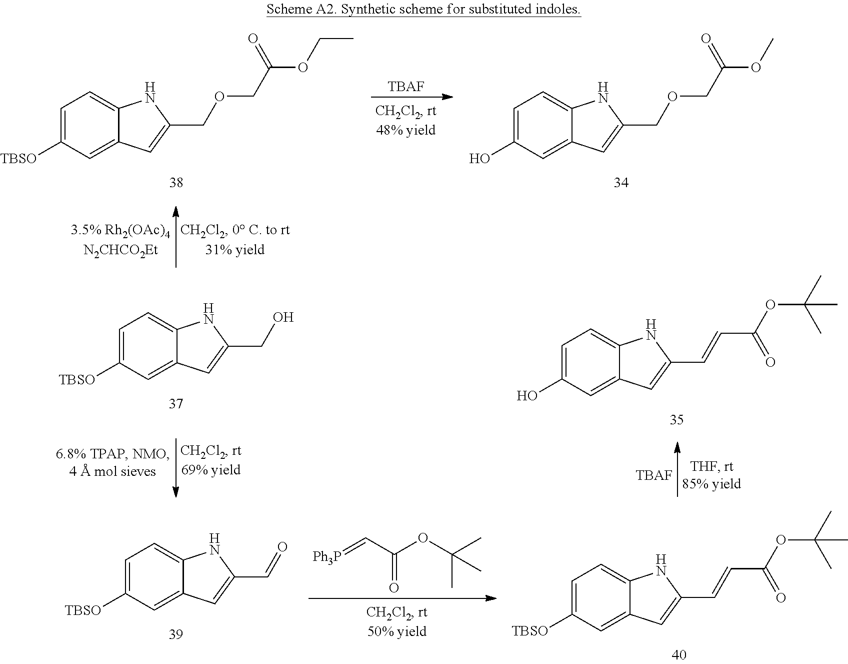

In various embodiments, the compound of Formula II is a compound of Formula III:

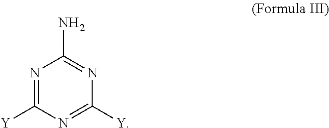

In various embodiments, the compound of Formula III has the structure:

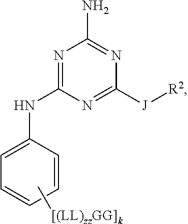

wherein
J is O or NH;
k is an integer from 1 to 5; and
R² is selected from the group consisting of C₆₋₁₀ aryl, C₆₋₁₀ heteroaryl, and combinations thereof, each of which is optionally substituted.

In various embodiments, the compound of Formula III has the structure:

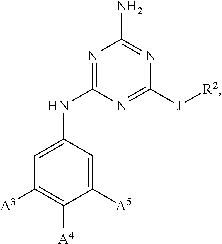

wherein A³, A⁴, A⁵ are each independently selected from the group consisting of H, F, CF₃, CN, OCF₃, SO₂R, SO₂N(R)₂, C(═O)R, and C(═O)OR, C(═O)(CH₂)ₘOCH₃, O(CH₂)ₘOCH₃, (C₅-C₁₀)heteroaryl, and (C₃-C₁₈)heterocycloalkyl, wherein the heteroaryl and heterocycloalkyl are each optionally substituted, and wherein each occurrence of R is independently selected from the group consisting of hydrogen, (C₁-C₆) hydrocarbyl, (C₅-C₁₀) heteroaryl, and (C₃-C₁₈)heterocycloalkyl, m is an integer from 0 to 10, and n is an integer from 0 to 5.

In various embodiments, in the compound of Formula III, A⁴ is selected from the group consisting of CN, SO₂NH₂, and C(═O)NHCH₃. In various embodiments, J is O.

In various embodiments, in the compound of Formula III, R² is selected from the group consisting of:

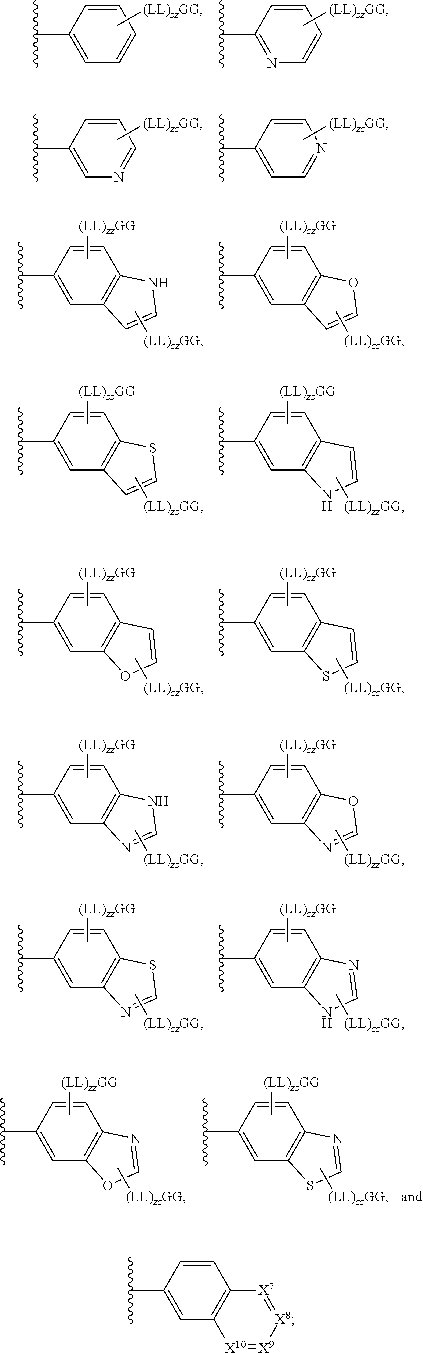

wherein X¹, X, X′, and X¹⁰ are each independently N, NH, or C—Y; and
wherein each -(LL)_{zz}GG moiety represents at least one substituent.

In various embodiments, in the compound of Formula III, R² is selected from the group consisting of

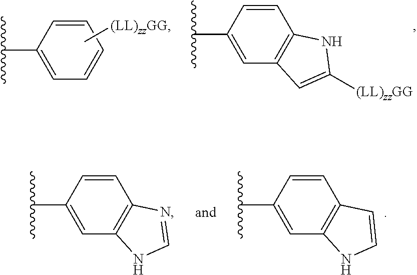

In various embodiments, in the compound of Formula III, R² is selected from the group consisting of

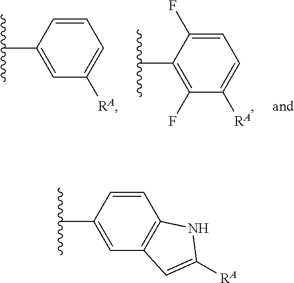

wherein R^{A} is selected from the group consisting of H, CO₂H, NH₂, OH, OCH₃, CH₂OCH₂COOH, and trans C═C(H)(COOH).

In various embodiments, in the compound of Formula III, R² is selected from the group consisting of:

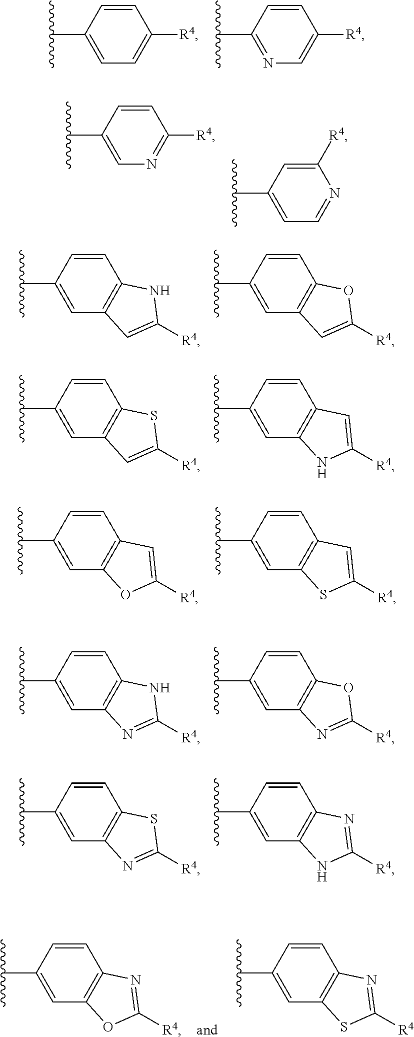

wherein
R⁴ is

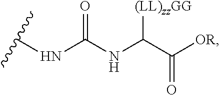

each LL is independently selected from the group consisting of —CH₂—, —NH—, —C(—O)—, and —O—;
R is C₁₄ alkyl or H;
zz is an integer from 2 to 6; and
GG is optionally substituted phenyl.

In various embodiments, when R⁴ is

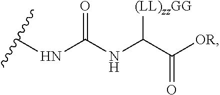

(LL)_{zz}GG is selected from the group consisting of (CH₂)₂Ph, (CH₂)₃Ph, CH₂C(═O)NHPh, and CH₂(C═O)Ph.

In various embodiments, when R⁴ is

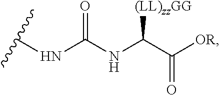

(LL)_{zz}GG is selected from the group consisting of (CH₂)₂Ph, (CH₂)₃Ph, CH₂C(═O)NHPh, and CH₂(C═O)Ph.

In various embodiments, when R⁴ is

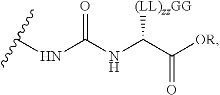

(LL)_{zz}GG is selected from the group consisting of (CH₂)₂Ph, (CH₂)₃Ph, CH₂C(═O)NHPh, and CH₂(C═O)Ph.

### Selective Pyrrolopyrimidine-Based JAK2 Inhibitors

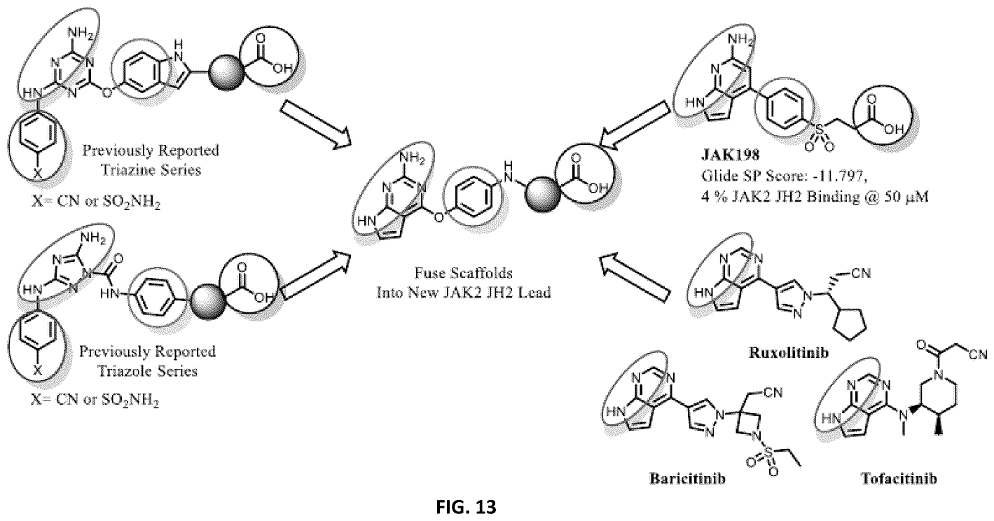

Structural elements that promote binding to the JAK2 JH2 domain have been identified (FIG. 13). The primary pharmacophore is a hydrogen-bonding donor-acceptor-donor motif (FIG. 13, magenta/long oval), manifested as a diamino-triazole or a diaminotriazine scaffold that participates in hydrogen bonding to the hinge region. These heterocycles are connected to a phenyl or indole motif (FIG. 13, green/circle around phenyl) and engage in cation-π interactions with Lys581. The aromatics are further linked to a carboxylic acid (FIG. 13, blue/circle around carboxylic acid) that forms hydrogen bonds with nearby residues and in many cases forms a salt-bridge with Arg715 which is not conserved in the JAK2 JH1 domain. Various structural elements were utilized as linkers of the aromatics to the carboxylic acid, which minimally affected binding to the JH2 domain (FIG. 13, blue spheres). Finally, the triazines and triazoles possess an aryl sulfonamide or aryl nitrile moiety (FIG. 13, red/circle around phenyl with X substituent) that extends into solvent.

With this guidance, we have reconsidered previously reported compounds from a virtual screen, in particular, the highest scoring analogue JAK198 (FIG. 13). Though it and analogues were predicted to bind strongly to JAK2 JH2 by docking with Glide, they exhibited little or no binding to 2 JAK2 JH2 when tested using a fluorescence polarization (FP) assay. JAK198 possesses three of the four pharmacophore elements described above: the hydrogen-bond donor-accept-or-donor motif (FIG. 13, magenta), the phenyl ring (FIG. 13, green), and the terminal carboxylic acid (FIG. 13, blue). The aryl nitrile or sulfonamide moiety is absent in JAK198 and could explain, in part, the poor binding. However, the JAK2 inhibitors ruxolitinib, baricitinib, and tofacitinib each possess a related donor-acceptor motif within their pyrrolopyrimidine scaffolds (FIG. 13, magenta). While these inhibitors act on the JH1 domain of JAK2, the pyrrolopyrimidine core should be viable for binding to the hinge region of both JH1 and JH2. Therefore, it was expected that a pyrrolopyrimidine could be incorporated into new JAK2 JH2 ligands. We initially sought to modify JAK198 to better mimic the triazines described above. An aminopyrrolopyrimidine core was selected because it possessed the desired donor-acceptor-donor motif (FIG. 13, magenta). This core could then be attached to a phenyl group via an ether linkage instead to better mimic the triazine series. Since the indole hydrogen is hydrogen-bonded to Asn678 in this series, we also sought to attach an amino group to the phenyl group (FIG. 13, green). This resultant aniline could then provide a synthetic handle to connect the terminal carboxylic acid through various linkers, completing the redesign of JAK198 into a new series. For the synthesis of the new compounds, we started from s1 the pyrrolopyrimidine and built to the right (Scheme B1). Commercially available 4-chloro-7H-pyrrolo[2,3-d]pyrimidin-2-amine (1) was subjected to an SNAr reaction with 4-nitrophenol.

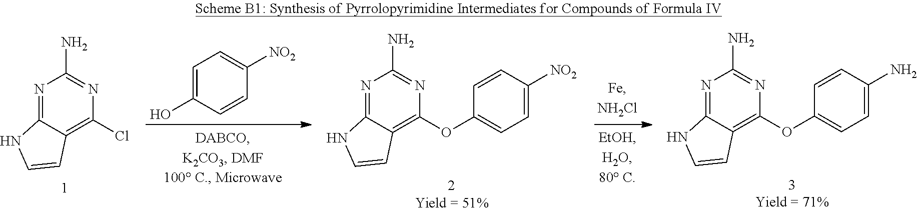 [SMILES: Nc1nc(Cl)c2cc[nH]c2n1 ; O=[N+]([O-])c1ccc(O)cc1 ; Nc1nc(Oc2ccc([N+](=O)[O-])cc2)c2cc[nH]c2n1 ; Nc1ccc(Oc2nc(N)nc3[nH]ccc23)cc1]

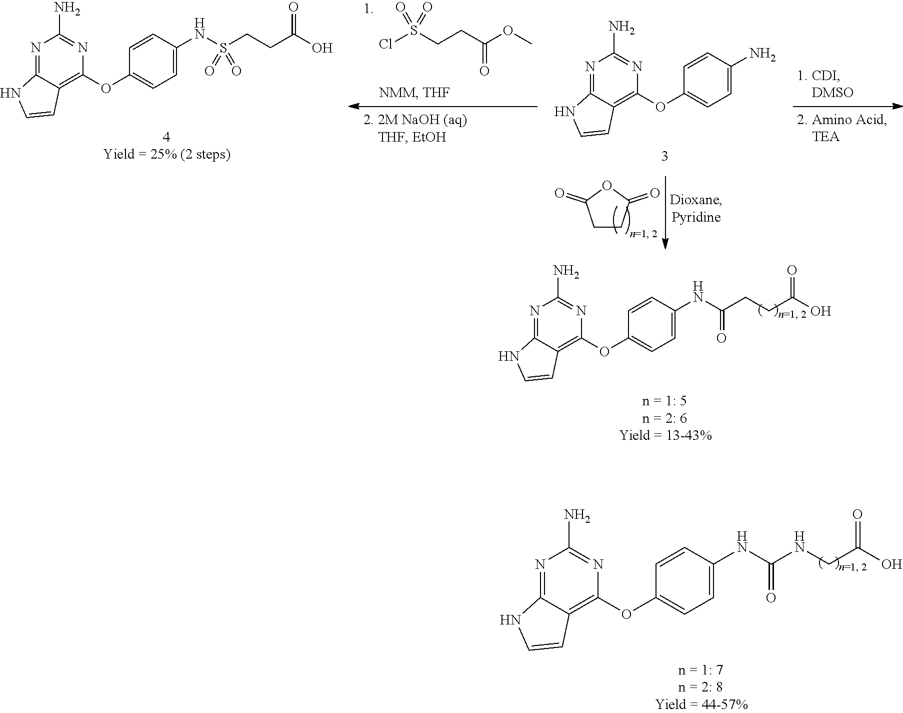

Harsh conditions were required to facilitate this substitution reaction, including microwave irradiation at 103° C. for 1.5 h and the use of DABCO as a catalyst. Even under these conditions, the nitro group was required to activate the phenol in conjunction with potassium carbonate to deprotonate the phenol to enable conversion to product. Less acidic phenols required stronger bases, which likely also resulted in deprotonation of the pyrrolopyrimidine. This would produce a negative charge on the pyrrolopyrimidine and thus deactivate it to nucleophilic attack, explaining the poor conversion to product. Upon completion of the SNAr, intermediate 2 was subject to a Bechamp reduction to reduce the nitro group into aniline 3 using ammonium chloride as the proton source.

Aniline 3 was then used to synthesize analogues terminating in carboxylic acids through various linkers (Scheme B1). This includes sulfonamide 4, aimed at mimicking the sulfone motif of JAK198, made by treating 3 with the corresponding sulfonyl chloride followed by saponification using NaOH. A pair of amide analogues (5, 6) was also made by treating aniline 3 with cyclic anhydrides. Finally, two urea analogues (7, 8) were prepared by coupling 3 to unprotected glycine or β-alanine using CDI.

The binding affinities of compounds 4-8 to the wild-type (WT) JAK2 JH2 domain were then evaluated using a t1 previously described fluorescence polarization assay (Table A²), and the non-selective JAK2 JH2 ligand JNJ7706621 was used as a positive control. Upon comparison to the previously reported analogue JAK198, the sulfonamide analogue 4 showed a binding of 18% at 50 μM. Replacement of the sulfonamide by an amide yielded significant improvement; analogue 5 has a K_{d} of 18.8±0.2 μM, comparable to those of previously reported triazines. Curiously, the extension of the alkyl chain of 5 by a single methylene unit to give 6 significantly reduced the binding to JAK2 JH2. Furthermore, conversion of the amides 5 and 6 to the corresponding ureas 7 and 8 gave equivalent binding. Overall, the progress made by 5 and 7 illustrates that a few modest changes can transform a false hit from virtual screening into a novel series of JAK2 JH2 binders.

Given these promising results, analogues 5 and 7 were used for further development. Since the urea-containing 7 incorporates a glycine residue, a simple diversification strategy was pursued by replacement of glycine with other amino acids. This strategy not only allowed access to a wide variety of commercially available substituents but also enabled direction of the substituents toward different regions of the JAK2 JH2 domain. These include a pocket adjacent to Phe595 and Phe594, which are implicated in V617F hyperactivation, or a groove defined by Asn673, Cys675, Arg715, and Trp718. Accessing either of these regions brings advantages.

The former feature does not exist in known crystal structures of the JAK2 JH1 domain, as it is blocked by a salt-bridge between Lys882 and Glu898. In the latter case, residues Asn673, 153 Cys675, and Arg715 in the JAK2 JH2 domain are replaced with Asp976, Ala978, and Pro1017 in JAK2 JH1. As such, accessing either of these features could improve binding affinity and selectivity for the JAK2 JH2 domain over the JH1 domain.

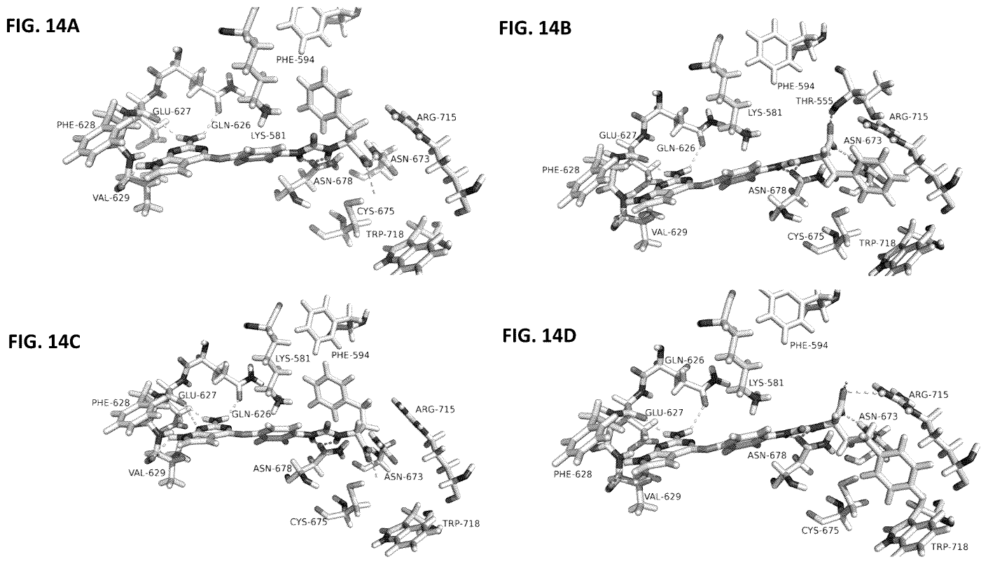

To gauge whether the added substituents could access these features, de novo design was employed using BOMB, generating D- and L-phenylalanyl and D- and L-homophenylalanyl analogues of compound 7. The predicted orientations of the substituents in the JAK2 JH2 domain are shown in FIGS. 14A-14D. Generally, the D-isomers placed the phenyl group toward Phe594, whereas the L-isomers did the opposite, placing the phenyl group toward Trp718. As such, there was motivation to pursue both enantiomers. The chiral integrity of these amino acids was verified by attachment of the chiral resolving agents (R)- or(S)-1-phenylethan-1-amine to analogue 11 using HBTU.

Differences in the NMR of the two synthesized diastereomers were compared, and no racemization was observed. These analogues were thus tested for their binding to the JAK2 JH2 domain (Table A3). The results showed that analogues expressing the L-amino acid gave stronger binding compared to their D-analogues. The L-phenylalanine 9 had an improved binding affinity by a log unit, while the L-homophenylalanine 11 exhibited almost a two log unit improvement. Conversely, the D-phenylalanine 10 yielded only around a 2-fold improvement in binding affinity, whereas the D-homophenylalanine 12 had weaker affinity than the original glycyl analogue (7). These data indicate that the phenethyl group is an important addition for improving binding affinity and that there is a strong dependence on the stereochemistry of the attachment.

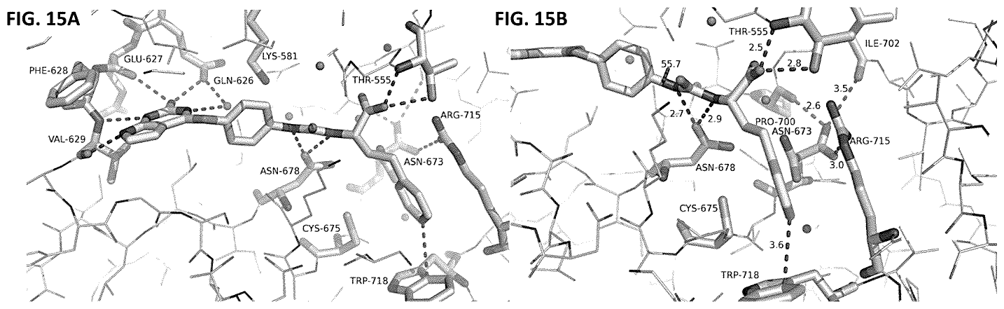

To fully elucidate the binding mode of analogue 11, we obtained a crystal structure for its complex with JAK2 JH2 (FIGS. 15A-15B). It shows an extensive network of hydrogen-bonding, cation-x, and aryl-aryl interactions. As expected, the pyrrolopyrimidine subunit is hydrogen-bonded with the hinge region of the JAK2 JH2 domain (FIG. 15A) in similar fashion to our previously reported triazine and triazole analogues. There is a tight cation-x interaction between the phenoxy ring and Lys581. Interestingly, the urea moiety, while initially installed to mimic the hydrogen bonding found with the planar indole, possesses a dihedral angle of 55.7° in the crystal, out-of-plane with the attached phenolic ether (FIG. 15B); however, it is also hydrogen-bonded to Asn678 as for the indole. Importantly, the crystal structure confirmed that the amino-acid side chain of 11 is directed toward Trp718 (FIG. 15B), as predicted with BOMB. The terminal phenyl group participates in a T-shaped aryl-aryl interaction with Trp718, and it is also parallel to Arg715, forming a second cation-x interaction. The distance between the ipso carbon of the phenyl group and Ne of Arg715 is 3.6 Å. These striking interactions explain the origin of the much-enhanced binding for 11 (Table A3). Addition of the phenethyl group also rearranges the side chain of Asn673 to form new hydrogen bonds with Pro700, Ile702, and Arg715. Two orientations of Asn673 appear in the crystal, reflecting some flexibility in this region.

To seek further gains in affinity, additional side chains were screened (Table A4). Extension of the phenyl group of 11 with a methylene (13) or an ether oxygen (14) yielded modest losses in affinity. Tying the phenyl group into biaryl motifs (analogues 15-17) yielded further losses in affinity, similar to that for phenylalanine analogue 9. This indicated that some flexibility is desired to properly position the phenyl group near Trp718. To further enrich the SAR, L-amino acids containing heteroatoms were also explored, namely the histidine (18), glutamine (19), and asparagine (20) alternatives. Glutamine derivative 19 showed activity similar to that of glycine (7), whereas deletion of a single methylene unit to produce the asparagine 20 yielded a 5-fold improvement in binding affinity. A possible explanation is that the side chain of glutamine 19 is extending out to solvent and is not interacting with the binding site, whereas interactions are enabled with the asparagine variant (20), possibly a hydrogen bond between Asn673 and the carbonyl oxygen of 20. To build on this observation, we created a hybrid analogue by adding the asparagine carbonyl group of 20 to homophenylalanine analogue 11. This yielded the best JAK2 JH2 binder, 21, which exhibited 96 nM affinity. The best previously reported result was 346 nM for a triazole analogue.

The potent analogues 11 and 21 were then evaluated for selectivity by also measuring their binding to the V617F mutant of JAK2 JH2 and the WT JH1 domain (Table A5). As expected, the binding affinities are similar for WT and V617F JAK2 JH2 domains, and these analogues show much weaker binding to JAK2 JH1, with Kd values of ˜35 μM. As such, the selectivities of these compounds for the JAK2 JH2 domain over the JH1 domain were greater than 100-fold. Indeed, analogue 21 is the most selective compound found to-date, with a 360-fold preference for binding the JAK2 JH2 domain over the JH1 domain. The selectivity of these analogues was also examined through docking with Glide SP. For the JAK2 JH2 WT and JH2 V617F domains, analogues docked and scored similarly for both, consistent with their similar binding affinities. The carboxylate of each compound formed a salt-bridge with Arg715 and/or a hydrogen bond with Thr555. Additionally, the terminal phenyl group was often found to form an aryl-aryl interaction with Trp718, as seen in FIGS. 15A-15B for analogue 11. In contrast, analogues were found to score considerably worse in the JAK2 JH1 domain by >2.5 kcal/mol. Without being bount by theory, this difference in score can be attributed to the difference in binding poses between JAK2 JH2 and JH1. If they had similar poses, the carboxylate on the ligands would be located near Asp976 and Asp994, which would be highly repulsive. Instead, the carboxylate of the ligand was predicted to either interact with Lys882 or Arg980, orienting the terminal phenyl away from Trp1020 (JAK2 JH2 equivalent Trp718). Since analogues 11 and 21 were found to be potent and highly selective for the JAK2 JH2 domain, we proceeded with preliminary evaluation of the effects of these compounds on kinase activity in human erythroleukemia (HEL) cells. These cells express only the V617F mutant of JAK2 and, as such, constitutively phosphorylate STAT5. Since the JAK2 JH2 domain is intracellular, we also decided to synthesize and test methyl ester prodrugs of 11 and in case the carboxylic acids impeded cell permeability.

The JAK2 JH2 affinity for both prodrugs was found to be 18% at 50 μM, indicating the importance of the carboxylic acid for binding. Upon treatment of HEL cells with analogues 11, 21, and their respective methyl esters, no inhibition of phosphorylation of STAT5 was observed via Western blot at concentrations up to 75-80 μM after 3-h incubation. Ruxolitinib was used as a positive control and did yield complete inhibition of STAT5 phosphorylation at 2 μM. Thus, the four compounds are not penetrating the cells, or their binding is insufficient to affect the constitutive activation of the V617F JAK2 in HEL cells. Analogues 11, 21, and their corresponding methyl esters were thus assayed in preliminary PAMPA experiments to explore further if permeability is a problem with these compounds. The two carboxylic acids were found to be impermeable in these assays, and the methyl ester of analogue 21 was also impermeable, likely due to its poor solubility. The methyl ester of analogue 11 was found to have a permeability of 3.57×10⁻⁶ cm/s, a value between the medium-(3.08×10⁻⁶ cm/s) and high-permeability controls (4.53× 10⁻⁶ cm/s) diclofenac and chloramphenicol, respectively. Preliminary LCMS of the cell extracts indicates that the methyl ester of 11 is hydrolyzed to 11, but that 11 is present in relatively minute quantities in the cell lysates as compared to the medium, indicating poor permeability. PAMPA does not appear to be a good predictor of cell permeability for this series, a phenomenon more clearly illustrated in our more 00 developed triazole series. Permeability does appear to be a major contributing factor for poor cellular activity of the triazole series.

Nevertheless, the present work sets the stage for additional advances by providing molecules that bind strongly and selectively to JAK2 JH2 and a structural basis for their activity. Selective targeting of the JAK2 JH2 domain is of therapeutic interest due to its reported potential to circumvent negative side-effects associated with conventional targeting of the JAK2 JH1 domain. Given the structural similarity between these two domains, it is challenging to discover JAK2 JH2-selective ligands. Herein, a combination of structure-based and de novo design strategies was employed to transform a false hit from virtual screening into a new series of selective JAK2 JH2 binders. The convergent design approach was successful in yielding compounds with both greater potency and selectivity than previously reported.

In various embodiments, the compound of Formula II is a compound of Formula IV:

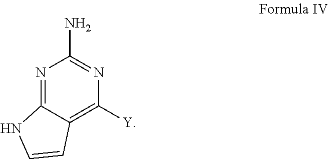

In various embodiments, the compound of Formula IV, has the structure:

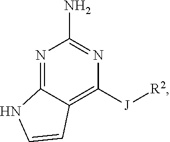

wherein
J is O or NH;
R² is selected from the group consisting of C₆₋₁₀ aryl, C₆₋₁₀ heteroaryl, and combinations thereof, each of which is optionally substituted.

In various embodiments, in the compound of Formula IV, J is O.

In various embodiments, in the compound of Formula IV, J is NH.

In various embodiments, in the compound of Formula IV, R² is selected from the group consisting of:

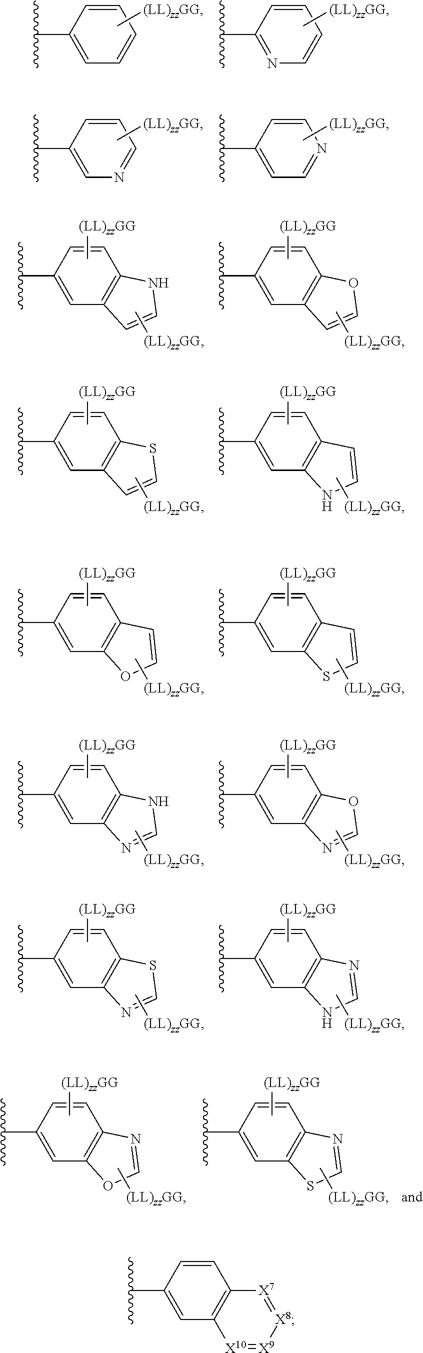

wherein X⁷, X⁸, X⁹, and X¹⁰ are each independently N, NH, or C—Y; and

wherein each -(LL)_{zz}GG moiety represents at least one substituent.

In various embodiments, in the compound of Formula IV, R² is selected from the group consisting of

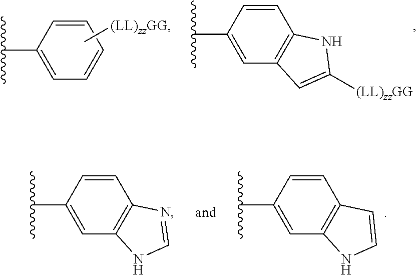

In various embodiments, in the compound of Formula IV, R² is selected from the group consisting of

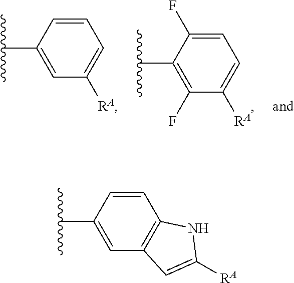

wherein R^{A} is selected from the group consisting of —H, —CO₂H, —NH₂, —OH, —OCH₃, —CH₂OCH₂COOH, and trans —C═C(H)COOH.

In various embodiments, in the compound of Formula IV, R² is selected from the group consisting of:

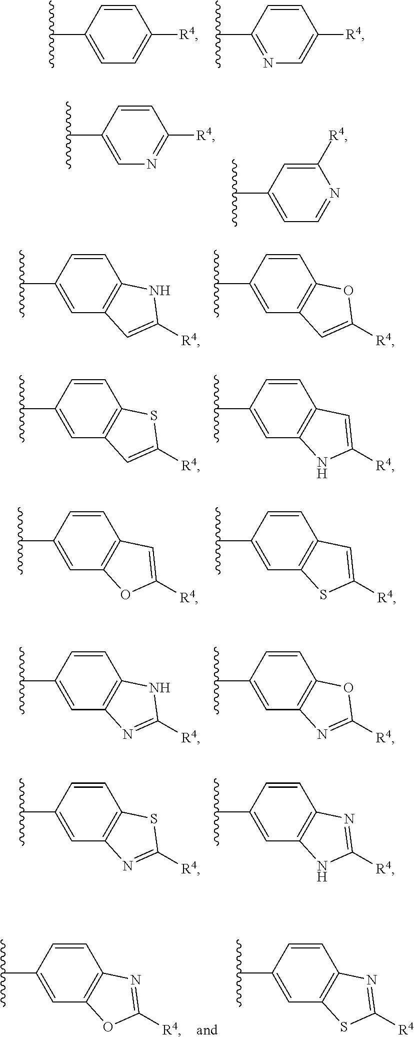

wherein
R⁴ is

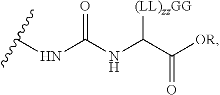

each LL is independently selected from the group consisting of —CH₂—, —NH—, —C(═O)—, and —O—;
R is C₁₋₄ alkyl or H;
zz is an integer from 2 to 6; and
GG is optionally substituted phenyl.

In various embodiments, (LL)_{zz}GG in R⁴ is selected from the group consisting of (CH₂)₂Ph, (CH₂)₃Ph, CH₂C(═O)NHPh, and CH₂ (C═O)Ph.

In various embodiments, (LL)_{zz}GG in R⁴ is selected from the group consisting of

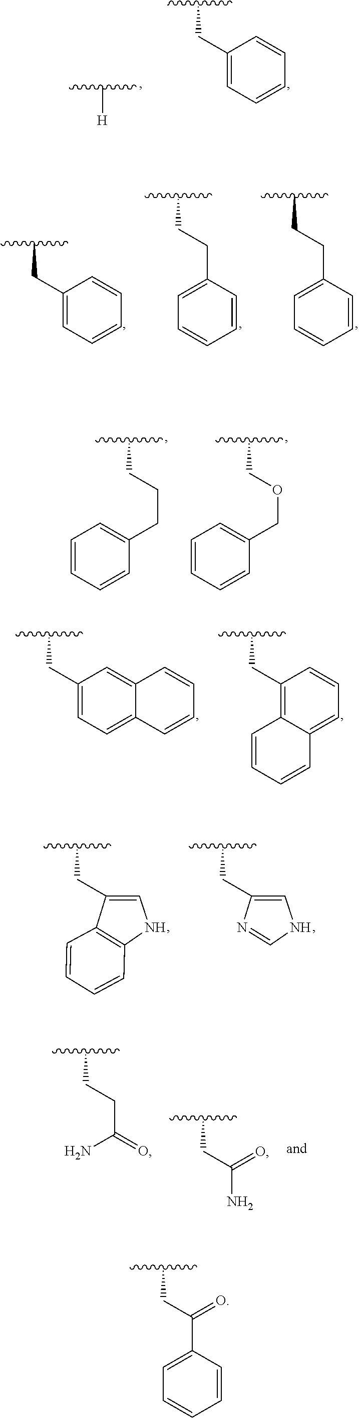

In various embodiments, the compound of Formula IV is selected from the group consisting of

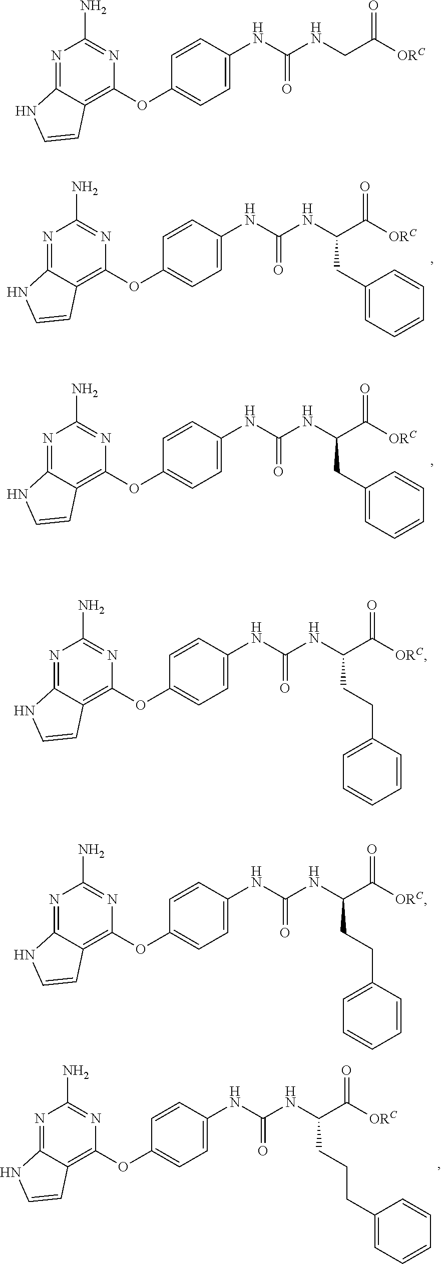

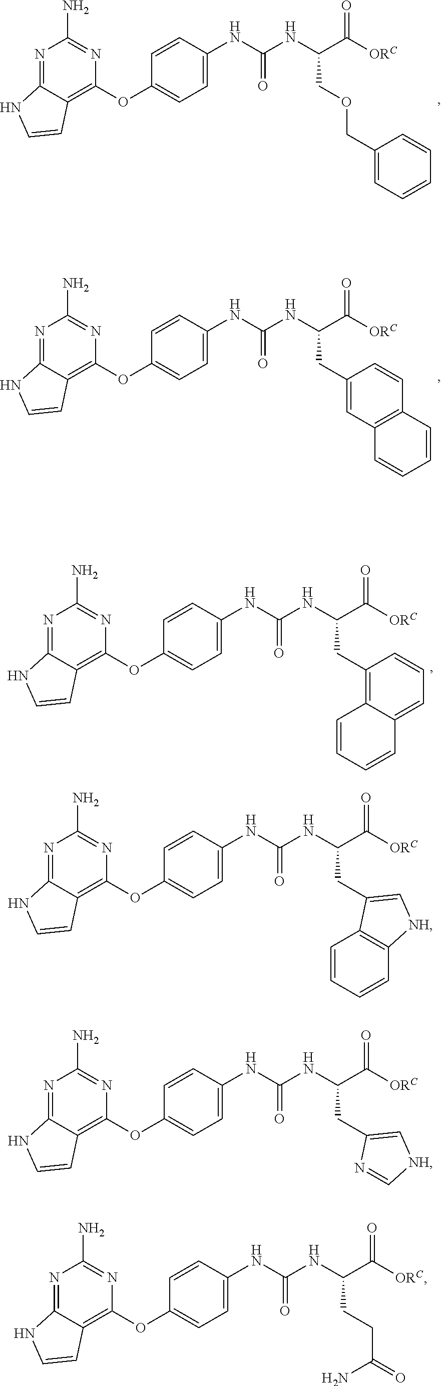

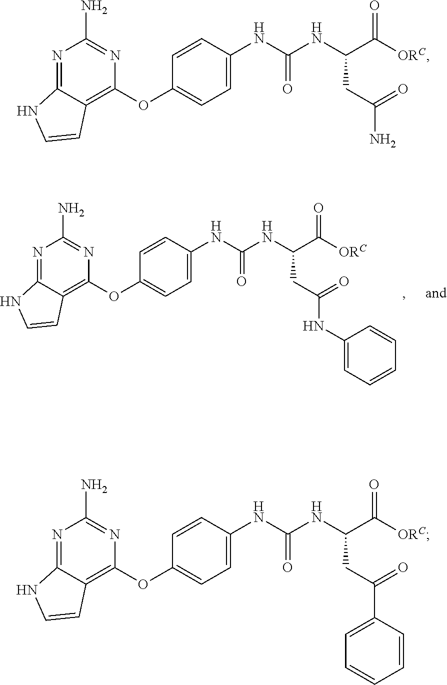

wherein RC is H, CH₃, ethyl, propyl, iso-propyl, n-butyl, sec-butyl, or t-butyl.

In various embodiments, the compound of Formula IV is selected from the group consisting of:

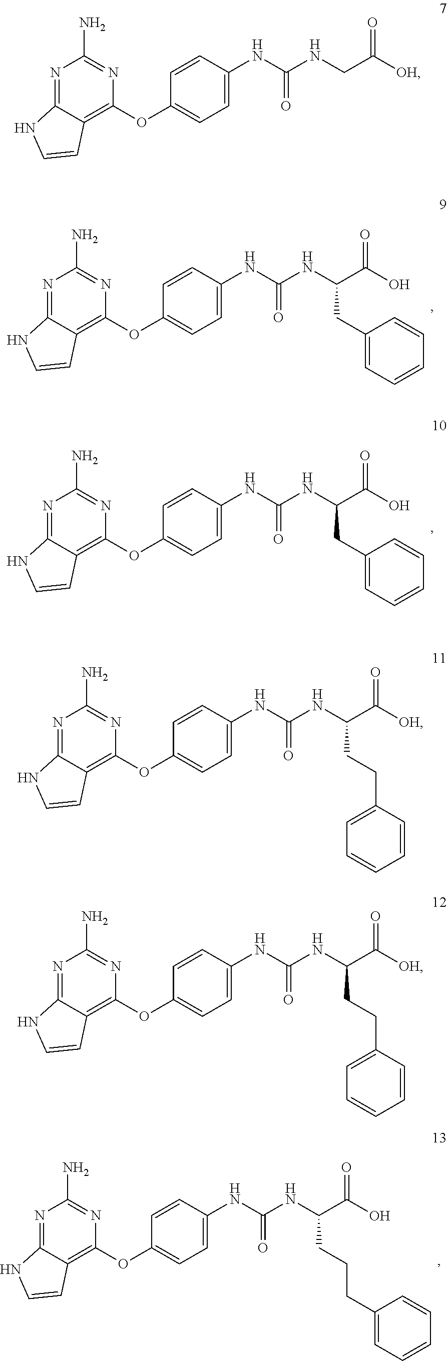

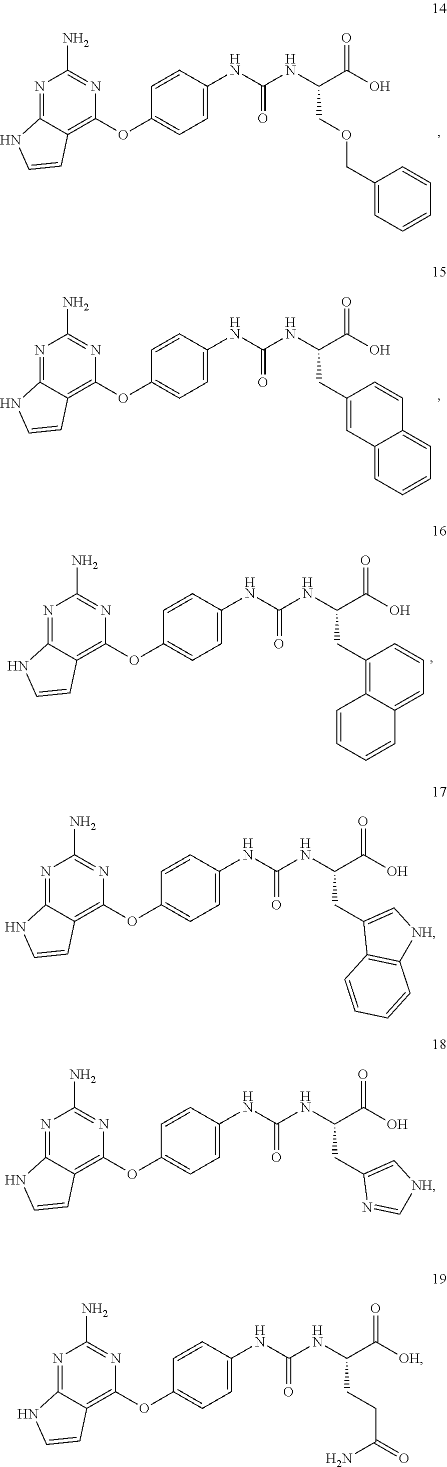 [SMILES: Nc1nc(Oc2ccc(NC(=O)N[C@@H](COCc3ccccc3)C(=O)O)cc2)c2cc[nH]c2n1 ; Nc1nc(Oc2ccc(NC(=O)N[C@@H](Cc3ccc4ccccc4c3)C(=O)O)cc2)c2cc[nH]c2n1 ; Nc1nc(Oc2ccc(NC(=O)N[C@@H](Cc3cccc4ccccc34)C(=O)O)cc2)c2cc[nH]c2n1 ; Nc1nc(Oc2ccc(NC(=O)N[C@@H](Cc3c[nH]c4ccccc34)C(=O)O)cc2)c2cc[nH]c2n1 ; Nc1nc(Oc2ccc(NC(=O)N[C@@H](Cc3c[nH]cn3)C(=O)O)cc2)c2cc[nH]c2n1 ; NC(=O)CC[C@H](NC(=O)Nc1ccc(Oc2nc(N)nc3[nH]ccc23)cc1)C(=O)O]

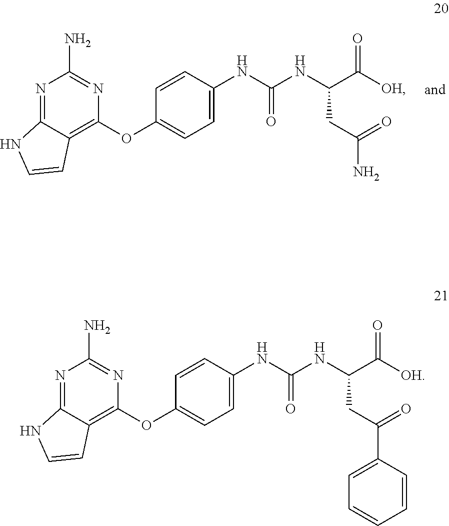 [SMILES: NC(=O)C[C@H](NC(=O)Nc1ccc(Oc2nc(N)nc3[nH]ccc23)cc1)C(=O)O ; Nc1nc(Oc2ccc(NC(=O)N[C@@H](CC(=O)c3ccccc3)C(=O)O)cc2)c2cc[nH]c2n1]

Compounds of Formula IV, in some embodiments, have the following activities against JAK2 JH2 domain, JH1 domain, and/or V617F JH2 domain:

TABLE A2
Binding of Initial Pyrrolopyrimidines to WT JAK2 JH2^{a}

| 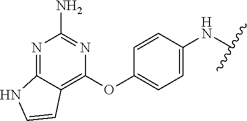 [SMILES: *Nc1ccc(Oc2nc(N)nc3[nH]ccc23)cc1] | Cmpd | JAK2 JH2 WT K_{d} (μM) |
|---|---|---|
| 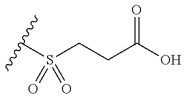 [SMILES: *S(=O)(=O)CCC(=O)O] | 4 | 18% @ 50 |
| 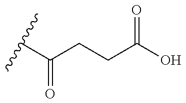 [SMILES: *C(=O)CCC(=O)O] | 5 | 18.8 ± 0.2 |
| 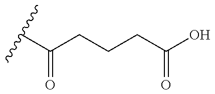 [SMILES: *C(=O)CCCC(=O)O] | 6 | 27% @ 50 |
| 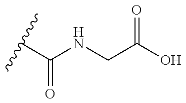 [SMILES: *C(=O)NCC(=O)O] | 7 | 19.7 ± 2.3 |
| 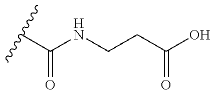 [SMILES: *C(=O)NCCC(=O)O] | 8 | 30% @ 50 |
| — | JNJ7706621 | 0.46 ± 0.2 |
| — | JAK198 | 4% @ 50 |

^{a}Binding affinities (K_{d}) using a fluorescence polarization (FP) assay were measured for analogues that exhibited greater than 50% binding at 50 μM, whereas weaker binders are shown only as % binding at 50 μM. Kd values are represented as averages of two assays in quadruplicate ± SEM.

TABLE A3
Binding of D- and L-Pyrrolopyrimidine Analogues to WT JAK2 JH2^{a}

| 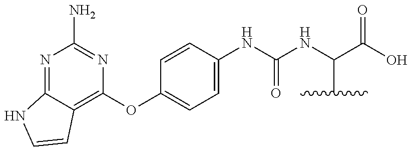 [SMILES: *C(NC(=O)Nc1ccc(Oc2nc(N)nc3[nH]ccc23)cc1)C(=O)O] | Cmpd | JAK2 JH2 WT K_{d} (μM) |
|---|---|---|
| 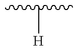 [SMILES: *[H]] | 7 | 19.7 ± 2.3 |
| 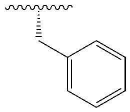 | 9 | 2.22 ± 0.10 |
| 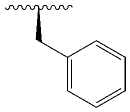 | 10 | 9.9 ± 1.7 |
| 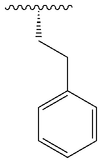 | 11 | 0.327 ± 0.22 |
| 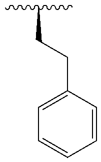 | 12 | 46% @ 50 |
| — | JNJ7706621 | 0.46 ± 0.12 |

^{a}Binding affinities (K_{d}) using a fluorescence polarization (FP) assay were measured for analogues that exhibited greater than 50% binding at 50 μM, whereas weaker binders are shown only as % binding at 50 μM. Kd values are represented as averages of two assays in quadruplicate ± SEM.

TABLE A4
Binding Affinities (Kd) of Derivatives of Analogue 11-21 to WT JAK2 JH2^{a}

| 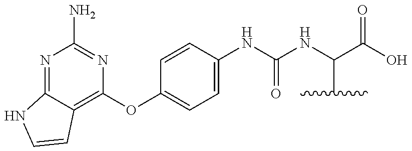 [SMILES: *C(NC(=O)Nc1ccc(Oc2nc(N)nc3[nH]ccc23)cc1)C(=O)O] | Cmpd | JAK2 JH2 WT K_{d} (μM) |
|---|---|---|
| 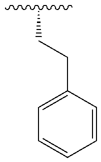 | 11 | 0.327 ± 0.22 |
| 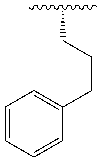 | 13 | 0.587 ± 0.035 |
| 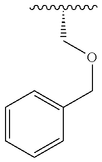 | 14 | 0.590 ± 0.021 |
| 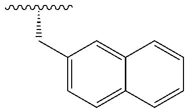 | 15 | 0.880 ± 0.007 |
| 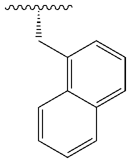 | 16 | 3.08 ± 0.1 |
| 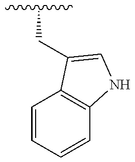 | 17 | 1.45 ± 0.06 |
| 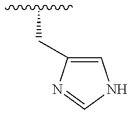 | 18 | 4.40 ± 0.21 |
| 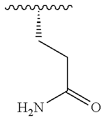 | 19 | 16.48 ± 0.41 |
| 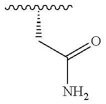 | 20 | 2.95 ± 0.1 |
| 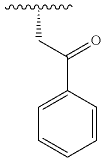 | 21 | 0.096 ± 0.005 |

^{a}Binding affinities (K_{d}) using a fluorescence polarization (FP) assay were measured for analogues that exhibited greater than 50% binding at 50 μM, whereas weaker binders are shown only as % binding at 50 μM. Kd values are represented as averages of two assays in quadruplicate ± SEM.

TABLE A5
Binding Affinities (Kd) of Analogues 11 and
21 to WT JAK2 JH2, V617F JH2, and JH1^{a}

| | JAK2 JH2 WT | JAK2 V617F | JH1 WT | Selectivity |
|---|---|---|---|---|
| Cmpd | (μM) | (μM) | (μM)) | (JH2 vs. JH1) |
| 11 | 0.327 ± 0.22 | 0.179 ± 0.019 | 36.1 ± 2.0 | 110-fold |
| 21 | 0.096 ± 0.005 | 0.106 ± 0.004 | 32.24 ± 0.51 | 360-fold |

^{a}Binding affinities (K_{d}) using a fluorescence polarization (FP) assay were measured for analogues that exhibited greater than 50% binding at 50 μM, whereas weaker binders are shown only as % binding at 50 μM. Kd values are represented as averages of two assays in quadruplicate ± SEM.

Compounds of Formula I, Formula II, Formula III, and Formula IV are, in various embodiments, selective inhibitors of the JAK2 JH2 domain. JAK1, JAK2, JAK3, and TYK2 are members of the Janus family of non-receptor tyrosine kinases, which are activated by and mediate the cellular responses induced by binding of a variety of cytokines to specific cytokine receptors. Cytokine-induced activation of the JAK-STAT signaling pathway and other intracellular pathways play important roles in the control of cell proliferation, hematopoiesis, and immune functions. In addition to a canonical tyrosine kinase domain (JH1) located in the N-terminal region, JAK proteins contain a pseudokinase domain designated JH2. Though JH2 domains have an ATP-binding site, they show little or no catalytic activity. However, JH2 domains do have a regulatory function for the JH1 kinase activity such that mutations in JH2 can cause hyperactivation leading to numerous diseases and cancer. In particular, the single point-mutation Val617Phe (V617F) in JAK2 JH2 is responsible for the majority of myeloproliferative disorders including polycythemia vera, myelofibrosis, and essential thrombocythemia. Though undesirable side effects such as anemia may occur upon inhibition of JAK2 JH1 kinase activity, mutagenesis studies have raised the possibility of selective reversal of the activating effect of V617F by displacement of ATP from JAK2 JH2.

In certain non-limiting embodiments of the disclosure, the compounds contemplated herein bind to the JAK2 JH2 ATP binding site with selectivity over the corresponding JAK2 JH1 ATP binding site. In certain embodiments, the compounds contemplated herein selectively reverse the activating effect of the V617F mutation in JAK2 JH2.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, HC, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

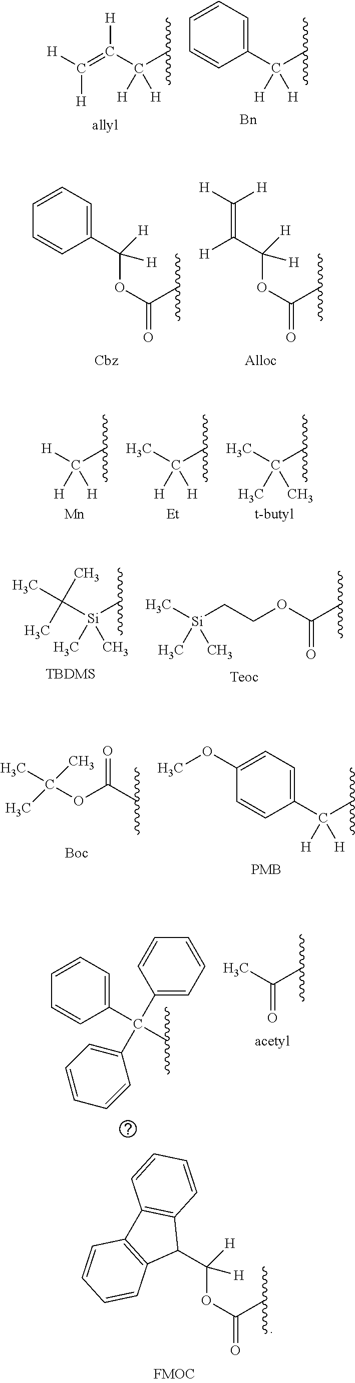

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans) buccal, (trans) urethral, vaginal (e.g., trans- and perivaginally), (intra) nasal and (trans) rectal, intravesical, intrapulmonary, intraduodenal, intragastrical, intrathecal, subcutaneous, intramuscular, intradermal, intra-arterial, intravenous, intrabronchial, inhalation, and topical administration.

Methods of Treatment, Amelioration, and/or Prevention

The compounds of Formula I, Formula II, Formula III, and/or Formula IV are useful for treating, ameliorating, and/or preventing myeloproliferative neoplasms (MPNs). Examples of MPNs that can be treated, a meliorated, and/or prevented with the compounds of Formula I, Formula II, Formula III, and/or Formula IV include chronic myelogenous leukemia (CML), polycythemia vera, primary myelofibrosis (also called chronic idiopathic myelofibrosis), essential thrombocythemia, chronic neutrophilic leukemia, and chronic eosinophilic leukemia.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats myeloproliferative neoplasms.

In certain embodiments, administering the compound(s) described herein to the subject allows for administering a lower dose of the additional therapeutic agent as compared to the dose of the additional therapeutic agent alone that is required to achieve similar results in treating a myeloproliferative neoplasm in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating myeloproliferative neoplasms, and/or with an additional therapeutic agents that reduce or ameliorate the symptoms and/or side-effects of therapeutic agent used in the treatment of a myeloproliferative neoplasms. These additional therapeutic agents may comprise compounds that are commercially available or synthetically accessible to those skilled in the art. When the additional therapeutic agents useful for treating myeloproliferative neoplasms are used, these additional therapeutic agents are known to treat, or reduce the symptoms of a myeloproliferative neoplasm.

In non-limiting examples, the compounds described herein can be used in combination with one or more of the following therapeutic agents useful for treating myeloproliferative neoplasms: Adriamycin PFS (Doxorubicin Hydrochloride), Adriamycin RDF (Doxorubicin Hydrochloride), Arsenic Trioxide, Azacitidine Cerubidine (Daunorubicin Hydrochloride), Clafen (Cyclophosphamide), Cyclophosphamide, Cytarabine, Cytosar-U (Cytarabine), Cytoxan (Cyclophosphamide), Dacogen (Decitabine), Dasatinib, Daunorubicin Hydrochloride, Decitabine Doxorubicin Hydrochloride, Etoposide Phosphate, Gleevec (Imatinib Mesylate), Imatinib Mesylate, Jakafi (Ruxolitinib Phosphate), Nilotinib, Rubidomycin (Daunorubicin Hydrochloride), Ruxolitinib Phosphate, Sprycel (Dasatinib), Tarabine PFS (Cytarabine), Tasigna (Nilotinib), Trisenox (Arsenic Trioxide), and Vidaza (Azacitidine).

In certain embodiments, the compounds described herein can be used in combination with radiation therapy. In other embodiments, the combination of administration of the compounds described herein and application of radiation therapy is more effective in myeloproliferative neoplasms than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a myeloproliferative neoplasm. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a myeloproliferative neoplasm in the patient. An effective amount of the therapeutic compound necessary to achieve a therapeutic effect may vary according to factors such as the state of the disease or disorder in the patient; the age, sex, and weight of the patient; and the ability of the therapeutic compound to treat a myeloproliferative neoplasm in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% a-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described hrein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a myeloproliferative neoplasm in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Structure-Based Design

BOMB (Biochemical and Organic Model Builder) was utilized to model in silico various 6-membered ring substituents for the optimization of 2. The generated protein-ligand complexes were energy-minimized with MCPRO using the OPLS-AA/M force field for proteins and OPLS/CMIA for ligands. Docking methods were also used starting from the crystal structures of 1 in complex with JH2 WT (PDB ID 5USZ) and JH1 (PDB ID: 5USY). The crystal structure of 11 in complex with JH2 WT (PDB ID: 7TOP) was eventually used in retrospective analyses. The desired ligands for docking were generated with Maestro and LigPrep using the OPLS3 force field and Epik. They were docked with Glide SP.

### Competitive Fluorescence Polarization (FP) Assay

Binding affinities (Kd) with the JAK2 JH2 domain were evaluated using the previously developed FP assay and compound 1 as a control. An initial screening is conducted at 50 μM, and binding affinities (Kd's) are measured for those exhibiting >50% binding at 50 μM (Table 1). Replacement of the oxazole in 2 with 6-membered aromatic rings produced compounds with a binding range of 0.129 to 1.4 μM. Conversion of the carboxylate of compound 7 to a methyl ester (8) or just hydrogen (9) resulted in large loss of binding affinity, and the addition of a benzyloxy group gave compounds (11-14) with an affinity range of 0.033 to 0.075 μM. Selectivity measurements were conducted subsequently for the most potent JAK2 JH2 ligands.

In a flat black bottom 96 well plate (Corning), 200 μL of FP buffer were added to column 1 (blank), 150 μL to column 2, and 140 μL to columns 3-12. 10 μL of 2.96 μM of JAK2-JH2 WT (3.52 μM for JAK2-JH2-VF, and 6.93 μM for JAK2-JH1), were added to columns 3-12, followed by the addition of 2 μL of DMSO to columns 1-3. 2 μL of inhibitor in DMSO at different concentrations were added from column 4 to 12. 50 μL of 24 nM of tracer were added to columns 2-12. Fluorescence polarization was measured at λ_{exc}=485±20 nm, λₑₘ=535±25 nm for 1 hour. Experiments were carried out by quadruplicates in three independent experiments. Data were analyzed by a least-squares non-linear fit, generated using Prism 7 in order to determine the compound's IC₅₀. K_{d} values for each inhibitor are calculated using the following equation based on the IC₅₀, K_{d} of the tracer (K_{d}^{t}), total (Lₜ) and bound (L_{b}) tracer, as well as total protein concentration (Pₜ).¹³

$${K}_{d}^{I} = \frac{{L}_{b} I {C}_{50} {K}_{d}^{t}}{{P}_{t} {L}_{t} + {L}_{b} ( {P}_{t} - {L}_{t} + {L}_{b} - {K}_{d}^{t} )}$$

### Parallel Artificial Membrane Permeation Assay (PAMPA)

To gauge the permeability of selected compounds, the Parallel Artificial Membrane Permeation Assay (PAMPA) was used. It simulates a cell membrane with a mix of dodecane and 4% lecithin applied to a filter plate. Theophylline, diclofenac, and chloramphenicol were used as low, medium, and high permeability controls, respectively.

Initially, the permeability of compound 7 and its derivatives 8 and 9 were examined to determine the effect of the carboxylic acid. As shown in Table 2, all three compounds were measured to be essentially impermeable, with PAMPA values close or equal to 0. For 7, this problem was thought to be associated with the anionic and polar groups of the ligand, however, the result for the methyl ester 8 indicates that the carboxylate is not the only source of poor permeability. For the biphenyl analog 9, poor aqueous solubility may have contributed to the observed poor permeability.

Nevertheless, compound 11 yielded a permeability value above the low control (theophylline) despite the presence of the carboxylic acid, possibly due to increased lipophilicity contributed by the benzyloxy group. The m-benzyloxy analog 12 was tested as well, and it exhibited a 3-fold increase in permeability compared to its isomer 11. From this result, it became apparent that different substitution patterns at the right side of the ligand were significantly affecting permeability, and the presence of the carboxylic acid can be tolerated. At this point we wanted to explore the effects of other polar moieties on permeability. The sulfonamide moiety in 1-12 is solvent exposed in their complexes so its modification was not expected to have large effects on binding, while its 3-dimentionality can contribute to aqueous solubility and slow the rate of membrane penetration. As an initial study, a carboxylic amide arose as a synthetically tractable alternative with less polar surface area and molecular weight. Consequently, compounds 13 and 14 were synthesized. Compound 13, the N-methyl amide analog of 11, exhibited similar binding affinity for WT JAK2 JH2 (Table 1), but surprisingly almost 2-fold reduced permeability (Table 2). Compound 14, the N-methyl amide analog of 12, exhibited 2-fold improved binding affinity (0.044 μM) without significantly different permeability compared to 12.

TABLE 2
Permeability Measured with PAMPA for Selected Compounds^{a}

| Compound | 7 | 8 | 9 | 11 | 12 | 13 | 14 |
|---|---|---|---|---|---|---|---|
| Per- | 0.015 | 0.09 | 0.000 | 0.488 | 1.15 | 0.24 | 0.971 |
| mebaility | | | | | | | |
| (× 10⁻⁶ | | | | | | | |
| cm/s) | | | | | | | |
| TPSA | 192.6 | 181.6 | 155.3 | 201.8 | 201.8 | 170.7 | 170.7 |
| (Å²) | | | | | | | |

^{a}Experiments were performed in duplicate for each compound. Controls: theophylline (low control) 0.27-0.33 × 10⁻⁶ cm/s, diclofenac (medium control) 2.91-3.13 × 10⁻⁶ cm/s, and chloramphenicol (high control) 4.36-4.99 × 10⁻⁶ cm/s.

### Generation of Recombinant Baculoviruses

The isolated JH1 and JH2 domains of human JAK2 were expressed in baculovirus-infected Sf9 insect cells and purified similar to the procedure reported previously. The two reported JH2 domain constructs contained residues 536-812 (with either mutations W659A, W777A, F794H, or mutations W777A, F794H, V617F), followed by a C-terminal thrombin cleavage site and 6×His-tag. The reported JH1 domain construct included an N-terminal 6×His-tag, followed by a TEV cleavage site and residues 840-1132. Recombinant bacmid and baculoviruses were generated using the Bac-to-Bac baculovirus expression system (Invitrogene). DH10Bac competent cells were transformed with recombinant pFastBac plasmid containing the gene of interest to generate the recombinant expression bacmid. P1, P2 and P3 baculovirus stocks were produced according to the manufacturer's instructions. Sf9 cells were grown in HyClone SFX-Insect cell culture media (GE Healthcare) at 27° C.

### JAK2 Protein Expression and Purification

Sf9 cells were grown in HyClone SFX-Insect cell culture media to a density of 2.5-4.0×10⁶ cells/mL, followed by transfection with P3 baculovirus stock. After incubation for 48 h at 27° C., cells were harvested and separated from the supernatant by centrifugation (4000 rpm, 30 mins). Purification of the JH1 and JH2 domains of JAK2 was performed in an identical manner. Cell pellets were resuspended in lysis buffer composed of 20 mM Tris pH 8.0, 500 mM NaCl, 20% glycerol, 0.25 mM TCEP, and complete EDTA free protease inhibitor (Roche). Cells were lysed by sonication, followed by pressure homogenization using an Emulsiflux cell disruptor (Avestin). Lysate was separated from cell debris by centrifugation (45 min, 16,500 rpm). Ni-NTA agarose beads (Qiagen) were added in batch mode, and incubated for 2 h at 4° C. Beads were washed with lysis buffer containing 10 mM imidazole, and JAK2 protein was eluted with lysis buffer containing 200 mM imidazole. The eluate was dialyzed overnight at 4° C. using a MWCO 3.5 kDa Slide-A-Lyzer dialysis cassette (Thermo Fisher Scientific) against a low salt dialysis buffer composed of 20 mM Tris pH 8.0, 25 mM NaCl, 20% glycerol, and 0.25 mM TCEP. The dialysis product was filtered through a 0.45 μm membrane and loaded onto a pre-equilibrated Mono Q HR 16/19 column (GE Healthcare) linked to an ÄKTA pure protein purification system (GE Healthcare). Protein was eluted applying a linear gradient starting with dialysis buffer and ending with dialysis buffer containing 500 mM NaCl. JAK2 fractions were pooled and applied to a Superdex 75 10/300 (GE Healthcare) pre-equilibrated with a buffer composed of 20 mM Tris pH 8.0, 100 mM NaCl, 10% glycerol, and 1.0 mM TCEP. Purified protein was aliquoted, flash-frozen in liquid nitrogen, and stored at −80° C.

### Cell Culture and Western Blot Analysis

Human cell lines HEL (containing the JAK2 V617F mutant) and TF-1 (containing wild-type JAK2) were obtained from the ATCC (Philadelphia, PA) and were cultured in RPMI media (ATCC modification; containing L-glutamine, HEPES, Sodium Pyruvate, high glucose and low sodium bicarbonate) in 100 mm culture dishes. RPMI media for TF-1 cell culture was supplemented with an additional 2 ng/mL GM-CSF. To measure inhibition of STAT5 phosphorylation, approximately 1×10⁶ cells per well in a 6-well plate were incubated for 1 hour with different concentrations of inhibitor added from a concentrated DMSO stock. Cells were lysed and equal amounts of protein as determined by a BCA assay from each experiment were run by 12% SDS-PAGE. For Western blots, membranes were incubated for 1 hour at room temperature with anti-STAT5 (Cell Signaling Technology, #25656S) and anti-phospho-STAT5 (Y694) (Abcam, ab32364) followed by incubation with Anti-rabbit IgG HRP-linked antibody (Cell Signaling Technology, #7074) for 1 hour. Signal was detected on an iBright FL1000 Imaging System after addition of BioRad Clarity Max Western ECL substrate onto the membrane.

### Fluorescence Polarization (FP) Assays:

Determination of Tracer Affinity with JAK2-JH2-WT, JAK2-JH2-V617F, and JAK2-JH1.

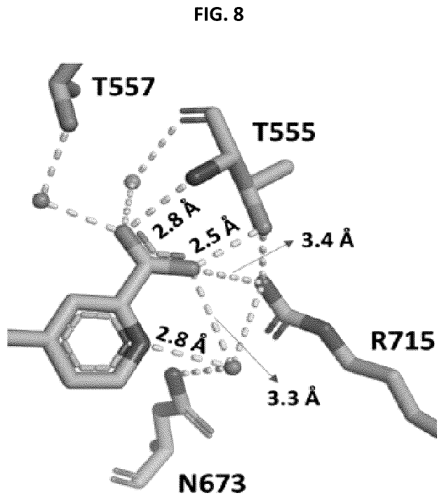

In a flat black bottom 96 well plate (Corning), the buffer (20 mM Tris-HCl pH 8.0, 150 mM NaCl, 20% Glycerol, 0.5 mM TCEP, 0.01% Tween 20) is added-200 μL to column 1 (blank), 295 μL to column 2, 150 μL to columns 3-12. 5 μL of protein (179.0 μM JAK2-JH2-WT, 154.7 μM JAK2-JH2-V617F, 126.3 μM JAK2-JH1) were added to column 2. 150 μL was transferred, using a multichannel pipette, from column 2 to 3, 3 to 4, 4 to 5, until reaching the last column to make a serial dilutions (1:2). 50 μL of 24.0 nM tracer were added from columns 2-12 and fluorescence polarization was measured at λ_{exc}=485±20 nm, λₑₘ=535±25 nm using an Infinite F500 plate reader until no FP variation was observed. From the lowest and highest FP values (tracer free and tracer fully bound to JAK) fraction of ligand bound to the protein to ligand total (L_{b}/Lₜ) was calculated for each concentration of the JAK2-JH2-WT, JAK2-JH2-V617F, and JAK2-JH1 (FIGS. 8A-8B). Experiments were carried out by quadruplicates in three independent experiments. The data provided a typical saturation-binding curve and K_{d} was calculated fitting the results to the Hill equation using Prism 7.

### Cloning and Expression of Full-Length JAK2.

Full-length cDNA encoding human JAK2 (residues 1-1132) wild-type (NP_001309123) and V617F mutant with C-terminal FLAG-Tag (DYKDDDDK) were amplified by PCR, and subcloned into a modified pOptiVec expression vector (Invitrogen). JAK2 constructs were expressed in HEK293T cells grown at 37° C. at 5% CO₂ in DMEM (Gibco) supplemented with 10% (v/v) FBS (Gibco) and 1% (v/v) penicillin-streptomycin (Gibco). HEK-293T cells were transiently transfected using Lipofectamin 2000 (Invitrogen) according to manufacturer's instructions. 36 h post-transfection, cells were washed twice with ice-cold PBS, and lysed with ice-cold lysis buffer (50 mM HEPES pH 7.4, 150 mM NaCl, 10% (v/v) glycerol, 1% (v/v) Triton-X 100, 1 mM EDTA, 1 mM EGTA, 25 mM NaF, 1.5 mM MgCl₂, 1.0 mM Na₃VO₄, Roche complete mini EDTA-free protease inhibitor cocktail mixture). The lysate was centrifuged (20 min, 13000×g), and the lysate supernatant was flash-frozen in liquid nitrogen and stored at −80° C.

### Immunoprecipitation and In Vitro Kinase Assay.

JAK2 protein was immunoprecipitated from the lysate supernatant by adding anti-FLAG M2 antibody (Sigma-Aldrich, no. F1804) and protein G-PLUS agarose (Santa Cruz Biotechnology, no. sc-2002) followed by incubation overnight while rocking at 4° C. Immunoprecipitates were washed four times with wash buffer (50 mM HEPES pH 7.4, 150 mM NaCl, 10% (v/v) glycerol, 0.1% (v/v) Triton-X 100, 1 mM EDTA, 1 mM EGTA, 25 mM NaF, 1.5 mM MgCl₂, 1.0 mM Na₃VO₄, Roche complete mini EDTA-free protease inhibitor cocktail mixture), and once with kinase reaction buffer (50 mM HEPES pH 7.4, 150 mM NaCl, 0.5 mM DTT, 5 mM MnCl₂). The [γ-³²P]ATP in vitro kinase activity assay was performed based on a previously published protocols. Washed immunoprecipitates were divided into equal parts, centrifuged, their residual solvent was removed, and the resulting pellets were resuspended in 25 μL kinase reaction buffer containing different concentrations of 1 or 10, followed by incubation for 1 h at 4° C. The autophosphorylation reaction of JAK2 was initiated by adding 25 μL of phosphorylation mixture consisting of kinase reaction buffer supplemented with 10 μM cold ATP, and 2 μCi (for JAK2 V617F) or 5 μCi (for JAK2 wild-type) of [γ-32P]ATP (Easy Tides, PerkinElmer) per reaction. The mixture was allowed to react for 15 min at 30° C. (within the linear range of kinase activity), and stopped by putting on ice and adding 18 μL of reducing Laemmli sample buffer (4x). Samples were heated at 95° C. for 5 min, and run by 7.5% SDS-PAGE. Gels were rocked in a solution of 10% glycerol, 20% ethanol for 30 min, dried with a vacuum drier, and autoradiographed using a phosphor imager. To calculate IC₅₀ values, phosphor autoradiography was quantified using ImageJ, and curves were plotted with Prism 8.0 (GraphPad Software Inc., La Jolla, CA).

### General Synthetic Methods

Reagents and solvents were obtained from commercial supplies and used without further purification. Reactions were monitored by thin-layer chromatography (TLC) using Merck pre-coated silica gel plates (analytical, SiO₂-60, F254). TLC plates were visualized under UV light (254 nm/365 nm). Organic solutions were concentrated under reduced pressure on a Büchi rotary evaporator.

Low resolution mass analysis of intermediates was performed with an Agilent 6120 Quadrupole LC/MS instrument via electrospray ionization. Chromatographic purification with flash column chromatography was performed with a Teledyne ISCO Combiflash® Rf+ automated system employing RediSep Normal Phase Silica (particle size: 35-70 μm; pore size: 60 Å) or RediSep Gold Normal Phase Silica (particle size: 20-40 μm; pore size: 60 Å) disposable cartridge columns. RediSep Gold C₁₈ reusable columns (particle size: 20-40 μm spherical; pore size: 100 Å) were employed for reverse phase chromatography.

### Preparative Reverse Phase HPLC Systems:

System A: a Shimadzu Prominence system equipped with LC-20AP pumps, CBM-20A Communications BUS module, SPD-20A UV/vis detector, SIL-10AP autosampler, FRC-10A fractions collector and a Waters SymmetryPrep™ C8, 19×300 mm column (particle size: 7 μm; pore size: 100 Å) with a gradient of 0.1% formic acid in water and 0.1% formic acid in acetonitrile as the mobile phase. (Purification of Compounds 3-6).

System B: an Agilent 1260 Infinity II system equipped with G7161A Preparative Binary Pump., G7115A Diode Array Detector WR., G7157A Preparative Autosampler, G7159B Agilent Preparative Open-Bed Fraction Collector and an Agilent 100 Å C18, 21.2×100 mm, 5 μm particle size preparative column with a gradient of 0.1% formic acid in water and 0.1% formic acid in acetonitrile as the mobile phase. Purity assessment was conducted with the same system, using an Agilent C18 Scalar column, 100 Å, 4.6×100 mm, 5 μM particle size, with a gradient of 0.1% formic acid in water and 0.1% formic acid in acetonitrile as the mobile phase. (Purification of Compounds 3i, 4i, 7, 12-14).

Nuclear magnetic resonance (NMR) spectra were recorded on an Agilent DD2 400 (¹H NMR, ¹³C NMR recorded at 400, and 101 MHz, respectively), an Agilent DD2 500 (1H NMR, ¹³C NMR recorded at 500, and 126 MHz, respectively), or an Agilent DD2 600 (1H NMR, ¹³C NMR recorded at 600, and 151 MHz, respectively). All spectra were recorded at room temperature. Chemical shifts are reported in ppm relative to deuterated solvent as an internal standard (8H DMSO-d₆ 2.50 ppm, δ_{C} DMSO-d₆ 39.52 ppm; 8H Methanol-d₆ 3.31 ppm, δ_{C} Methanol-d₆ 49.00 ppm δ_{H} Acetone-d₆ 2.05 ppm, δ_{C} Acetone-d₆ 29.84 ppm, 206.26 ppm; δ_{H} Chloroform-d 7.26 ppm, δ_{C} Chloroform-d 77.16 ppm) with the following convention for describing multiplicity (s=singlet, d=doublet, t=triplet, q=quartet, quint=quintet, m=multiplet, br=broad signal, dd=doublet of doublets, etc).

### High Resolution Mass Spectroscopy (HRMS) Instruments

A) a Waters Xevo QTOF with a Z-spray electrospray ionization source. Purity was determined on a Shimadzu Prominence HPLC equipped with an Agilent Poroshell 120 SB-C18 2.7 μm column, using 0.1% TFA in water and 0.1% TFA in acetonitrile as the mobile phase. (Compounds 3-6)

B) a Shimadzu Scientific Instruments QToF 9030 LC-MS system, equipped with a Nexera LC-40D xs UHPLC, consisting of a CBM-40 Lite system controller, a DGU-405 Degasser Unit, two LC-40D XS UHPLC pumps, a SIL-40C XS autosampler and a Column Oven CTO-40S. UV data was collected with a Shimadzu Nexera HPLC/UHPLC Photodiode Array Detector SPD M-40 in the range of 190-800 nm. Mass spectra were subsequently recorded with the quadrupole time-of-flight (QToF) 9030 mass spectrometer. The samples were held at 4° C. in the autosampler compartment. 0.3 μL of each spiked solution were injected into a sample loop and separated on a Shim-pack Scepter C18-120, 1.9 μm, 2.1×100 mm Column, equilibrated at 40° C. in a column oven. A binary gradient was used: Solvent A: Water, HPLC grade Chromasolv, with 0.1% Formic Acid, Solvent B: Acetonitrile, HPLC grade Chromasolv, with 0.1% Formic Acid. The ionization source was run in “ESI” mode, with the electrospray needle held at +4.5 kV. Nebulizer Gas was at 2 L/min, Heating Gas Flow at 10 L/min and the Interface at 300° C. Dry Gas was at 10 L/min, the Desolvation Line at 250° C. and the heating block at 400° C. Mass spectra were recorded in the range of 50 to 2000 m/z in the positive ion mode. Measurements and data post-processing were performed with LabSolutions 5.97 Realtime Analysis and PostRun. (Compounds 7-14). The purity of all the compounds was determined to be >95% by integration of the UV traces. The samples showed no minor peaks above 3%.

### Synthetic Strategy for Compounds of Formula I

In various embodiments, the general approach can be illustrated with the retrosynthesis shown in Scheme 1A. We had previously established the conditions for carrying out the key urea formation reaction, through a 1H-[1,2,4]triazole-3,5-diamine (Fragment A) and a phenylcarbamate (Fragment B).

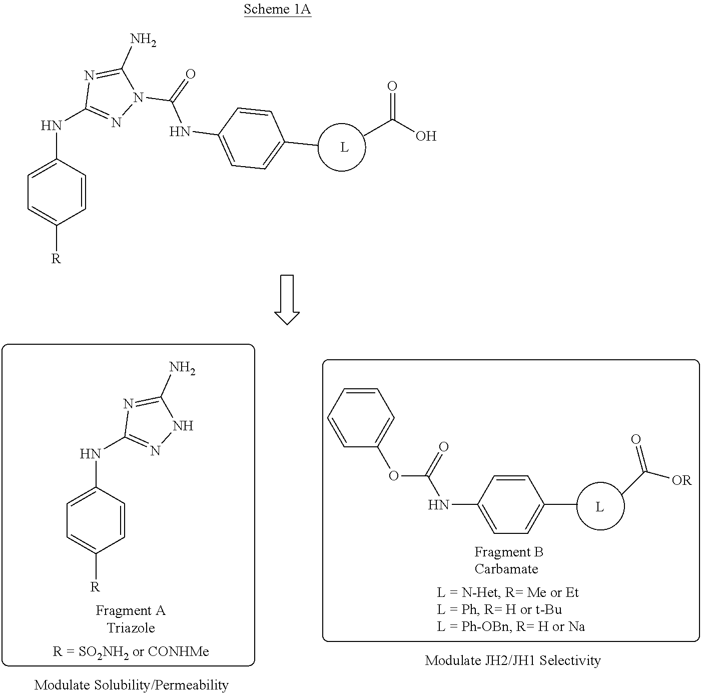

Although not highly regioselective for the desired 1H-triazole, the reaction has the advantage of great tolerance to functional groups (polar amines and sulfonamide moieties of the triazole fragment, and free carboxylic acid/carboxylate salts of the carbamate fragment), which enabled the synthesis of various substrates, by coupling either ester precursors or directly the free carboxylates to the triazolyl fragments.

One of the challenges accompanying the synthesis of this series was the preparation of the corresponding aniline precursors. Suzuki coupling of an appropriate p-amino-phenyl boronic acid and an aryl/heteroaryl bromide ester was convenient for preparing these substrates, due to the ease of the method and the availability of various aryl and heteroaryl bromides and boronic acids. To this end, we screened various conditions, both benchtop and microwave reactions, and palladium sources to establish an efficient method for the C—C (sp²-sp²) bond formation for the desired substrates. Use of [1,1′-bis(diphenylphosphino) ferrocene] dichloro palladium in N,N-Dimethylformamide provided optimal results among the conditions screened. Applying this protocol, the synthesis of aryl-heteroaryl carboxylate analogs 3-6 proceeded through Method A (Scheme 2A), where Boc-protected amines were prepared via Suzuki coupling and were subsequently deprotected to form the corresponding biaryl carbamate esters. Upon urea formation, the esters were converted to carboxylic acids via hydrolysis mediated by Li-salts, as previously reported. Synthesis of simple analogs 7-9 required only two-steps (carbamate and urea formation-Method B, Scheme 2A), since the diphenyl aniline precursors were commercially available.

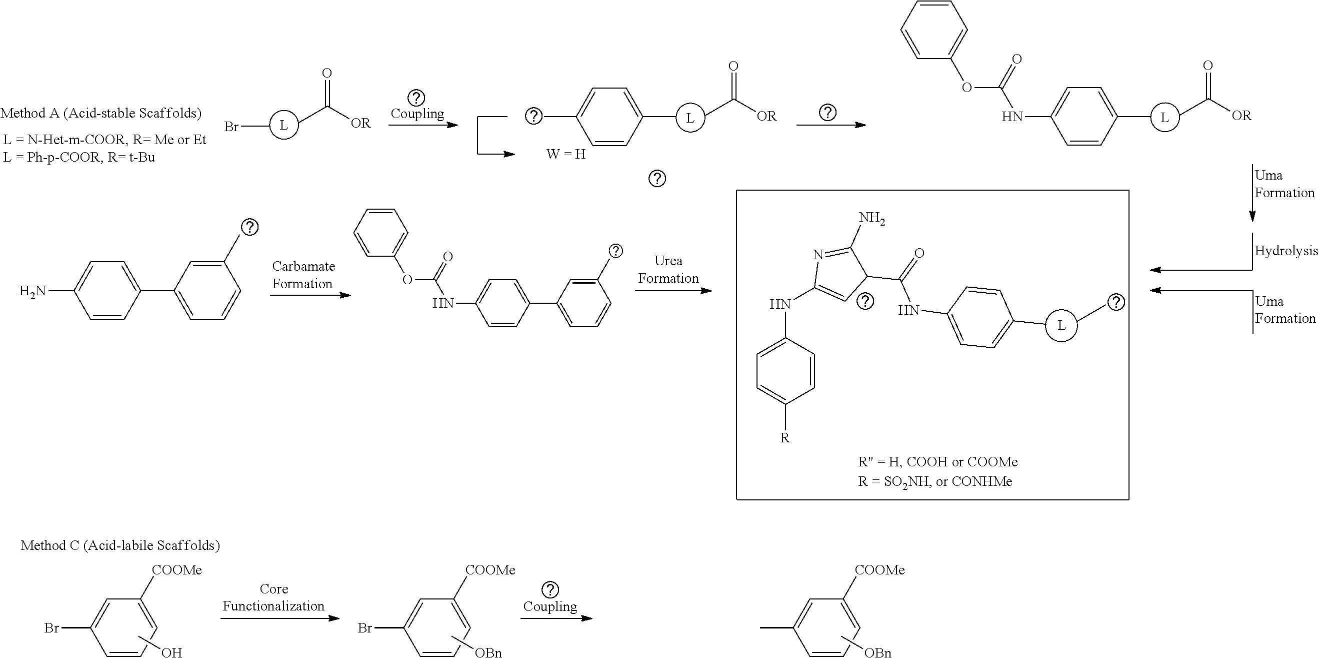

Compound 10 was a special case. Although 4-(p-aminophenyl) benzoic acid is commercially available, efforts to proceed with Method B proved fruitless, since the corresponding carbamate was unstable. We therefore had to adopt a slightly different strategy. Starting with 4-bromobenzoic acid, we installed a t-Butyl ester and then followed the steps of Method A. Reaching the last step, the t-Bu group appeared to be quite sturdy and required strong acidic conditions to afford the hydrolysis. The urea core remained unaffected under stirring with neat trifluoroacetic acid, providing carboxylate analog 10.

None of these routes was viable for the synthesis of diphenyl-benzyloxy analogs. Firstly, it was observed that the benzyloxy moiety was acid-sensitive and thus would be incompatible with the current Boc-protection strategy. Furthermore, core functionalization with the OBn moiety late in the synthesis would require a very complex protection strategy and add burdensome steps to the synthesis. To overcome this challenge, we opted to carry-out the Suzuki reaction directly with the unprotected aniline. This proved to be viable, though much longer reaction times were required.

An additional challenge was the hydrolysis of the ester. Hydrolysis mediated with Li salts was dependent on the presence of an α- or β-heteroatom adjacent to the carbonyl group. In addition, hydrolysis with Li salts became less efficient as substrates became more complex. The most convenient solution was to carry out hydrolysis earlier in the synthesis. The aniline methyl ester precursors seemed ideal for hydrolysis using strong bases, since carbamates and ureas are unstable to hydroxide bases. Proceeding with this strategy followed by carbamate and urea formation of the free carboxylates of the benzyloxy-phenyl analogs, the desired compounds 11-14 were prepared.

### Crystal Structure Determination and Refinement

Crystals of JH2 in complex with compounds 6 and 11 were obtained using co-crystallization methods described previously. X-ray data on frozen crystals were collected on a Rigaku MicroMax-007HF+Xray generator (Cu rotating anode; λ=1.54 Å) equipped with a Rigaku Saturn 944+CCD detector. Data sets were processed with XDS. Phases were determined by molecular replacement with the program PHASER using a protein chain from the previously solved structure PDB 60CC as the initial search model. All model building into electron density was performed with COOT and the structures were refined using Phenix Refine. For each refinement, 5% of all reflections were omitted and used for the calculation of Rfree. Successive rounds of simulated annealing, XYZ coordinate, and individual B-factor refinement were performed until acceptable R factors, geometry statistics, and Ramachandran statistics were achieved. All atomic coordinates and structure factors and have been deposited in the Protein Data Bank with PDB ID codes 7SZW and 7TOP. All figures were prepared by PYMOL.

### Lead Optimization

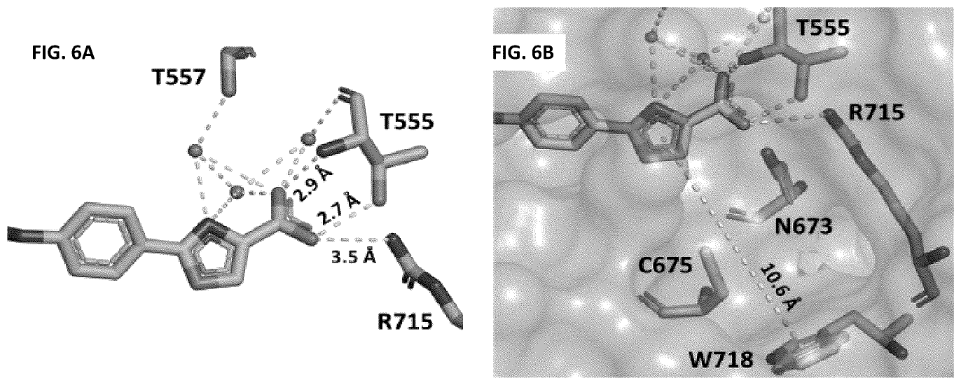

Compound 2 was previously identified as a strong JAK2 JH2 binder, with a binding affinity of 0.346±0.034 UM and 19-fold selectivity over JH1. With this starting point, we aimed to further optimize the affinity and selectivity to a level that would be propitious for testing in cells. The first step was to find alternatives for the oxazole ring that could lead to improved interactions of the attached carboxylic acid with T555, T557, and R715. Guided by the JH2 structure in complex with 2 (PDB: 60CC), some goals were envisioned: (A) improve the distance and the directionality of the hydrogen bond between the carboxylate moiety and R715 (FIG. 3A) and (B) project a substituent into the groove terminating in W718. This feature seemed readily accessible to our series, wherein W718 is 10.6 Å from the center of the oxazole ring of compound 2 (FIG. 6B).

### Ring Optimization

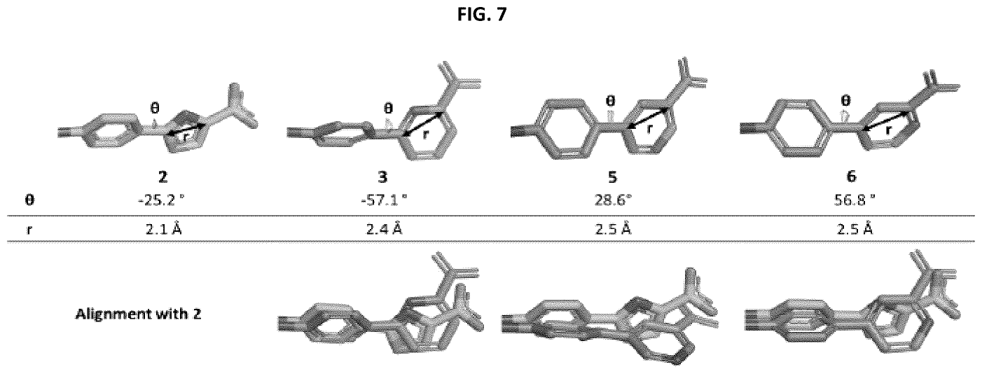

Seeking alternatives to the oxazole ring, we decided to explore 6-membered N-heterocycles. Modeling with BOMB revealed promising pyridine and pyrazine analogs. As seen in FIG. 7, pyridine heterocycles presented the opportunity to project the carboxylic acid moiety closer to the T555-T557-R715 cluster (FIG. 7, distances r) and at different directionality (FIG. 4, dihedrals 0). Utilizing these heterocycles, we could also alter the ring electronics without inducing substantial changes to the core (FIG. 7, alignments).

The 2-pyridyl analog, 3, which positions the N-atom similarly to the oxazole ring (FIG. 7, alignment) exhibited a 3-fold loss in binding affinity compared to 2 (Table 1), indicating possible disruption of the elaborate hydrogen-bonding network that the oxazolic-N was facilitating. The pyrazine analog 4 could participate in a similar hydrogen-bonding network and provide an additional hydrogen-bond acceptor; however, it turned out to be an even weaker binder. Moving the N around the ring, the 3-pyridyl analog 5 appeared promising bringing the carboxylate closer to R715 at 2.8 Å, and it yielded a Kd of 0.652±0.045 μM. The 4-pyridyl analog 6 optimized the N-position in the ring with a K_{d} of 0.394±0.044 μM, similar to that for 2. A crystal structure of 6 bound to JAK2 JH2 was obtained and showed an extensive hydrogen-bonding network including a hydrogen bond between the pyridyl-N and a water molecule that bridges with R715 and N673 (FIG. 8).

The biphenyl-3-carboxylate analog 7 was also prepared as a reference for the SAR on the 6-membered rings. Curiously, 7 exhibited enhanced binding with a K_{d} of 0.129±0.002. An explanation may be that the rigid, electron-rich biphenyl carboxylate improves the cation-π interaction with K581, which is discussed further below. We then sought to quantify the importance of the carboxylate group by preparing the methyl ester 8 and the unsubstituted biphenyl analog 9. Both compounds were found to lose about 3 orders of magnitude in binding affinity compared to 7 (Table 1). Then, in order to test placement of the carboxylate group closer to R715, the para isomer of 7, 10, was prepared. The binding affinity of 10, 0.155±0.013 μM, turned out to be very similar to that of 7; however, its synthesis required 3 additional steps owing to the instability of the carbamate of the unprotected carboxylic acid. Therefore, 7 was selected for further optimization.

### Growing Towards W718

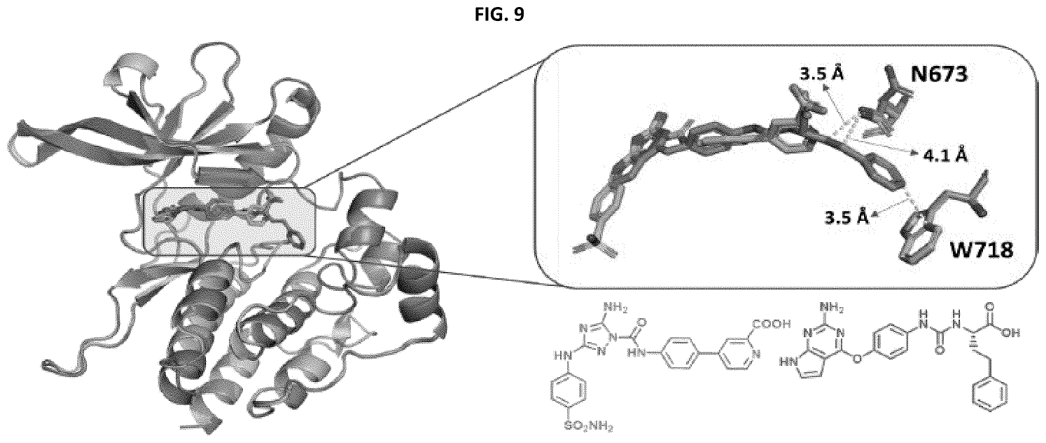

In the pyrrolopyrimidine series described herein, we identified a phenethyl group as a viable substituent to project into the W718 pocket. To consider addition of a similar group for the diaminotriazole series, the crystal structures of 6 (FIG. 9, cyan) and a pyrrolopyrimidine ligand (FIG. 9, magenta) bound to WT JAK2 JH2 were overlaid. The protein backbones align with an RMSD of 0.178 Å and upon closer inspection, the best position for a phenethyl or isosteric group to be placed should be ortho to the carboxylic acid.

In the crystal structure of JAK2 JH2 with the pyrrolopyrimidine ligand, N673 was resolved in two different conformations, either “inward” close to the ligand, or pointing “outward”, to accommodate the phenethyl group (FIG. 9). The inwards conformation was in close vicinity to the phenyethyl moiety, 4.1 Å away from the geminal (benzylic), and 3.5 Å away from the vicinal carbon to phenyl. As such, it seemed that a benzyloxy group might form a hydrogen bond with the terminal amino group of N673 and extend the phenyl group to W718. Thus, the benzyloxy derivative 11 was prepared. It gave a Kad of 0.03710±0.0005 UM with WT JAK2 JH2, almost 3.5x more potent than 7.

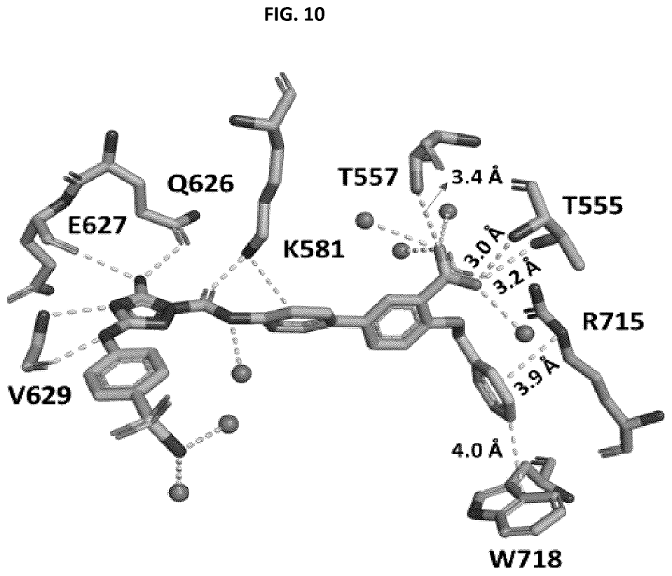

Crystallographic characterization of 11 in complex with JH2 WT (PDB ID: 7TOP) confirmed the design. The benzyloxy moiety is projected to the bottom of the W718 pocket (FIG. 10). It is engaged in an edge-to-face aryl-aryl interaction W718, while R715 lies parallel to the benzyloxy substituent and forms a cation-x interaction with the terminal phenyl ring. These features justify the boost in binding affinity (FIG. 10). The usual cation-x interaction with K581 is also present, and there is an extensive hydrogen bonding network for the carboxylate group with four water molecules, T555, and T557. In this structure, N673 is solely in the “outward” conformation forming hydrogen bonds to neighboring P700, 1702, and R715.

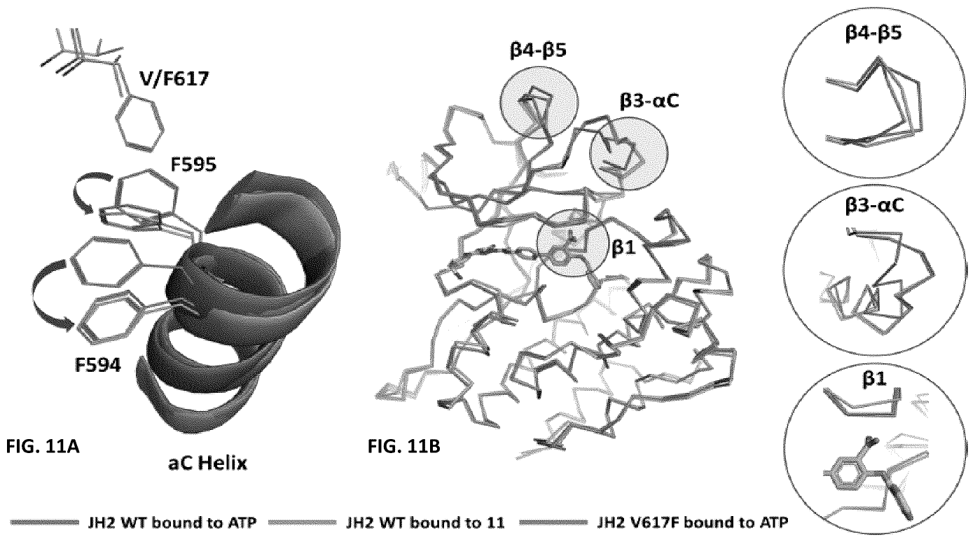

Without being bound by theory, due its relative rigidity and size, 11 also distorts the structure of JAK2 JH2 upon binding, most prominently in the β3-αC and β4-β5 loops. As illustrated in FIGS. 11A-11B, binding of 11 causes a domino effect, shifting F595 and F594, and the β3-αC and β4-β5 loops into a conformation previously observed in the crystal structure of the V617F mutant of JAK2 JH2 with ATP (FIG. 11A). In addition, the carboxylate moiety of 11 pushes the backbone of residues Q553-G554-T555 (β1 region) upwards (FIG. 11B). The observed structural changes are likely induced by the compound rather than arising from crystal packing effects, as soaking 11 with crystals of apo JAK2 JH2 was unsuccessful; co-crystallization was needed to generate the protein-ligand complex in a new space group. Finally, given the success with 11, we wanted to explore its meta-benzyloxy isomer, 12. Though a better binder than 7, 12 turned out to be a 2-fold weaker binder than 11 with a Kd of 0.075±0.015 UM for WT JAK2 JH2 (Table 1).

### Selectivity of Compounds of Formula I

An important next step was to evaluate the selectivity of compound 6 and the potent compounds 11-14 with regard to WT JH1, WT JH2, and the V617F JH2 domains. Kd results (Table 3) were obtained via the competitive fluorescence polarization (FP) assay. As expected, the compounds did not show any significant selectivity for the V617F JH2 variant compared to WT JH2, since the two domains are identical in the vicinity of the ATP-binding site. An exception to this was compound 6, which exhibited 3-fold selectivity toward the mutant JH2. On the other hand, the binding affinities of the tested compounds for the JH1 domain showed only minimal variation throughout lead optimization, ranging from 3-8 μM (Table 3). In combination with the increased affinity for the WT JAK2 JH2, high selectivity was achieved. Compound 6 showed ca. 20-fold selectivity for JAK2 JH2, similar to the parent 2 (ca. 19-fold). Appending the benzyloxy moiety had a profound impact on the selectivity, with the sulfonamide 11 as ca. 155-fold selective and its N-methyl amide analog 13 as ca. 200-fold selective. The corresponding m-benzyloxy analogs 12 and 14 are less selective, ca. 45-fold for 12 and 134-fold for 14.

TABLE 3
Selectivity of Compounds of Formula I

| | | | | JH2/JH1 |
|---|---|---|---|---|
| Compound | WT JH1 | WT JH2 | V617F JH2 | Selectivity |
| 2 (JAK190) | 6.6 ± 0.90 | 0.346 ± 0.034 | 0.478 ± 0.135 | 19x |
| 6 (JAK249) | 8.1 ± 1.2 | 0.394 ± 0.044 | 0.131 ± 0.020 | 20x |
| 11 (JAK315) | 5.75 ± 0.23 | 0.03710 ± 0.0005 | 0.033 ± 0.001 | 155x |
| 12 (JAK344) | 3.36 ± 0.54 | 0.075 ± 0.015 | 0.062 ± 0.001 | 45x |
| 13 (JAK335) | 6.81 ± 0.93 | 0.0334 ± 0.0033 | 0.043 ± 0.005 | 200x |
| 14 (JAK359) | 5.91 ± 0.47 | 0.044 ± 0.003 | 0.050 ± 0.002 | 134x |

In various embodiments, the compound of Formula I has a JH2/JH1 selectivity of about 10× to about 200×, or 10, 20, 30, 40, 50, 60, 70, 80, 90, 100, 110, 120, 130, 140, 150, 160, 170, 180, 190, or about 200×, or a JH2/JH1 selectivity of greater than 200×. A designation such as “10×” as used herein, for example, means a 10-fold selectivity for JH2 over JH1.

### Cell Assay Results

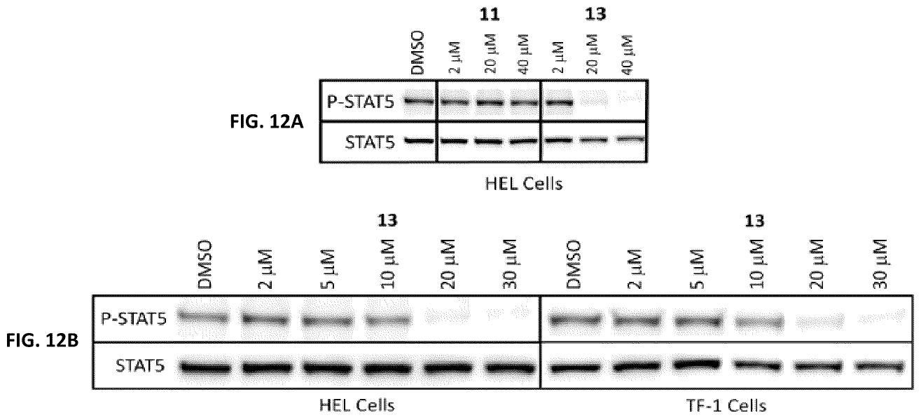

The improved affinity for the WT JAK21 JH2, the large selectivity window for binding to the JH2 domain in preference to the JH1 domain, and the promising permeability results for compounds 11-14 encouraged us to proceed with initial studies in cells. Our first goal was to test if 11 and 13 were active in HEL cells, which express only the V617F JAK2 variant. Compound 13 showed complete inhibition of STAT-5 phosphorylation at 20 μM, and proved to be more active than 11, which did not show any inhibition up to 40 μM with 1-hour incubation (FIG. 12A). The increased potency of compound 13 was surprising given the PAMPA results and the similarity in K_{d}'s with the JH1 and JH2 domains. The PAMPA model, of course, provides a highly simplified model of a cell membrane. For reference, some cell experiments were also carried out with ruxolitinib, an ATP-competitive inhibitor of the JAK2 kinase domain, and 1, as a non-selective JAK2 kinase/pseudokinase binder. Ruxolitinib was tested at only one concentration (2 μM), and it showed complete inhibition of STAT-5 phosphorylation for both HEL and TF-1 cells. These results indicated that it has an IC₅₀ below 2 μM, which is in accord with literature reports of IC₅₀ values of 274 nM and 30-100 nM. Compound 1 exhibited the same inhibition patterns for HEL and TF-1 cells, with an estimated IC₅₀ between 1-5 μM for HEL cells.

Given 13's cellular activity, its effects in HEL cells and against TF1 cells (expressing only WT JAK2) were examined in more detail. The compound was tested at a range of concentrations 2-30 μM and the results indicated that inhibition was observed between 10-20 μM for both HEL and TF-1 cells (FIG. 12B). The time-dependence of the activity of 13 was also investigated with experiments covering 3-48 h of incubation. At 3-24 h no difference was observed in inhibition compared to the 1-h incubation, suggesting that equilibrium of 13 within the cells was achieved. Given these results, it is unclear whether the effect of 13 is due to JH2 and/or JH1-binding. For the m-benzyloxy analogs 12 (sulfonamide) and 14 (N-Me amide), no inhibition was observed in HEL cells up to 25 μM for 12 and up to 30 μM for 14. Thus, the cell assay results are sensitive to the structural details with only 13 among 11-14 showing inhibition of phosphorylation in HEL cells.

### Synthesis of Substituted 1H-1,2,4-Triazoles

General Method A: Suzuki Coupling of Bromides with Phenylboronic Acids

In a flame-dried vial equipped with a rubber septum was added appropriate bromide (1.61-1.94 mmol, 1.0 eq.), appropriate boronic acid (1.0 eq.) and cesium carbonate (2.0-3.0 eq.) in 8-9.4 mL anhydrous dimethylformamide (˜4.7-5.0 mL/mmol). The mixture was degassed under vigorous stirring for 20-30 min, and subsequently Pd(dppf)Cl₂ (0.10 eq.) was added. The mixture was flushed with nitrogen and was heated to 60° C. for the indicated time. Unless otherwise stated, solvent was evaporated under reduced pressure, and the residue was diluted with 40 mL ethyl acetate. The organic phase was washed with water (3 times×10 mL) and with brine (1 time×10 mL). The product was additionally back-extracted from the aqueous phase with ethyl acetate (3 times×20 mL). The combined organic phase was dried under sodium sulfate, and after solvent evaporation the mixture was chromatographed using a gradient of ethyl acetate in hexanes.

### General Method B: Deprotection of Boc-Anilines

Appropriate Boc-aniline (1.0 eq) was dissolved in anhydrous dichloromethane (2.6 mL/mmol). The reaction mixture was cooled to 0° C. and trifluoroacetic acid (34.0 eq. unless otherwise stated) was added dropwise. At the end of the addition, the reaction was warmed to rt and was allowed to run for the indicated time. The pH was then adjusted to ˜ 8 with a sat. solution of sodium bicarbonate (addition at 0° C.). The aqueous phase was extracted with ethyl acetate (5 times×20 mL). The combined organic phase was washed with brine (2 times×20 mL), dried over sodium sulfate, and solvent was evaporated to afford the title product.

### General Method C: Carbamate Formation

Appropriate aniline (1.0 eq.) was suspended in a mixture of tetrahydrofuran/water (˜2:1, as indicated). The mixture was cooled at 0° C. and sodium bicarbonate (1.2-2.2 eq.) was added. A solution of phenyl chloroformate (1.05 eq.) in tetrahydrofuran (1 mL/mmol) was slowly added to the mixture. Upon addition, the reaction was allowed to run at 0° C. for the indicated time, at which point TLC indicated completion. Unless otherwise stated, the mixture was diluted with 30-40 mL ethyl acetate, and was washed with water (3 times×10 mL) and brine (2 times×10 mL). The aqueous phase was back-extracted with ethyl acetate 2 times, and the combined organic phase was washed with brine and dried over sodium sulfate. Solvent was evaporated to afford the title products.

### General Method D: Urea Formation

Mixture A: Unless otherwise stated, in a flame-dried vial, fragment A1 or A2 (1.0 eq) was suspended in anhydrous dioxane (1.4-6.8 mL/mmol) and the mixture was heated for 5-10 min at 100° C., followed by the addition of triethylamine (1.0-2.1 eq). The mixture was allowed to stir for another 10 min and subsequently was added dropwise to Mixture B. The vial was rinsed with anhydrous dioxane (3 mL/mmol substrate) which was also added to Mixture B.

Mixture B: In a flame-dried vial equipped with a rubber septum, appropriate carbamate (1.0 eq.) in anhydrous dioxane (1.9-6.8 mL/mmol) was heated at 100° C. for 10 min, under nitrogen. 1.0-2.1 eq. triethylamine was added, followed by the addition of Mixture A.

The reaction ran at 100-110° C. for the time indicated in each case. Workup details are reported in the individual procedures.

### General Method E: Hydrolysis of Esters Mediated by Li Salts

Appropriate ester (1.0 eq.) was suspended in a mixture of acetonitrile (43 mL per mmol of ester) and 2 vol % water. 1,5-Diazabicyclo[4.3.0]non-5-ene [DBN] (3.0 eq.) and lithium bromide (10.0 eq.) were added to the reaction, and then the mixture was allowed to stir at rt for the indicated time, when MS indicated completion.

Workup: Solvent was evaporated under a stream of nitrogen and an amount of water (29 mL/mmol of substrate) was added to the residue. The pH was adjusted to ˜4 (using 1N HCl and sat. sodium bicarbonate solution), and the mixture was kept at low temperature overnight (−20° C.). The mixture was then centrifuged, and the precipitate was collected, dried, and purified with reverse phase HPLC(System A), using batches of 10 mg from the crude mixture. HPLC fractions were lyophilized to afford the title product.

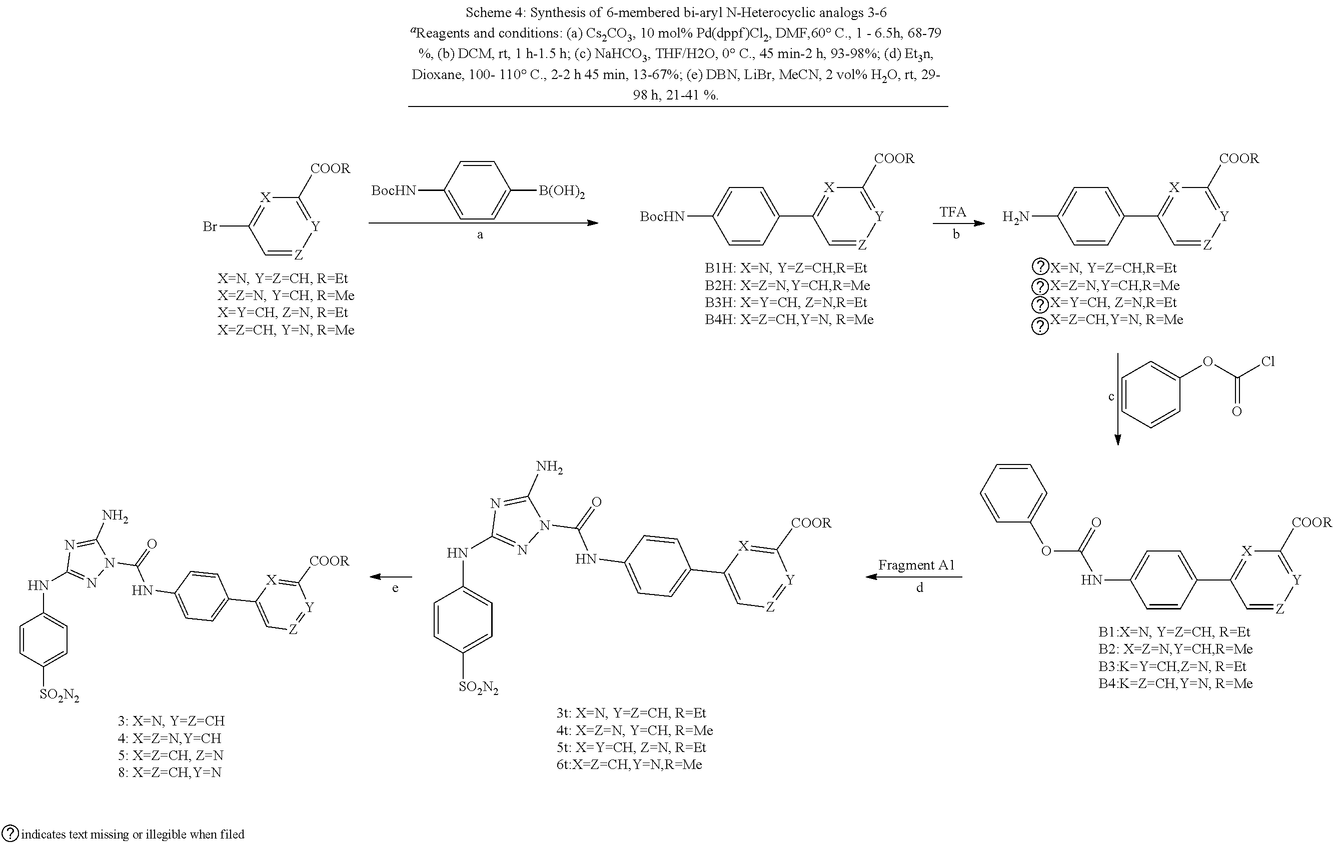

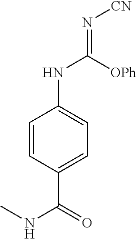 [SMILES: CNC(=O)c1ccc(N/C(=N/C#N)Oc2ccccc2)cc1]

### Phenyl N′-cyano-N-(4-(methylcarbamoyl)phenyl)carbamimidate (Fragment A2i)

4-amino-N-methylbenzamide (1.00 g, 6.66 mmol) was mixed with diphenyl N-cyanocarbonimidate (2.38 g, 9.99 mmol). The reaction was allowed to run for 28 h, and processed as described in General Method A. Off-white solid. Yield: 1.07 g, 55%. HRMS (ESI) m/z calculated for C₁₆H₁₄N₄O₂ [M+H]⁺=295.1190. found: 295.1190. 1H NMR (600 MHz, DMSO-d₆) δ 11.02 (s, 1H), 8.42 (q, J=4.4 Hz, 1H), 7.87-7.83 (m, 2H), 7.58-7.53 (m, 2H), 7.48-7.43 (m, 2H), 7.35-7.28 (m, 3H), 2.78 (d, J=4.5 Hz, 3H). ¹³C NMR (151 MHz, DMSO-d₆) δ 165.89, 151.55, 138.50, 131.69, 129.87, 127.78, 126.30, 122.78, 120.70, 117.22, 113.00, 26.23.

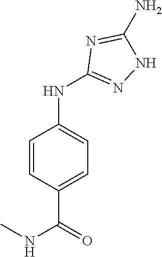 [SMILES: CNC(=O)c1ccc(Nc2n[nH]c(N)n2)cc1]

### 4-((5-amino-1H-1,2,4-triazol-3-yl)amino)-N-methylbenzamide (Fragment A2)

Phenyl N′-cyano-N-(4-(methylcarbamoyl)phenyl)carbamimidate (588.6 mg, 2.0 mmol) and hydrazine (120 μL, 4.0 mmol) were mixed and allowed to react for 3h, as described in General Method B. Off-white solid. Yield: 355.3 mg, 76.5%. HRMS (ESI) m/z calculated for C₁₀H₁₂N₆O [M+H]⁺=233.1145. found: 233.1148. 1H NMR (400 MHZ, DMSO-d₆) δ 11.25 (s, 1H), 9.00 (s, 1H), 8.11 (q, J=4.2 Hz, 1H), 7.67 (d, J=8.8 Hz, 2H), 7.50 (d, J=8.8 Hz, 2H), 5.93 (s, 2H), 2.74 (d, J=4.5 Hz, 3H). ¹³C NMR (151 MHz DMSO-d₆) δ 166.51, 157.46, 155.44, 145.04, 127.85, 124.07, 114.45, 26.10.

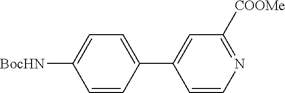 [SMILES: COC(=O)c1cc(-c2ccc(NC(=O)OC(C)(C)C)cc2)ccn1]

### Methyl 4-(4-((tert-butoxycarbonyl)amino)phenyl)picolinate

In a flame-dried vial equipped with a rubber septum was added methyl 4-bromopicolinate (399.7 mg, 1.85 mmol), (4-((tert-butoxycarbonyl)amino)phenyl)boronic acid (438.6 mg, 1.85 mmol) and cesium carbonate (1206 mg, 3.70 mmol) in 9 mL anhydrous dimethylformamide. The mixture was degassed under vigorous stirring for 20 min, and subsequently Pd(dppf)Cl₂ (139.0 mg, 0.19 mmol) was added. The mixture was flushed with nitrogen and was heated to 60° C. for 1h 25 min, when TLC indicated completion. Solvent was evaporated under reduced pressure, and the residue was diluted with 40 mL ethyl acetate. The organic phase was washed with water (3 times×10 mL) and with brine (1 time×10 mL). The product was additionally back-extracted from the aqueous phase with ethyl acetate (3 times x 20 mL). The combined organic phase was dried under sodium sulfate, and after solvent evaporation the mixture was chromatographed using a gradient of ethyl acetate in hexanes. White Solid. Yield: 416.0 mg, 68%. HRMS (ESI) m/z calculated for C₁₈H₂₀N₂O₄ [M+H]⁺=329.1496. found: 329.1489. 1H NMR (600 MHZ, DMSO-d₆) δ 9.63 (s, 1H), 8.70 (d, J=5.1 Hz, 1H), 8.26 (d, J=1.5 Hz, 1H), 7.92 (dd, J=5.1, 1.8 Hz, 1H), 7.82-7.78 (m, 2H), 7.63 (d, J=8.6 Hz, 2H), 3.91 (s, 3H), 1.49 (s, 9H). ¹³C NMR (151 MHZ, DMSO-d₆) δ 165.40, 152.65, 150.37, 148.29, 147.85, 141.26, 129.36, 127.42, 123.72, 121.22, 118.43, 79.46, 52.48, 28.09.

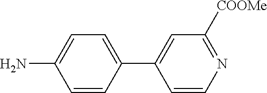 [SMILES: COC(=O)c1cc(-c2ccc(N)cc2)ccn1]

### Methyl 4-(4-aminophenyl)picolinate

Methyl 4-(4-((tert-butoxycarbonyl)amino)phenyl)picolinate (413.0 mg, 1.26 mmol) was dissolved in 3.2 mL anhydrous dichloromethane. The reaction mixture was cooled to 0° C. and trifluoroacetic acid (3.2 mL, 41.79 mmol) was added dropwise. At the end of the addition, the reaction was warmed to rt and was allowed to run for 1h 10 min. The pH was then adjusted to ˜ 8 with a sat. solution of sodium bicarbonate (addition at 0° C.). The aqueous phase was extracted with ethyl acetate (5 times×20 mL), and the combined organic phase was dried over sodium sulfate and solvent was evaporated to afford the title product. Light-brown solid. Yield: 481.5 mg (solid still wet), quant. MS (ESI) m/z=229.1 [M+H]⁺. ¹H NMR (600 MHZ, DMSO-d₆) δ 8.59 (d, J=5.2 Hz, 1H), 8.17 (d, J=1.5 Hz, 1H), 7.81 (dd, J=5.2, 1.9 Hz, 1H), 7.59 (d, J=8.6 Hz, 2H), 6.68 (d, J=8.6 Hz, 2H), 5.64 (s, 2H), 3.89 (s, 3H). ¹³C NMR (151 MHZ, DMSO-d₆) δ 165.62, 150.82, 150.14, 148.50, 148.10, 127.72, 122.42, 122.29, 120.11, 114.12, 52.39.

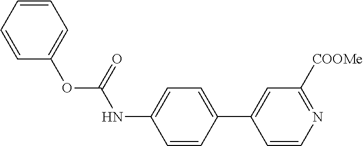 [SMILES: COC(=O)c1cc(-c2ccc(NC(=O)Oc3ccccc3)cc2)ccn1]

### Methyl 4-(4-((phenoxycarbonyl)amino)phenyl)picolinate

Methyl 4-(4-aminophenyl)picolinate (287.6 mg, 1.26 mmol) was suspended in 3.1 mL tetrahydrofuran and 1.3 mL water. The mixture was cooled at 0° C. and sodium bicarbonate (126.0 mg, 1.50 mmol) was added. A solution of phenyl chloroformate (0.17 mL, 1.32 mmol) in 1.3 mL tetrahydrofuran was slowly added to the mixture. Upon addition, the reaction was allowed to run at 0° C. for 45 min, at which point TLC indicated completion. The mixture was diluted with 25 mL ethyl acetate, and was washed with water (3 times×8 mL) and brine (2 times×8 mL). The aqueous phase was back-extracted with ethyl acetate and the combined organic phase was dried over sodium sulfate. Solvent was evaporated under reduced pressure to afford the title product as a pale-yellow solid. Yield: 408.0 mg, 93%. MS (ESI) m/z=349.1 [M+H]⁺. ¹H NMR (600 MHZ, DMSO-d₆) δ 10.51 (s, 1H), 8.73 (d, J=5.1 Hz, 1H), 8.31-8.28 (m, 1H), 7.95 (dd, J=5.1, 1.7 Hz, 1H), 7.88 (d, J=8.7 Hz, 2H), 7.69 (d, J=8.6 Hz, 2H), 7.45 (t, J=7.8 Hz, 2H), 7.30-7.24 (m, 3H), 3.91 (s, 3H).

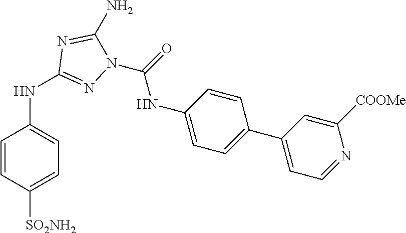 [SMILES: COC(=O)c1cc(-c2ccc(NC(=O)n3nc(Nc4ccc(S(N)(=O)=O)cc4)nc3N)cc2)ccn1]

### Methyl 4-(4-(5-amino-3-((4-sulfamoylphenyl)amino)-1H-1,2,4-triazole-1-carboxamido)phenyl)picolinate

Mixture A: In a flame-dried vial, 4-((5-amino-1H-1,2,4-triazol-3-yl)amino) benzenesulfonamide (81.4 mg, 0.32 mmol) was suspended in 0.6 mL anhydrous dioxane and the mixture was heated for a 5-10 min at 100° C. followed by the addition of triethylamine 44 μL (0.32 mmol). The mixture was allowed to stir for another 10 min and subsequently was added dropwise to Mixture B. The vial was rinsed with 0.6 mL anhydrous dioxane, which was also added to Mixture B.

Mixture B: In a flame-dried vial equipped with a rubber septum, methyl 4-(4-((phenoxycarbonyl)amino)phenyl)picolinate (110.0 mg, 0.32 mmol) in 0.6 mL anhydrous dioxane was heated at 100° C. for 10 min, under nitrogen. 44 μL (0.32 mmol) triethylamine was added, followed by the addition of Mixture A.

The reaction was heated at 110° C. for 2h 45 min. Solvent was evaporated and 20-30 mL of water were added. The aqueous layer was washed with ethyl acetate 3-4 times. The two phases were separated and the water layer was collected and dried to afford the title product. Pale-yellow solid. Yield: 108.2 mg, 67%. HRMS (ESI) m/z calculated for C₂₂H₂₀N₈O₅S [M+H]⁺=509.1350. found: 509.1354. 1H NMR (600 MHZ, DMSO-d₆) δ 9.80 (s, 1H), 9.72 (s, 1H), 8.76 (d, J=5.1 Hz, 1H), 8.34 (d, J=1.4 Hz, 1H), 8.01 (dd, J=5.1, 1.7 Hz, 1H), 7.95 (d, J=8.8 Hz, 2H), 7.90 (d, J=8.7 Hz, 2H), 7.84 (d, J=8.8 Hz, 2H), 7.72 (d, J=8.8 Hz, 2H), 7.48 (s, 2H), 7.16 (s, 2H), 3.93 (s, 3H). ¹³C NMR (151 MHZ, DMSO-d₆) δ 165.36, 156.76, 155.95, 150.47, 148.97, 148.37, 147.65, 143.83, 138.92, 134.93, 131.66, 127.37, 126.81, 124.06, 121.75, 121.52, 116.24, 52.52.

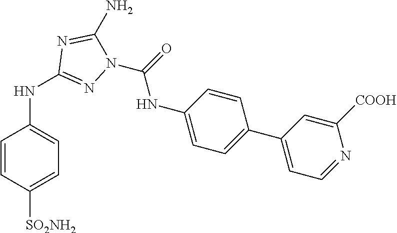 [SMILES: Nc1nc(Nc2ccc(S(N)(=O)=O)cc2)nn1C(=O)Nc1ccc(-c2ccnc(C(=O)O)c2)cc1]

### 4-(4-(5-amino-3-((4-sulfamoylphenyl)amino)-1H-1,2,4-triazole-1-carboxamido)phenyl)picolinic acid (JAK249)

Methyl 4-(4-(5-amino-3-((4-sulfamoylphenyl)amino)-1H-1,2,4-triazole-1-carboxamido)phenyl)picolinate (40.0 mg, 0.08 mmol) was suspended in 3.4 mL acetonitrile containing 68 μL water. DBN (30 μL, 0.24 mmol) was added, followed by the addition of lithium bromide (68.1 mg, 0.79 mmol). The reaction was allowed to stir at rt for 30h. Solvent was evaporated and the crude mixture was suspended in 2.0 mL of water. The mixture was acidified to pH ˜4 with HCl 1N, and was kept at low temperature (−20° C.) overnight. The precipitate was collected by centrifugation and was dried. Batches of 10 mg from the crude mixture were subsequently purified with preparative reverse phase HPLC with a Waters SymmetryPrep™ C8, 19×300 mm column (particle size: 7 μm; pore size: 100 Å) with a gradient of 0.1% formic acid in water and 0.1% formic acid in acetonitrile as the mobile phase, followed by lyophilization, to afford the title product. Yellow solid. Yield: 13.0 mg, 33% (Purity: 97%). ¹H NMR (600 MHZ, DMSO-d₆) δ 9.78 (s, 1H), 9.71 (s, 1H), 8.69 (s, 1H), 8.30 (s, 1H), 7.94-7.87 (m, 5H), 7.84 (d, J=8.1 Hz, 2H), 7.72 (d, J=8.3 Hz, 2H), 7.47 (s, 2H), 7.15 (s, 2H) [Proton of the COOH moiety was not observed]. ¹³C NMR (151 MHZ, DMSO-d₆) δ 166.82, 163.60, 156.73, 155.92, 149.73, 148.96, 147.28, 143.82, 138.64, 134.91, 132.21, 127.24, 126.80, 122.78, 121.76, 120.96, 116.23.

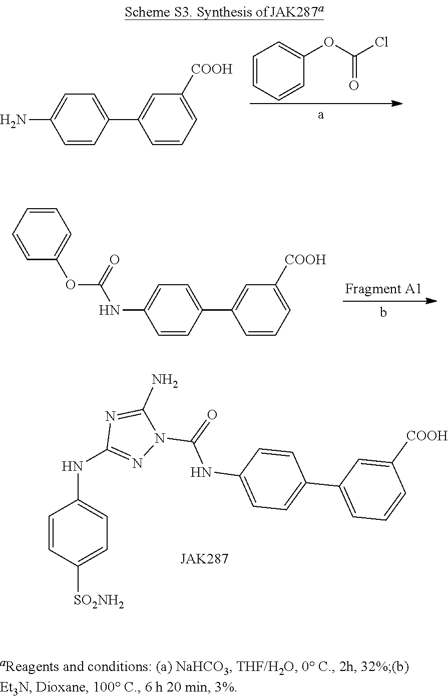 [SMILES: Nc1ccc(-c2cccc(C(=O)O)c2)cc1 ; O=C(Cl)Oc1ccccc1 ; O=C(Nc1ccc(-c2cccc(C(=O)O)c2)cc1)Oc1ccccc1 ; Nc1nc(Nc2ccc(S(N)(=O)=O)cc2)nn1C(=O)Nc1ccc(-c2cccc(C(=O)O)c2)cc1]

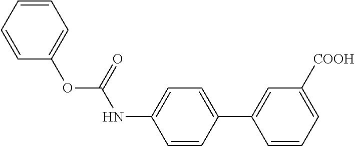 [SMILES: O=C(Nc1ccc(-c2cccc(C(=O)O)c2)cc1)Oc1ccccc1]

### 4′-((phenoxycarbonyl)amino)-[1,1′-biphenyl]-3-carboxylic acid

4′-amino-[1, l′-biphenyl]-3-carboxylic acid (705.8 mg, 3.31 mmol) was suspended in 6.6 mL tetrahydrofuran containing 3.3 mL water. The mixture was brought to 0° C. followed by the addition of sodium bicarbonate (611.6 mg, 7.28 mmol). The mixture was allowed to stir for 5-10 min and then a solution of phenyl chloroformate (0.44 mL, 3.48 mmol) in 3.3 mL tetrahydrofuran was added dropwise. The reaction was allowed to stir for 2 h at 0° C., at which point TLC indicated completion. Water (10 mL) was added to the reaction, followed by methanol, until cloud point. The solid was filtered out and the filtrate was collected and further processed as follows: 25 mL water was added and the pH was adjusted to ˜4 with HCl 1N. The aqueous phase was extracted with ethyl acetate (3 times×40 mL). The combined organic layer was washed with brine (2 times×30 mL) and was dried over sodium sulfate. Solvent was evaporated under reduced pressure to afford the title product. Pale-pink solid. Yield: 348.8 mg, 32%. HRMS (ESI) m/z calculated for C₂₀H₁₅NO₄ [M+H]⁺=334.1074. found: 334.1100. 1H NMR (600 MHz, DMSO-d₆) δ 10.39 (s, 1H), 8.17 (s, 1H), 7.90 (dt, J=7.9, 1.6 Hz, 2H), 7.70 (d, J=8.6 Hz, 2H), 7.64 (d, J=8.5 Hz, 2H), 7.57 (t, J=7.7 Hz, 1H), 7.44 (t, J=7.8 Hz, 2H), 7.26 (dd, J=17.5, 7.7 Hz, 3H). ¹³C NMR (151 MHZ, DMSO-d₆) δ 167.32, 151.70, 150.50, 139.98, 138.54, 133.74, 131.63, 130.61, 129.47, 129.28, 127.85, 127.29, 126.87, 125.51, 121.99, 118.87.

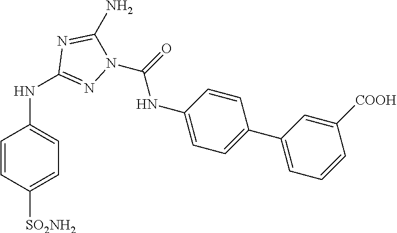 [SMILES: Nc1nc(Nc2ccc(S(N)(=O)=O)cc2)nn1C(=O)Nc1ccc(-c2cccc(C(=O)O)c2)cc1]

### 4′-(5-amino-3-((4-sulfamoylphenyl)amino)-1H-1,2,4-triazole-1-carboxamido)-[1,1′-biphenyl]-3-carboxylic acid (JAK287)

Mixture A: In a flame-dried vial, 4-((5-amino-1H-1,2,4-triazol-3-yl)amino) benzenesulfonamide (89.8 mg, 0.35 mmol) was suspended in 0.5 mL anhydrous dioxane and the mixture was heated for a 10-15 min at 80-100° C., followed by the addition of triethylamine (50 μL, 0.35 mmol). The mixture was allowed to stir for another 10 min and subsequently was added dropwise to Mixture B. The vial was rinsed with 2.0 mL anhydrous dioxane, which was also added to Mixture B.

Mixture B: In a flame-dried vial equipped with a rubber septum, 4′-((phenoxycarbonyl)amino)-[1,1′-biphenyl]-3-carboxylic acid (117.7 mg, 0.35 mmol) in 0.8 mL anhydrous dioxane was heated at 100° C. for 10 min, under nitrogen. Triethylamine (100 μL, 0.70 mmol) was added, followed by the addition of Mixture A.

The reaction was heated at 100° C. for 6h 20 min. Solvent was evaporated under a stream of nitrogen and water was added. The crude mixture was acidified to pH ˜4 with HCl 1N, and the precipitate was collected by centrifugation and dried. The crude mixture was purified in batches of 20 mg using preparative reverse phase HPLC, an Agilent 5 Prep-C18 21× 100 mm column with a gradient of 0.1% formic acid in water and 0.1% formic acid in acetonitrile as the mobile phase. White solid. Yield: 43.7 mg, 25% (Purity: 90-95%)/5.6 mg, 3% (Purity: 97.6%). HRMS (ESI) m/z calculated for C₂₂H₁₉N₇O₅S [M+H]⁺=494.1241. found: 494.1260. 1H NMR (500 MHZ, DMSO-d₆) δ 9.70 (d, J=5.3 Hz, 2H), 8.21 (s, 1H), 7.95-7.90 (m, 2H), 7.83 (t, J=8.7 Hz, 4H), 7.75 (d, J=8.6 Hz, 2H), 7.72 (d, J=8.8 Hz, 2H), 7.58 (t, J=7.7 Hz, 1H), 7.46 (s, 2H), 7.15 (s, 2H). ¹³C NMR (126 MHZ, DMSO-d₆) δ 167.54, 156.72, 155.91, 149.04, 143.86, 139.70, 137.02, 135.22, 134.90, 132.68, 130.31, 129.17, 128.04, 126.98, 126.95, 126.82, 121.96, 116.24.

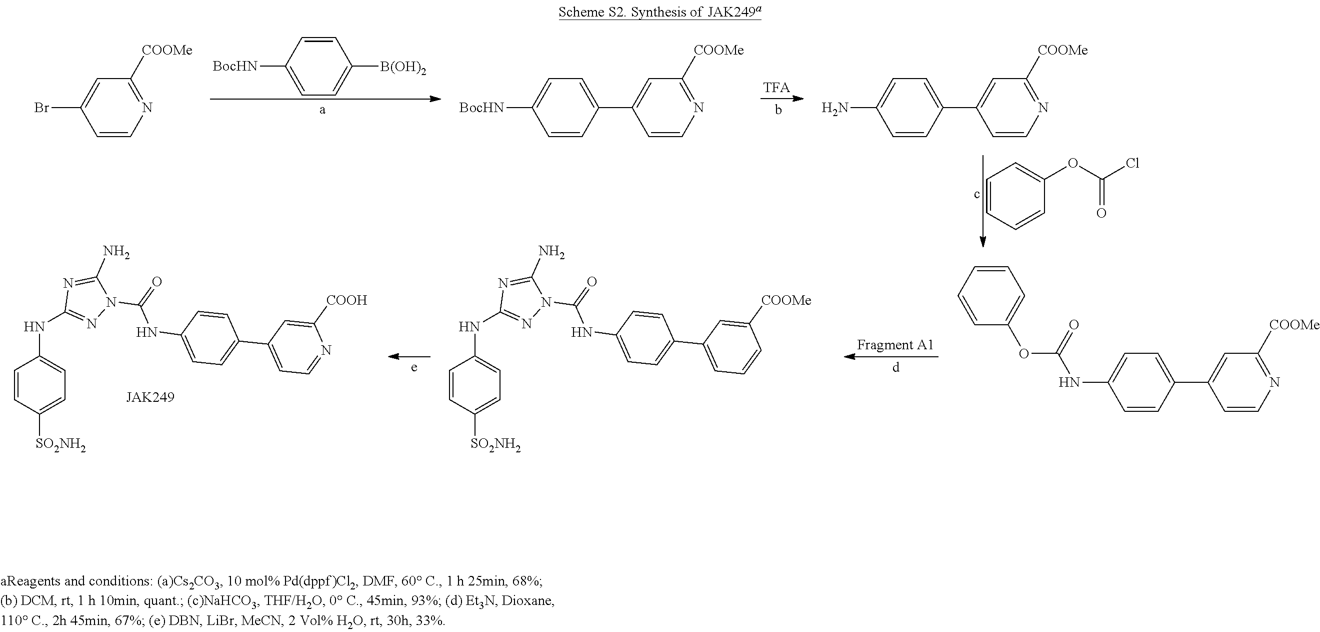

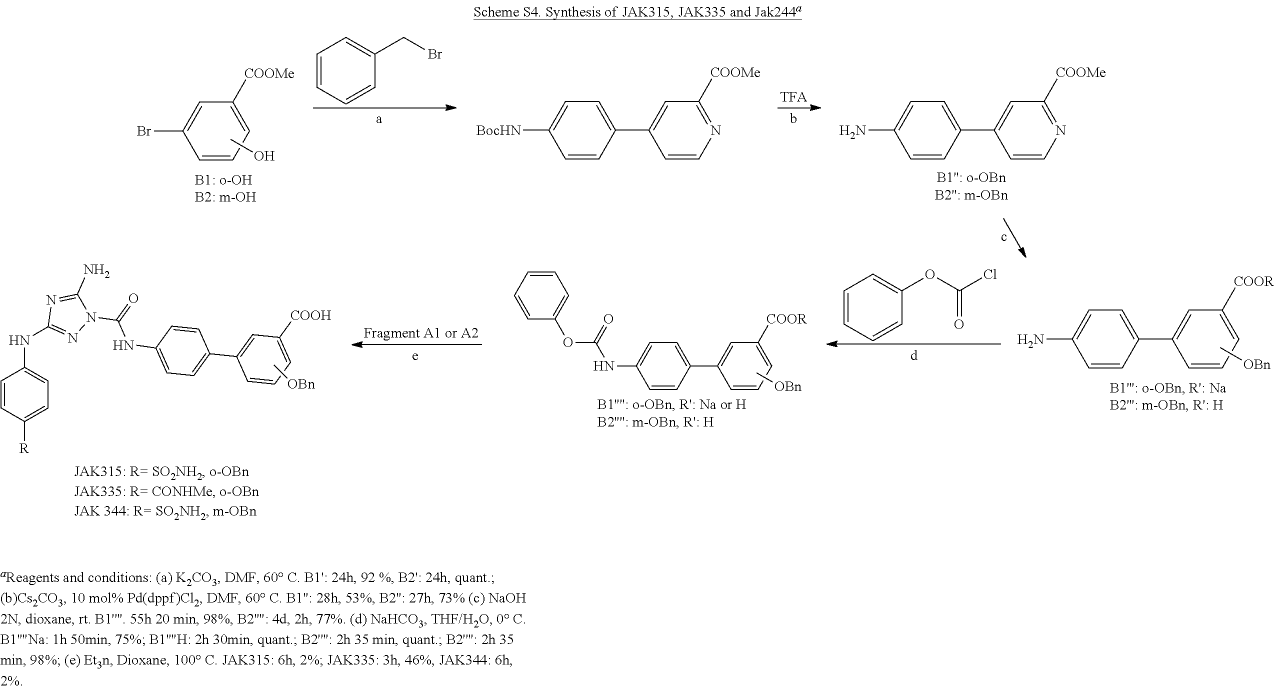

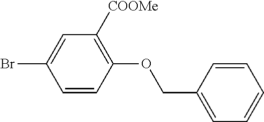 [SMILES: COC(=O)c1cc(Br)ccc1OCc1ccccc1]

### Methyl 2-(benzyloxy)-5-bromobenzoate (B1′)

In an oven-dried vial, methyl 5-bromo-2-hydroxybenzoate (462.1 mg, 2.00 mmol) was combined with potassium carbonate (346.9 mg, 2.51 mmol) in 1.4 mL anhydrous dimethylformamide. The mixture was allowed to stir until homogenized and subsequently benzyl bromide (0.2 mL, 1.67 mmol) was added dropwise. When the addition was complete, the reaction was heated at 60° C. for 24 h, at which point TLC indicated consumption of the starting material. Then solvent was evaporated and the mixture was diluted with 30 mL ethyl acetate. The organic phase was washed with water (3 times×10 mL), NaOH 2N(2 times x 10 mL), brine (2 times×10 mL) and was dried over sodium sulfate. The mixture was filtered and solvent was evaporated under reduced pressure to afford the title product. Light-yellow oil. Yield: 591.9 mg, 92%. HRMS (ESI) m/z calculated for C₁₅H₁₃BrO₃ [M+H]⁺=321.0121. found: 321.0113. 1H NMR (500 MHZ, DMSO-d₆) δ 7.79 (d, J=2.1 Hz, 1H), 7.70 (dd, J=8.9, 2.1 Hz, 1H), 7.47 (d, J=7.4 Hz, 2H), 7.40 (t, J=7.5 Hz, 2H), 7.32 (t, J=7.3 Hz, 1H), 7.22 (d, J=9.0 Hz, 1H), 5.22 (s, 2H), 3.81 (s, 3H). ¹³C NMR (126 MHZ, DMSO-d₆) δ 164.72, 156.39, 136.51, 135.82, 132.84, 128.40, 127.74, 126.98, 122.43, 116.53, 111.57, 69.90, 52.20.

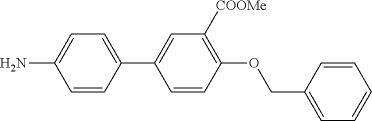 [SMILES: COC(=O)c1cc(-c2ccc(N)cc2)ccc1OCc1ccccc1]

### Methyl 4′-amino-4-(benzyloxy)-[1,1′-biphenyl]-3-carboxylate (B1″)

In a flame-dried vial equipped with a rubber septum was added methyl 2-(benzyloxy)-5-bromobenzoate (515.7 mg, 1.61 mmol), 4-aminophenylboronic acid hydrochloride (279.2 mg, 1.61 mmol) and cesium carbonate (1573.7 mg, 4.83 mmol) in 8 mL anhydrous dimethylformamide. The mixture was degassed under vigorous stirring for 20 min, and subsequently Pd(dppf)Cl₂ (117.8 mg, 0.16 mmol) was added. The mixture was purged with nitrogen and was heated at 60° C. for 28h, when TLC indicated completion. Solvent was evaporated under reduced pressure, and the residue was diluted with ethyl acetate. The organic phase was washed with water (2 times×30 mL) and with brine (2 times×30 mL). The organic phase was then concentrated under reduced pressure, and the mixture was chromatographed using a gradient of ethyl acetate in hexanes to afford the title product. White solid. Yield: 285.4 mg, 53%. HRMS (ESI) m/z calculated for C₂₁H19NO₃ [M+H]⁺=334.1438. found: 334.1431. 1H NMR (500 MHZ, DMSO-d₆) δ 7.80 (d, J=2.0 Hz, 1H), 7.68 (dd, J=8.7, 2.2 Hz, 1H), 7.50 (d, J=7.6 Hz, 2H), 7.40 (t, J=7.5 Hz, 2H), 7.31 (d, J=8.1 Hz, 3H), 7.23 (d, J=8.7 Hz, 1H), 6.63 (d, J=8.2 Hz, 2H), 5.22 (s, 2H), 5.20 (s, 2H), 3.83 (s, 3H). ¹³C NMR (126 MHz, DMSO-d₆) δ 166.24, 155.41, 148.20, 137.05, 133.22, 130.04, 128.36, 127.60, 127.29, 126.94, 126.77, 126.01, 120.78, 114.66, 114.25, 69.74, 51.90.

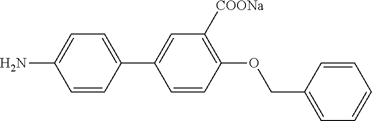

### Sodium 4′-amino-4-(benzyloxy)-[1,1′-biphenyl]-3-carboxylate (B1′″)

Methyl 4′-amino-4-(benzyloxy)-[1,1′-biphenyl]-3-carboxylate (71.3 mg, 0.21 mmol) was mixed with dioxane (5 mL) and NaOH 2N(1.1 mL, 2.10 mmol) was added dropwise. The reaction was allowed to stir at rt for 55h 20 min. Then the mixture was neutralized with HCl 2N(at 0° C.) and solvent was evaporated under reduced pressure. The residue was then chromatographed using a gradient of methanol in dichloromethane to afford the title product. Pale-white solid. Yield: 70.1 mg, 98%. HRMS (ESI) m/z calculated for C₂₀H₁₆NNaO₃ [M+H]⁺=342.1101. found: 342.1094. 1H NMR (500 MHZ, DMSO-d₆) δ 7.76 (d, J=2.5 Hz, 1H), 7.62 (dd, J=8.7, 2.5 Hz, 1H), 7.50 (d, J=7.3 Hz, 2H), 7.39 (t, J=7.5 Hz, 2H), 7.35-7.27 (m, 3H), 7.18 (d, J=8.7 Hz, 1H), 6.65-6.60 (m, 2H), 5.21 (s, 2H). ¹³C NMR (151 MHz, DMSO-d₆) δ 167.58, 155.15, 148.11, 137.18, 133.13, 129.27, 128.33, 127.60, 127.17, 127.09, 126.74, 126.27, 122.60, 114.47, 114.25, 69.74.

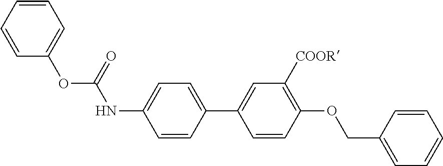

### R′=Na: Sodium 4-(benzyloxy)-4′-((phenoxycarbonyl)amino)-[1,1′-biphenyl]-3-carboxylate (B1″″Na)

### R′=H: 4-(benzyloxy)-4′-((phenoxycarbonyl)amino)-[1,1′-biphenyl]-3-carboxylic acid (B1″″H)

### Method A, for R′=Na: Sodium 4-(benzyloxy)-4′-((phenoxycarbonyl)amino)-[1,1′-biphenyl]-3-carboxylate (B1″″Na)

Sodium 4′-amino-4-(benzyloxy)-[1,1′-biphenyl]-3-carboxylate (67.9 mg, 0.20 mmol) was suspended in a mixture of tetrahydrofuran (1.0 mL) and water (0.5 mL). The mixture was cooled to 0° C. and sodium bicarbonate (20.2 mg, 0.24 mmol) was added, followed by the slow addition of a solution of phenyl chloroformate (26 μL, 0.21 mmol) in 0.5 mL tetrahydrofuran. The reaction was allowed to stir for 1h 50 min at 0° C., at which point TLC indicated consumption of the starting material. The reaction mixture was then diluted with ethyl acetate (20 mL), and was washed with brine (3 times×2-5 mL). The organic phase was collected, solvent was evaporated under reduced pressure, and then the residue was dried in high vacuum overnight to afford the title product. Pale-yellow solid. Yield: 69.3 mg, 75%. HRMS (ESI) m/z calculated for C₂₇H₂₀NNaO₅ [M+H]⁺=462.1312. found: 462.1299. 1H NMR (600 MHZ, DMSO-d₆) δ 10.34 (s, 1H), 7.69 (d, J=2.3 Hz, 1H), 7.57 (s, 4H), 7.56-7.53 (m, 1H), 7.51 (d, J=7.1 Hz, 2H), 7.43 (tt, J=7.6, 2.2 Hz, 2H), 7.39-7.35 (m, 2H), 7.32-7.28 (m, 1H), 7.26 (tt, J=7.5, 1.0 Hz, 1H), 7.25-7.21 (m, 2H), 7.10 (d, J=8.7 Hz, 1H), 5.19 (s, 2H).

### Method B, for R′=H: 4-(benzyloxy)-4′-((phenoxycarbonyl)amino)-[1,1′-biphenyl]-3-carboxylic acid (B1″″H)

Sodium 4′-amino-4-(benzyloxy)-[1,1′-biphenyl]-3-carboxylate (173.5 mg, 0.51 mmol) was suspended in a mixture of tetrahydrofuran (4.0 mL) and water (2.0 mL). The mixture was cooled to 0° C. and sodium bicarbonate (51.4 mg, 0.61 mmol) was added, followed by the slow addition of a solution of phenyl chloroformate (68 μL, 0.54 mmol) in 2.0 mL tetrahydrofuran. The reaction was allowed to stir for 2h 30 min at 0° C., at which point TLC indicated consumption of the starting material. The reaction mixture was then acidified to pH˜4 with HCl 1N and then it was extracted with ethyl acetate (50 mL). The aqueous layer was removed, and the organic phase was washed with water (3 times×5 mL), brine (2 times×5 mL). The organic phase was collected, solvent was evaporated under reduced pressure and then the residue was dried in high vacuum overnight to afford the title product. Pale-yellow solid. Yield: 362.4 mg, quant. (solid contains some solvent remnants). HRMS (ESI) m/z calculated for C₂₇H₂₁NO₅ [M+H]⁺=440.1492. found: 440.1482. 1H NMR (600 MHZ, DMSO-d₆) δ 12.79 (s, 1H), 10.36 (s, 1H), 7.89 (d, J=2.6 Hz, 1H), 7.76 (dd, J=8.7, 2.6 Hz, 1H), 7.65-7.57 (m, 4H), 7.53-7.49 (m, 2H), 7.46-7.42 (m, 2H), 7.42-7.37 (m, 2H), 7.34-7.30 (m, 1H), 7.29-7.25 (m, 2H), 7.25-7.22 (m, 2H), 5.25 (s, 2H).

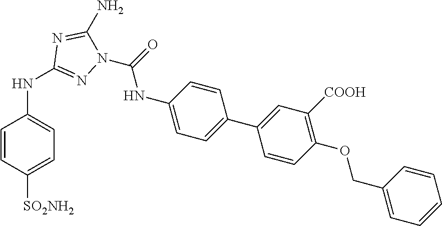 [SMILES: Nc1nc(Nc2ccc(S(N)(=O)=O)cc2)nn1C(=O)Nc1ccc(-c2ccc(OCc3ccccc3)c(C(=O)O)c2)cc1]

### 4′-(5-amino-3-((4-sulfamoylphenyl)amino)-1H-1,2,4-triazole-1-carboxamido)-4-(benzyloxy)-[1,1′-biphenyl]-3-carboxylic acid (JAK315)

Mixture A: In a flame-dried vial, 4-((5-amino-1H-1,2,4-triazol-3-yl)amino) benzenesulfonamide (35.8 mg, 0.14 mmol) was suspended in 0.8 mL anhydrous dioxane and the mixture was heated for a 10-15 min at 100° C., followed by the addition of triethylamine (20 μL, 0.14 mmol). The mixture was allowed to stir for another 10 min and subsequently it was added dropwise to Mixture B. The vial was rinsed with 0.5 mL anhydrous dioxane, which was also added to the Mixture B.

Mixture B: In a flame-dried vial equipped with a rubber septum, sodium 4-(benzyloxy)-4′-((phenoxycarbonyl)amino)-[1,1′-biphenyl]-3-carboxylate (65.0 mg, 0.14 mmol) in 2.0 mL anhydrous dioxane was heated at 80° C. for 10 min, under nitrogen. Triethylamine (20 μL, 0.14 mmol) was added, followed by the addition of Mixture A. The reaction was heated at 100° C. for 6 h. The mixture was then allowed to rest and the supernatant was removed. The residue was triturated with methanol and was dried. Water (1.0 mL) was added and the mixture was acidified to pH˜4 with HCl 1N. The precipitate was collected by centrifugation and was subsequently purified with reversed-phase chromatography using a RediSep Gold C₁₈ reusable column (particle size: 20-40 μm spherical; pore size: 100 Å) with a gradient of 0.1% formic acid in water and 0.1% formic acid in acetonitrile as the mobile phase. White Solid. Yield: 1.7 mg, 2% (Purity: 95.9%). HRMS (ESI) m/z calculated for C₂₉H₂₅N₇O₆S [M+H]⁺=600.1660. found: 600.1670. 1H NMR (600 MHZ, DMSO-d₆) δ 9.69 (s, 1H), 9.66 (s, 1H), 7.89 (s, 1H), 7.83 (d, J=8.7 Hz, 2H), 7.79-7.74 (m, 3H), 7.72 (d, J=8.8 Hz, 2H), 7.68 (d, J=8.5 Hz, 2H), 7.52 (d, J=7.4 Hz, 2H), 7.44 (s, 2H), 7.40 (t, J=7.5 Hz, 2H), 7.32 (t, J=7.3 Hz, 1H), 7.25 (d, J=8.6 Hz, 1H), 7.14 (s, 2H), 5.25 (s, 2H) [Proton of the COOH moiety was not observed]. ¹³C NMR (151 MHz, DMSO-d₆) δ 167.72, 156.69, 156.05, 155.89, 149.03, 143.86, 137.13, 136.29, 134.98, 134.88, 131.67, 129.84, 128.36, 128.00, 127.62, 127.11, 126.80, 126.35, 121.97, 116.22, 114.49, 69.71 (one carbon peak overlaps with the rest in the aromatic region).

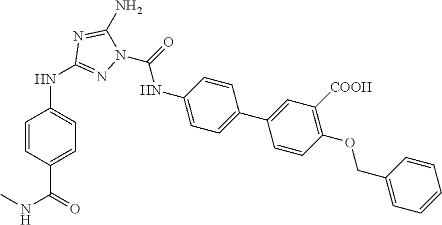 [SMILES: CNC(=O)c1ccc(Nc2nc(N)n(C(=O)Nc3ccc(-c4ccc(OCc5ccccc5)c(C(=O)O)c4)cc3)n2)cc1]

### 4′-(5-amino-3-((4-(methylcarbamoyl)phenyl)amino)-1H-1,2,4-triazole-1-carboxamido)-4-(benzyloxy)-[1,1′-biphenyl]-3-carboxylic acid (JAK335)

Mixture A: In a flame-dried vial, 4-((5-amino-1H-1,2,4-triazol-3-yl)amino)-N-methylbenzamide (58.1 mg, 0.25 mmol) was suspended in 1.7 mL anhydrous dioxane and the mixture was heated for a 10-15 min at 100° C., followed by the addition of triethylamine (35 μL, 0.25 mmol). The mixture was allowed to stir for another 10 min and subsequently was added dropwise to Mixture B. The vial was rinsed with 1.0 mL anhydrous dioxane, which was also added to Mixture B.

Mixture B: In a flame-dried vial equipped with a rubber septum, 4-(benzyloxy)-4′-((phenoxycarbonyl)amino)-[1,1′-biphenyl]-3-carboxylic acid (109.9 mg, 0.25 mmol) in 1.0 mL anhydrous dioxane was heated at 100° C. for 5 min, under nitrogen. Triethylamine (70 μL, 0.50 mmol) was added, followed by the addition of Mixture A. The reaction was heated at 100° C. for 3 h. The reaction mixture was then dried, the residue was triturated with ethyl acetate and it was dried again. Water (1.0 mL) was added and the mixture was acidified to pH˜4 with HCl 1N. The precipitate was collected by centrifugation and it was subsequently purified using preparative reverse-phase HPLC, with an Agilent 5 Prep-C18 21×100 mm column with a gradient of 0.1% formic acid in water and 0.1% formic acid in acetonitrile as the mobile phase. White Solid. Yield: 66.1 mg, 46% (Purity: 95.6%). HRMS (ESI) m/z calculated for C₃₁H₂₇N₇O₅ [M+H]⁺=578.2146. found: 578.2144. 1H NMR (500 MHZ, DMSO-d₆) δ 9.67 (s, 1H), 9.51 (s, 1H), 8.20 (q, J=4.7 Hz, 1H), 7.89 (s, 1H), 7.83-7.75 (m, 5H), 7.73 (d, J=8.7 Hz, 2H), 7.67 (d, J=8.5 Hz, 2H), 7.52 (d, J=7.3 Hz, 2H), 7.44-7.37 (m, 4H), 7.32 (t, J=7.1 Hz, 1H), 7.26 (d, J=8.7 Hz, 1H), 5.25 (s, 2H), 2.76 (d, J=4.4 Hz, 3H) [Proton of the COOH moiety was not observed]. ¹³C NMR (126 MHZ, DMSO-d₆) δ 168.08, 166.44, 156.86, 155.85, 149.06, 143.43, 137.23, 136.34, 135.02, 131.66, 129.36, 128.35, 128.01, 127.86, 127.60, 127.13, 126.74, 126.34, 125.72, 121.76, 116.00, 114.47, 114.29, 69.71, 26.19.

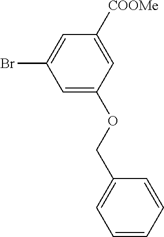 [SMILES: COC(=O)c1cc(Br)cc(OCc2ccccc2)c1]

### Methyl 3-(benzyloxy)-5-bromobenzoate (B2′)

In an oven-dried vial, methyl 3-bromo-5-hydroxybenzoate (1.71 g, 7.40 mmol) was combined with potassium carbonate (1.28 g, 9.26 mmol) in 5 mL anhydrous dimethylformamide. The mixture was allowed to stir until it was homogenized and subsequently benzyl bromide (0.73 mL, 6.17 mmol) was added dropwise. When the addition was complete, the reaction was heated at 60° C. and stirred for 24 h, at which point TLC indicated consumption of the starting material. Solvent was evaporated and the mixture was diluted with 100 mL ethyl acetate. The organic phase was washed with water (4 times×30-40 mL), NaOH 2N (5 times×30-40 mL), brine (4 times×30 mL) and was dried over sodium sulfate. The mixture was filtered, solvent was evaporated under reduced pressure, and the residue was further dried under high vacuum overnight to afford the title product. Gold-yellow oil. Yield: 2.00 g, quant. HRMS (ESI) m/z calculated for C₁₅H₁₃BrO₃ [M+H]⁺=321.0121. found: 321.0108. 1H NMR (600 MHZ, DMSO-d₆) δ 7.64 (t, J=1.5 Hz, 1H), 7.57-7.54 (m, 1H), 7.52 (dd, J=2.4, 1.3 Hz, 1H), 7.47-7.44 (m, 2H), 7.40 (td, J=6.8, 6.4, 1.6 Hz, 2H), 7.37-7.32 (m, 1H), 5.20 (s, 2H), 3.85 (s, 3H). ¹³C NMR (151 MHZ, DMSO-d₆) δ 164.75, 159.31, 136.22, 132.57, 128.51, 128.07, 127.78, 123.99, 122.47, 122.34, 114.65, 69.91, 52.62.

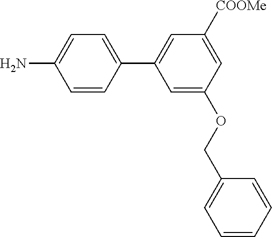 [SMILES: COC(=O)c1cc(OCc2ccccc2)cc(-c2ccc(N)cc2)c1]

### Methyl 4′-amino-5-(benzyloxy)-[1,1′-biphenyl]-3-carboxylate (B2″)

In a flame-dried vial equipped with a rubber septum was added methyl 3-(benzyloxy)-5-bromobenzoate (610.0 mg, 1.9 mmol), 4-aminophenylboronic acid hydrochloride (329.5 mg, 1.9 mmol) and cesium carbonate (1857.2 mg, 5.7 mmol) in 9 mL anhydrous dimethylformamide. The mixture was degassed under vigorous stirring for 20 min, and subsequently Pd(dppf)Cl₂ (139.0 mg, 0.19 mmol) was added. The mixture was flushed with nitrogen and was heated to 60° C. for 27h, when TLC indicated completion. Solvent was evaporated under reduced pressure, and the residue was diluted with ethyl acetate. The organic phase was washed with water (2 times×30 mL), brine (2 times×30 mL) and the organic phase was collected and concentrated under reduced pressure. The crude mixture was further purified with chromatography, using a gradient of ethyl acetate in hexanes, to afford the title product. Pale-yellow solid. Yield: 463.1 mg, 73%. HRMS (ESI) m/z calculated for C₂₁H19NO₃ [M+H]⁺=334.1438. found: 334.1435. 1H NMR (500 MHZ, DMSO-d₆) δ 7.69 (t, J=1.4 Hz, 1H), 7.52-7.46 (m, 2H), 7.45-7.37 (m, 6H), 7.37-7.31 (m, 1H), 6.67-6.62 (m, 2H), 5.33 (s, 2H), 5.23 (s, 2H), 3.86 (s, 3H). ¹³C NMR (126 MHZ, DMSO-d₆) δ 166.16, 158.88, 149.05, 142.64, 136.87, 131.29, 128.44, 127.86, 127.65, 127.37, 125.80, 118.54, 116.58, 114.14, 112.11, 69.45, 52.23.

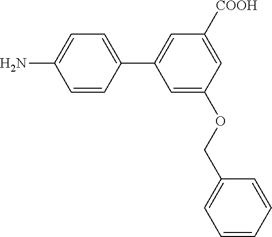 [SMILES: Nc1ccc(-c2cc(OCc3ccccc3)cc(C(=O)O)c2)cc1]

### 4′-amino-5-(benzyloxy)-[1,1′-biphenyl]-3-carboxylic acid (B2′″)

Methyl 4′-amino-5-(benzyloxy)-[1,1′-biphenyl]-3-carboxylate (449.0 mg, 1.35 mmol) was mixed with dioxane (32 mL) and NaOH 2N(6.8 mL, 13.5 mmol) was added dropwise. The reaction was allowed to stir at rt for 4d 2 h. Then the mixture was neutralized with HCl 2N(at 0° C.) and solvent was evaporated under reduced pressure. The residue was chromatographed using a gradient of methanol in dichloromethane. The collected material was dried and then it was suspended in water (20 mL). The mixture was acidified to pH˜4 with HCl 1 N and subsequently was extracted with ethyl acetate (80 mL). The organic layer was washed with brine and was dried over sodium sulfate. The mixture was filtered, solvent was evaporated and the residue was dried under high vacuum overnight to afford the title product. Light-yellow solid. Yield: 330.2 mg, 77%. HRMS (ESI) m/z calculated for C₂₀H₁₇NO₃ [M+H]⁺=320.1281. found: 320.1282. 1H NMR (500 MHZ, DMSO-d₆) δ 7.69 (s, 1H), 7.49 (d, J=7.3 Hz, 2H), 7.39 (ddd, J=9.5, 7.9, 4.3 Hz, 6H), 7.34 (t, J=7.3 Hz, 1H), 6.64 (d, J=8.5 Hz, 2H), 5.22 (s, 2H) [(protons of the NH₂ and COOH were not observed)]. ¹³C NMR (126 MHz, DMSO-d₆) δ 167.24, 158.81, 148.95, 142.43, 136.97, 132.49, 128.45, 127.84, 127.64, 127.34, 126.05, 118.79, 116.23, 114.16, 112.25, 69.40.

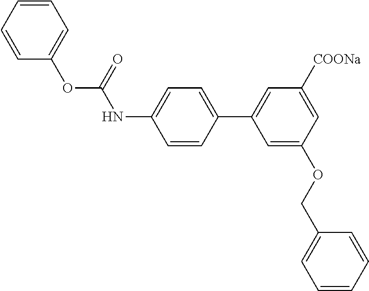

### Sodium 5-(benzyloxy)-4′-((phenoxycarbonyl)amino)-[1,1′-biphenyl]-3-carboxylate (B2″″)

4′-amino-5-(benzyloxy)-[1,1′-biphenyl]-3-carboxylic acid (325.0 mg, 1.02 mmol) was suspended in a mixture of tetrahydrofuran (5 mL) and water (2.5 mL). The mixture was cooled to 0° C. and sodium bicarbonate (188.2 mg, 2.24 mmol) was added, followed by the slow addition of a solution of phenyl chloroformate (0.13 mL, 1.07 mmol) in 2.5 mL tetrahydrofuran. The reaction run for 2h 35 min at 0° C., at which point TLC indicated consumption of the starting material. Ethyl acetate (80 mL) was added, the organic phase was washed with brine (3 times×30 mL), solvent was evaporated under reduced pressure and the residue was dried in high vacuum overnight. The residue was washed with water, it was dried over the Buchner, and then it was suspended in ethyl acetate. The mixture was filtered and the filtrate was dried again in high vacuum to afford the title product. Product: beige solid. Yield: 463.0 mg, 98%. ¹H NMR (500 MHz, DMSO-d₆) δ 10.37 (s, 1H), 7.78 (s, 1H), 7.70 (d, J=8.6 Hz, 2H), 7.62 (d, J=8.5 Hz, 2H), 7.52 (d, J=11.9 Hz, 2H), 7.49 (s, 2H), 7.43 (dt, J=14.8, 7.8 Hz, 4H), 7.34 (t, J=7.3 Hz, 1H), 7.30-7.22 (m, 3H), 5.25 (s, 2H).

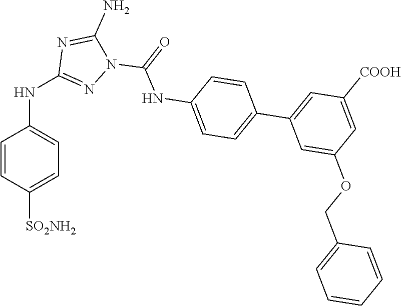 [SMILES: Nc1nc(Nc2ccc(S(N)(=O)=O)cc2)nn1C(=O)Nc1ccc(-c2cc(OCc3ccccc3)cc(C(=O)O)c2)cc1]

### 4′-(5-amino-3-((4-sulfamoylphenyl)amino)-1H-1,2,4-triazole-1-carboxamido)-5-(benzyloxy)-[1,1′-biphenyl]-3-carboxylic acid (JAK344)

Mixture A: In a flame-dried vial, 4-((5-amino-1H-1,2,4-triazol-3-yl)amino) benzenesulfonamide (63.6 mg, 0.25 mmol) was suspended in 1.0 mL anhydrous dioxane and the mixture was heated for a 5-10 min at 100° C., followed by the addition of triethylamine (35 μL, 0.25 mmol). The mixture was allowed to stir for another 5 min and subsequently it was added dropwise to Mixture B. The vial was rinsed with 1.0 mL anhydrous dioxane, which was also added to the Mixture B.

Mixture B: In a flame-dried vial equipped with a rubber septum, sodium 5-(benzyloxy)-4′-((phenoxycarbonyl)amino)-[1,1′-biphenyl]-3-carboxylate (115.4 mg, 0.25 mmol) in 1.0 mL anhydrous dioxane was heated at 100° C. for 5-10 min, under nitrogen. Triethylamine (35 μL, 0.25 mmol) was added, followed by the addition of Mixture A. The reaction was heated at 100° C. for 6h. Solvent was evaporated, water was added to the residue and the mixture was acidified to pH˜4 with HCl 1N. The precipitate was collected by centrifugation and was subsequently purified with preparative reverse-phase HPLC, using an Agilent 5 Prep-C18 21×100 mm column with a gradient of 0.1% formic acid in water and 0.1% formic acid in acetonitrile as the mobile phase. Product: white solid. Yield: 3.1 mg, 2% (Purity: 96.7%). HRMS (ESI) m/z calculated for C₂₉H25N706S [M+H]⁺=600.1660. found: 600.1671. 1H NMR (600 MHZ, DMSO-d₆) δ 9.70 (d, J=6.1 Hz, 2H), 7.84 (d, J=8.6 Hz, 2H), 7.82-7.76 (m, 3H), 7.76-7.69 (m, 4H), 7.50 (d, J=6.7 Hz, 3H), 7.46 (s, 2H), 7.41 (t, J=7.2 Hz, 3H), 7.34 (t, J=7.3 Hz, 1H), 7.15 (s, 2H), 5.23 (s, 2H) [Proton of the COOH moiety was not observed]. ¹³C NMR (126 MHZ, DMSO-d₆) δ 167.94, 158.64, 156.71, 155.91, 149.04, 143.87, 140.41, 137.16, 136.85, 135.63, 134.90, 128.46, 127.82, 127.68, 126.92, 126.82, 121.88, 119.82, 116.24, 115.39, 114.54, 113.84, 69.39.

### Ethyl 6-(4-((tert-butoxycarbonyl)amino)phenyl)picolinate (B1ii)

According to General Method A: Ethyl 6-bromopicolinate (425.7 mg, 1.85 mmol) was mixed with (4-((tert-butoxycarbonyl)amino)phenyl)boronic acid (438.6 mg, 1.85 mmol), and cesium carbonate (1206 mg, 3.70 mmol, 2.0 eq.) in 9 mL anhydrous dimethylformamide. The mixture was degassed under vigorous stirring for 20 min, and subsequently Pd(dppf)Cl₂ (139.0 mg, 0.19 mmol) was added. The reaction ran for 6.5 h, when TLC and LCMS indicated completion. Product: white solid. Yield: 500.2 mg, 79%. HRMS (ESI) m/z calculated for C₁₉H₂₂N₂O₄ [M+H]⁺=343.1652. found: 343.1659. 1H NMR (600 MHZ, DMSO-d₆) δ 9.58 (s, 1H), 8.13 (d, J=7.9 Hz, 1H), 8.07-8.03 (m, 2H), 8.02 (t, J=7.8 Hz, 1H), 7.93 (d, J=7.6 Hz, 1H), 7.60 (d, J=8.6 Hz, 2H), 4.38 (q, J=7.1 Hz, 2H), 1.49 (s, 9H), 1.36 (t, J=7.1 Hz, 3H). ¹³C NMR (151 MHZ, DMSO-d₆) δ 164.8, 156.0, 152.7, 147.6, 141.0, 138.4, 131.3, 127.3, 122.8, 122.7, 118.0, 79.4, 61.2, 28.1, 14.2.

### Methyl 6-(4-((tert-butoxycarbonyl)amino)phenyl)pyrazine-2-carboxylate (B2ii)

According to General Method A: Methyl 6-bromopyrazine-2-carboxylate (401.5 mg, 1.85 mmol) was mixed with (4-((tert-butoxycarbonyl)amino)phenyl)boronic acid (438.6 mg, 1.85 mmol), and cesium carbonate (1206 mg, 3.70 mmol, 2.0 eq.) in 9 mL anhydrous dimethylformamide. The mixture was degassed under vigorous stirring for 20 min, and subsequently Pd(dppf)Cl₂ (139.0 mg, 0.19 mmol) was added. The reaction ran for 4 h, when TLC and LCMS indicated completion. Slightly-modified workup: Solvent was evaporated under reduced pressure, and the residue was diluted with 70 mL ethyl acetate. The organic phase was washed with water (3 times×30 mL) and with brine (1 time×30 mL). The product was additionally back-extracted from the aqueous phase with ethyl acetate (3 times x 20 mL). The combined organic phase was dried under sodium sulfate, and after solvent evaporation the mixture was chromatographed using a gradient of ethyl acetate in hexanes. Product: off-white solid. Yield: 467.4 mg, 77%. MS (ESI) m/z=330.1 [M+H]⁺. ¹H NMR (600 MHZ, DMSO-d₆) δ 9.67 (s, 1H), 9.42 (s, 1H), 9.07 (s, 1H), 8.14-8.10 (m, 2H), 7.65 (d, J=8.7 Hz, 2H), 3.96 (s, 3H), 1.50 (s, 9H). ¹³C NMR (151 MHz, DMSO-d₆) δ 164.3, 152.6, 150.9, 144.5, 142.8, 142.0, 141.9, 128.4, 127.7, 118.2, 79.5, 52.8, 28.1.

### Ethyl 5-(4-((tert-butoxycarbonyl)amino)phenyl)nicotinate (B3ii)

According to General Method A: Ethyl 5-bromonicotinate (425.7 mg, 1.85 mmol) was mixed with (4-((tert-butoxycarbonyl)amino)phenyl)boronic acid (438.6 mg, 1.85 mmol), and cesium carbonate (1206 mg, 3.70 mmol, 2.0 eq.) in 9 mL anhydrous dimethylformamide. The mixture was degassed under vigorous stirring for 20 min, and subsequently Pd(dppf)Cl₂ (139.0 mg, 0.19 mmol) was added. The reaction ran for 6 h, when TLC and LCMS indicated completion. Slightly-modified workup: Solvent was evaporated under reduced pressure, and the residue was diluted with 80 mL ethyl acetate. The organic phase was washed with water (3 times×20 mL) and with brine (1 time×20 mL). The product was additionally back-extracted from the aqueous phase with ethyl acetate (3 times×20 mL). The combined organic phase was dried under sodium sulfate, and after solvent evaporation the mixture was chromatographed using a gradient of ethyl acetate in hexanes to afford the title product. Product: white-pink solid. Yield: 439.2 mg, 69%. HRMS (ESI) m/z calculated for C₁₉H₂₂N₂O₄ [M+H]⁺=343.1652. found: 343.1662. 1H NMR (600 MHZ, DMSO-d₆) δ 9.56 (s, 1H), 9.10 (d, J=2.2 Hz, 1H), 9.01 (d, J=1.9 Hz, 1H), 8.41 (t, J=2.2 Hz, 1H), 7.73-7.70 (m, 2H), 7.61 (d, J=8.5 Hz, 2H), 4.38 (q, J=7.1 Hz, 2H), 1.49 (s, 9H), 1.36 (t, J=7.1 Hz, 3H). ¹³C NMR (151 MHz, DMSO-d₆) δ 164.8, 152.7, 151.2, 148.0, 140.2, 135.2, 133.5, 129.2, 127.4, 125.9, 118.5, 79.3, 61.3, 28.1, 14.1.

### Methyl 4-(4-((tert-butoxycarbonyl)amino)phenyl)picolinate (B4ii)

According to General Method A: Methyl 4-bromopicolinate (399.7 mg, 1.85 mmol) was mixed with (4-((tert-butoxycarbonyl)amino)phenyl)boronic acid (438.6 mg, 1.85 mmol), and cesium carbonate (1206 mg, 3.70 mmol, 2.0 eq.) in 9 mL anhydrous dimethylformamide. The mixture was degassed under vigorous stirring for 20 min, and subsequently Pd(dppf)Cl₂ (139.0 mg, 0.19 mmol) was added. The reaction ran for 1 h 25 min, when TLC indicated completion. Product: white solid. Yield: 416.0 mg, 68%. HRMS (ESI) m/z calculated for C₁₈H₂₀N₂O₄ [M+H]⁺=329.1496. found: 329.1489. 1H NMR (600 MHZ, DMSO-d₆) δ 9.63 (s, 1H), 8.70 (d, J=5.1 Hz, 1H), 8.26 (d, J=1.5 Hz, 1H), 7.92 (dd, J=5.1, 1.8 Hz, 1H), 7.82-7.78 (m, 2H), 7.63 (d, J=8.6 Hz, 2H), 3.91 (s, 3H), 1.49 (s, 9H). ¹³C NMR (151 MHZ, DMSO-d₆) δ 165.4, 152.7 150.4, 148.3, 147.9, 141.3, 129.4, 127.4, 123.7, 121.2, 118.4, 79.5, 52.5, 28.1.

### Ethyl 6-(4-aminophenyl)picolinate (B1i)

According to General Method B: Ethyl 6-(4-((tert-butoxycarbonyl)amino)phenyl)picolinate (B1ii) (500.0 mg, 1.46 mmol) reacted with trifluoroacetic acid (3.8 mL, 49.6 mmol) in 3.8 mL anhydrous dichloromethane for 1 h. Product: light brown solid. 402.5 mg of material were isolated and carried to the next step without further purification. HRMS (ESI) m/z calculated for C₁₄H₁₄N₂O₂ [M+H]⁺=243.1128. found: 243.1124.

¹H NMR (600 MHZ, DMSO-d₆) δ 7.98 (dd, J=8.1, 0.7 Hz, 1H), 7.91 (t, J=7.8 Hz, 1H), 7.88-7.84 (m, 2H), 7.80 (dd, J=7.6, 0.7 Hz, 1H), 6.68-6.64 (m, 2H), 5.53 (s, 2H), 4.36 (q, J=7.1 Hz, 2H), 1.35 (t, J=7.1 Hz, 3H). ¹³C NMR (151 MHz, DMSO-d₆) δ 165.0, 156.9, 150.5, 147.4, 137.9, 127.8, 124.9, 121.5, 121.4, 113.7, 61.1, 14.2.

### Methyl 6-(4-aminophenyl)pyrazine-2-carboxylate (B2i)

According to General Method B: Methyl 6-(4-((tert-butoxycarbonyl)amino)phenyl)pyrazine-2-carboxylate (B2ii) (465.2 mg, 1.41 mmol) reacted with trifluoroacetic acid (3.7 mL, 48.3 mmol) in 3.7 mL anhydrous dichloromethane for 1.5 h. Product: light brown solid. 397 mg of material were isolated and carried to the next step without further purification. HRMS (ESI) m/z calculated for C₁₂H₁₁N₃O₂ [M+H]⁺=230.0924. found: 230.0909.

¹H NMR (600 MHZ, DMSO-d₆) δ 9.27 (s, 1H), 8.91 (s, 1H), 7.94-7.90 (m, 2H), 6.70-6.67 (m, 2H), 5.74 (s, 2H), 3.93 (s, 3H). ¹³C NMR (151 MHZ, DMSO-d₆) δ 164.6, 151.8, 151.4, 143.5, 141.8, 141.2, 128.2, 121.6, 113.8, 52.7.

### Ethyl 5-(4-aminophenyl)nicotinate (B3i)

According to General Method B: Ethyl 5-(4-((tert-butoxycarbonyl)amino)phenyl)nicotinate (B3ii) (437.0 mg, 1.28 mmol) reacted with trifluoroacetic acid (3.3 mL, 43.1 mmol) in 3.3 mL anhydrous dichloromethane for 1 h 10 min. Product: light yellow solid. 325.4 mg of material were isolated and carried to the next step without further purification. HRMS (ESI) m/z calculated for C₁₄H₁₄N₂O₂ [M+H]⁺=243.1128. found: 243.1120. 1H NMR (600 MHZ, DMSO-d₆) δ 9.01 (d, J=2.2 Hz, 1H), 8.91 (d, J=1.7 Hz, 1H), 8.31 (t, J=2.1 Hz, 1H), 7.50-7.46 (m, 2H), 6.72-6.65 (m, 2H), 5.45 (s, 2H), 4.37 (q, J=7.1 Hz, 2H), 1.35 (t, J=7.1 Hz, 3H). ¹³C NMR (151 MHz, DMSO-d₆) δ 164.9, 150.4, 149.6, 146.8, 136.0, 132.4, 127.6, 125.8, 122.4, 114.3, 61.2, 14.1.

### Methyl 4-(4-aminophenyl)picolinate (B4i)

According to General Method B: Methyl 4-(4-((tert-butoxycarbonyl)amino)phenyl)picolinate (B4ii) (413.0 mg, 1.26 mmol) reacted with trifluoroacetic acid (3.2 mL, 41.8 mmol) in 3.2 mL anhydrous dichloromethane for 1 h 10 min. Product: light brown solid. 481.5 mg of material were isolated and carried to the next step without further purification. MS (ESI) m/z=229.1 [M+H]⁺. ¹H NMR (600 MHZ, DMSO-d₆) δ 8.59 (d, J=5.2 Hz, 1H), 8.17 (d, J=1.5 Hz, 1H), 7.81 (dd, J=5.2, 1.9 Hz, 1H), 7.62-7.56 (m, 2H), 6.71-6.65 (m, 2H), 5.64 (s, 2H), 3.89 (s, 3H). ¹³C NMR (151 MHz, DMSO-d₆) δ 165.6, 150.8, 150.1, 148.5, 148.1, 127.7, 122.4, 122.3, 120.1, 114.1, 52.4.

### Ethyl 6-(4-((phenoxycarbonyl)amino)phenyl)picolinate (B1)

According to General Method C: Ethyl 6-(4-aminophenyl)picolinate (B1i) (353.7 mg, 1.46 mmol) was suspended in 3.0 mL tetrahydrofuran and 1.5 mL water. Upon mixing with the other reagents, the reaction was allowed to run at 0° C. for 55 min. Product: pale yellow solid. Yield: 517.8 mg, 98%. MS (ESI) m/z=363.1 [M+H]⁺. ¹H NMR (600 MHz, DMSO-d₆) δ 10.47 (s, 1H), 8.17 (d, J=8.0 Hz, 1H), 8.14-8.11 (m, 2H), 8.04 (t, J=7.8 Hz, 1H), 7.95 (d, J=7.6 Hz, 1H), 7.67 (d, J=8.6 Hz, 2H), 7.46-7.43 (m, 2H), 7.30-7.24 (m, 3H), 4.39 (q, J=7.1 Hz, 2H), 1.36 (t, J=7.1 Hz, 3H). 13C NMR (151 MHZ, DMSO-d₆) δ 164.8, 155.8, 151.7, 150.4, 147.6, 140.1, 138.5, 132.3, 129.5, 127.5, 125.6, 123.0, 122.9, 122.0, 118.4, 61.2, 14.2.

### Methyl 6-(4-((phenoxycarbonyl)amino)phenyl)pyrazine-2-carboxylate (B2)

According to General Method C: Methyl 6-(4-aminophenyl)pyrazine-2-carboxylate (B2i) (323.0 mg, 1.41 mmol) was suspended in 3.0 mL tetrahydrofuran and 1.5 mL water. Upon mixing with the other reagents, the reaction was allowed to run at 0° C. for 2 h. Product: dark yellow solid. 515.8 mg of material were isolated and carried to the next step without further purification. MS (ESI) m/z=350.1 [M+H]⁺. ¹H NMR (600 MHZ, DMSO-d₆) δ 10.55 (s, 1H), 9.45 (s, 1H), 9.09 (s, 1H), 8.20-8.18 (m, 2H), 7.71 (d, J=8.7 Hz, 2H), 7.47-7.43 (m, 2H), 7.30-7.24 (m, 3H), 3.96 (s, 3H). ¹³C NMR (151 MHz, DMSO-d₆) δ 164.3, 151.7, 150.7, 150.4, 144.6, 143.1, 142.1, 140.9, 129.5, 129.3, 127.9, 125.6, 122.0, 118.6, 52.8.

### Ethyl 5-(4-((phenoxycarbonyl)amino)phenyl)nicotinate (B3)

According to General Method C: Ethyl 5-(4-aminophenyl)nicotinate (B3i) (310.1 mg, 1.28 mmol) was suspended in 2.6 mL tetrahydrofuran and 1.3 mL water. Upon mixing with the other reagents, the reaction was allowed to run at 0° C. for 73 min. The back-extraction step was excluded. Product: pale yellow solid. Yield: 452.9 mg, 98%. MS (ESI) m/z=363.1 [M+H]⁺.

¹H NMR (600 MHZ, DMSO-d₆) δ 10.45 (s, 1H), 9.13 (d, J=2.2 Hz, 1H), 9.03 (d, J=1.8 Hz, 1H), 8.45 (t, J=2.1 Hz, 1H), 7.82-7.79 (m, 2H), 7.67 (d, J=8.5 Hz, 2H), 7.48-7.42 (m, 2H), 7.31-7.23 (m, 3H), 4.39 (q, J=7.1 Hz, 2H), 1.36 (t, J=7.1 Hz, 3H). ¹³C NMR (151 MHz, DMSO-d₆) δ 164.8, 151.7, 151.2, 150.4, 148.2, 139.3, 135.1, 133.7, 130.2, 129.5, 127.7, 126.0, 125.6, 122.0, 118.9, 61.3, 14.1.

### Methyl 4-(4-((phenoxycarbonyl)amino)phenyl)picolinate (B4)

According to General Method C: Methyl 4-(4-aminophenyl)picolinate (B4i) (287.6 mg, 1.26 mmol) was suspended in 3.1 mL tetrahydrofuran and 1.3 mL water. Upon mixing with the other reagents, the reaction was allowed to run at 0° C. for 45 min. Product: pale yellow solid. Yield: 408.0 mg, 93%. MS (ESI) m/z=349.1 [M+H]⁺. ¹H NMR (600 MHz, DMSO-d₆) δ 10.51 (s, 1H), 8.73 (d, J=5.1 Hz, 1H), 8.31-8.28 (m, 1H), 7.95 (dd, J=5.1, 1.7 Hz, 1H), 7.91-7.86 (m, 2H), 7.69 (d, J=8.6 Hz, 2H), 7.48-7.41 (m, 2H), 7.30-7.24 (m, 3H), 3.91 (s, 3H). ¹³C NMR (151 MHZ, DMSO-d₆) δ 165.3, 151.7, 150.41, 150.38, 148.3, 147.8, 140.3, 130.4, 129.5, 127.7, 125.6, 123.9, 122.0, 121.4, 118.8, 52.5.

### Ethyl 6-(4-(5-amino-3-((4-sulfamoylphenyl)amino)-1H-1,2,4-triazole-1-carboxamido)phenyl)picolinate (3i)

According to General Method D: Ethyl 6-(4-((phenoxycarbonyl)amino)phenyl)picolinate (B1) (116.0 mg, 0.32 mmol) reacted at 100° C. for 2 h (1.9 mL/mmol anhydrous dioxane, 1 eq. triethylamine). Solvent was evaporated and 20-30 mL of water were added. The aqueous layer was washed with ethyl acetate 3-4 times. The two phases were separated and the aqueous layer was collected and concentrated in vacuo. The residue was further purified with preparative reverse phase HPLC, using an Agilent 5 Prep-C18 21.2×100 mm column with a gradient of 0.1% formic acid in water and 0.1% formic acid in acetonitrile as the mobile phase (System B). Product: off-white solid. Yield: 46.7 mg, 28%. HRMS (ESI) m/z calculated for C₂₃H₂₂N₈O₅S [M+H]⁺=523.1507. found: 523.1520. 1H NMR (600 MHZ, DMSO-d₆) δ 9.76 (s, 1H), 9.71 (s, 1H), 8.23 (d, J=7.8 Hz, 1H), 8.21-8.17 (m, 2H), 8.07 (t, J=7.8 Hz, 1H), 7.98 (d, J=7.7 Hz, 1H), 7.88-7.85 (m, 2H), 7.85-7.81 (m, 2H), 7.72 (d, J=8.9 Hz, 2H), 7.47 (br s, 2H), 7.15 (s, 2H), 4.40 (q, J=7.1 Hz, 2H), 1.37 (t, J=7.1 Hz, 3H). ¹³C NMR (151 MHz, DMSO-d₆) δ 164.8, 156.7, 155.9, 155.7, 149.0, 147.7, 143.8, 138.63, 138.57, 134.9, 133.5, 127.2, 126.8, 123.2, 123.1, 121.5, 116.2, 61.3, 14.2.

### Methyl 6-(4-(5-amino-3-((4-sulfamoylphenyl)amino)-1H-1,2,4-triazole-1-carboxamido)phenyl)pyrazine-2-carboxylate (4i)

According to General Method D: Methyl 6-(4-((phenoxycarbonyl)amino)phenyl)pyrazine-2-carboxylate (B2) (111.8 mg, 0.32 mmol), reacted at 110° C. for 2 h (1.9 mL/mmol anhydrous dioxane, 1 eq. triethylamine). The reaction was monitored with TLC in long wave and with LCMS. The reaction mixture was dried and was chromatographed using normal phase chromatography and a gradient of acetonitrile in dichloromethane. Additional purification was necessary with preparative reverse phase HPLC, using an Agilent 5 Prep-C18 21.2×100 mm column with a gradient of 0.1% formic acid in water and 0.1% formic acid in acetonitrile as the mobile phase (System B). Product: light yellow solid. 21.5 mg, 13%. MS (ESI) m/z=510.2 [M+H]⁺. ¹H NMR (600 MHz, DMSO-d₆) δ 9.83 (s, 1H), 9.72 (s, 1H), 9.51 (s, 1H), 9.13 (s, 1H), 8.26 (d, J=8.2 Hz, 2H), 7.93 (d, J=8.4 Hz, 2H), 7.84 (d, J=8.6 Hz, 2H), 7.72 (d, J=8.7 Hz, 2H), 7.49 (br s, 2H), 7.16 (s, 2H), 3.97 (s, 3H). ¹³C NMR (151 MHZ, DMSO-d₆) δ 164.3, 156.8, 156.0, 150.6, 148.9, 144.8, 143.8, 143.3, 142.1, 139.5, 134.9, 130.5, 127.5, 126.8, 121.5, 116.2, 52.8.

### Ethyl 5-(4-(5-amino-3-((4-sulfamoylphenyl)amino)-1H-1,2,4-triazole-1-carboxamido)phenyl)nicotinate (5i)

According to General Method D: Ethyl 5-(4-((phenoxycarbonyl)amino)phenyl)nicotinate (B3) (116.0 mg, 0.32 mmol) reacted at 100° C. for 2 h 45 min (1.9 mL/mmol anhydrous dioxane, 1 eq. triethylamine). Solvent was evaporated and 20-30 mL of water were added. The aqueous layer was washed with ethyl acetate 3-4 times. The two phases were separated and the aqueous layer was collected and concentrated in vacuo to afford the title product. Product: white solid. Yield: 111.6 mg, 67%. HRMS (ESI) m/z calculated for C₂₃H₂₂N₈O₅S [M+H]⁺=523.1507. found: 523.1506.

¹H NMR (500 MHZ, DMSO-d₆) δ 9.75 (s, 1H), 9.70 (s, 1H), 9.18 (d, J=2.1 Hz, 1H), 9.06 (d, J=1.7 Hz, 1H), 8.50 (t, J=2.0 Hz, 1H), 7.87 (s, 4H), 7.83 (d, J=8.8 Hz, 2H), 7.72 (d, J=8.8 Hz, 2H), 7.46 (br s, 2H), 7.15 (s, 2H), 4.40 (q, J=7.1 Hz, 2H), 1.37 (t, J=7.1 Hz, 3H). ¹³C NMR (151 MHz, DMSO-d₆) δ 164.8, 156.8, 156.0, 151.4, 149.1, 148.4, 143.9, 137.9, 135.0, 134.9, 133.9, 131.5, 127.4, 126.8, 126.0, 122.0, 116.3, 61.4, 14.1.

### Methyl 4-(4-(5-amino-3-((4-sulfamoylphenyl)amino)-1H-1,2,4-triazole-1-carboxamido)phenyl)picolinate (6i)

According to General Method D: Methyl 4-(4-((phenoxycarbonyl)amino)phenyl)picolinate (B4) (81.4 mg, 0.32 mmol) reacted at 110° C. for 2 h 45 min (1.9 mL/mmol anhydrous dioxane, 1 eq. triethylamine). Solvent was evaporated and 20-30 mL of water were added. The aqueous layer was washed with ethyl acetate 3-4 times. The two phases were separated and the aqueous layer was collected and concentrated in vacuo to afford the title product. Product: pale yellow solid. Yield: 108.2 mg, 66%. HRMS (ESI) m/z calculated for C₂₂H₂₀N₈O₅S [M+H]⁺=509.1350. found: 509.1354.

¹H NMR (600 MHz, DMSO-d₆) δ 9.80 (s, 1H), 9.72 (s, 1H), 8.76 (d, J=5.1 Hz, 1H), 8.34 (d, J=1.4 Hz, 1H), 8.01 (dd, J=5.1, 1.7 Hz, 1H), 7.95 (d, J=8.8 Hz, 2H), 7.90 (d, J=8.7 Hz, 2H), 7.86-7.81 (m, 2H), 7.72 (d, J=8.8 Hz, 2H), 7.48 (br s, 2H), 7.16 (s, 2H), 3.93 (s, 3H). ¹³C NMR (151 MHz, DMSO-d₆) δ 165.4, 156.8, 156.0, 150.5, 149.0, 148.4, 147.7, 143.8, 138.9, 134.9, 131.7, 127.4, 126.8, 124.1, 121.8, 121.5, 116.2, 52.5.

### 6-(4-(5-amino-3-((4-sulfamoylphenyl)amino)-1H-1,2,4-triazole-1-carboxamido)phenyl)picolinic acid (3)

According to General Method E: Ethyl 6-(4-(5-amino-3-((4-sulfamoylphenyl)amino)-1H-1,2,4-triazole-1-carboxamido)phenyl)picolinate (3i) (40.8 mg, 0.078 mmol) was hydrolyzed according to the conditions reported. Reaction time: 52 h 10 min. Product: white solid. Yield: 16.0 mg, 41% (Purity: 95%). HRMS (ESI) m/z calculated for C₂₁H₁₈N₈O₅S [M+H]⁺=495.1194. found: 495.1178.

¹H NMR (600 MHZ, DMSO-d₆) δ 9.76 (s, 1H), 9.71 (s, 1H), 8.24 (d, J=8.1 Hz, 2H), 8.19 (d, J=7.8 Hz, 1H), 8.04 (t, J=7.7 Hz, 1H), 7.96 (d, J=7.4 Hz, 1H), 7.86 (d, J=8.4 Hz, 2H), 7.84 (d, J=9.0 Hz, 2H), 7.72 (d, J=8.3 Hz, 2H), 7.47 (s, 2H), 7.15 (s, 2H) [Proton of the COOH moiety was not observed]. ¹³C NMR (151 MHZ, DMSO-d₆) δ 166.4, 156.7, 155.9, 155.4, 149.0, 143.8, 138.5, 138.4, 134.9, 133.6, 127.2, 126.8, 122.8, 122.7, 121.4, 116.2 (one carbon peak overlaps with the rest in the aromatic region).

### 6-(4-(5-amino-3-((4-sulfamoylphenyl)amino)-1H-1,2,4-triazole-1-carboxamido)phenyl)pyrazine-2-carboxylic acid (4)

According to General Method E: Methyl 6-(4-(5-amino-3-((4-sulfamoylphenyl)amino)-1H-1,2,4-triazole-1-carboxamido)phenyl)pyrazine-2-carboxylate (4i) (17.3 mg, 0.034 mmol) was hydrolyzed according to the conditions reported. Reaction time: 32 h. Product: pale white solid. Yield: 4.6 mg, 27% (Purity: 96%). HRMS (ESI) m/z calculated for C₂₀H17N905S [M+H]⁺=496.1146. found: 496.1130.

¹H NMR (600 MHz, DMSO-d₆) δ 9.80 (s, 1H), 9.71 (s, 1H), 9.42 (s, 1H), 9.07 (s, 1H), 8.27 (d, J=8.1 Hz, 2H), 7.91 (d, J=7.8 Hz, 2H), 7.84 (d, J=8.2 Hz, 2H), 7.73 (d, J=8.0 Hz, 2H), 7.48 (br s, 2H), 7.15 (s, 2H) [Proton of the COOH moiety was not observed]. ¹³C NMR (151 MHZ, DMSO-d₆) δ 165.5, 156.8, 156.0, 150.3, 149.0, 144.6, 143.8, 143.6, 143.3, 139.3, 134.9, 131.0, 127.5, 126.8, 121.5, 116.3.

### 5-(4-(5-amino-3-((4-sulfamoylphenyl)amino)-1H-1,2,4-triazole-1-carboxamido)phenyl)nicotinic acid (5)

According to General Method E: Ethyl 5-(4-(5-amino-3-((4-sulfamoylphenyl)amino)-1H-1,2,4-triazole-1-carboxamido)phenyl)nicotinate (5i) (40.8 mg, 0.078 mmol) was hydrolyzed according to the conditions reported. Reaction time: 98 h. Product: Yellow Solid. Yield: 8.0 mg, 21% (Purity: 95%). HRMS (ESI) m/z calculated for C₂₁H18N805S [M+H]⁺=495.1194, found: 495.1193.

¹H NMR (600 MHZ, DMSO-d₆) δ 9.72 (s, 1H), 9.70 (s, 1H), 8.96 (s, 1H), 8.91 (s, 1H), 8.40 (s, 1H), 7.84 (d, J=8.0 Hz, 4H), 7.79 (d, J=8.4 Hz, 2H), 7.72 (d, J=8.4 Hz, 2H), 7.46 (br s, 2H), 7.15 (s, 2H) [Proton of the COOH moiety was not observed]. ¹³C NMR (151 MHZ, DMSO-d₆) δ 166.9, 164.1, 156.7, 155.9, 149.2, 149.0, 148.0, 143.9, 137.3, 134.9, 134.1, 133.8, 132.9, 127.1, 126.8, 122.0, 116.2.

### 4-(4-(5-amino-3-((4-sulfamoylphenyl)amino)-1H-1,2,4-triazole-1-carboxamido)phenyl)picolinic acid (6)

According to General Method E: Methyl 4-(4-(5-amino-3-((4-sulfamoylphenyl)amino)-1H-1,2,4-triazole-1-carboxamido)phenyl)picolinate (6i) (40.0 mg, 0.079 mmol) was hydrolyzed according to the conditions reported. Reaction time: 29 h 20 min. Product: yellow solid. Yield: 13.4 mg, 34% (Purity: 97%). HRMS (ESI) m/z calculated for C₂₁H₁₈N₈O₅S [M+H]⁺=495.1194. found: 495.1185.

¹H NMR (600 MHZ, DMSO-d₆) δ 9.78 (s, 1H), 9.71 (s, 1H), 8.69 (s, 1H), 8.30 (s, 1H), 7.94-7.87 (m, 5H), 7.84 (d, J=8.1 Hz, 2H), 7.72 (d, J=8.3 Hz, 2H), 7.47 (br s, 2H), 7.15 (s, 2H) [Proton of the COOH moiety was not observed]. ¹³C NMR (151 MHZ, DMSO-d₆) δ 166.8, 163.6, 156.7, 155.9, 149.7, 149.0, 147.3, 143.8, 138.6, 134.9, 132.2, 127.2, 126.8, 122.8, 121.8, 121.0, 116.2.

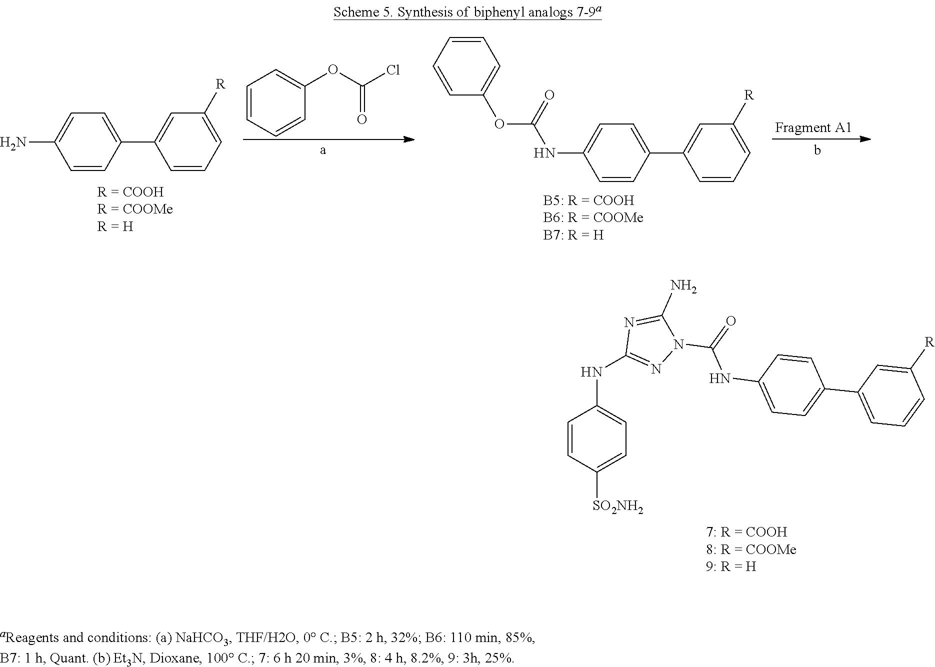

### 4′-((phenoxycarbonyl)amino)-[1,1′-biphenyl]-3-carboxylic acid (B5)

According to General Method C: 4′-amino-[1,1′-biphenyl]-3-carboxylic acid (705.8 mg, 3.31 mmol) was suspended in 6.6 mL tetrahydrofuran containing 3.3 mL water. The mixture was brought to 0° C. followed by the addition of sodium bicarbonate (611.6 mg, 7.28 mmol). The mixture was allowed to stir for 5-10 min and then a solution of phenyl chloroformate (0.44 mL, 3.48 mmol) in 3.3 mL tetrahydrofuran was added dropwise. The reaction was allowed to stir for 2 h at 0° C., at which point TLC indicated completion. Water (10 mL) was added to the reaction, followed by methanol until cloudy. The solid was filtered out and the filtrate was collected and further processed as follows: 25 mL water was added and the pH was adjusted to ˜4 with 1N HCl. The aqueous phase was extracted with ethyl acetate (3 times×40 mL). The combined organic layer was washed with brine (2 times×30 mL) and was dried over sodium sulfate. Solvent was evaporated under reduced pressure to afford the title product. Product: pale pink solid. Yield: 348.8 mg, 32%. HRMS (ESI) m/z calculated for C₂₀H₁₅NO₄ [M+H]⁺=334.1074. found: 334.1100.

¹H NMR (600 MHZ, DMSO-d₆) δ 10.39 (s, 1H), 8.17 (s, 1H), 7.90 (dt, J=7.9, 1.6 Hz, 2H), 7.70 (d, J=8.6 Hz, 2H), 7.64 (d, J=8.5 Hz, 2H), 7.57 (t, J=7.7 Hz, 1H), 7.44 (t, J=7.8 Hz, 2H), 7.34-7.19 (m, 3H). ¹³C NMR (151 MHZ, DMSO-d₆) δ 167.3, 151.7, 150.5, 140.0, 138.5, 133.7, 131.6, 130.6, 129.5, 129.3, 127.9, 127.3, 126.9, 125.5, 122.0, 118.9.

### Methyl 4′-((phenoxycarbonyl)amino)-[1,1′-biphenyl]-3-carboxylate (B6)

According to General Method C: Methyl 4′-amino-[1, l′-biphenyl]-3-carboxylate (752.4 mg, 3.31 mmol) was suspended in a mixture of tetrahydrofuran/water 2:1 mixture (2 mL THF/mmol of substrate). The mixture was cooled to 0° C. and sodium bicarbonate (333.5 mg, 3.97 mmol) was added. A solution of phenyl chloroformate (0.44 mL, 3.48 mmol) in tetrahydrofuran (3.3 ml) was added dropwise to the mixture. Upon addition, the reaction was allowed to run at 0° C. for 110 min, at which point TLC indicated completion. The mixture was diluted with 65 mL ethyl acetate and was washed with water (3 times×20 mL) and brine (2 times×20 mL). The combined organic phase was then dried over sodium sulfate. After filtration, solvent was evaporated under reduced pressure and the solid was further dried under high vacuum overnight to afford the title product. Product: white solid. Yield: 982.1 mg, 85%. HRMS (ESI) m/z calculated for C₂₁H17NO+ [M+H]⁺=348.1230. found: 348.1228. 1H NMR (500 MHZ, DMSO-d₆) δ 10.39 (s, 1H), 8.18 (t, J=1.7 Hz, 1H), 7.98-7.88 (m, 2H), 7.72-7.68 (m, 2H), 7.64 (d, J=8.7 Hz, 2H), 7.61 (t, J=7.8 Hz, 1H), 7.49-7.40 (m, 2H), 7.30-7.22 (m, 3H), 3.89 (s, 3H). ¹³C NMR (126 MHZ, DMSO-d₆) δ 166.2, 151.7, 150.5, 140.2, 138.6, 133.5, 131.1, 130.3, 129.48, 129.45, 127.7, 127.3, 126.6, 125.5, 122.0, 118.9, 52.3.

### Phenyl [1,1′-biphenyl]-4-ylcarbamate (B7)

According to General Method C: [1,1′-biphenyl]-4-amine (560.2 mg, 3.31 mmol) was suspended in a mixture of tetrahydrofuran/water 2:1 mixture (2 mL THF/mmol of substrate). The mixture was cooled to 0° C. and sodium bicarbonate (333.5 mg, 3.97 mmol) was added. A solution of phenyl chloroformate (0.44 mL, 3.48 mmol) in tetrahydrofuran (3.3 mL) was added dropwise to the mixture. Upon addition, the reaction was allowed to run at 0° C. for 1 h, at which point TLC indicated completion. The mixture was diluted with 65 mL ethyl acetate, and was washed with water (3 times×20 mL) and brine (2 times×20 mL). The combined organic phase was then dried over sodium sulfate. After filtration, solvent was evaporated to afford the title product. Product: brown solid. Yield: 954.2 mg, Quant. HRMS (ESI) m/z calculated for C₁₉H15NO₂ [M+H]⁺=290.1176. found: 290.1173. 1H NMR (600 MHZ, DMSO-d₆) § 10.35 (s, 1H), 7.67-7.64 (m, 4H), 7.61 (d, J=8.6 Hz, 2H), 7.46-7.42 (m, 4H), 7.35-7.31 (m, 1H), 7.29-7.26 (m, 1H), 7.26-7.23 (m, 2H). ¹³C NMR (151 MHZ, DMSO-d₆) δ 151.7, 150.5, 139.6, 138.1, 134.7, 129.5, 128.9, 127.1, 127.0, 126.3, 125.5, 122.0, 118.8.

### 4′-(5-amino-3-((4-sulfamoylphenyl)amino)-1H-1,2,4-triazole-1-carboxamido)-[1,1′-biphenyl]-3-carboxylic acid (7)

According to General Method D: Mixture A: In a flame-dried vial, Fragment A¹ (89.8 mg, 0.35 mmol) was suspended in 0.5 mL anhydrous dioxane and the mixture was heated for a 10-15 min at 80-100° C., followed by the addition of triethylamine (50 μL, 0.35 mmol). The mixture was allowed to stir for another 10 min and subsequently was added dropwise to Mixture B. The vial was rinsed with 2.0 mL anhydrous dioxane, which was also added to Mixture B.

Mixture B: In a flame-dried vial equipped with a rubber septum, 4′-((phenoxycarbonyl)amino)-[1, l′-biphenyl]-3-carboxylic acid (B5) (117.7 mg, 0.35 mmol) in 0.8 mL anhydrous dioxane was heated at 100° C. for 10 min, under nitrogen. Triethylamine (100 μL, 0.72 mmol) was added, followed by the addition of Mixture A. The reaction ran at 100° C. for 6 h 20 min. Solvent was evaporated under a stream of nitrogen and water was added. The crude mixture was acidified to pH ˜4 with 1N HCl and the precipitate was collected by centrifugation and dried. The crude mixture was purified in batches of 20 mg using preparative reverse phase HPLC, an Agilent 5 Prep-C18 21.2×100 mm column with a gradient of 0.1% formic acid in water and 0.1% formic acid in acetonitrile as the mobile phase (System B). Product: white solid. Yield: 43.7 mg, 25% (Purity: 90-95%)/5.6 mg, 3% (Purity: 97.6%). HRMS (ESI) m/z calculated for C₂₂H19N705S [M+H]⁺=494.1241. found: 494.1260.

¹H NMR (500 MHZ, DMSO-d₆) δ 9.71 (s, 1H), 9.70 (s, 1H), 8.21 (s, 1H), 7.95-7.90 (m, 2H), 7.86-7.80 (m, 4H), 7.75 (d, J=8.6 Hz, 2H), 7.72 (d, J=8.8 Hz, 2H), 7.58 (t, J=7.7 Hz, 1H), 7.46 (br s, 2H), 7.15 (s, 2H) [Proton of the COOH moiety was not observed]. ¹³C NMR (126 MHz, DMSO-d₆) δ 167.5, 156.7, 155.9, 149.0, 143.9, 139.7, 137.0, 135.2, 134.9, 132.7, 130.3, 129.2, 128.0, 126.98, 126.95, 126.8, 122.0, 116.2.

### Methyl 4′-(5-amino-3-((4-sulfamoylphenyl)amino)-1H-1,2,4-triazole-1-carboxamido)-[1,1′-biphenyl]-3-carboxylate (8)

According to General Method D: Mixture A: In a flame-dried vial, Fragment A1 (63.6 mg, 0.25 mmol) was suspended in 1.0 mL anhydrous dioxane and the mixture was heated for 5-10 min at 100° C., followed by the addition of triethylamine (35 μL, 0.25 mmol). The mixture was allowed to stir for another 5-10 min and subsequently was added dropwise to Mixture B.

Mixture B: In a flame-dried vial equipped with a rubber septum, methyl 4′-((phenoxycarbonyl)amino)-[1, l′-biphenyl]-3-carboxylate (B6) (86.8 mg, 0.25 mmol) in 0.5 mL anhydrous dioxane was heated at 100° C. for 10 min, under nitrogen. Triethylamine (35 μL, 0.25 mmol) was added, followed by the addition of Mixture A. The reaction ran at 100° C. for 4 h. Solvent was evaporated and the crude mixture was chromatographed using a gradient of methanol in dichloromethane, to afford the title product. Product: White Solid.

Yield: 10.4 mg, 8.2% (Purity: 98.3%). HRMS (ESI) m/z calculated for C₂₃H21N705S [M+H]⁺=508.1398. found: 508.1391.

¹H NMR (600 MHZ, DMSO-d₆) δ 9.72 (s, 1H), 9.72 (s, 1H), 8.22 (t, J=1.8 Hz, 1H), 7.99 (ddd, J=7.7, 1.8, 1.0 Hz, 1H), 7.95 (dt, J=7.8, 1.3 Hz, 1H), 7.86-7.81 (m, 4H), 7.79-7.74 (m, 2H), 7.74-7.69 (m, 2H), 7.63 (t, J=7.7 Hz, 1H), 7.46 (br s, 2H), 7.16 (s, 2H), 3.90 (s, 3H). ¹³C NMR (151 MHz, DMSO-d₆) δ 166.2, 156.7, 155.9, 149.0, 143.8, 140.1, 137.2, 134.9, 134.8, 131.2, 130.4, 129.5, 127.9, 127.0, 126.79, 126.76, 122.0, 116.2, 52.3.

### N-([1,1′-biphenyl]-4-yl)-5-amino-3-((4-sulfamoylphenyl)amino)-1H-1,2,4-triazole-1-carboxamide (9)

According to General Method D: Mixture A: In a flame-dried vial, Fragment A1 (89.0 mg, 0.35 mmol) was suspended in 0.9 mL anhydrous dioxane and the mixture was heated for a 10-15 min at 100° C., followed by the addition of triethylamine (49 μL, 0.35 mmol). The mixture was allowed to stir for another 10 min and subsequently was added dropwise to Mixture B.

Mixture B: In a flame-dried vial equipped with a rubber septum, phenyl [1,1′-biphenyl]-4-ylcarbamate (B7) (101.0 mg, 0.35 mmol) in 0.3 mL anhydrous dioxane was heated at 100° C. for 10 min, under nitrogen. Triethylamine (49 μL, 0.35 mmol) was added, followed by the addition of Mixture A. The reaction ran at 100° C. for 3 h. Solvent was evaporated and the crude mixture was chromatographed using a gradient of methanol in dichloromethane, to afford the title product. Product: white solid. Yield: 39.6 mg, 25% (Purity: 96%). HRMS (ESI) m/z calculated for C₂₁H19N703S [M+H]⁺=450.1343. found: 450.1340. 1H NMR (500 MHZ, DMSO-d₆) δ 9.69 (s, 1H), 9.68 (s, 1H), 7.85-7.81 (m, 2H), 7.80-7.76 (m, 2H), 7.75-7.67 (m, 6H), 7.51-7.41 (m, 4H), 7.38-7.34 (m, 1H), 7.15 (s, 2H). ¹³C NMR (126 MHz, DMSO-d₆) δ 156.7, 155.9, 149.1, 143.9, 139.5, 136.6, 136.1, 134.9, 129.0, 127.3, 126.8, 126.4, 121.9, 116.2 (one carbon peak overlaps with the rest in the aromatic region).

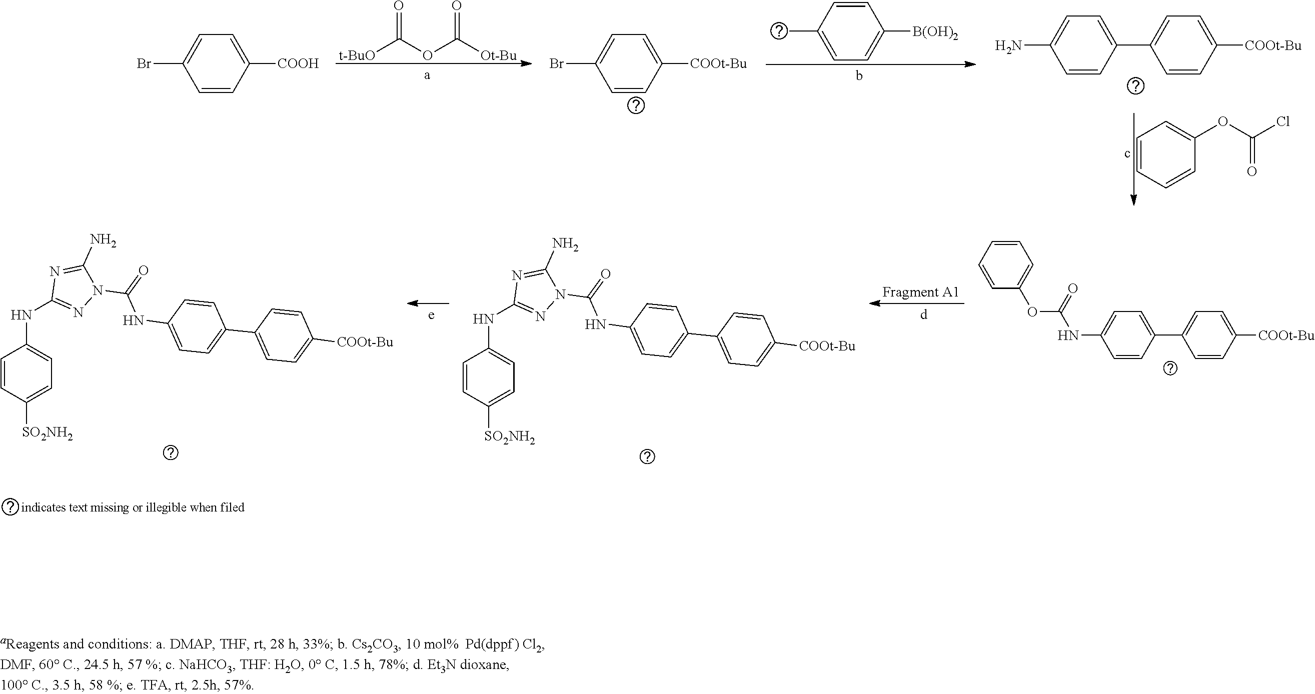

### Tert-butyl 4-bromobenzoate (B8ii)

In a round-bottom flask equipped with a rubber septum, a catalytic amount of 4-dimethylaminopyridine (DMAP) (0.7 g, 5.7 mmol) was added to a mixture of 4-bromobenzoic acid (5.0 g, 24.9 mmol) in tetrahydrofuran (47 mL). The mixture was allowed to stir at room temperature for 10 min, and subsequently di-tert-butyl dicarbonate (16.6 g, 76.1 mmol) was added portion wise, while vortexing the mixture from time to time. Once the addition was complete, the reaction was allowed to run at room temperature for 28 h. The solvent was evaporated and the crude mixture was chromatographed using a gradient of ethyl acetate in hexanes to afford the title product. Product: light yellow oil. Yield: 2.13 g, 33%. Product could not be detected in the MS.

¹H NMR (600 MHz, DMSO-d₆) δ 7.83-7.80 (m, 2H), 7.72-7.69 (m, 2H), 1.53 (s, 9H). ¹³C NMR (151 MHz, DMSO-d₆) δ 164.2, 131.7, 131.0, 130.4, 126.9, 81.2, 27.7.

### tert-butyl 4′-amino-[1,1′-biphenyl]-4-carboxylate (B8i)

According to General Method A: In a flame-dried vial equipped with a rubber septum was added tert-butyl 4-bromobenzoate (B8ii) (498.7 mg, 1.94 mmol), 4-aminophenylboronic acid hydrochloride (336.4 mg, 1.94 mmol) and cesium carbonate (1896.3 mg, 5.82 mmol, 3.0 eq.) in 9.4 mL anhydrous dimethylformamide. The mixture was degassed under vigorous stirring for 30 min, and subsequently Pd(dppf)Cl₂ (141.9 mg, 0.194 mmol) was added. The mixture was flushed with nitrogen and was heated to 60° C. for 24.5 h, when TLC indicated completion. Slightly-modified workup: Solvent was evaporated under reduced pressure, and the crude mixture was directly purified with chromatography using a gradient of ethyl acetate in hexanes, to afford the title product. Product: pale yellow solid. Yield: 298.9 mg, 57%. HRMS (ESI) m/z calculated for C₁₇H₁₉NO₂ [M+H]⁺=270.1489. found: 270.1489. ¹H NMR (600 MHz, DMSO-d₆) δ 7.90-7.85 (m, 2H), 7.67-7.64 (m, 2H), 7.46-7.43 (m, 2H), 6.67-6.63 (m, 2H), 5.40 (s, 2H), 1.55 (s, 9H). ¹³C NMR (151 MHZ, DMSO-d₆) δ 165.0, 149.4, 144.8, 129.6, 128.2, 127.6, 125.6, 124.9, 114.1, 80.3, 27.9.

### Tert-butyl 4′-((phenoxycarbonyl)amino)-[1,1′-biphenyl]-4-carboxylate (B8)

According to General Method C: Tert-butyl 4′-amino-[1,1′-biphenyl]-4-carboxylate (B8i) (293.0 mg, 1.09 mmol) in tetrahydrofuran: water (2.2:1.1 mL) reacted with phenyl chloroformate for 1.5 h. The mixture was processed without the back-extraction step. Product: Pale Yellow Solid. Yield: 332.8 mg, 78%. HRMS (ESI) m/z calculated for C₂₄H23NO₄ [M+H]⁺=390.1700. found: 390.1697. ¹H NMR (600 MHz, DMSO-d₆) δ 10.42 (s, 1H), 7.97-7.94 (m, 2H), 7.80-7.77 (m, 2H), 7.75-7.72 (m, 2H), 7.64 (d, J=8.7 Hz, 2H), 7.46-7.42 (m, 2H), 7.29-7.23 (m, 3H), 1.56 (s, 9H). ¹³C NMR (151 MHz, DMSO-d₆) δ 164.8, 151.7, 150.5, 143.8, 139.0, 133.3, 129.7, 129.5, 129.4, 127.5, 126.2, 125.5, 122.0, 118.8, 80.7, 27.8.

### Tert-butyl 4′-(5-amino-3-((4-sulfamoylphenyl)amino)-1H-1,2,4-triazole-1-carboxamido)-[1,1′-biphenyl]-4-carboxylate (10i)

According to General Method D: Mixture A: In a flame-dried vial, Fragment A1 (63.6 mg, 0.25 mmol) was suspended in 1.0 mL anhydrous dioxane and the mixture was heated for 5 min at 100° C., followed by the addition of triethylamine (35 μL, 0.25 mmol). The mixture was allowed to stir for another 5 min and subsequently it was added dropwise to Mixture B.

Mixture B: In a flame-dried vial equipped with a rubber septum, tert-butyl 4′-((phenoxycarbonyl)amino)-[1,1′-biphenyl]-4-carboxylate (B8) (97.4 mg, 0.25 mmol) in 1.0 mL anhydrous dioxane was heated at 100° C. for 5 min, under nitrogen. Triethylamine (35 μL, 0.25 mmol) was added, followed by the addition of Mixture A. The reaction ran at 100° C. for 3.5 h, at which point solvent was evaporated. The crude mixture was purified with normal-phase chromatography using a gradient of methanol in dichloromethane. Product: white solid. Yield: 79.7 mg, 58%. HRMS (ESI) m/z calculated for C₂₆H27N705S [M+H]⁺=550.1867. found: 550.1867. 1H NMR (600 MHZ, DMSO-d₆) δ 9.73 (s, 1H), 9.70 (s, 1H), 8.00-7.95 (m, 2H), 7.86-7.82 (m, 6H), 7.80-7.77 (m, 2H), 7.74-7.70 (m, 2H), 7.46 (br s, 2H), 7.15 (s, 2H), 1.57 (s, 9H). ¹³C NMR (151 MHZ, DMSO-d₆) δ 164.8, 156.7, 155.9, 149.0, 143.9, 143.7, 137.6, 134.9, 134.6, 129.9, 129.7, 127.2, 126.8, 126.4, 121.8, 116.2, 80.7, 27.8.

### 4′-(5-amino-3-((4-sulfamoylphenyl)amino)-1H-1,2,4-triazole-1-carboxamido)-[1,1′-biphenyl]-4-carboxylic acid (10)

Tert-butyl 4′-(5-amino-3-((4-sulfamoylphenyl)amino)-1H-1,2,4-triazole-1-carboxamido)-[1,1′-biphenyl]-4-carboxylate (10i) (44.0 mg, 0.08 mmol) was suspended in trifluoroacetic acid (1.84 mL, 24.0 mmol). The mixture was allowed to stir at room temperature for 2.5 h, at which point it was dried under a stream of nitrogen. Subsequently water (2.0 mL) was added and the pH was adjusted to ˜4 using acetic acid and triethylamine. The residue was collected after centrifugation, and it was dried. Finally, the residue was triturated, aided by sonication and vortexing, with ethyl ether (2 times) and with a mixture of ethyl ether and acetonitrile (5 times) to afford the title product. Product: white solid. Yield: 22.6 mg, 57% Purity: 96.6%. HRMS (ESI) m/z calculated for C₂₂H19N703S [M+H]+=494.1241. found: 494.1238.

¹H NMR (600 MHZ, DMSO-d₆) δ 12.95 (s, 1H), 9.72 (s, 1H), 9.70 (s, 1H), 8.06-7.98 (m, 2H), 7.86-7.82 (m, 6H), 7.81-7.78 (m, 2H), 7.74-7.70 (m, 2H), 7.46 (br s, 2H), 7.15 (s, 2H). ¹³C NMR (151 MHZ, DMSO-d₆) δ 167.2, 156.7, 155.9, 149.0, 143.9, 143.6, 137.5, 134.9, 134.7, 130.0, 129.4, 127.2, 126.8, 126.4, 121.8, 116.2.

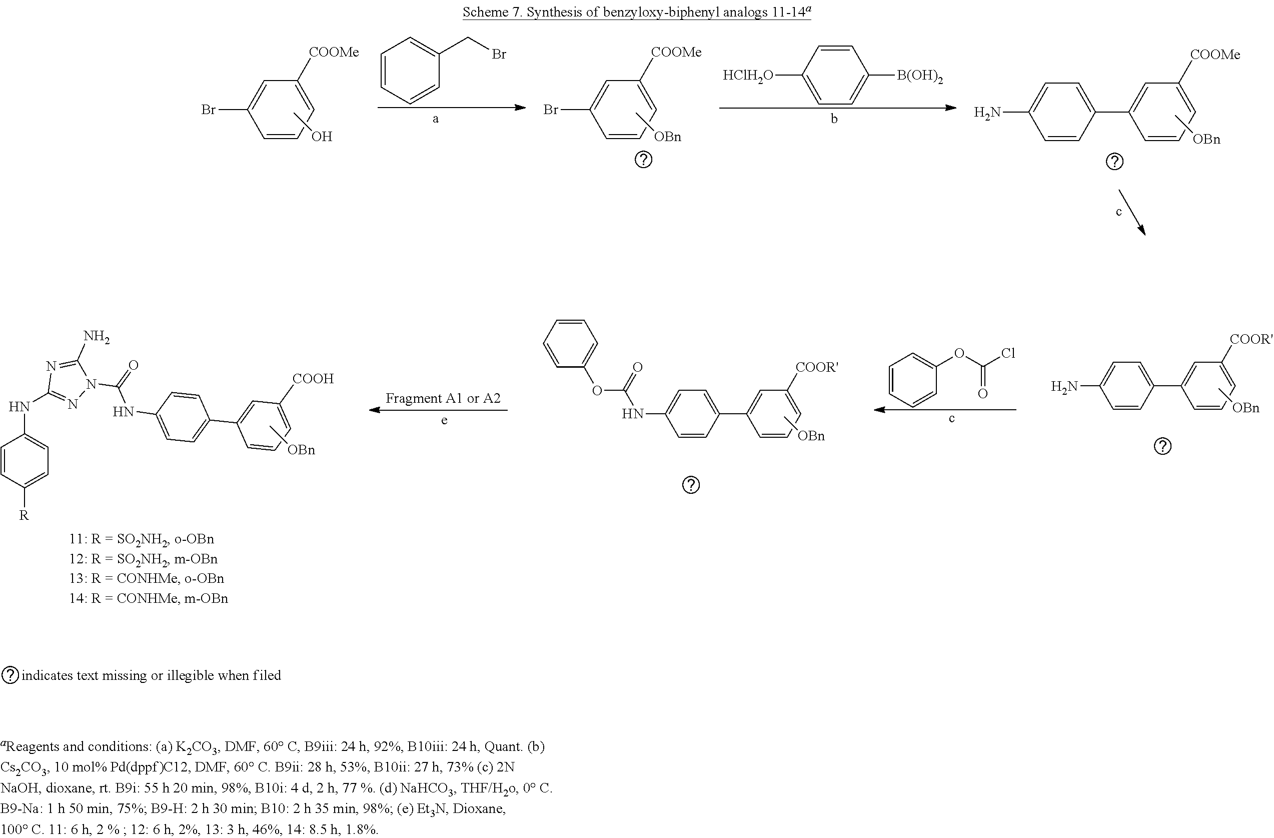

### Methyl 2-(benzyloxy)-5-bromobenzoate (B9iii)

In an oven-dried vial, methyl 5-bromo-2-hydroxybenzoate (462.1 mg, 2.00 mmol) was combined with potassium carbonate (346.9 mg, 2.51 mmol) in 1.4 mL anhydrous dimethylformamide. The mixture was allowed to stir until homogenized and subsequently benzyl bromide (0.2 mL, 1.67 mmol) was added dropwise. When the addition was complete, the reaction was heated at 60° C. for 24 h, at which point TLC indicated consumption of the starting material. Then solvent was evaporated and the mixture was diluted with 30 mL ethyl acetate. The organic phase was washed with water (3 times×10 mL), 2N NaOH (2 times x 10 mL), brine (2 times×10 mL) and was dried over sodium sulfate. The mixture was filtered and solvent was evaporated under reduced pressure to afford the title product. Product: Light Yellow Oil. Yield: 591.9 mg, 92%. HRMS (ESI) m/z calculated for C₁₅H₁₃BrO₃ [M+H]⁺=321.0121. found: 321.0113.

¹H NMR (500 MHz, DMSO-d₆) δ 7.79 (d, J=2.1 Hz, 1H), 7.70 (dd, J=8.9, 2.1 Hz, 1H), 7.47 (d, J=7.4 Hz, 2H), 7.40 (t, J=7.5 Hz, 2H), 7.32 (t, J=7.3 Hz, 1H), 7.22 (d, J=9.0 Hz, 1H), 5.22 (s, 2H), 3.81 (s, 3H). ¹³C NMR (126 MHz, DMSO-d₆) δ 164.7, 156.4, 136.5, 135.8, 132.8, 128.4, 127.7, 127.0, 122.4, 116.5, 111.6, 69.9, 52.2.

### Methyl 3-(benzyloxy)-5-bromobenzoate (B10iii)

In an oven-dried vial, methyl 3-bromo-5-hydroxybenzoate (1.71 g, 7.40 mmol) was combined with potassium carbonate (1.28 g, 9.26 mmol) in 5 mL anhydrous dimethylformamide. The mixture was allowed to stir until it was homogenized and subsequently benzyl bromide (0.73 mL, 6.17 mmol) was added dropwise. When the addition was complete, the reaction was heated at 60° C. and stirred for 24 h, at which point TLC indicated consumption of the starting material. Solvent was evaporated and the mixture was diluted with 100 mL ethyl acetate. The organic phase was washed with water (4 times×30-40 mL), 2N NaOH (5 times×30-40 mL), brine (4 times×30 mL) and was dried over sodium sulfate. The mixture was filtered, solvent was evaporated under reduced pressure, and the residue was further dried under high vacuum overnight to afford the title product. Product: Gold-yellow oil. Yield: 2.00 g, Quant. HRMS (ESI) m/z calculated for C₁₅H₁₃BrO₃ [M+H]+=321.0121. found: 321.0108.

¹H NMR (600 MHz, DMSO-d₆) δ 7.64 (t, J=1.5 Hz, 1H), 7.57-7.54 (m, 1H), 7.52 (dd, J=2.4, 1.3 Hz, 1H), 7.47-7.44 (m, 2H), 7.42-7.38 (m, 2H), 7.37-7.32 (m, 1H), 5.20 (s, 2H), 3.85 (s, 3H). ¹³C NMR (151 MHz, DMSO-d₆) δ 164.8, 159.3, 136.2, 132.6, 128.5, 128.1, 127.8, 124.0, 122.5, 122.3, 114.7, 69.9, 52.6.

### Methyl 4′-amino-4-(benzyloxy)-[1,1′-biphenyl]-3-carboxylate (B9ii)

According to General Method A: In a flame-dried vial equipped with a rubber septum was added methyl 2-(benzyloxy)-5-bromobenzoate (B9iii) (515.7 mg, 1.61 mmol), 4-aminophenylboronic acid hydrochloride (279.2 mg, 1.61 mmol) and cesium carbonate (1573.7 mg, 4.83 mmol, 3.0 eq) in 8 mL anhydrous dimethylformamide. The mixture was degassed under vigorous stirring for 20 min, and subsequently Pd(dppf)Cl₂ (117.8 mg, 0.16 mmol) was added. The mixture was purged with nitrogen and was heated at 60° C. for 28 h when TLC indicated completion. Solvent was evaporated under reduced pressure, and the residue was diluted with ethyl acetate. The organic phase was washed with water (2 times x 30 mL) and with brine (2 times×30 mL). The organic phase was then concentrated under reduced pressure, and the mixture was chromatographed using a gradient of ethyl acetate in hexanes to afford the title product. Product: White Solid. Yield: 285.4 mg, 53%. HRMS (ESI) m/z calculated for C₂₁H₁₉NO₃ [M+H]⁺=334.1438. found: 334.1431.

¹H NMR (500 MHZ, DMSO-d₆) δ 7.80 (d, J=2.0 Hz, 1H), 7.68 (dd, J=8.7, 2.2 Hz, 1H), 7.50 (d, J=7.6 Hz, 2H), 7.40 (t, J=7.5 Hz, 2H), 7.35-7.27 (m, 3H), 7.23 (d, J=8.7 Hz, 1H), 6.63 (d, J=8.2 Hz, 2H), 5.22 (s, 2H), 5.20 (s, 2H), 3.83 (s, 3H). ¹³C NMR (126 MHZ, DMSO-d₆) δ 166.2, 155.4, 148.2, 137.1, 133.2, 130.0, 128.4, 127.6, 127.3, 127.0, 126.8, 126.0, 120.8, 114.7, 114.3, 69.7, 51.9.

### Methyl 4′-amino-5-(benzyloxy)-[1,1′-biphenyl]-3-carboxylate (B10ii)

According to General Method A: In a flame-dried vial equipped with a rubber septum was added methyl 3-(benzyloxy)-5-bromobenzoate (B10iii) (610.0 mg, 1.9 mmol), 4-aminophenylboronic acid hydrochloride (329.5 mg, 1.9 mmol) and cesium carbonate (1857.2 mg, 5.7 mmol, 3.0 eq) in 9 mL anhydrous dimethylformamide. The mixture was degassed under vigorous stirring for 20 min, and subsequently Pd(dppf)Cl2 (139.0 mg, 0.19 mmol) was added. The mixture was flushed with nitrogen and was heated to 60° C. for 27 h, when TLC indicated completion. Solvent was evaporated under reduced pressure, and the residue was diluted with ethyl acetate. The organic phase was washed with water (2 times×30 mL), brine (2 times×30 mL) and the organic phase was collected and concentrated under reduced pressure. The crude mixture was further purified with chromatography, using a gradient of ethyl acetate in hexanes, to afford the title product. Product: Pale Yellow Solid. Yield: 463.1 mg, 73%. HRMS (ESI) m/z calculated for C₂₁H19NO₃ [M+H]⁺=334.1438. found: 334.1435.

¹H NMR (500 MHZ, DMSO-d₆) δ 7.69 (t, J=1.4 Hz, 1H), 7.52-7.46 (m, 2H), 7.45-7.37 (m, 6H), 7.37-7.31 (m, 1H), 6.67-6.62 (m, 2H), 5.33 (s, 2H), 5.23 (s, 2H), 3.86 (s, 3H). ¹³C NMR (126 MHz, DMSO-d₆) δ 166.2, 158.9, 149.1, 142.6, 136.9, 131.3, 128.4, 127.9, 127.7, 127.4, 125.8, 118.5, 116.6, 114.1, 112.1, 69.5, 52.2.

### Sodium 4′-amino-4-(benzyloxy)-[1,1′-biphenyl]-3-carboxylate (B9i)

Methyl 4′-amino-4-(benzyloxy)-[1,1′-biphenyl]-3-carboxylate (B9ii) (71.3 mg, 0.21 mmol) was mixed with dioxane (5 mL) and 2N NaOH (1.06 mL, 2.12 mmol) was added dropwise. The reaction was allowed to stir at rt for 55 h 20 min. Then the mixture was neutralized (pH ˜7) with 2N HCl (at 0° C.) and solvent was evaporated under reduced pressure. The residue was then chromatographed using a gradient of methanol in dichloromethane to afford the title product. Product: Pale White Solid. Yield: 70.1 mg, 98%. HRMS (ESI) m/z calculated for C₂₀H17NO₃ [M+Na]⁺=342.1106. found: 342.1094. ¹H NMR (500 MHZ, DMSO-d₆) δ 7.76 (d, J=2.5 Hz, 1H), 7.62 (dd, J=8.7, 2.5 Hz, 1H), 7.50 (d, J=7.3 Hz, 2H), 7.39 (t, J=7.5 Hz, 2H), 7.35-7.27 (m, 3H), 7.18 (d, J=8.7 Hz, 1H), 6.65-6.60 (m, 2H), 5.21 (s, 2H). ¹³C NMR (151 MHZ, DMSO-d₆) δ 167.6, 155.2, 148.1, 137.2, 133.1, 129.3, 128.3, 127.6, 127.2, 127.1, 126.7, 126.3, 122.6, 114.5, 114.3, 69.7.

### 4′-amino-5-(benzyloxy)-[1,1′-biphenyl]-3-carboxylic acid (B10i)

Methyl 4′-amino-5-(benzyloxy)-[1,1′-biphenyl]-3-carboxylate (B10ii) (449.0 mg, 1.35 mmol) was mixed with dioxane (32 mL) and 2N NaOH (6.75 mL, 13.5 mmol) was added dropwise. The reaction was allowed to stir at rt for 4 d 2 h. Then the mixture was neutralized with 2N HCl (at 0° C.) and solvent was evaporated under reduced pressure. The residue was chromatographed using a gradient of methanol in dichloromethane. The collected material was dried and then it was suspended in water (20 mL). The mixture was acidified to pH˜4 with 1N HCl and subsequently was extracted with ethyl acetate (80 mL). The organic layer was washed with brine and was dried over sodium sulfate. The mixture was filtered, the solvent was evaporated, and the residue was dried under high vacuum overnight to afford the title product. Product: light yellow solid. Yield: 330.2 mg, 77%. HRMS (ESI) m/z calculated for C₂₀H₁₇NO₃ [M+H]⁺=320.1281. found: 320.1282.

¹H NMR (500 MHz, DMSO-d₆) δ 7.69 (s, 1H), 7.49 (d, J=7.3 Hz, 2H), 7.43-7.36 (m, 6H), 7.34 (t, J=7.3 Hz, 1H), 6.64 (d, J=8.5 Hz, 2H), 5.22 (s, 2H) [(protons of the NH₂ and COOH were not observed)]. ¹³C NMR (126 MHZ, DMSO-d₆) δ 167.2, 158.8, 149.0, 142.4, 137.0, 132.5, 128.5, 127.8, 127.6, 127.3, 126.1, 118.8, 116.2, 114.2, 112.3, 69.4.

### Sodium 4-(benzyloxy)-4′-((phenoxycarbonyl)amino)-[1,1′-biphenyl]-3-carboxylate (B9-Na)

According to General Method C: Sodium 4′-amino-4-(benzyloxy)-[1,1′-biphenyl]-3-carboxylate (B9i) (67.9 mg, 0.20 mmol) was suspended in a mixture of tetrahydrofuran (1.0 mL) and water (0.5 mL). The mixture was cooled to 0° C. and sodium bicarbonate (20.2 mg, 0.24 mmol) was added, followed by the slow addition of a solution of phenyl chloroformate (26 μL, 0.21 mmol) in 0.5 mL tetrahydrofuran. The reaction was allowed to stir for 1 h 50 min at 0° C., at which point TLC indicated consumption of the starting material. The reaction mixture was then diluted with ethyl acetate (20 mL), and was washed with brine (3 times×2-5 mL). The organic phase was collected, solvent was evaporated under reduced pressure, and then the residue was dried in high vacuum overnight to afford the title product. Product: pale-yellow solid. Yield: 69.3 mg, 75%. HRMS (ESI) m/z calculated for C₂₇H21NO₅ [M+Na]+=462.1312 found: 462.1299.

¹H NMR (600 MHZ, DMSO-d₆) δ 10.34 (s, 1H), 7.69 (d, J=2.3 Hz, 1H), 7.62-7.56 (m, 4H), 7.56-7.53 (m, 1H), 7.51 (d, J=7.1 Hz, 2H), 7.46-7.40 (m, 2H), 7.39-7.35 (m, 2H), 7.32-7.28 (m, 1H), 7.26 (tt, J=7.5, 1.0 Hz, 1H), 7.25-7.21 (m, 2H), 7.10 (d, J=8.7 Hz, 1H), 5.19 (s, 2H). ¹³C NMR (151 MHZ, DMSO-d₆) δ 169.6, 155.0, 151.7, 150.5, 137.6, 137.5, 134.3, 131.7, 129.4, 128.3, 127.4, 127.14, 127.05, 126.5, 125.4, 122.0, 118.7, 114.31, 114.25, 69.6.

### 4-(benzyloxy)-4′-((phenoxycarbonyl)amino)-[1,1′-biphenyl]-3-carboxylic acid (B9-H)

According to General Method C: Sodium 4′-amino-4-(benzyloxy)-[1,1′-biphenyl]-3-carboxylate (B9i) (173.5 mg, 0.51 mmol) was suspended in a mixture of tetrahydrofuran (4.0 mL) and water (2.0 mL). The mixture was cooled to 0° C. and sodium bicarbonate (51.4 mg, 0.61 mmol) was added, followed by the slow addition of a solution of phenyl chloroformate (68 μL, 0.54 mmol) in 2.0 mL tetrahydrofuran. The reaction was allowed to stir for 2 h 30 min at 0° C., at which point TLC indicated consumption of the starting material. The reaction mixture was then acidified to pH˜4 with 1N HCl and then it was extracted with ethyl acetate (50 mL). The aqueous layer was removed, and the organic phase was washed with water (3 times×5 mL) and brine (2 times×5 mL). The organic phase was collected, solvent was evaporated under reduced pressure and then the residue was dried in high vacuum overnight to afford the title product. Product: pale yellow solid. Yield: 362.4 mg of material were isolated and carried to the next step without further purification. HRMS (ESI) m/z calculated for C₂₇H21NO₅ [M+H]⁺=440.1493. found: 440.1482. 1H NMR (600 MHZ, DMSO-d₆) δ 12.79 (s, 1H), 10.36 (s, 1H), 7.89 (d, J=2.6 Hz, 1H), 7.76 (dd, J=8.7, 2.6 Hz, 1H), 7.65-7.57 (m, 4H), 7.53-7.49 (m, 2H), 7.46-7.42 (m, 2H), 7.42-7.37 (m, 2H), 7.34-7.30 (m, 1H), 7.29-7.25 (m, 2H), 7.25-7.22 (m, 2H), 5.25 (s, 2H). ¹³C NMR (151 MHZ, DMSO-d₆) δ 167.4, 156.1, 151.7, 150.5, 137.9, 137.0, 133.5, 131.9, 130.4, 129.4, 128.4, 128.1, 127.7, 127.1, 126.7, 125.5, 122.5, 122.0, 118.7, 114.5, 69.7.

### Sodium 5-(benzyloxy)-4′-((phenoxycarbonyl)amino)-[1,1′-biphenyl]-3-carboxylate (B10)

According to General Method C: 4′-amino-5-(benzyloxy)-[1, l′-biphenyl]-3-carboxylic acid (B10i) (325.0 mg, 1.02 mmol) was suspended in a mixture of tetrahydrofuran (5 mL) and water (2.5 mL). The mixture was cooled to 0° C. and sodium bicarbonate (188.2 mg, 2.24 mmol) was added, followed by the slow addition of a solution of phenyl chloroformate (0.13 mL, 1.07 mmol) in 2.5 mL tetrahydrofuran. The reaction ran for 2 h 35 min at 0° C., at which point TLC indicated consumption of the starting material. Ethyl acetate (80 mL) was added, the organic phase was washed with brine (3 times×30 mL), solvent was evaporated under reduced pressure and the residue was dried in high vacuum overnight. The residue was washed with water, it was dried over the Buchner, and then it was suspended in ethyl acetate. The mixture was filtered and the filtrate was dried again in high vacuum to afford the title product. Product: Beige Solid. Yield: 463.0 mg, 98%. HRMS (ESI) m/z calculated for C₂₇H₂₁NO₅ [M+H]⁺=440.1493. found: 440.1487.

¹H NMR (500 MHz, DMSO-d₆) δ 10.37 (s, 1H), 7.78 (s, 1H), 7.70 (d, J=8.6 Hz, 2H), 7.62 (d, J=8.5 Hz, 2H), 7.54-7.47 (m, 4H), 7.43 (dt, J=14.8, 7.8 Hz, 4H), 7.34 (t, J=7.3 Hz, 1H), 7.30-7.22 (m, 3H), 5.25 (s, 2H). ¹³C NMR (126 MHz, DMSO-d₆) δ 167.0, 158.9, 151.7, 150.5, 141.4, 138.7, 136.8, 133.5, 132.7, 129.4, 128.5, 127.9, 127.7, 127.3, 125.5, 121.9, 119.6, 118.8, 117.3, 113.6, 69.5.

### 4′-(5-amino-3-((4-sulfamoylphenyl)amino)-1H-1,2,4-triazole-1-carboxamido)-4-(benzyloxy)-[1,1′-biphenyl]-3-carboxylic acid (11)

According to General Method D: Mixture A: In a flame-dried vial, Fragment A1 (35.8 mg, 0.14 mmol) was suspended in 0.8 mL anhydrous dioxane and the mixture was heated for a 10-15 min at 100° C., followed by the addition of triethylamine (20 μL, 0.14 mmol). The mixture was allowed to stir for another 10 min and subsequently it was added dropwise to Mixture B. The vial was rinsed with 0.5 mL anhydrous dioxane, which was also added to the Mixture B.

Mixture B: In a flame-dried vial equipped with a rubber septum, sodium 4-(benzyloxy)-4′-((phenoxycarbonyl)amino)-[1,1′-biphenyl]-3-carboxylate (B9-Na) (65.0 mg, 0.14 mmol) in 2.0 mL anhydrous dioxane was heated at 80° C. for 10 min, under nitrogen. Triethylamine (20 μL, 0.14 mmol) was added, followed by the addition of Mixture A. The reaction ran at 100° C. for 6 h. The mixture was then allowed to settle and the supernatant was removed. The residue was triturated with methanol and was dried. Water (1.0 mL) was added and the mixture was acidified to pH˜4 with 1N HCl. The precipitate was collected by centrifugation and was subsequently purified with reversed-phase chromatography using a RediSep Gold C₁₈ reusable column (particle size: 20-40 μm spherical; pore size: 100 Å) with a gradient of 0.1% formic acid in water and 0.1% formic acid in acetonitrile as the mobile phase. Product: White Solid. Yield: 1.7 mg, 2% (Purity: 95.9%). HRMS (ESI) m/z calculated for C₂₉H₂₅N₇O₆S [M+H]⁺=600.1660. found: 600.1670.

¹H NMR (600 MHz, DMSO-d₆) δ 9.69 (s, 1H), 9.66 (s, 1H), 7.89 (s, 1H), 7.83 (d, J=8.7 Hz, 2H), 7.79-7.74 (m, 3H), 7.72 (d, J=8.8 Hz, 2H), 7.68 (d, J=8.5 Hz, 2H), 7.52 (d, J=7.4 Hz, 2H), 7.44 (br s, 2H), 7.40 (t, J=7.5 Hz, 2H), 7.32 (t, J=7.3 Hz, 1H), 7.25 (d, J=8.6 Hz, 1H), 7.14 (s, 2H), 5.25 (s, 2H) [Proton of the COOH moiety was not observed]. ¹³C NMR (151 MHz, DMSO-d₆) δ 167.7, 156.7, 156.1, 155.9, 149.0, 143.9, 137.1, 136.3, 135.0, 134.9, 131.7, 129.8, 128.4, 128.0, 127.6, 127.1, 126.8, 126.4, 122.0, 116.2, 114.5, 69.7 (one carbon peak overlaps with the rest in the aromatic region).

### 4′-(5-amino-3-((4-sulfamoylphenyl)amino)-1H-1,2,4-triazole-1-carboxamido)-5-(benzyloxy)-[1,1′-biphenyl]-3-carboxylic acid (12)

According to General Method D: Mixture A: In a flame-dried vial, Fragment A1 (63.6 mg, 0.25 mmol) was suspended in 1.0 mL anhydrous dioxane and the mixture was heated for a 5-10 min at 100° C., followed by the addition of triethylamine (35 μL, 0.25 mmol). The mixture was allowed to stir for another 5 min and subsequently it was added dropwise to Mixture B. The vial was rinsed with 1.0 mL anhydrous dioxane, which was also added to the Mixture B. Mixture B: In a flame-dried vial equipped with a rubber septum, sodium 5-(benzyloxy)-4′-((phenoxycarbonyl)amino)-[1,1′-biphenyl]-3-carboxylate (B10) (115.4 mg, 0.25 mmol) in 1.0 mL anhydrous dioxane was heated at 100° C. for 5-10 min, under nitrogen. Triethylamine (35 μL, 0.25 mmol) was added, followed by the addition of Mixture A. The reaction ran at 100° C. for 6 h. Solvent was evaporated, water was added to the residue and the mixture was acidified to pH˜4 with 1N HCl. The precipitate was collected by centrifugation and was subsequently purified with preparative reverse-phase HPLC, using an Agilent 5 Prep-C18 21.2×100 mm column with a gradient of 0.1% formic acid in water and 0.1% formic acid in acetonitrile as the mobile phase (System B). Product: White Solid. Yield: 3.1 mg, 2% (Purity: 96.7%). HRMS (ESI) m/z calculated for C₂₉H25N706S [M+H]⁺=600.1660. found: 600.1671. 1H NMR (600 MHZ, DMSO-d₆) δ 9.70 (s, 1H), 9.69 (s, 1H), 7.84 (d, J=8.6 Hz, 2H), 7.82-7.76 (m, 3H), 7.76-7.69 (m, 4H), 7.54-7.48 (m, 3H), 7.46 (br s, 2H), 7.44-7.38 (m, 3H), 7.34 (t, J=7.3 Hz, 1H), 7.15 (s, 2H), 5.23 (s, 2H) [Proton of the COOH moiety was not observed]. ¹³C NMR (126 MHZ, DMSO-d₆) δ 167.9, 158.6, 156.7, 155.9, 149.0, 143.9, 140.4, 137.2, 136.9, 135.6, 134.9, 128.5, 127.8, 127.7, 126.9, 126.8, 121.9, 119.8, 116.2, 115.4, 114.5, 113.8, 69.4.

### 4′-(5-amino-3-((4-(methylcarbamoyl)phenyl)amino)-1H-1,2,4-triazole-1-carboxamido)-4-(benzyloxy)-[1,1′-biphenyl]-3-carboxylic acid (13)

According to General Method D: Mixture A: In a flame-dried vial, Fragment A² (58.1 mg, 0.25 mmol) was suspended in 1.7 mL anhydrous dioxane and the mixture was heated for a 10-15 min at 100° C., followed by the addition of triethylamine (35 μL, 0.25 mmol). The mixture was allowed to stir for another 10 min and subsequently was added dropwise to Mixture B. The vial was rinsed with 1.0 mL anhydrous dioxane, which was also added to Mixture B.

Mixture B: In a flame-dried vial equipped with a rubber septum, 4-(benzyloxy)-4′-((phenoxycarbonyl)amino)-[1,1′-biphenyl]-3-carboxylic acid (B9-H) (109.9 mg, 0.25 mmol) in 1.0 mL anhydrous dioxane was heated at 100° C. for 5 min, under nitrogen. Triethylamine (70 μL, 0.50 mmol) was added, followed by the addition of Mixture A. The reaction ran at 100° C. for 3 h. The reaction mixture was then dried, the residue was triturated with ethyl acetate and it was dried again. Water (1.0 mL) was added and the mixture was acidified to pH˜4 with 1N HCl. The precipitate was collected by centrifugation and it was subsequently purified using preparative reverse-phase HPLC, with an Agilent 5 Prep-C18 21.2×100 mm column with a gradient of 0.1% formic acid in water and 0.1% formic acid in acetonitrile as the mobile phase (System B). Product: White Solid. Yield: 66.1 mg, 46% (Purity: 95.6%). HRMS (ESI) m/z calculated for C₃₁H₂₇N₇O₅ [M+H]⁺=578.2146. found: 578.2144.

¹H NMR (500 MHZ, DMSO-d₆) δ 9.67 (s, 1H), 9.51 (s, 1H), 8.20 (q, J=4.7 Hz, 1H), 7.89 (s, 1H), 7.83-7.75 (m, 5H), 7.73 (d, J=8.7 Hz, 2H), 7.67 (d, J=8.5 Hz, 2H), 7.52 (d, J=7.3 Hz, 2H), 7.44-7.37 (m, 4H), 7.32 (t, J=7.1 Hz, 1H), 7.26 (d, J=8.7 Hz, 1H), 5.25 (s, 2H), 2.76 (d, J=4.4 Hz, 3H) [Proton of the COOH moiety was not observed]. ¹³C NMR (126 MHz, DMSO-d₆) δ 168.1, 166.4, 156.9, 155.9, 149.1, 143.4, 137.2, 136.3, 135.0, 131.7, 129.4, 128.4, 128.0, 127.9, 127.6, 127.1, 126.7, 126.3, 125.7, 121.8, 116.0, 114.5, 114.3, 69.7, 26.2.

### 4′-(5-amino-3-((4-(methylcarbamoyl)phenyl)amino)-1H-1,2,4-triazole-1-carboxamido)-5-(benzyloxy)-[1,1′-biphenyl]-3-carboxylic acid (14)

According to General Method D: Mixture A: In a flame-dried vial, Fragment A² (41.5 mg, 0.18 mmol) was suspended in 1.0 mL anhydrous dioxane and the mixture was heated for a 5-10 min at 100° C., followed by the addition of triethylamine (25 μL, 0.18 mmol). The mixture was allowed to stir for another 5 min and subsequently it was added dropwise to Mixture B. The vial was rinsed with 1.0 mL anhydrous dioxane, which was also added to the Mixture B.

Mixture B: In a flame-dried vial equipped with a rubber septum, sodium 5-(benzyloxy)-4′-((phenoxycarbonyl)amino)-[1, l′-biphenyl]-3-carboxylate (B10) (83.1 mg, 0.18 mmol) in 1.0 mL anhydrous dioxane was heated at 100° C. for 5-10 min, under nitrogen. Triethylamine (25 μL, 0.18 mmol) was added, followed by the addition of Mixture A. The reaction ran at 100° C. for 8.5 h. Solvent was evaporated, and the residue was triturated with ethyl acetate and it was dried. Then water was added and the mixture was acidified to pH˜4 with 1N HCl. The precipitate was collected by centrifugation and was subsequently purified with preparative reverse-phase HPLC, using an Agilent 5 Prep-C18 21.2×100 mm column with a gradient of 0.1% formic acid in water and 0.1% formic acid in acetonitrile as the mobile phase (System B). Product: off-white solid. Yield: 1.9 mg, 1.8% (Purity: 98.6%). HRMS (ESI) m/z calculated for C₃₁H₂₇N₇O₅ [M+H]⁺=578.2146. found: 578.2145. 1H NMR (500 MHZ, DMSO-d₆) δ 9.70 (s, 1H), 9.51 (s, 1H), 8.22 (q, J=4.2 Hz, 1H), 7.83-7.77 (m, 5H), 7.76-7.71 (m, 4H), 7.54-7.49 (m, 3H), 7.47-7.39 (m, 5H), 7.34 (t, J=7.1 Hz, 1H), 5.24 (s, 2H), 2.77 (d, J=4.2 Hz, 3H) [Proton of the COOH moiety was not observed]. ¹³C NMR (126 MHz, DMSO-d₆) δ 167.9, 166.4, 158.7, 156.9, 155.9, 149.0, 143.4, 140.6, 137.1, 137.0, 135.3, 128.4, 128.0, 127.8, 127.7, 126.9, 125.7, 121.6, 119.8, 116.0, 115.9, 113.8, 69.4, 26.2 (one carbon peak overlaps with the rest in the aromatic region).

### General Procedure for Preparation of Triazines

Reagents and solvents were obtained from commercial supplies and used without further purification. Reactions were monitored by thin-layer chromatography (TLC) using Merck pre-coated silica gel plates (analytical, SiO₂-60, F254). TLC plates were visualized under U.V. light (254 nm). Organic solutions were concentrated under reduced pressure on a Büchi rotary evaporator. Microwave reactions were performed using a Biotage® Initiator⁺ microwave synthesizer using the standard settings. Flash column chromatography was performed on a CombiflashR Rf+ (Teledyne Isco, Lincoln, NE) with RediSep RF GOLDR (silica gel, particle size 20-40 μm) prepared cartridges. Preparative reverse phase HPLC was utilized for the purification of 33i-j, 331, and 33n-o using an Agilent 1260 Infinity II system equipped with G7161A Preparative Binary Pump., G7115A Diode Array Detector WR., G7157A Preparative Autosampler, G7159B Agilent Preparative Open-Bed Fraction Collector and an Agilent 5 Prep-C18 21× 100 mm column with a gradient of 0.1% formic acid in water and 0.1% formic acid in acetonitrile as the mobile phase. Purity assessment for 33i-j, 331, and 33n-o was conducted with the same system, using an Agilent Prep-C18 4.6×100 mm, 5 μM particle size, scaler column with a gradient of 0.1% formic acid in water and 0.1% formic acid in acetonitrile as the mobile phase.

Nuclear magnetic resonance (NMR) spectra were recorded either on an Agilent DD₂ 400 (¹H NMR, ¹³C NMR recorded at 400, and 101 MHz, respectively), an Agilent DD₂ 500 (¹H NMR, ¹³C NMR recorded at 500, and 126 MHz, respectively), or an Agilent DD₂ 600 (1H NMR, ¹³C NMR recorded at 600, and 151 MHz, respectively). All spectra were recorded at room temperature, 62° C. or 80° C. as noted. Chemical shifts are reported in ppm relative to deuterated solvent as an internal standard (8H DMSO-d₆ 2.50 ppm, δ_{C} DMSO-d₆ 39.52 ppm; δ_{H} Methanol-d₆ 3.31 ppm, δ_{C} Methanol-d₆ 49.00 ppm δ_{H} Acetone-d₆ 2.05 ppm, δ_{C} Acetone-d₆ 29.84 ppm, 206.26 ppm; 8H Chloroform-d 7.26 ppm, δ_{C} Chloroform-d 77.16 ppm) with the following convention for describing multiplicity (s=singlet, d=doublet, t=triplet, q=quartet, quint=quintet, m=multiplet, br=broad signal, dd=doublet of doublets, etc). High resolution mass spectroscopy (HRMS) measurements of assayed compounds were recorded using a Waters Acquity UPLCR coupled to a Waters XevoR QTOF mass spectrometer equipped with a Waters ZSpray™ electrospray ionization source.

Mass spectrometric measurements for compounds 35-40, 33i-j, 331, and 33n-o were performed with a Shimadzu Scientific Instruments QToF 9030 LC-MS system, equipped with a Nexera LC-40D xs UHPLC, consisting of a CBM-40 Lite system controller, a DGU-405 Degasser Unit, two LC-40D XS UHPLC pumps, a SIL-40C XS autosampler and a Column Oven CTO-40S. UV data was collected with a Shimadzu Nexera HPLC/UHPLC Photodiode Array Detector SPD M-40 in the range of 190-800 nm. Mass spectra were subsequently recorded with the quadrupole time-of-flight (QToF) 9030 mass spectrometer. The samples were held at 4 deg C. in the autosampler compartment. 0.3 μL of each spiked solution were injected into a sample loop and separated on a Shim-pack Scepter C18-120, 1.9 μm, 2.1×100 mm Column, equilibrated at 40 deg C. in a column oven. A binary gradient was used:

Solvent A: Water, HPLC grade Chromasolv, with 0.1% Formic Acid

Solvent B: Acetonitrile, HPLC grade Chromasolv, with 0.1% Formic Acid

The ionization source was run in “ESI” mode, with the electrospray needle held at +4.5 kV. Nebulizer Gas was at 2 L/min, Heating Gas Flow at 10 L/min and the Interface at 300 deg C. Dry Gas was at 10 L/min, the Desolvation Line at 250 deg C. and the heating block at 400 deg C. Mass spectra were recorded in the range of 50 to 2000 m/z in positive ion mode. Measurements and data post-processing were performed with LabSolutions 5.97 Realtime Analysis and PostRun.

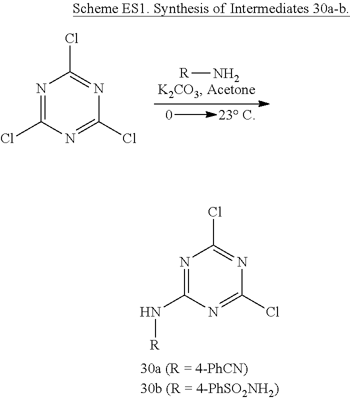

### General Procedure A: Synthesis of Intermediates 30a-b.

To a solution of cyanuric chloride (2.710 mmol, 1.0 eq.) in acetone (10 ml) at 0° C. was added potassium carbonate (5.420 mmol, 2.0 eq.) and the solution was stirred 20 minutes. The amino derivative (2.710 mmol, 1.0 eq.) was added portion wise and the reaction was allowed to warm at room temperature and stir overnight. The solvent was removed under reduced pressure. The product was collected by filtration, washed with H₂O and dried under vacuum.

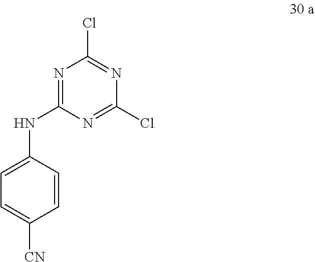 [SMILES: N#Cc1ccc(Nc2nc(Cl)nc(Cl)n2)cc1]

4-((4,6-dichloro-1,3,5-triazin-2-yl)amino) benzonitrile (30a) (0.578 g, 81% yield) ¹H NMR (400 MHZ, Methanol-d₄) δ 7.87 (d, J=8.8 Hz, 2H), 7.75 (d, J=8.9 Hz, 2H). HRMS (ESI) calcd for [M+H]⁺C₁₀H₆C₁₂N₅ 266.0001. found 266.0006.

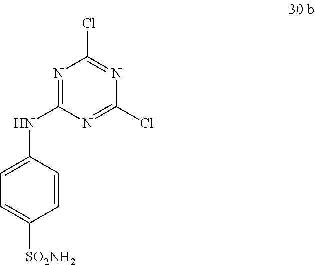 [SMILES: NS(=O)(=O)c1ccc(Nc2nc(Cl)nc(Cl)n2)cc1]

4-((4,6-dichloro-1,3,5-triazin-2-yl)amino) benzenesulfonamide (30b) (2.720 g, 31% yield) ¹H NMR (500 MHz, DMSO-d₆) δ 11.42 (s, 1H), 7.86-7.82 (m, 2H), 7.79-7.75 (m, 2H), 7.33 (s, 2H). HRMS (ESI) calcd for [M+H]⁺C₉H₈C₁₂N₅O₂S 319.9776. found 319.9771.

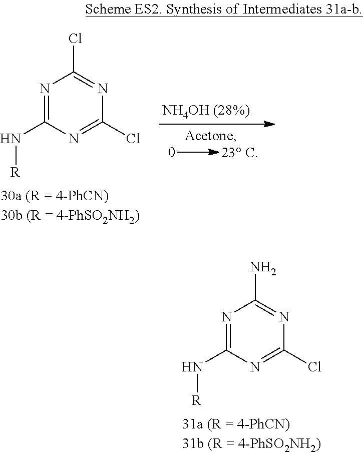

### General Procedure B: Synthesis of Intermediates 31a-b.

To a solution of 30 (14.5 mmol) in acetone (100 ml) at 0° C. was added NH₄OH 28% (1 ml). The reaction was allowed to warm at room temperature and stir overnight. The precipitated product was collected by filtration and washed with H₂O.

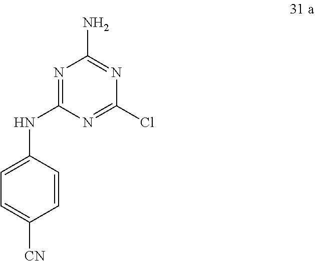 [SMILES: N#Cc1ccc(Nc2nc(N)nc(Cl)n2)cc1]

4-((4-amino-6-chloro-1,3,5-triazin-2-yl)amino) benzonitrile (31a) (3.034 g, 85% yield) 1H NMR (400 MHz, Acetone-d₆) δ 9.25 (s, 1H), 8.06 (d, J=8.6 Hz, 2H), 7.71 (d, J=8.7 Hz, 2H), 7.15 (s, 1H), 7.00 (s, 1H). HRMS (ESI) calcd for [M+H]⁺C₁₀H₈ClN₆ 247.0501. found 247.0504.

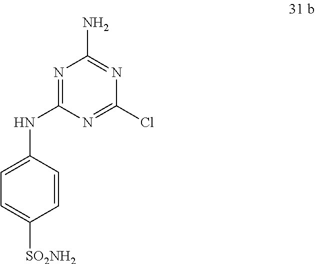 [SMILES: Nc1nc(Cl)nc(Nc2ccc(S(N)(=O)=O)cc2)n1]

4-((4-amino-6-chloro-1,3,5-triazin-2-yl)amino) benzenesulfonamide (31b) (0.131 g, 70% yield) ¹H NMR (400 MHZ, DMSO-d₆) δ 10.30 (s, 1H), 7.89 (d, J=8.5 Hz, 2H), 7.80-7.67 (m, 4H), 7.25 (s, 2H). HRMS (ESI) calcd for [M+H]⁺C₉H₁₀ClN₆O₂S 301.0274. found 301.0269.

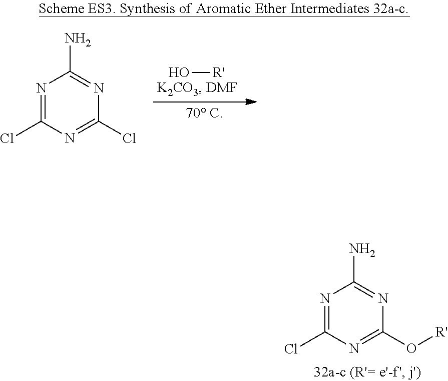

### General Procedure C: Synthesis of Aromatic Ether Intermediates 32a-c.

A solution of 4,6-dichloro-1,3,5-triazin-2-amine (1.212 mmol, 1.0 eq.), functionalized phenol with (1.212 mmol, 1.0 eq.) and K₂CO₃ (3.636 mmol, 3.0 eq.) in DMF (2 mL) was stirred at 70° C. for 18 hours. The reaction was cooled to room temperature, poured into H₂O and extracted with EtOAc. The organic layer was dried over Na₂SO₄ and the solvent was removed under reduced pressure. The crude mixture was loaded on silica to give the desired aromatic ether derivative after column chromatography.

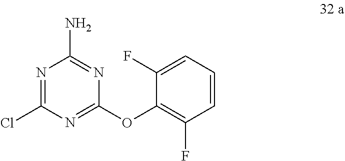 [SMILES: Nc1nc(Cl)nc(Oc2c(F)cccc2F)n1]

4-chloro-6-(2,6-difluorophenoxy)-1,3,5-triazin-2-amine (32a) (0.213 g, 68% yield) ¹H NMR (400 MHZ, DMSO-d₆) δ 8.37 (bs, 1H), 8.30 (bs, 1H), 7.46-7.22 (m, 3H). HRMS (ESI) calcd for [M+H]⁺C₉H₆ClF₂N₄O 259.0198. found 259.0193.

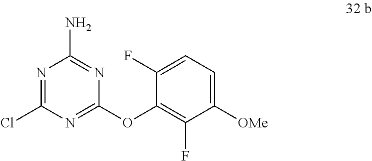 [SMILES: COc1ccc(F)c(Oc2nc(N)nc(Cl)n2)c1F]

4-chloro-6-(2,6-difluoro-3-methoxyphenoxy)-1,3,5-triazin-2-amine (32b) (0.164 g, 47% yield) ¹H NMR (400 MHZ, DMSO-d₆) δ 8.38 (bs, 1H), 8.32 (bs, 1H), 7.25 (td, J=9.7, 2.1 Hz, 1H), 7.14 (td, J=9.3, 4.9 Hz, 1H), 3.87 (s, 3H). HRMS (ESI) calcd for [M+H]⁺C₁₀H₈ClF₂N₄O₂ 289.0304. found 289.0299.

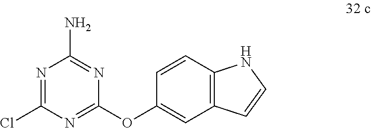 [SMILES: Nc1nc(Cl)nc(Oc2ccc3[nH]ccc3c2)n1]

4-((1H-indol-5-yl)oxy)-6-chloro-1,3,5-triazin-2-amine (32c) (0.214 g, 68%) ¹H NMR (400 MHz, DMSO-d₆) δ 11.19 (s, 1H), 8.01 (bs, 1H), 7.99 (bs, 1H), 7.46-7.37 (m, 2H), 7.33 (d, J=2.3 Hz, 1H), 6.91 (dd, J=8.7, 2.3 Hz, 1H), 6.43 (t, J=2.4 Hz, 1H). HRMS (ESI) calcd for [M+H]⁺C₁₁H₉ClN₅O 262.0496. found 262.0498.

### General Method for the Synthesis of the Aromatic Ether Analogs

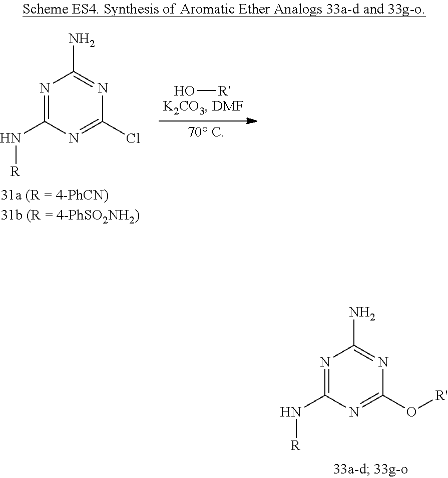

### General Procedure C: Synthesis of Aromatic Ether Analogs 32a-d and 33g-o.

A solution of 4-((4-amino-6-chloro-1,3,5-triazin-2-yl)amino) derivative (0.20 mmol), functionalized phenol (0.30 mmol) and K₂CO₃ (0.60 mmol) in DMF (2 mL) was stirred at 70° C. for 18 hours. The reaction was cooled to room temperature, poured into H₂O and extracted with EtOAc. The organic layer was dried over Na₂SO₄ and the solvent removed under reduced pressure. The crude mixture was loaded on silica to give the desired aromatic ether derivative after column chromatography.

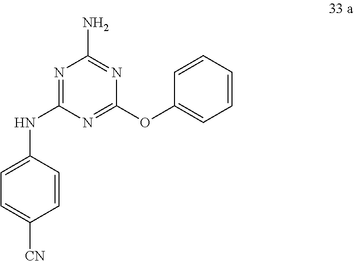 [SMILES: N#Cc1ccc(Nc2nc(N)nc(Oc3ccccc3)n2)cc1]

4-((4-amino-6-phenoxy-1,3,5-triazin-2-yl)amino) benzonitrile (33a) (62% yield) ¹H NMR (400 MHZ, Acetone-d₆) δ 9.01 (s, 1H), 7.94 (d, J-7.2 Hz, 2H), 7.58 (d, J=8.3 Hz, 2H), 7.47 (t, J=7.5 Hz, 2H), 7.31 (t, J=7.4 Hz, 1H), 7.20 (d, J=7.9 Hz, 2H), 6.67 (d, J=16.3 Hz, 2H). ¹³C NMR (101 MHz, Acetone-d₆) δ 170.02, 166.83, 153.66, 145.04, 133.49, 130.21, 126.09, 122.98, 120.43, 119.73, 105.43. HRMS (ESI) calcd. For [M+H]⁺C₁₆H₁₃N₆O 305.1151. found 305.1155.

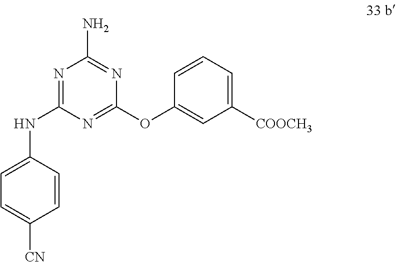 [SMILES: COC(=O)c1cccc(Oc2nc(N)nc(Nc3ccc(C#N)cc3)n2)c1]

Methyl 3-((4-amino-6-((4-cyanophenyl)amino)-1,3,5-triazin-2-yl)oxy) benzoate (33b′) (59% yield) ¹H NMR (600 MHz, Acetone-d₆) δ 9.04 (s, 1H), 7.95 (d, J=7.6 Hz, 3H), 7.79 (s, 1H), 7.62 (t, J=7.9 Hz, 1H), 7.58 (d, J=7.6 Hz, 2H), 7.49 (d, J=7.7 Hz, 1H), 6.74 (d, J=50.9 Hz, 1H), 3.90 (s, 3H). ¹³C NMR (151 MHz, Acetone-d₆) δ 169.94, 166.58, 153.67, 133.51, 132.56, 130.58, 127.84, 127.03, 123.85, 120.53, 120.46, 119.70, 105.61, 52.60. HRMS (ESI) calcd. For [M+H]⁺C₁₈H₁₅N₆O₃ 363.1206. found 363.1203.

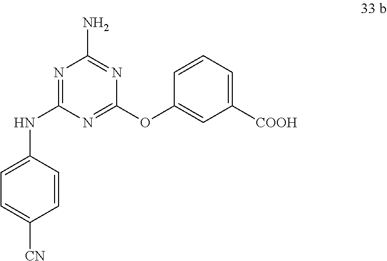 [SMILES: N#Cc1ccc(Nc2nc(N)nc(Oc3cccc(C(=O)O)c3)n2)cc1]

3-((4-amino-6-((4-cyanophenyl)amino)-1,3,5-triazin-2-yl)oxy) benzoic acid (33b) This compound was obtained by hydrolysis of 33b′ (45% yield) ¹H NMR (600 MHZ, DMSO-d₆) δ 13.19 (s, 1H), 10.02 (s, 1H), 7.84 (d, J=7.7 Hz, 3H), 7.68 (s, 1H), 7.62 (d, J=7.0 Hz, 2H), 7.57 (t, J=7.8 Hz, 1H), 7.49 (d, J=7.4 Hz, 1H), 7.38 (s, 2H). ¹³C NMR (151 MHz, DMSO-d₆) δ 170.67, 168.21, 166.66, 165.32, 152.17, 144.20, 132.70, 129.88, 126.19, 122.71, 119.45, 119.35, 103.33. HRMS (ESI) calcd for [M+H]⁺C₁₇H₁₃N₆O₃ 349.1049. found 349.1043.

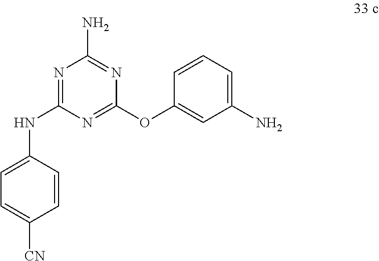 [SMILES: N#Cc1ccc(Nc2nc(N)nc(Oc3cccc(N)c3)n2)cc1]

4-((4-amino-6-(3-aminophenoxy)-1,3,5-triazin-2-yl)amino) benzonitrile (33c) (26% yield) ¹H NMR (600 MHZ, Acetone-d₆) δ 9.00 (s, 1H), 7.97 (s, 2H), 7.59 (d, J=8.1 Hz, 2H), 7.10 (t, J=8.0 Hz, 1H), 6.60 (d, J=7.8 Hz, 3H), 6.48 (s, 1H), 6.37 (d, J=7.8 Hz, 1H), 4.84 (s, 2H). ¹³C NMR (151 MHz, Acetone-d₆) δ 170.09, 166.85, 154.63, 150.73, 145.15, 133.51, 130.37, 120.44, 119.80, 112.15, 110.75, 108.76, 105.33. HRMS (ESI) calcd for [M+H]⁺C₁₆H₁₄N₇O 320.1260. found 320.1256.

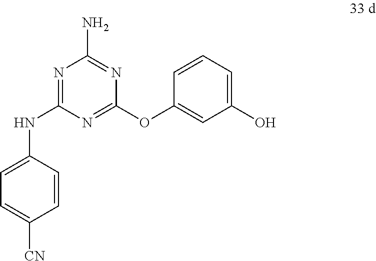 [SMILES: N#Cc1ccc(Nc2nc(N)nc(Oc3cccc(O)c3)n2)cc1]

4-((4-amino-6-(3-hydroxyphenoxy)-1,3,5-triazin-2-yl)amino) benzonitrile (33d) (23% yield). ¹H NMR (600 MHZ, Acetone-d₆) δ 9.03 (s, 1H), 8.68 (s, 1H), 7.97 (s, 2H), 7.59 (d, J=7.8 Hz, 2H), 7.26 (t, J=7.9 Hz, 1H), 6.78 (d, J=7.8 Hz, 1H), 6.66 (d, J=8.9 Hz, 3H). ¹³C NMR (151 MHz, Acetone-d₆) δ 172.39, 170.04, 166.84, 159.23, 154.60, 145.08, 133.50, 130.62, 120.45, 119.76, 113.91, 110.34, 105.41. HRMS (ESI) calcd. For [M+H]⁺C₁₆H₁₃N₆O₂ 321.1100. found 321.1095.

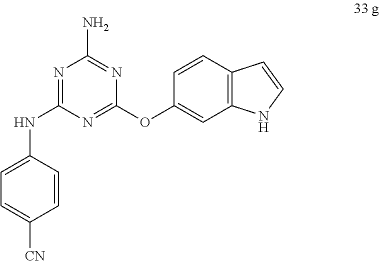 [SMILES: N#Cc1ccc(Nc2nc(N)nc(Oc3ccc4cc[nH]c4c3)n2)cc1]

4-((4-((1H-indol-6-yl)oxy)-6-amino-1,3,5-triazin-2-yl)amino) benzonitrile (33g) (54% yield) ¹H NMR (400 MHZ, Acetone-d₆) § 10.36 (s, 1H), 8.99 (s, 1H), 7.94 (s, 1H), 7.60 (d, J=8.5 Hz, 1H), 7.49 (d, J=7.4 Hz, 2H), 7.40 (s, 1H), 7.23 (s, 1H), 6.86 (d, J=8.5 Hz, 1H), 6.63 (s, 2H), 6.55 (s, 1H). ¹³C NMR (151 MHZ, Acetone-d₆) δ 170.09, 166.86, 148.96, 145.16, 137.17, 133.44, 126.73, 126.32, 121.15, 120.38, 119.76, 115.15, 105.51, 105.27, 102.40. HRMS (ESI) calcd for [M+H]⁺C₁₈H₁₄N₇O 344.1260. found 344.1265.

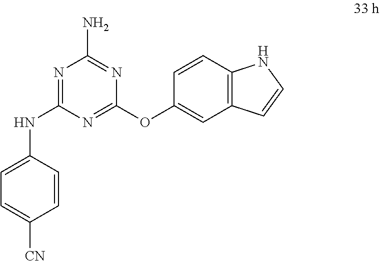 [SMILES: N#Cc1ccc(Nc2nc(N)nc(Oc3ccc4[nH]ccc4c3)n2)cc1]

4-((4-((1H-indol-5-yl)oxy)-6-amino-1,3,5-triazin-2-yl)amino) benzonitrile (33h) (36% yield) ¹H NMR (400 MHZ, Acetone-d₆) § 10.40 (s, 1H), 8.96 (s, 1H), 8.02-7.83 (m, 2H), 7.47 (d, J=8.5 Hz, 3H), 7.43 (s, 1H), 7.34 (d, J=2.2 Hz, 1H), 6.92 (dd, J=8.7, 2.2 Hz, 1H), 6.60 (s, 2H), 6.51 (t, J=2.5 Hz, 1H). ¹³C NMR (101 MHZ, Acetone-d₆) δ 170.09, 166.86, 146.95, 145.18, 134.88, 133.41, 129.26, 127.00, 120.39, 119.77, 117.05, 113.40, 112.33, 105.21, 102.64. HRMS (ESI) calcd. for [M+H]⁺C₁₈H₁₄N₇O 344.1260. found 344.1258.

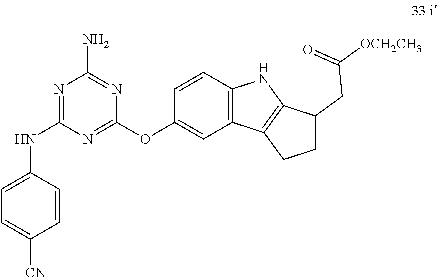 [SMILES: CCOC(=O)CC1CCc2c1[nH]c1ccc(Oc3nc(N)nc(Nc4ccc(C#N)cc4)n3)cc21]

Ethyl 2-(7-((4-amino-6-((4-cyanophenyl)amino)-1,3,5-triazin-2-yl)oxy)-1,2,3,4-tetrahydrocyclopenta[b]indol-3-yl) acetate (33i′) (52% yield) To a flame dried flask equipped with a stir bar was added 51.9 mg of 4-((4-amino-6-chloro-1,3,5-triazin-2-yl)amino) benzonitrile (31a) (0.210 mmol), 55.9 mg of ethyl 2-(7-hydroxy-1,2,3,4-tetrahydrocyclopenta[b]indol-3-yl) acetate (0.216 mmol, 1.02 eq., commercially available) and 88.4 mg of potassium carbonate (0.640 mmol, 3.04 eq.). The flask was flushed with nitrogen, at which point 3 mL of DMF were added and the solution was heated to 70° C. overnight for 15 hours. The mixture was then concentrated in vacuo and purified via column chromatography (0-100% EtOAc in Hexanes), yielding 50.4 mg of ethyl 2-(7-((4-amino-6-((4-cyanophenyl)amino)-1,3,5-triazin-2-yl)oxy)-1,2,3,4-tetrahydrocyclopenta[b]indol-3-yl) acetate (33i′) (0.107 mmol, 51.6% yield) as a colorless oil. ¹H NMR (500 MHZ, Methanol-d₄) δ 7.66 (br s, 2H), 7.40-7.25 (m, 3H), 7.10 (d, J=2.3 Hz, 1H), 6.81 (dd, J=8.7, 2.3 Hz, 1H), 4.18 (q, J=7.1 Hz, 2H), 3.60 (p, J=6.9 Hz, 1H), 2.84-2.67 (m, 4H), 2.56 (dd, J=15.9, 7.6 Hz, 1H), 2.22-2.12 (m, 1H), 1.25 (t, J=7.1 Hz, 3H). Note: H of the COOH moiety was not observed. ¹³C NMR (126 MHZ, Methanol-d₄) δ 174.34, 173.42, 169.98, 166.88, 148.49, 146.79, 145.32, 140.48, 133.65, 125.84, 120.80, 120.17, 119.67, 115.63, 112.93, 111.92, 105.48, 61.66, 40.53, 36.91, 36.77, 24.13, 14.56. MS (ESI) calcd for [M+H]⁺C₂₅H₂₄N₇O₃ 470.2. found 470.1; [M+Na]⁺492.2. found 492.1; [M−H]⁺468.2. found 468.2.

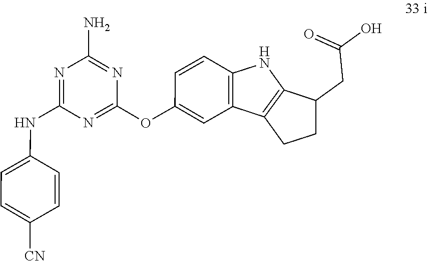 [SMILES: N#Cc1ccc(Nc2nc(N)nc(Oc3ccc4[nH]c5c(c4c3)CCC5CC(=O)O)n2)cc1]

2-(7-((4-amino-6-((4-cyanophenyl)amino)-1,3,5-triazin-2-yl)oxy)-1,2,3,4-tetrahydrocyclopenta[b]indol-3-yl) acetic acid (33i) (55% yield) To a flask equipped with a stir bar was added 35.9 mg of 33i′ (0.0765 mmol), 3 mL of THF, and 3 mL of EtOH. 270 μL of 2M NaOH (aq.) (0.540 mmol, 7.06 eq.) was then added and the solution was allowed to stir under ambient atmosphere and temperatures overnight for 15.5 hours. The solution was quenched with 2 M HCl (aq.) to pH 4 and concentrated in vacuo. The residue was then purified by reverse-phase HPLC Retention Time=13.396 min (5% B-100% B over 20 minutes), yielding 18.6 mg of compound 33i (0.0421 mmol, 55.1% yield) as a white solid. ¹H NMR (500 MHZ, DMSO-d₆) δ 10.71 (s, 1H), 9.89 (s, 1H), 7.87 (d, J=8.4 Hz, 2H), 7.55 (d, J=8.4 Hz, 2H), 7.30 (d, J=8.7 Hz, 1H), 7.22 (br s, 1H), 7.19 (br s, 1H), 7.06 (d, J=2.2 Hz, 1H), 6.77 (dd, J=8.7, 2.3 Hz, 1H), 3.50 (p, J=7.0, 6.5 Hz, 1H), 2.77-2.58 (m, 4H), 2.39 (dd, J=16.0, 8.7 Hz, 1H), 2.14-2.03 (m, 1H). ¹³C NMR (126 MHZ, DMSO-d₆) δ ¹³C

NMR (126 MHz, dmso) δ 173.49, 171.65, 168.31, 165.44, 147.64, 145.15, 144.43, 138.47, 132.65, 124.11, 119.39, 117.37, 114.43, 112.12, 110.58, 103.07, 48.60, 35.60, 35.09, 23.11. HRMS (ESI) calcd for [M+H]⁺C₂₃H₂₀N₇O₃ 442.1628. found 442.1643.

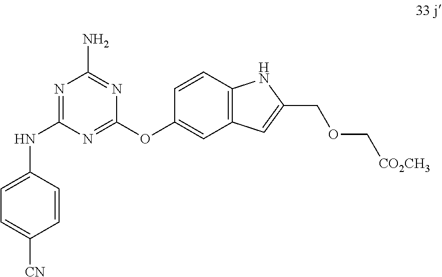 [SMILES: COC(=O)COCc1cc2cc(Oc3nc(N)nc(Nc4ccc(C#N)cc4)n3)ccc2[nH]1]

Methyl 2-((5-((4-amino-6-((4-cyanophenyl)amino)-1,3,5-triazin-2-yl)oxy)-1H-indol-2-yl) methoxy) acetate (33j′) (60% yield) Methyl 2-((5-hydroxy-1H-indol-2-yl) methoxy) acetate (34) (37.2 mg, 0.158 mmol, 1 eq.) was combined with 0.8 mL anhydrous dimethylformamide and potassium carbonate (69.7 mg, 0.5 mmol, 3.2 eq.) in a flame-dried pressure vial equipped with a rubber septum. The mixture was stirred at rt for 5-10 min, then was heated at 60° C. and stirred for another 5-10 min. 4-((4-amino-6-chloro-1,3,5-triazin-2-yl)amino) benzonitrile (31a) (41.4 mg, 0.168 mmol 1.1 eq.) was added, and the reaction stirred at 60° C. for 11 h. Afterwards, solvent was evaporated in vacuo and the residue was purified with normal-phase column chromatography (dichloromethane/methanol). Methyl 2-((5-((4-amino-6-((4-cyanophenyl)amino)-1,3,5-triazin-2-yl)oxy)-1H-indol-2-yl) methoxy) acetate (33j′) was isolated as a white solid. Yield: 42.5 mg, 60%. The corresponding carboxylic acid 33j was also identified as one of the reaction products, and was isolated as a pale white solid. Yield: 15 mg, 22%. ¹H NMR (600 MHz, DMSO-d₆) δ 11.27 (s, 1H), 9.92 (s, 1H), 7.87 (brs, 2H), 7.55 (brs, 2H), 7.36 (d, J=8.7 Hz, 1H), 7.28 (d, J=2.2 Hz, 1H), 7.23 (brs, 2H), 6.90 (dd, J=8.7, 2.3 Hz, 1H), 6.41 (d, 1H), 4.69 (s, 2H), 4.17 (s, 2H), 3.66 (s, 3H). ¹³C NMR (151 MHz, DMSO-d₆) δ 171.63, 170.44, 168.33, 165.44, 145.39, 144.42, 136.21, 134.27, 133.01, 132.67, 127.79, 119.39, 116.26, 112.35, 111.68, 103.09, 101.75, 66.30, 65.39, 51.50. [M+H]: 446.1571. found 446.1586, and 468.1405 [M+Na⁺].

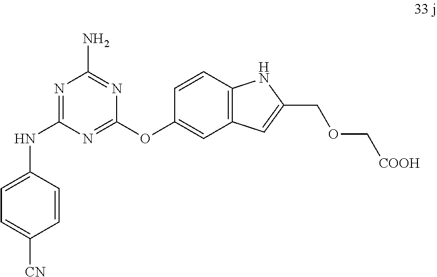 [SMILES: N#Cc1ccc(Nc2nc(N)nc(Oc3ccc4[nH]c(COCC(=O)O)cc4c3)n2)cc1]

2-((5-((4-amino-6-((4-cyanophenyl)amino)-1,3,5-triazin-2-yl)oxy)-1H-indol-2-yl) methoxy) acetic acid (33j) (27% yield) Methyl 2-((5-((4-amino-6-((4-cyanophenyl)amino)-1,3,5-triazin-2-yl)oxy)-1H-indol-2-yl) methoxy) acetate (33j′) (20 mg, 0.045 mmol, 1.0 eq.) was suspended in 4.5 mL dioxane. Sodium hydroxide (0.58 mL of a 2M aqueous solution, 1.16 mmol, 25.8 eq.) was added dropwise, and the reaction was allowed to stir at rt for 1h, when TLC indicated completion. Subsequently the reaction was concentrated to dryness, and 1.5 mL water was added. The pH was adjusted to ˜4, the precipitate was collected with centrifugation and removal of the supernatant water, followed by drying under a nitrogen stream. Then the residue was purified using a preparatory HPLC column with a gradient of acetonitrile with 0.1% formic acid/water with 0.1% formic acid, to afford 33j as a pale-white solid. Yield 5.3 mg, 27%. Purity 99%. ¹H NMR (600 MHZ, DMSO-d₆) δ 11.77 (s, 1H), 9.92 (s, 1H), 7.90 (d, J=5.2 Hz, 2H), 7.57 (d, J=6.9 Hz, 2H), 7.37 (d, J=8.6 Hz, 1H), 7.30-7.15 (m, 3H), 6.86 (dd, J=8.6, 2.3 Hz, 1H), 6.33 (s, 1H), 4.67 (s, 2H), 3.84 (s, 2H). Note: H of the COOH moiety was not observed. ¹³C NMR (151 MHZ, DMSO-d₆) δ 172.58, 171.63, 168.32, 165.47, 145.21, 144.45, 137.90, 134.14, 132.70, 127.95, 119.43, 119.40, 115.76, 112.12, 111.62, 103.08, 100.49, 68.93, 65.33. [M+H]: 432.1415. found 432.1429, and 454.1247 [M+Na⁺].

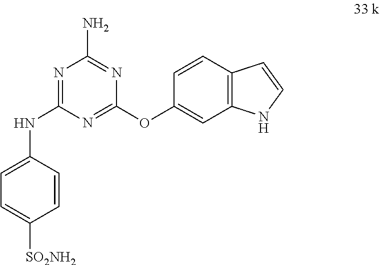 [SMILES: Nc1nc(Nc2ccc(S(N)(=O)=O)cc2)nc(Oc2ccc3cc[nH]c3c2)n1]

4-((4-((1H-indol-6-yl)oxy)-6-amino-1,3,5-triazin-2-yl)amino) benzenesulfonamide (33k) (38% yield) ¹H NMR (400 MHZ, Acetone-d₆) δ 10.35 (s, 1H), 8.89 (s, 1H), 7.91 (s, 2H), 7.67 (d, J=7.0 Hz, 2H), 7.59 (d, J=8.4 Hz, 1H), 7.38 (s, 1H), 7.22 (s, 1H), 6.86 (d, J=8.3 Hz, 1H), 6.58 (s, 2H), 6.54 (s, 1H), 6.41 (s, 2H). ¹³C NMR (101 MHZ, Acetone-d₆) δ 170.05, 166.96, 148.96, 144.16, 138.00, 137.15, 127.61, 126.67, 126.28, 121.08, 119.85, 115.16, 105.44, 102.40. HRMS (ESI) calcd for [M+H]⁺C₁₇H17N703S 398.1035. found 398.1040.

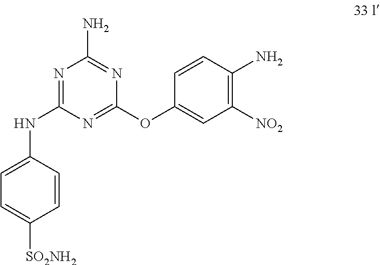 [SMILES: Nc1nc(Nc2ccc(S(N)(=O)=O)cc2)nc(Oc2ccc(N)c([N+](=O)[O-])c2)n1]

4-((4-amino-6-(4-amino-3-nitrophenoxy)-1,3,5-triazin-2-yl)amino) benzenesulfonamide (33l′) (63% yield) To a flame dried flask equipped with a stir bar 4-((4-amino-6-chloro-1,3,5-triazin-2-yl)amino) benzenesulfonamide (31b) was added (301.9 mg, 1.00 mmol), 4-amino-3-nitrophenol (154.4 mg, 1.00 mmol, 1.00 eq.) and potassium carbonate (416.7 mg, 3.02 mmol, 3.00 eq.). The flask was flushed with nitrogen, at which point 4 mL of DMF were added and the solution was heated to 70° C. overnight for 16 hours. The mixture was then concentrated in vacuo, suspended in acetone, filtered, and washed with acetone. The supernatant was then collected, concentrated in vacuo, and suspended in dichloromethane. This mixture was then filtered, washed with dichloromethane, and the precipitate was isolated, yielding 263.1 mg (0.629 mmol) of (33l′) as a burnt yellow solid. ¹H NMR (500 MHz, Acetone-d₆) δ 9.00 (s, 1H), 7.91 (br s, 2H), 7.84 (s, 1H), 7.74 (d, J=8.5 Hz, 2H), 7.35 (d, J=8.5 Hz, 1H), 7.16 (d, J=9.1 Hz, 1H), 7.14 (s, 2H), 6.72 (br s, 2H), 6.48 (br s, 2H). ¹³C NMR (126 MHZ, Acetone-d₆) δ 172.42, 169.87, 166.78, 144.77, 143.89, 142.52, 138.20, 132.24, 131.10, 127.60, 120.51, 120.02, 118.34. MS (ESI) calcd for [M+H]⁺C₁₅H₁₅N₈O₅S 419.1. found 419.1.

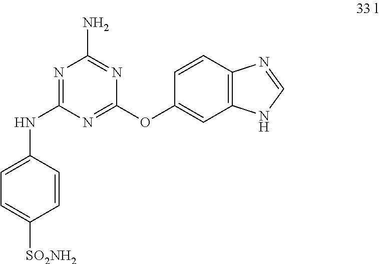 [SMILES: Nc1nc(Nc2ccc(S(N)(=O)=O)cc2)nc(Oc2ccc3nc[nH]c3c2)n1]

4-((4-((1H-benzo[d]imidazol-5-yl)oxy)-6-amino-1,3,5-triazin-2-yl)amino) benzenesulfonamide (331) (29% yield) To a flame dried microwave vessel equipped with a stir bar, 4-((4-amino-6-(4-amino-3-nitrophenoxy)-1,3,5-triazin-2-yl)amino) benzenesulfonamide (25.9 mg, 0.0779 mmol) was added SnCl₂ (38.8 mg, 0.205 mmol, 3.31 eq.). The vessel was purged with nitrogen and 2 mL of formic acid were added. The vessel was then microwaved for 15 minutes at 130° C. The contents of the flask were then concentrated in vacuo and purified on reverse phase HPLC, yielding 7.1 mg of 4-((4-((1H-benzo[d]imidazol-5-yl)oxy)-6-amino-1,3,5-triazin-2-yl)amino) benzenesulfonamide (0.0178 mmol, 28.8% yield) as a white solid. ¹H NMR (600 MHZ, DMSO-d₆) δ 9.81 (s, 1H), 8.31 (s, 1H), 7.80 (s, 2H), 7.62 (d, J=8.6 Hz, 1H), 7.57 (s, 2H), 7.41 (d, J=2.3 Hz, 1H), 7.24 (s, 1H), 7.17 (s, 3H), 7.06 (dd, J=8.6, 2.3 Hz, 1H). ¹³C NMR (151 MHZ, DMSO-d₆) δ 171.36, 168.26, 165.43, 163.01, 147.61, 142.91, 142.81, 136.80, 126.16, 118.91, 116.96. HRMS (ESI) calcd for [M+H]⁺C₁₆H₁₅N₈O₃S 399.0988. found 399.0981.

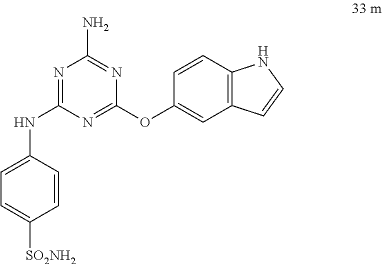 [SMILES: Nc1nc(Nc2ccc(S(N)(=O)=O)cc2)nc(Oc2ccc3[nH]ccc3c2)n1]

4-((4-((1H-indol-5-yl)oxy)-6-amino-1,3,5-triazin-2-yl)amino) benzenesulfonamide (33m) (44% yield) ¹H NMR (600 MHZ, Acetone-d₆) § 10.39 (s, 1H), 8.86 (s, 1H), 7.90 (s, 2H), 7.66 (s, 2H), 7.46 (d, J=8.5 Hz, 1H), 7.42 (s, 1H), 7.34 (s, 1H), 6.93 (d, J=8.0 Hz, 1H), 6.54 (s, 2H), 6.51 (s, 1H), 6.40 (s, 2H). ¹³C NMR (101 MHZ, Acetone-d₆) δ 170.22, 167.12, 147.11, 144.37, 138.10, 135.01, 129.41, 127.78, 127.15, 120.01, 117.24, 113.50, 112.46, 102.80. HRMS (ESI) calcd for [M+H]⁺C₁₇H₁₇N₇O₃S 398.1035. found 398.1029.

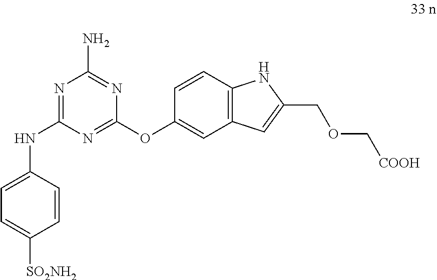 [SMILES: Nc1nc(Nc2ccc(S(N)(=O)=O)cc2)nc(Oc2ccc3[nH]c(COCC(=O)O)cc3c2)n1]

2-((5-((4-amino-6-((4-sulfamoylphenyl)amino)-1,3,5-triazin-2-yl)oxy)-1H-indol-2-yl) methoxy) acetic (33n) (12.5% yield) Methyl 2-((5-hydroxy-1H-indol-2-yl) methoxy) acetate (34) (34 mg, 0.14 mmol, 1.0 eq.) was combined with 0.7 mL anhydrous dimethylformamide and potassium carbonate (58 mg, 0.42 mmol, 3.0 eq.) in a flame-dried pressure vial equipped with a rubber septum. The mixture was stirred at rt for 5-10 min, then was heated at 60° C. and stirred for another 5-10 min. 4-((4-amino-6-chloro-1,3,5-triazin-2-yl)amino) benzenesulfonamide (31b) (42.1 mg, 0.14 mmol 1.0 eq.) was added, and the reaction stirred at 60° C. for 37 h. The mixture was concentrated and was subjected to normal-phase column chromatography (Dichloromethane/Methanol). The fractions containing the title product were collected and dried. The residue was further processed: 1 mL water was added and the pH was adjusted to ˜4. The precipitate was collected with centrifugation and removal of the supernatant water, followed by drying under a nitrogen stream. Then the residue was purified using a preparatory HPLC column with a gradient of acetonitrile with 0.1% formic acid/water with 0.1% formic acid, to afford the title compound as a beige solid. ¹H NMR (600 MHZ, DMSO-d₆) δ 11.28 (s, 1H), 9.78 (s, 1H), 7.83 (s, 2H), 7.58 (d, 2H), 7.36 (d, J=8.6 Hz, 1H), 7.27 (d, J=2.2 Hz, 1H), 7.24-7.07 (m, 4H), 6.89 (dd, J=8.6, 2.1 Hz, 1H), 6.39 (s, 1H), 4.68 (s, 2H), 4.05 (s, 2H). Note: H of the COOH moiety was not observed. ¹³C NMR (151 MHz, DMSO-d₆) δ 171.61, 171.60, 168.33, 165.52, 145.38, 143.05, 136.76, 136.60, 134.20, 127.84, 126.23, 118.94, 116.15, 112.25, 111.61, 101.42, 66.55, 65.29. HRMS (ESI) calcd for [M+H]⁺C₂₀H₂₀N₇O₆S 486.1196. found 486.1218, and 508.1037 [M+Na⁺], 95.5% purê.

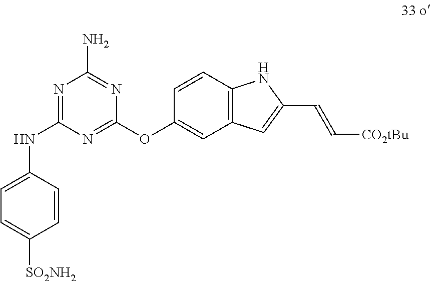 [SMILES: CC(C)(C)OC(=O)/C=C/c1cc2cc(Oc3nc(N)nc(Nc4ccc(S(N)(=O)=O)cc4)n3)ccc2[nH]1]

(E)-(4-amino-6-((2-(3-(tert-butoxy)-3-oxoprop-1-en-1-yl)-1H-indol-5-yl)oxy)-1,3,5-triazin-2-yl) (4-sulfamoylphenyl) amide (330′) (62% yield) To a flask dried in a vacuum dessicator equipped with a stir bar and containing 17.2 mg (0.0663 mmol) of tert-butyl (E)-3-(5-hydroxy-1H-indol-2-yl) acrylate (35) was added 21.1 mg (0.070 mmol, 1.06 eq.) of 4-((4-amino-6-chloro-) 1,3,5-triazin-2-yl)amino) benzenesulfonamide (31b) and 28.9 mg (0.209 mmol, 3.15 eq.) of potassium carbonate. The flask was purged with nitrogen, at which point 3 mL of DMF was added. The solution was then heated to 70° C. and stirred overnight. The solution was then concentrated in vacuo, partitioned between EtOAc and water, and extracted with EtOAc. The organic layer was dried with Na₂SO₄, filtered, and concentrated in vacuo. The residue was purified via column chromatography (10:1 CH₂Cl₂: MeOH) yielding 21.4 mg (0.0410 mmol, 61.7% yield) of (330′) as a white solid. ¹H NMR (400 MHZ, Methanol-d₄) δ 7.73 (d, J=5.6 Hz, 2H), 7.64 (d, J=5.6 Hz, 2H), 7.57 (d, J=16.0 Hz, 1H), 7.40 (d, J=8.8 Hz, 1H), 7.34 (d, J=2.2 Hz, 1H), 7.02 (dd, J=8.8, 2.3 Hz, 1H), 6.79 (s, 1H), 6.36 (d, J=16.0 Hz, 1H), 1.55 (s, 9H). ¹³C NMR (101 MHZ, Methanol-d₄) δ 173.21, 169.99, 168.26, 166.99, 147.46, 144.44, 137.86, 137.58, 136.56, 135.15, 129.97, 127.84, 120.31, 119.98, 118.72, 114.25, 112.77, 109.06, 81.71, 28.47. MS (ESI) calcd. For [M−H]⁻C₂₄H₂₄N₇O₅S 522.2. found 522.2.

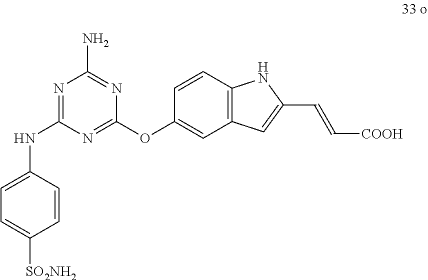 [SMILES: Nc1nc(Nc2ccc(S(N)(=O)=O)cc2)nc(Oc2ccc3[nH]c(/C=C/C(=O)O)cc3c2)n1]

(E)-3-(5-((4-amino-6-((4-sulfamoylphenyl)amino)-1,3,5-triazin-2-yl)oxy)-1H-indol-2-yl) acrylic acid (33o) (2 46% yield) To a flask equipped with a stir bar and 18.0 mg (0.0344 mmol) of (E)-(4-amino-6-((2-(3-(tert-butoxy)-3-oxoprop-1-en-1-yl)-1H-indol-5-yl)oxy)-1,3,5-triazin-2-yl) (4-sulfamoylphenyl) amide (33o′) was added 2 mL of CH₂Cl₂ and 2 mL of TFA. The solution was stirred at room temperature for 1 hour, at which point the mixture was concentrated in vacuo. The residue was then purified by reverse phase HPLC to yield the title compound (330) as a white solid. ¹H NMR (400 MHZ, DMSO-d₆) δ 11.66 (s, 1H), 9.82 (s, 1H), 7.84 (d, J=5.4 Hz, 2H), 7.59 (d, J=8.5 Hz, 2H), 7.50 (d, J=15.9 Hz, 1H), 7.38 (d, J=8.8 Hz, 1H), 7.34 (d, J=2.3 Hz, 1H), 7.24 (br s, 1H), 7.19 (br s, 3H), 7.00 (dd, J=8.7, 2.3 Hz, 1H), 6.81 (s, 1H), 6.48 (d, J=16.1 Hz, 1H). Note: H of the COOH moiety was not observed. ¹³C NMR (101 MHz, DMSO-d₆) δ 171.49, 168.29, 165.50, 145.76, 143.02, 136.79, 135.56, 135.43, 128.17, 126.24, 118.94, 118.69, 112.89, 111.82, 107.15. HRMS (ESI) calcd. For [M+H]⁺C₂₀H₁₈N₇O₅S 468.1085. found 468.1075. Retention Time=10.530 min (5% B-100% B over 20 minutes), 96.4% pure.

### General Synthetic Procedures for Pyrrolopyrimidines

Reagents and solvents were obtained from commercial supplies and used without further purification. Reactions were monitored by thin-layer chromatography (TLC) using Merck pre-coated silica gel plates (analytical, SiO₂-60, F254). TLC plates were visualized under U.V. light (254 nm). Organic solutions were concentrated under reduced pressure on a Büchi rotary evaporator. Flash column chromatography was performed on a Combiflash® Rf+ (Teledyne Isco, Lincoln, NE) with RediSep RF GOLDR (silica gel, particle size 20-40 μm for normal phase or C₁₈ for reverse phase) prepared cartridges. Preparative reverse phase HPLC was utilized for the purification of some analogues using an Agilent 1260 Infinity II system equipped with G7161A Preparative Binary Pump., G7115A Diode Array Detector WR., G7157A Preparative Autosampler, G7159B Agilent Preparative Open-Bed Fraction Collector and an Agilent 5 Prep-C18 50×21 mm column with a gradient of 0.1% formic acid in water and 0.1% formic acid in acetonitrile as the mobile phase.

Purity assessment was conducted with the same system, using an Agilent Prep-C18 4.6×100 mm, 5 μM particle size, scaler column with a gradient of 0.1% formic acid in water and 0.1% formic acid in acetonitrile as the mobile phase. Nuclear magnetic resonance (NMR) spectra were recorded on an Agilent DD₂ 400 (¹H NMR, ¹³C NMR recorded at 400, and 101 MHz, respectively), an Agilent DD₂ 500 (¹H NMR, ¹³C NMR recorded at 500, and 126 MHz, respectively), or an Agilent DD₂ 600 (¹H NMR, ¹³C NMR recorded at 600, and 151 MHz, respectively). All spectra were recorded at room temperature.

Chemical shifts are reported in ppm relative to deuterated solvent as an internal standard (δ_{H} DMSO-d₆ 2.50 ppm, δ_{C} DMSO-d₆ 39.52 ppm; 8H Methanol-d₆ 3.31 ppm, &c Methanol-d₆ 49.00 ppm; 8H Acetone-d₆ 2.05 ppm, δ_{C} Acetone-d₆ 29.84 ppm, 206.26 ppm; 8H Chloroform-d 7.26 ppm, δ_{C} Chloroform-d 77.16 ppm) with the following convention for describing multiplicity (s=singlet, d=doublet, t=triplet, q=quartet, quint=quintet, m=multiplet, br=broad signal, dd=doublet of doublets, etc). High resolution mass spectroscopy (HRMS) measurements of assayed compounds were recorded using a Waters Acquity UPLCR coupled to a Waters XevoR QTOF mass spectrometer equipped with a Waters ZSpray™ electrospray ionization source. Microwave reactions were performed using a Biotage® Initiator⁺ microwave synthesizer using the standard settings.

### Synthetic Scheme of Intermediate 3 (4-(4-aminophenoxy)-7H-pyrrolo[2,3-d]pyrimidin-2-amine)

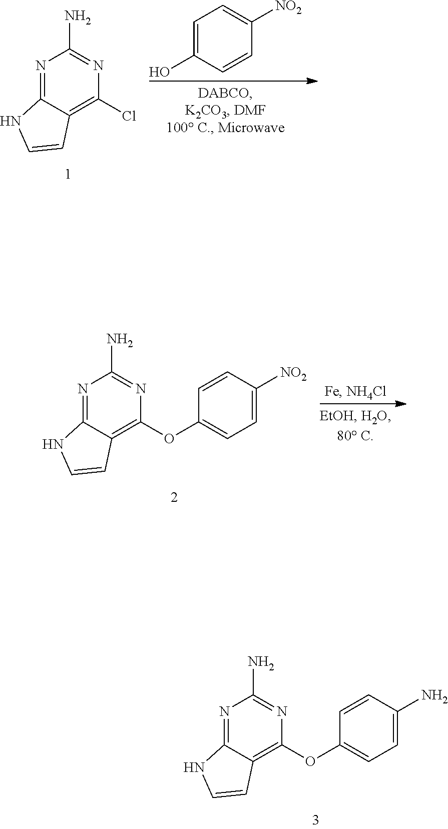 [SMILES: Nc1nc(Cl)c2cc[nH]c2n1 ; O=[N+]([O-])c1ccc(O)cc1 ; Nc1nc(Oc2ccc([N+](=O)[O-])cc2)c2cc[nH]c2n1 ; Nc1ccc(Oc2nc(N)nc3[nH]ccc23)cc1]

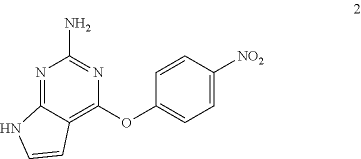 [SMILES: Nc1nc(Oc2ccc([N+](=O)[O-])cc2)c2cc[nH]c2n1]

4-(4-nitrophenoxy)-7H-pyrrolo[2,3-d]pyrimidin-2-amine (2): To an oven dried microwave vessel equipped with a stir bar was added 602.2 mg (3.57 mmol) of 4-chloro-7H-pyrrolo[2,3-d]pyrimidin-2-amine (1), 498.2 mg (3.58 mmol, 1.00 eq.) of 4-nitrophenol, 400.9 mg (3.57 mmol, 1.00 eq.) of DABCO, and 1483.5 mg (10.73 mmol, 3.01 eq.) of K2CO₃. The vessel was purged with nitrogen and 10 mL of DMF was added. The mixture was heated to 100° C. for 1.5 hrs, at which point the solution was triturated. Acetone and methanol were added and the contents were again triturated. The organic supernatants were combined, concentrated in vacuo, and purified via column chromatography (10% methanol in DCM). The product was collected and triturated with methanol. The residue was then collected, yielding 498.3 mg (1.84 mmol, 51.4% yield) of 4-(4-nitrophenoxy)-7H-pyrrolo[2,3-d]pyrimidin-2-amine (2) as a yellow orange solid. ¹H NMR (500 MHZ, Acetone-d₆) δ 8.33 (d, J=9.2 Hz, 2H), 7.53 (d, J=9.1 Hz, 2H), 7.04 (d, J=3.6 Hz, 1H), 6.34 (d, J=3.6 Hz, 1H), 5.62 (s, 1H); ¹³C NMR (126 MHz, Acetone-d₆) δ 162.4, 159.6, 145.4, 126.8, 125.9, 123.3, 121.8, 121.7, 99.12, 99.07; MS (ESI) m/z=272.0 [M+H]⁺, m/z=270.0 [M−H]⁺.

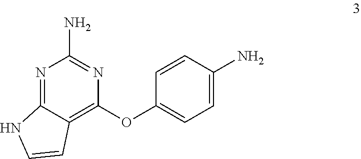 [SMILES: Nc1ccc(Oc2nc(N)nc3[nH]ccc23)cc1]

4-(4-aminophenoxy)-7H-pyrrolo[2,3-d]pyrimidin-2-amine (3): To a flask equipped with a stir bar was added 473.6 mg (1.75 mmol) of 4-(4-nitrophenoxy)-7H-pyrrolo[2,3-d]pyrimidin-2-amine (2), 487.6 mg (8.73 mmol, 4.99 eq.) of iron granules, and 469.3 mg (8.77 mmol, 5.01 eq.) of ammonium chloride. The flask was purged with nitrogen, at which point 2 mL of H2O and 2 mL of EtOH were added. The solution was heated to 80° C. and stirred overnight. The solution was then filtered and the precipitate was washed with acetone. The filtrate was collected, concentrated in vacuo, and purified via column chromatography (10% methanol in DCM), yielding 308.6 mg (1.28 mmol, 73.3% yield) of 4-(4-aminophenoxy)-7H-pyrrolo[2,3-d]pyrimidin-2-amine (3) as a white solid. ¹H NMR (400 MHZ, Methanol-d₄) δ 6.93 (d, J=8.8 Hz, 2H), 6.80-6.73 (m, 3H), 5.82 (d, J-3.6 Hz, 1H); ¹³C NMR (101 MHz, Methanol-d₄) δ 165.3, 160.7, 156.8, 146.6, 146.4, 123.4, 121.2, 117.1, 100.5, 99.0; MS (ESI) m/z=242.0 [M+H]⁺, m/z=264.0 [M+Na]⁺.

### Synthetic Scheme for Analogues 4-8

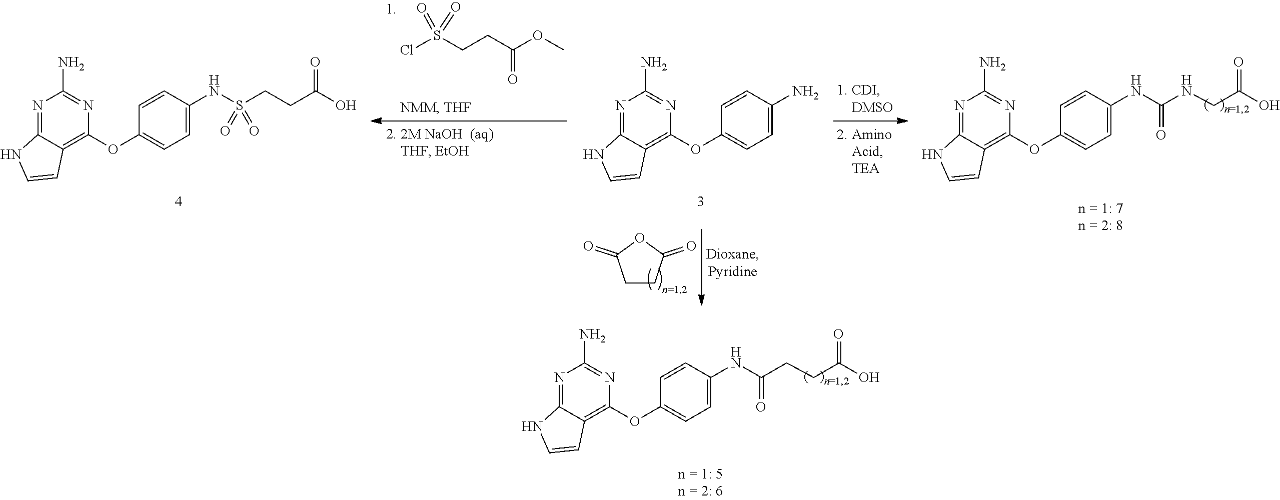

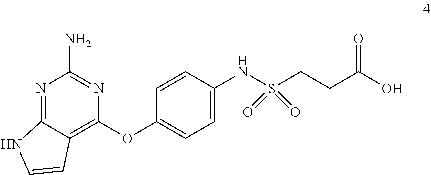 [SMILES: Nc1nc(Oc2ccc(NS(=O)(=O)CCC(=O)O)cc2)c2cc[nH]c2n1]

3-(N-(4-((2-amino-7H-pyrrolo[2,3-d]pyrimidin-4-yl)oxy)phenyl)sulfamoyl) propanoic acid (4): Step 1: To an oven dried flask equipped with a stir bar was added 21.8 mg (0.0904 mmol) of 4-(4-aminophenoxy)-7H-pyrrolo[2,3-d]pyrimidin-2-amine (3). The solution was purged with nitrogen and 2 mL of THF were added, followed by 30 μL (27.6 mg, 0.273 mmol, 3.02 eq.) of N-methylmorpholine. The solution was cooled to 0° C., at which point 20 μL (28.6 mg, 0.153 mmol, 1.70 eq.) of methyl 3-(chlorosulfonyl) propanoate was added dropwise. The solution was allowed to warm to room temperature and stirred for 1 hour. The solution was quenched with H₂O and concentrated in vacuo. The residue was purified via column chromatography (10% methanol in DCM) yielding 28.8 mg (0.0736 mmol, 81.4% yield) of methyl 3-(N-(4-((2-amino-7H-pyrrolo[2,3-d]pyrimidin-4-yl)oxy)phenyl)sulfamoyl) propanoate as a colorless oil. ¹H NMR (400 MHZ, Methanol-d₄) δ 7.31 (d, J=8.9 Hz, 2H), 7.19 (d, J=8.9 Hz, 2H), 6.86 (d, J=3.6 Hz, 1H), 6.09 (d, J=3.6 Hz, 1H), 3.68 (s, 3H), 3.44 (t, J=7.3 Hz, 2H), 2.82 (t, J=7.3 Hz, 2H); ¹³C NMR (101 MHZ, Methanol-d₄) δ 172.4, 164.4, 160.7, 156.9, 151.4, 136.3, 123.9, 123.0, 121.7, 99.9, 99.3, 52.7, 47.6, 29.3; MS (ESI) m/z=392.1 [M+H]⁺, m/z=390.1 [M−H]⁺, m/z=414.0 [M+Na]⁺.

Step 2: To a flask equipped with a stir bar was added 28.8 mg (0.0736 mmol) of methyl 3-(N-(4-((2-amino-7H-pyrrolo[2,3-d]pyrimidin-4-yl)oxy)phenyl)sulfamoyl) propanoate 2 mL of THF, and 2 mL of methanol. 260 μL (0.520 mmol, 7.07 eq.) of 2M NaOH (aq) were added and the solution was allowed to stir at room temperature for 3 hours. The solution was acidified using 260 μL of AcOH, then purified using reverse phase column chromatography (0 to 100% solvent B in solvent A), yielding 8.6 mg (0.0228 mmol, 31.0% yield) of 3-(N-(4-((2-amino-7H-pyrrolo[2,3-d]pyrimidin-4-yl)oxy)phenyl)sulfamoyl) propanoic acid (4) as a white solid. ¹H NMR (400 MHZ, DMSO-d₆) δ 11.17 (s, 1H), 8.28 (s, 1H), 7.24 (d, J=8.9 Hz, 2H), 7.18 (d, J=8.9 Hz, 2H), 6.89 (d, J=3.6 Hz, 1H), 6.06 (d, J=3.5 Hz, 1H), 6.02 (s, 2H), 3.30 (t, J=7.5 Hz, 2H), 2.58 (t, J=7.5 Hz, 2H); ¹³C NMR (101 MHZ, DMSO-d₆) δ 162.6, 159.8, 156.6, 149.8, 135.3, 123.0, 121.8, 120.8, 118.5, 98.5, 97.5, 43.7, 29.5; HRMS (ESI) m/z calculated for C₁₅H15N505S (M+H)⁺=378.0867. found=378.0859.

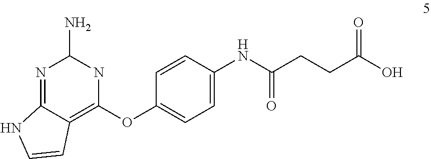

4-((4-((2-amino-7H-pyrrolo[2,3-d]pyrimidin-4-yl)oxy)phenyl)amino)-4-oxobutanoic acid (5): To an oven dried flask equipped with a stir bar was added 21.0 mg (0.0870 mmol) of 4-(4-aminophenoxy)-7H-pyrrolo[2,3-d]pyrimidin-2-amine (3) and 14.3 mg (0.143 mmol, 1.64 eq.) of succinic anhydride. The flask was purged with nitrogen, 2 mL of dioxane and 0.5 mL of pyridine were added, and the solution was allowed to stir at room temperature for 24 hours. The solution was acidified using 200 μL of acetic acid, concentrated in vacuo, and purified by reverse phase column chromatography (0 to 100% solvent B in solvent A), yielding 12.9 mg (0.0378 mmol, 43.4% yield) of (4-((4-((2-amino-7H-pyrrolo[2,3-d]pyrimidin-4-yl)oxy)phenyl)amino)-4-oxobutanoic acid (5) as a white solid. ¹H NMR (500 MHz, DMSO-d₆) δ 11.11 (s, 1H), 10.00 (s, 1H), 7.65-7.52 (m, 2H), 7.18-7.05 (m, 2H), 6.86 (dd, J=3.5, 2.0 Hz, 1H), 6.01 (dd, J=3.7, 1.9 Hz, 1H), 5.97 (s, 2H), 2.53 (dq, J=8.9, 4.7, 4.0 Hz, 4H); ¹³C NMR (126 MHZ, DMSO-d₆) δ 174.3, 170.5, 162.9, 159.8, 156.6, 148.6, 136.7, 122.5, 120.6, 120.5, 98.6, 97.5, 31.5, 29.4; HRMS (ESI) m/z calculated for C₁₆H₁₅N₅O₄ (M+H)⁺=342.1197. found=342.1196.

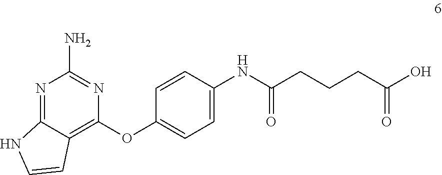 [SMILES: Nc1nc(Oc2ccc(NC(=O)CCCC(=O)O)cc2)c2cc[nH]c2n1]

5-((4-((2-amino-7H-pyrrolo[2,3-d]pyrimidin-4-yl)oxy)phenyl)amino)-5-oxopentanoic acid (6): To an oven dried flask equipped with a stir bar was added 22.7 mg (0.0941 mmol) of 4-(4-aminophenoxy)-7H-pyrrolo[2,3-d]pyrimidin-2-amine (3) and 16.4 mg (0.144 mmol, 1.53 eq.) of glutaric anhydride. The flask was purged with nitrogen, at which point 2 mL of dioxane and 0.5 mL of pyridine were added. The solution was stirred at room temperature overnight, acidified with 200 μL acetic acid, and concentrated in vacuo. The residue was purified using reverse phase column chromatography (0 to 100% solvent B in solvent A), yielding 4.3 mg (0.0121 mmol, 12.9% yield) of 5-((4-((2-amino-7H-pyrrolo[2,3-d]pyrimidin-4-yl)oxy)phenyl)amino)-5-oxopentanoic acid (6) as a white solid. ¹H NMR (500 MHz, DMSO-d₆) δ 11.13 (s, 1H), 9.97 (s, 1H), 7.60 (d, J=8.4 Hz, 2H), 7.13 (d, J=8.9 Hz, 2H), 6.87 (dd, J=3.5, 2.0 Hz, 1H), 6.03 (dd, J=3.6, 1.9 Hz, 1H), 5.98 (s, 2H), 2.35 (t, J=7.4 Hz, 2H), 2.27 (t, J=7.3 Hz, 2H), 1.82 (p, J=7.4 Hz, 2H); ¹³C NMR (126 MHz, DMSO-d₆) δ 174.2, 170.6, 162.4, 159.3, 156.1, 148.2, 136.2, 122.0, 120.20, 120.17, 98.1, 97.0, 35.4, 33.2, 20.6; HRMS (ESI) m/z calculated for C₁₇H₁₇N₅O₄ (M+H)⁺=356.1353. found=356.1344.

General Procedure for the Attachment of Amino Acid Ureas: To an oven dried flask equipped with a stir bar was added 4-(4-aminophenoxy)-7H-pyrrolo[2,3-d]pyrimidin-2-amine (1.0 eq.) and CDI (1.2-2.0 eq.). The vessel was purged with nitrogen, cooled to 0° C., and DMSO was added. The solution was allowed to warm to room temperature and stirred for 3 hours. The desired amino acid (1.0-3.0 eq.) was then added, followed by TEA (3.0-5.0 eq.). The solution was further stirred at room temperature overnight, at which point the solution was acidified using acetic acid and purified via reverse phase column chromatography or reverse phase

HPLC.

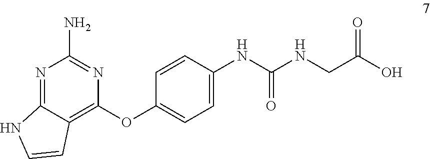 [SMILES: Nc1nc(Oc2ccc(NC(=O)NCC(=O)O)cc2)c2cc[nH]c2n1]

((4-((2-amino-7H-pyrrolo[2,3-d]pyrimidin-4-yl)oxy)phenyl) carbamoyl) glycine (7): 14.0 mg (0.0580 mmol) of 4-(4-aminophenoxy)-7H-pyrrolo[2,3-d]pyrimidin-2-amine (3), 20.5 mg (0.126 mmol, 2.18 eq.) of CDI, 0.5 mL of DMSO, 8.9 mg (0.119 mmol, 2.04 eq.) of glycine, and 40 μL (29.0 mg, 0.287 mmol, 4.94 eq.) of TEA. The solution was acidified with 100 μL acetic acid and purified via reverse phase column chromatography, yielding 8.7 mg (0.0254 mmol, 43.8% yield) of ((4-((2-amino-7H-pyrrolo[2,3-d]pyrimidin-4-yl)oxy)phenyl) carbamoyl) glycine (7) as a white solid. ¹H NMR (600 MHZ, DMSO-d₆) δ 11.12 (s, 1H), 8.92 (s, 1H), 7.41 (d, J=8.5 Hz, 2H), 7.06 (d, J=8.5 Hz, 2H), 6.86 (t, J=2.8 Hz, 1H), 6.41 (t, J=5.8 Hz, 1H), 6.12-5.90 (m, 3H), 3.74 (d, J=5.4 Hz, 2H). ¹³C NMR (151 MHz, DMSO-d₆) δ 172.3, 163.9, 159.4, 156.1, 155.3, 146.9, 137.6, 122.0, 120.1, 118.6, 98.2, 97.0, 42.1; HRMS (ESI) m/z calculated for C₁₅H₁₄N₆O₄ (M+H)⁺=343.1149. found=343.1141.

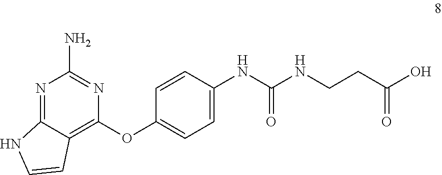 [SMILES: Nc1nc(Oc2ccc(NC(=O)NCCC(=O)O)cc2)c2cc[nH]c2n1]

3-(3-(4-((2-amino-7H-pyrrolo[2,3-d]pyrimidin-4-yl)oxy)phenyl) ureido) propanoic acid (8): 39.6 mg (0.164 mmol) of 4-(4-aminophenoxy)-7H-pyrrolo[2,3-d]pyrimidin-2-amine (3), 54.0 mg (0.333 mmol, 2.03 eq.) of CDI, 0.6 mL of DMSO, 29.6 mg (0.332 mmol, 2.03 eq.) of B-alanine, and 100 μL (72.6 mg, 0.717 mmol, 4.37 eq.) of TEA. The solution was acidified with 200 μL acetic acid and purified via reverse phase column chromatography, yielding 33.6 mg (0.0943 mmol, 57.4% yield) of ((4-((2-amino-7H-pyrrolo[2,3-d]pyrimidin-4-yl)oxy)phenyl) carbamoyl) propanoic acid (8) as a white solid. ¹H NMR (500 MHz, DMSO-d₆) δ 11.03 (s, 1H), 8.52 (s, 1H), 7.40 (d, J=8.6 Hz, 2H), 7.06 (d, J=8.6 Hz, 2H), 6.85 (t, J=2.7 Hz, 1H), 6.17 (t, J=6.1 Hz, 1H), 5.99 (dd, J=3.5, 1.8 Hz, 1H), 5.85 (s, 2H), 3.32 (q, J=6.2 Hz, 2H), 2.43 (t, J=6.5 Hz, 2H); ¹³C NMR (126 MHz, DMSO-d₆) δ 173.5, 162.6, 159.4, 156.1, 155.2, 146.9, 137.5, 122.0, 120.1, 118.7, 98.3, 97.0, 35.2, 34.7; HRMS (ESI) m/z calculated for C₁₆H₁₆N₆O₄ (M+H)⁺=357.1306. found=357.1306.

### Synthetic Scheme for Analogues 9-21

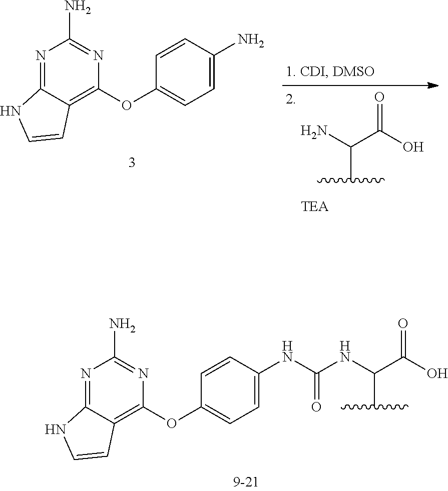 [SMILES: Nc1ccc(Oc2nc(N)nc3[nH]ccc23)cc1 ; *C(N)C(=O)O ; *C(NC(=O)Nc1ccc(Oc2nc(N)nc3[nH]ccc23)cc1)C(=O)O]

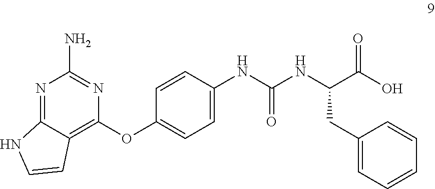 [SMILES: Nc1nc(Oc2ccc(NC(=O)N[C@@H](Cc3ccccc3)C(=O)O)cc2)c2cc[nH]c2n1]

((4-((2-amino-7H-pyrrolo[2,3-d]pyrimidin-4-yl)oxy)phenyl) carbamoyl)-L-phenylalanine (9): 14.7 mg (0.0609 mmol) of 4-(4-aminophenoxy)-7H-pyrrolo[2,3-d]pyrimidin-2-amine (3), 11.9 mg (0.0734 mmol, 1.21 eq.) of CDI, 0.4 mL of DMSO, 10.2 mg (0.0617 mmol, 1.01 eq.) of L-phenylalanine, and 35 μL (25.4 mg, 0.251 mmol, 4.12 eq.) of TEA. The solution was acidified with 50 μL acetic acid and purified via reverse phase column chromatography, yielding 9.9 mg (0.0229 mmol, 37.6% yield) of ((4-((2-amino-7H-pyrrolo[2,3-d]pyrimidin-4-yl)oxy)phenyl) carbamoyl)-L-phenylalanine (9) as a white solid. ¹H NMR (600 MHZ, DMSO-d₆) δ 11.12 (s, 1H), 8.73 (s, 1H), 7.37 (d, J=8.7 Hz, 2H), 7.31 (t, J=7.4 Hz, 2H), 7.25-7.21 (m, 3H), 7.06 (d, J=8.6 Hz, 2H), 6.86 (q, J=2.3 Hz, 1H), 6.33 (d, J=7.9 Hz, 1H), 6.00 (dd, J=3.4, 1.9 Hz, 1H), 5.97 (s, 2H), 4.47 (td, J=7.7, 5.1 Hz, 1H), 3.10 (dd, J=13.9, 5.1 Hz, 1H), 2.97 (dd, J=13.8, 7.6 Hz, 1H); ¹³C NMR (151 MHz, DMSO-d₆) δ 173.5, 162.6, 159.4, 156.1, 154.7, 147.0, 137.2, 137.1, 129.3, 128.3, 126.5, 122.1, 120.1, 118.6, 98.2, 97.0, 53.6, 37.3; HRMS (ESI) m/z calculated for C₂₂H₂₀N₆O₄ (M+H)⁺=433.1619. found=433.1612.

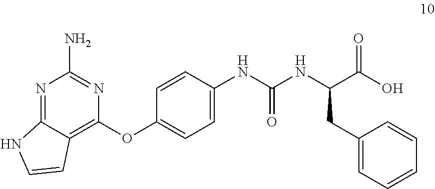 [SMILES: Nc1nc(Oc2ccc(NC(=O)N[C@H](Cc3ccccc3)C(=O)O)cc2)c2cc[nH]c2n1]

((4-((2-amino-7H-pyrrolo[2,3-d]pyrimidin-4-yl)oxy)phenyl) carbamoyl)-D-phenylalanine (10): 16.4 mg (0.0680 mmol) of 4-(4-aminophenoxy)-7H-pyrrolo[2,3-d]pyrimidin-2-amine (3), 14.1 mg (0.0870 mmol, 1.28 eq.) of CDI, 0.4 mL of DMSO, 11.9 mg (0.0720 mmol, 1.06 eq.) of D-phenylalanine, and 40 μL (29.0 mg, 0.287 mmol, 4.22 eq.) of TEA. The solution was acidified with 100 μL acetic acid and purified via reverse phase column chromatography, yielding 8.9 mg (0.0206 mmol, 30.3% yield) of ((4-((2-amino-7H-pyrrolo[2,3-d]pyrimidin-4-yl)oxy)phenyl) carbamoyl)-D-phenylalanine (10) as a white solid. ¹H NMR (400 MHZ, DMSO-d₆) δ 11.13 (s, 1H), 8.75 (s, 1H), 7.37 (d, J=8.9 Hz, 2H), 7.35-7.28 (m, 2H), 7.26-7.19 (m, 3H), 7.06 (d, J=8.9 Hz, 2H), 6.86 (dd, J=3.5, 2.2 Hz, 1H), 6.34 (d, J=8.0 Hz, 1H), 6.05-5.94 (m, 3H), 4.46 (td, J=7.7, 5.0 Hz, 1H), 3.10 (dd, J=13.8, 5.1 Hz, 1H), 2.96 (dd, J=13.8, 7.6 Hz, 1H); ¹³C NMR (101 MHz, DMSO-d₆) δ 173.6, 162.6, 159.4, 156.1, 154.8, 147.0, 137.3, 137.2, 129.3, 128.3, 126.6, 122.1, 120.1, 118.6, 98.2, 97.0, 53.6, 37.3; HRMS (ESI) m/z calculated for C₂₂H₂₀N₆O₄ (M+H)⁺=433.1619. found=433.1624.

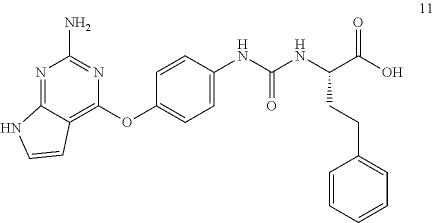 [SMILES: Nc1nc(Oc2ccc(NC(=O)N[C@@H](CCc3ccccc3)C(=O)O)cc2)c2cc[nH]c2n1]

(S)-2-(3-(4-((2-amino-7H-pyrrolo[2,3-d]pyrimidin-4-yl)oxy)phenyl) ureido)-4-phenylbutanoic acid (11): 48.8 mg (0.202 mmol) of 4-(4-aminophenoxy)-7H-pyrrolo[2,3-d]pyrimidin-2-amine (3), 65.9 mg (0.406 mmol, 2.01 eq.) of CDI, 0.8 mL of DMSO, 108.7 mg (0.607 mmol, 3.00 eq.) of L-homophenylalanine, and 120 μL (87.1 mg, 0.860 mmol, 4.26 eq.) of TEA. The solution was acidified with 200 μL acetic acid and purified via reverse phase column chromatography, yielding 57.6 mg (0.128 mmol, 63.8% yield) of(S)-2-(3-(4-((2-amino-7H-pyrrolo[2,3-d]pyrimidin-4-yl)oxy)phenyl) ureido)-4-phenylbutanoic acid (11) as a white solid. ¹H NMR (400 MHZ, DMSO-d₆) δ 11.12 (s, 1H), 8.72 (s, 1H), 7.42 (d, J=8.9 Hz, 2H), 7.29 (t, J=7.5 Hz, 2H), 7.26-7.15 (m, 3H), 7.08 (d, J=8.7 Hz, 2H), 6.86 (dd, J=3.5, 2.2 Hz, 1H), 6.59 (d, J=8.0 Hz, 1H), 6.01 (dd, J=3.6, 1.9 Hz, 1H), 5.97 (s, 2H), 4.17 (td, J=8.1, 4.8 Hz, 1H), 2.65 (t, J=7.9 Hz, 2H), 2.04 (dtd, J=13.3, 8.2, 4.9 Hz, 1H), 1.90 (dq, J=15.6, 7.8 Hz, 1H); ¹³C NMR (101 MHz, DMSO-d₆) δ 174.3, 162.6, 159.4, 156.1, 155.0, 147.0, 141.1, 137.2, 128.4, 128.3, 125.9, 122.1, 120.1, 118.7, 98.2, 97.0, 52.0, 33.7, 31.3; HRMS (ESI) m/z calculated for C₂₃H₂₂N₆O₄ (M+H)⁺=447.1775. found=447.1771.

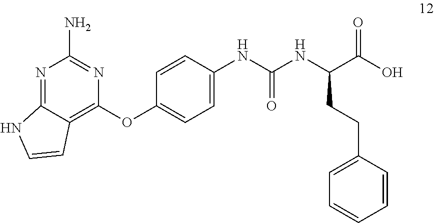 [SMILES: Nc1nc(Oc2ccc(NC(=O)N[C@H](CCc3ccccc3)C(=O)O)cc2)c2cc[nH]c2n1]

(R)-2-(3-(4-((2-amino-7H-pyrrolo[2,3-d]pyrimidin-4-yl)oxy)phenyl) ureido)-4-phenylbutanoic acid (12): 22.5 mg (0.0933 mmol) of 4-(4-aminophenoxy)-7H-pyrrolo[2,3-d]pyrimidin-2-amine (3), 19.1 mg (0.118 mmol, 1.26 eq.) of CDI, 1.5 mL of DMSO, 17.1 mg (0.0954 mmol, 1.02 eq.) of D-homophenylalanine, and 55 μL (39.9 mg, 0.394 mmol, 4.23 eq.) of TEA. The solution was acidified with 100 μL acetic acid and purified via reverse phase HPLC, yielding 11.4 mg (0.0255 mmol, 27.4% yield) of (R)-2-(3-(4-((2-amino-7H-pyrrolo[2,3-d]pyrimidin-4-yl)oxy)phenyl) ureido)-4-phenylbutanoic acid (12) as a white solid. ¹H NMR (400 MHZ, DMSO-d₆) δ 11.12 (s, 1H), 8.76 (s, 1H), 7.43 (d, J=8.6 Hz, 2H), 7.29 (t, J=7.5 Hz, 2H), 7.20 (dd, J=14.4, 7.3 Hz, 3H), 7.08 (d, J=8.5 Hz, 2H), 6.86 (t, J=2.7 Hz, 1H), 6.62 (d, J=7.9 Hz, 1H), 6.00 (t, J=2.6 Hz, 1H), 5.97 (s, 2H), 4.16 (td, J=8.0, 4.8 Hz, 1H), 2.65 (t, J=7.9 Hz, 2H), 2.04 (dtd, J=13.3, 8.1, 4.9 Hz, 1H), 1.91 (dq, J=15.1, 7.8 Hz, 1H); ¹³C NMR (101 MHz, DMSO-d₆) δ 174.3, 162.6, 159.4, 156.1, 155.0, 147.0, 141.2, 137.3, 128.4, 128.3, 125.9, 122.1, 120.1, 118.7, 98.2, 97.0, 52.1, 33.8, 31.3; HRMS (ESI) m/z calculated for C₂₃H₂₂N₆O₄ (M+H)⁺=447.1775. found=447.1764.

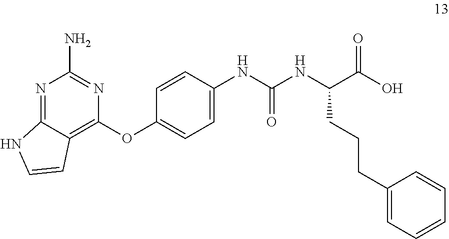 [SMILES: Nc1nc(Oc2ccc(NC(=O)N[C@@H](CCCc3ccccc3)C(=O)O)cc2)c2cc[nH]c2n1]

(S)-2-(3-(4-((2-amino-7H-pyrrolo[2,3-d]pyrimidin-4-yl)oxy)phenyl) ureido)-5-phenylpentanoic acid (13): 15.1 mg (0.0626 mmol) of 4-(4-aminophenoxy)-7H-pyrrolo[2,3-d]pyrimidin-2-amine (3), 12.4 mg (0.0765 mmol, 1.22 eq.) of CDI, 0.5 mL of DMSO, 11.6 mg (0.0600 mmol, 0.96 eq.) of(S)-2-amino-5-phenylpentanoic acid, and 40 μL (29.0 mg, 0.287 mmol, 4.58 eq.) of TEA. The solution purified via reverse phase column chromatography, yielding 4.8 mg (0.0104 mmol, 16.7% yield) of(S)-2-(3-(4-((2-amino-7H-pyrrolo[2,3-d]pyrimidin-4-yl)oxy)phenyl) ureido)-5-phenylpentanoic acid (13) as a white solid. ¹H NMR (400 MHZ, DMSO-d₆) δ 11.09 (s, 1H), 8.61 (s, 1H), 7.36 (d, J=8.6 Hz, 2H), 7.25 (t, J=7.5 Hz, 2H), 7.20-7.10 (m, 3H), 7.03 (d, J=8.6 Hz, 2H), 6.83 (q, J=2.3 Hz, 1H), 6.42 (d, J=8.0 Hz, 1H), 5.97 (dd, J=3.6, 1.8 Hz, 1H), 5.94 (s, 2H), 4.23-4.12 (m, 1H), 2.57 (h, J=5.9 Hz, 2H), 1.76-1.48 (m, 4H); ¹³C NMR (101 MHz, DMSO-d₆) δ 174.3, 162.6, 159.4, 156.1, 154.9, 147.0, 141.8, 137.2, 128.29, 128.28, 125.8, 122.1, 120.1, 118.6, 98.2, 97.0, 52.1, 34.7, 31.5, 27.2; HRMS (ESI) m/z calculated for C₂₄H₂₄N₆O₄ (M+H)⁺=461.1932. found=461.1927.

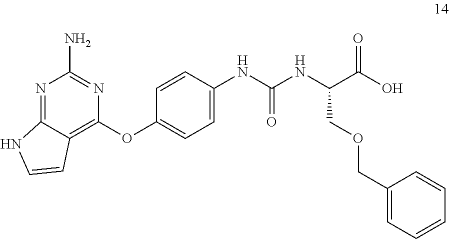 [SMILES: Nc1nc(Oc2ccc(NC(=O)N[C@@H](COCc3ccccc3)C(=O)O)cc2)c2cc[nH]c2n1]

N-((4-((2-amino-7H-pyrrolo[2,3-d]pyrimidin-4-yl)oxy)phenyl) carbamoyl)-O-benzyl-L-serine (14): 16.2 mg (0.0671 mmol) of 4-(4-aminophenoxy)-7H-pyrrolo[2,3-d]pyrimidin-2-amine (3), 11.4 mg (0.0703 mmol, 1.05 eq.) of CDI, 0.4 mL of DMSO, 13.4 mg (0.0686 mmol, 1.02 eq.) of O-benzyl-L-serine, and 40 μL (29.0 mg, 0.287 mmol, 4.27 eq.) of TEA. The solution was acidified with 50 μL acetic acid and purified via reverse phase column chromatography, yielding 3.2 mg (0.00692 mmol, 10.3% yield) of N-((4-((2-amino-7H-pyrrolo[2,3-d]pyrimidin-4-yl)oxy)phenyl) carbamoyl)-O-benzyl-L-serine (14) as a white solid. ¹H NMR (600 MHz, DMSO-d₆) δ 11.12 (s, 1H), 8.90 (s, 1H), 7.40 (d, J=8.7 Hz, 2H), 7.38-7.32 (m, 4H), 7.31-7.25 (m, 1H), 7.07 (d, J=8.6 Hz, 2H), 6.86 (t, J=2.9 Hz, 1H), 6.53 (d, J=8.4 Hz, 1H), 6.00 (dd, J=3.5, 1.9 Hz, 1H), 5.97 (s, 2H), 4.55 (d, J=12.1 Hz, 1H), 4.51 (d, J=12.1 Hz, 1H), 4.39 (dt, J=7.8, 3.4 Hz, 1H), 3.86 (dd, J=9.5, 3.7 Hz, 1H), 3.68 (dd, J=9.6, 3.5 Hz, 1H); ¹³C NMR (151 MHz, DMSO-d₆) δ 172.3, 162.5, 159.4, 156.1, 154.9, 147.0, 138.0, 137.2, 128.2, 127.50, 127.46, 122.1, 120.1, 118.6, 98.2, 97.0, 72.3, 70.4, 52.9; HRMS (ESI) m/z calculated for C₂₃H₂₂N₆O₅ (M+H)⁺=463.1724. found=463.1717.

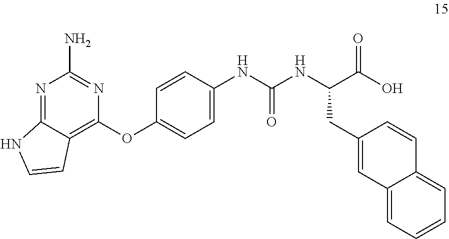 [SMILES: Nc1nc(Oc2ccc(NC(=O)N[C@@H](Cc3ccc4ccccc4c3)C(=O)O)cc2)c2cc[nH]c2n1]

(S)-2-(3-(4-((2-amino-7H-pyrrolo[2,3-d]pyrimidin-4-yl)oxy)phenyl) ureido)-3-(naphthalen-2-yl) propanoic acid (15): 15.0 mg (0.0622 mmol) of 4-(4-aminophenoxy)-7H-pyrrolo[2,3-d]pyrimidin-2-amine (4), 12.9 mg (0.0796 mmol, 1.28 eq.) of CDI, 0.4 mL of DMSO, 13.6 mg (0.0632 mmol, 1.02 eq.) of(S)-2-amino-3-(naphthalen-2-yl) propanoic acid, and 40 μL (29.0 mg, 0.287 mmol, 4.61 eq.) of TEA. The solution was acidified with 50 μL acetic acid and purified via reverse phase column chromatography, yielding 13.9 mg (0.0288 mmol, 46.3% yield) of(S)-2-(3-(4-((2-amino-7H-pyrrolo[2,3-d]pyrimidin-4-yl)oxy)phenyl) ureido)-3-(naphthalen-2-yl) propanoic acid (15) as a white solid. ¹H NMR (600 MHz, DMSO-d₆) δ 11.11 (s, 1H), 8.76 (s, 1H), 7.90-7.82 (m, 3H), 7.73 (s, 1H), 7.48 (pd, J=6.9, 1.4 Hz, 2H), 7.41 (d, J=8.3 Hz, 1H), 7.36 (d, J=8.7 Hz, 2H), 7.04 (d, J=8.7 Hz, 2H), 6.88-6.84 (m, 1H), 6.41 (d, J=7.9 Hz, 1H), 6.00 (dd, J=3.5, 1.9 Hz, 1H), 5.97 (s, 2H), 4.56 (td, J=7.6, 5.3 Hz, 1H), 3.27 (dd, J=13.8, 5.2 Hz, 1H), 3.15 (dd, J=13.8, 7.5 Hz, 1H); ¹³C NMR (151 MHz, DMSO-d₆) δ 173.6, 162.5, 159.4, 156.1, 154.8, 147.0, 137.2, 135.1, 133.0, 131.9, 127.74, 127.68, 127.63, 127.45, 127.43, 126.0, 125.5, 122.1, 120.1, 118.6, 98.2, 97.0, 53.7, 37.6; HRMS (ESI) m/z calculated for C₂₆H₂₂N₆O₄ (M+H)⁺=483.1775. found=483.1765.

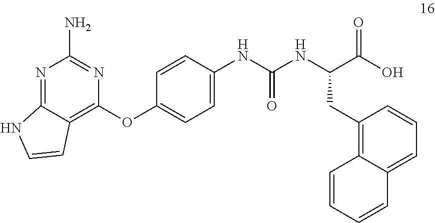 [SMILES: Nc1nc(Oc2ccc(NC(=O)N[C@@H](Cc3cccc4ccccc34)C(=O)O)cc2)c2cc[nH]c2n1]

(S)-2-(3-(4-((2-amino-7H-pyrrolo[2,3-d]pyrimidin-4-yl)oxy)phenyl) ureido)-3-(naphthalen-1-yl) propanoic acid (16): 17.2 mg (0.0713 mmol) of 4-(4-aminophenoxy)-7H-pyrrolo[2,3-d]pyrimidin-2-amine (3), 14.3 mg (0.0882 mmol, 1.24 eq.) of CDI, 0.4 mL of DMSO, 15.2 mg (0.0706 mmol, 0.99 eq.) of(S)-2-amino-3-(naphthalen-1-yl) propanoic acid, and 40 μL (29.0 mg, 0.287 mmol, 4.02 eq.) of TEA. The solution was acidified with 100 μL acetic acid and purified via reverse phase column chromatography, yielding 16.9 mg (0.0350 mmol, 49.1% yield) of(S)-2-(3-(4-((2-amino-7H-pyrrolo[2,3-d]pyrimidin-4-yl)oxy)phenyl) ureido)-3-(naphthalen-1-yl) propanoic acid (16) as a white solid. ¹H NMR (600 MHz, DMSO-d₆) δ 11.12 (s, 1H), 8.71 (s, 1H), 8.19 (d, J=8.4 Hz, 1H), 7.94 (dd, J=8.1, 1.3 Hz, 1H), 7.83 (d, J=8.1 Hz, 1H), 7.58 (ddd, J=8.4, 6.8, 1.4 Hz, 1H), 7.53 (ddd, J=8.0, 6.7, 1.2 Hz, 1H), 7.45 (dd, J=8.1, 7.0 Hz, 1H), 7.41 (dd, J=7.1, 1.3 Hz, 1H), 7.35 (d, J=8.9 Hz, 2H), 7.05 (d, J=8.9 Hz, 2H), 6.86 (dd, J=3.6, 2.2 Hz, 1H), 6.54 (d, J=8.0 Hz, 1H), 5.99 (dd, J=3.5, 1.9 Hz, 1H), 5.97 (s, 2H), 4.57 (td, J=8.2, 5.5 Hz, 1H), 3.60 (dd, J=14.1, 5.5 Hz, 1H), 3.36 (dd, J=14.1, 8.3 Hz, 1H); ¹³C NMR (151 MHZ, DMSO-d₆) δ 173.7, 162.5, 159.4, 156.1, 154.9, 147.1, 137.0, 133.51, 133.45, 131.7, 128.7, 127.6, 127.3, 126.2, 125.7, 125.4, 123.5, 122.1, 120.1, 118.8, 98.2, 97.0, 53.5, 34.9; HRMS (ESI) m/z calculated for C₂₆H₂₂N₆O₄ (M+H)⁺=483.1775. found=483.1796.

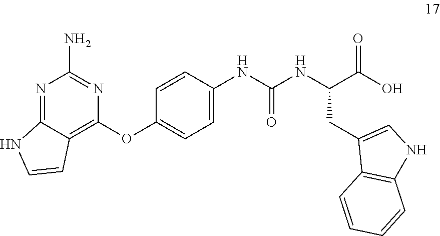 [SMILES: Nc1nc(Oc2ccc(NC(=O)N[C@@H](Cc3c[nH]c4ccccc34)C(=O)O)cc2)c2cc[nH]c2n1]

((4-((2-amino-7H-pyrrolo[2,3-d]pyrimidin-4-yl)oxy)phenyl) carbamoyl)-L-tryptophan (17): 16.1 mg (0.0667 mmol) of 4-(4-aminophenoxy)-7H-pyrrolo[2,3-d]pyrimidin-2-amine (3), 12.8 mg (0.0789 mmol, 1.18 eq.) of CDI, 0.4 mL of DMSO, 12.9 mg (0.0632 mmol, 0.95 eq.) of L-tryptophan, and 40 μL (29.0 mg, 0.287 mmol, 4.30 eq.) of TEA. The solution was acidified with 100 μL acetic acid and purified via reverse phase column chromatography, yielding 12.3 mg (0.0261 mmol, 39.1% yield) of ((4-((2-amino-7H-pyrrolo[2,3-d]pyrimidin-4-yl)oxy)phenyl) carbamoyl)-L-tryptophan (17) as a white solid. ¹H NMR (600 MHz, DMSO-d₆) δ 11.11 (s, 1H), 10.91 (s, 1H), 8.76 (s, 1H), 7.54 (d, J=7.9 Hz, 1H), 7.37 (d, J=8.7 Hz, 2H), 7.35 (d, J=8.1 Hz, 1H), 7.15 (d, J=2.2 Hz, 1H), 7.11-7.03 (m, 3H), 6.97 (t, J=7.4 Hz, 1H), 6.86 (dd, J=3.5, 2.2 Hz, 1H), 6.30 (d, J=7.9 Hz, 1H), 6.00 (dd, J=3.5, 1.9 Hz, 1H), 5.97 (s, 2H), 4.51 (q, J=6.6 Hz, 1H), 3.22 (dd, J=14.7, 5.3 Hz, 1H), 3.13 (dd, J=14.7, 6.7 Hz, 1H); ¹³C NMR (151 MHZ, DMSO-d₆) δ 174.0, 162.6, 159.4, 156.1, 154.8, 147.0, 137.2, 136.1, 127.5, 123.7, 122.1, 120.9, 120.1, 118.6, 118.4, 111.3, 109.4, 98.2, 97.0, 53.1, 27.6; HRMS (ESI) m/z calculated for C₂₄H₂₁N₇O₄ (M+H)⁺=472.1728. found-472.1725.

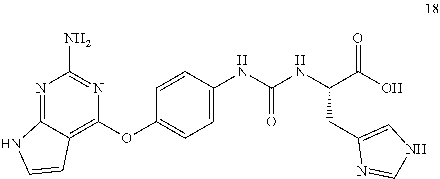 [SMILES: Nc1nc(Oc2ccc(NC(=O)N[C@@H](Cc3c[nH]cn3)C(=O)O)cc2)c2cc[nH]c2n1]

((4-((2-amino-7H-pyrrolo[2,3-d]pyrimidin-4-yl)oxy)phenyl) carbamoyl)-L-histidine (18): 14.5 mg (0.0601 mmol) of 4-(4-aminophenoxy)-7H-pyrrolo[2,3-d]pyrimidin-2-amine (3), 12.0 mg (0.0740 mmol, 1.23 eq.) of CDI, 0.4 mL of DMSO, 9.7 mg (0.0625 mmol, 1.04 eq.) of L-histidine, and 35 μL (25.4 mg, 0.251 mmol, 4.18 eq.) of TEA. The solution was acidified with 50 μL acetic acid and purified via reverse phase column chromatography, yielding 11.1 mg (0.0263 mmol, 43.7% yield) of ((4-((2-amino-7H-pyrrolo[2,3-d]pyrimidin-4-yl)oxy)phenyl) carbamoyl)-L-histidine (18) as a white solid. ¹H NMR (500 MHZ, DMSO-d₆) δ 11.11 (s, 1H), 8.90 (s, 1H), 7.63 (s, 1H), 7.39 (d, J=8.8 Hz, 2H), 7.06 (d, J=8.8 Hz, 2H), 6.91-6.83 (m, 2H), 6.46 (d, J=7.7 Hz, 1H), 5.99 (dd, J=3.6, 1.9 Hz, 1H), 5.97 (s, 2H), 4.41 (q, J=7.3, 6.8 Hz, 1H), 2.96 (d, J=5.9 Hz, 2H); ¹³C NMR (126 MHZ, DMSO-d₆) δ 173.6, 162.6, 159.4, 156.1, 154.8, 146.9, 137.3, 134.7, 122.0, 120.1, 118.6, 98.2, 97.0, 52.6, 29.4; HRMS (ESI) m/z calculated for C₁₉H₁₈N₈O₄ (M+H)⁺=423.1524. found=423.1519.

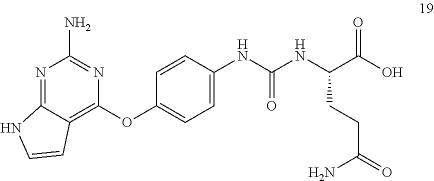 [SMILES: NC(=O)CC[C@H](NC(=O)Nc1ccc(Oc2nc(N)nc3[nH]ccc23)cc1)C(=O)O]

((4-((2-amino-7H-pyrrolo[2,3-d]pyrimidin-4-yl)oxy)phenyl) carbamoyl)-L-glutamine (19): 14.1 mg (0.0584 mmol) of 4-(4-aminophenoxy)-7H-pyrrolo[2,3-d]pyrimidin-2-amine (3), 12.4 mg (0.0765 mmol, 1.31 eq.) of CDI, 0.4 mL of DMSO, 8.6 mg (0.0588 mmol, 1.01 eq.) of L-glutamine, and 40 μL (29.1 mg, 0.287 mmol, 4.91 eq.) of TEA. The solution was acidified with 100 μL acetic acid and purified via reverse phase column chromatography, yielding 3.9 mg (0.0094 mmol, 16.1% yield) of ((4-((2-amino-7H-pyrrolo[2,3-d]pyrimidin-4-yl)oxy)phenyl) carbamoyl)-L-glutamine (19) as a white solid. ¹H NMR (500 MHZ, DMSO-d₆) δ 11.11 (t, J=2.1 Hz, 1H), 8.69 (s, 1H), 7.40 (d, J=9.0 Hz, 2H), 7.33 (br s, 1H), 7.07 (d, J=8.9 Hz, 2H), 6.86 (dd, J=3.6, 2.2 Hz, 1H), 6.78 (br s, 1H), 6.45 (d, J=7.9 Hz, 1H), 5.99 (dd, J=3.5, 1.9 Hz, 2H), 5.97 (s, 2H), 4.16 (td, J=8.1, 5.0 Hz, 1H), 2.22-2.08 (m, 2H), 2.04-1.93 (m, 1H), 1.85-1.72 (m, 1H); ¹³C NMR (126 MHz, DMSO-d₆) δ 174.1, 173.4, 162.5, 159.4, 156.1, 154.9, 147.0, 137.2, 122.1, 120.1, 118.7, 98.2, 97.0, 52.0, 31.2, 27.8; HRMS (ESI) m/z calculated for C₁₈H₁₉N₇O₅ (M+H)⁺=414.1520. found=414.1514.

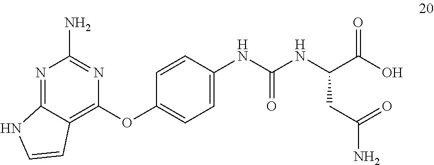 [SMILES: NC(=O)C[C@H](NC(=O)Nc1ccc(Oc2nc(N)nc3[nH]ccc23)cc1)C(=O)O]

((4-((2-amino-7H-pyrrolo[2,3-d]pyrimidin-4-yl)oxy)phenyl) carbamoyl)-L-asparagine (20): 17.7 mg (0.0734 mmol) of 4-(4-aminophenoxy)-7H-pyrrolo[2,3-d]pyrimidin-2-amine (3), 14.5 mg (0.0894 mmol, 1.22 eq.) of CDI, 0.4 mL of DMSO, 10.0 mg (0.0757 mmol, 1.03 eq.) of L-asparagine, and 40 μL (29.1 mg, 0.287 mmol, 3.91 eq.) of TEA. The solution was acidified with 100 μL acetic acid and purified via reverse phase column chromatography, yielding 14.3 mg (0.0358 mmol, 48.8% yield) of ((4-((2-amino-7H-pyrrolo[2,3-d]pyrimidin-4-yl)oxy)phenyl) carbamoyl)-L-asparagine (20) as a white solid. ¹H NMR (500 MHz, DMSO-d₆) δ 11.11 (s, 1H), 8.98 (s, 1H), 7.48 (br s, 1H), 7.41 (d, J=8.9 Hz, 2H), 7.06 (d, J=8.9 Hz, 2H), 6.94 (br s, 1H), 6.86 (dd, J=3.6, 2.1 Hz, 1H), 6.52 (d, J=8.4 Hz, 1H), 5.99 (dd, J=3.5, 1.9 Hz, 1H), 5.97 (s, 2H), 4.42 (dt, J=8.4, 5.2 Hz, 1H), 2.67 (dd, J=15.9, 5.7 Hz, 1H), 2.55 (dd, J=15.8, 4.8 Hz, 1H); ¹³C NMR (126 MHz, DMSO-d₆) δ 173.6, 171.9, 162.6, 159.4, 156.1, 154.9, 146.9, 137.4, 122.1, 120.1, 118.5, 98.2, 97.0, 49.2, 37.3; HRMS (ESI) m/z calculated for C₁₇H₁₇N₇O₅ (M+H)⁺=400.1364. found=400.1366.

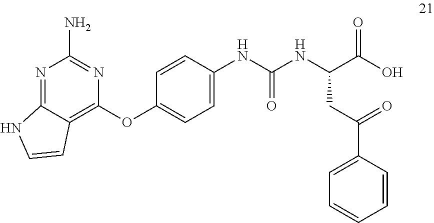 [SMILES: Nc1nc(Oc2ccc(NC(=O)N[C@@H](CC(=O)c3ccccc3)C(=O)O)cc2)c2cc[nH]c2n1]

(S)-2-(3-(4-((2-amino-7H-pyrrolo[2,3-d]pyrimidin-4-yl)oxy)phenyl) ureido)-4-oxo-4-phenylbutanoic acid (21): 15.0 mg (0.0622 mmol) of 4-(4-aminophenoxy)-7H-pyrrolo[2,3-d]pyrimidin-2-amine (3), 21.3 mg (0.131 mmol, 2.11 eq.) of CDI, 0.5 mL of DMSO, 42.9 mg (0.187 mmol, 3.00 eq.) of(S)-1-carboxy-3-oxo-3-phenylpropan-1-aminium chloride, and 40 μL (29.1 mg, 0.287 mmol, 4.61 eq.) of TEA. The solution was acidified with 100 μL acetic acid and purified via reverse phase column chromatography, yielding 11.6 mg (0.0252 mmol, 40.5% yield) of(S)-2-(3-(4-((2-amino-7H-pyrrolo[2,3-d]pyrimidin-4-yl)oxy)phenyl) ureido)-4-oxo-4-phenylbutanoic acid (21) as a white solid. ¹H NMR (500 MHZ, DMSO-d₆) δ 11.11 (s, 1H), 8.88 (s, 1H), 8.00 (d, J=7.0 Hz, 2H), 7.67 (t, J=7.4 Hz, 1H), 7.55 (t, J=7.8 Hz, 2H), 7.40 (d, J=9.0 Hz, 2H), 7.06 (d, J=9.0 Hz, 2H), 6.86 (dd, J=3.6, 2.2 Hz, 1H), 6.61 (d, J=8.4 Hz, 1H), 5.99 (dd, J=3.5, 2.0 Hz, 3H), 5.97 (s, 2H), 4.71 (dt, J=8.4, 4.9 Hz, 1H), 3.65 (dd, J=18.0, 5.3 Hz, 1H), 3.53 (dd, J=17.9, 4.7 Hz, 1H); ¹³C NMR (126 MHZ, DMSO-d₆) δ 197.9, 173.4, 162.6, 159.4, 156.1, 154.9, 147.0, 137.2, 136.1, 133.6, 128.8, 128.0, 122.1, 120.1, 118.6, 98.2, 97.0, 48.4, 40.9; HRMS (ESI) m/z calculated for C₂₃H₂₀N₆O₅ (M+H)⁺=461.1568. found=461.1569.

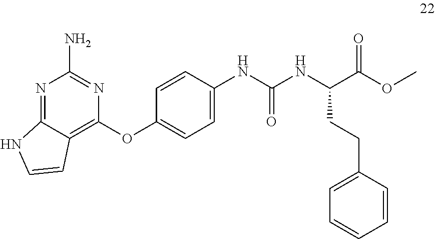 [SMILES: COC(=O)[C@H](CCc1ccccc1)NC(=O)Nc1ccc(Oc2nc(N)nc3[nH]ccc23)cc1]

Methyl(S)-2-(3-(4-((2-amino-7H-pyrrolo[2,3-d]pyrimidin-4-yl)oxy)phenyl) ureido)-4-phenylbutanoate (22): 14.6 mg (0.0605 mmol) of 4-(4-aminophenoxy)-7H-pyrrolo[2,3-d]pyrimidin-2-amine (3), 12.0 mg (0.0740 mmol, 1.22 eq.) of CDI, 0.4 mL of DMSO, 14.2 mg (0.0618 mmol, 1.02 eq.) of(S)-1-methoxy-1-oxo-4-phenylbutan-2-aminium chloride, and 35 μL (25.4 mg, 0.251 mmol, 4.15 eq.) of TEA. The solution was acidified with 100 μL acetic acid and purified via reverse phase column chromatography, yielding 11.0 mg (0.0239 mmol, 39.5% yield) of methyl(S)-2-(3-(4-((2-amino-7H-pyrrolo[2,3-d]pyrimidin-4-yl)oxy)phenyl) ureido)-4-phenylbutanoate (22) as a white solid. ¹H NMR (500 MHZ, Acetone-d₆) δ 10.22 (s, 1H), 8.19 (s, 1H), 7.54 (d, J=8.9 Hz, 2H), 7.33-7.22 (m, 4H), 7.21-7.17 (m, 1H), 7.09 (d, J=8.9 Hz, 2H), 6.92 (dd, J=3.6, 1.9 Hz, 1H), 6.23 (d, J=8.2 Hz, 1H), 6.18 (dd, J=3.6, 1.8 Hz, 1H), 5.42 (s, 2H), 4.48 (td, J=8.1, 4.9 Hz, 1H), 3.70 (s, 3H), 2.75 (t, J=8.0 Hz, 2H), 2.15 (dtd, J=13.5, 8.2, 5.0 Hz, 1H), 2.03-1.95 (m, 1H). ¹³C NMR (126 MHZ, Acetone-d₆) δ 174.1, 164.0, 162.4, 155.8, 148.8, 142.2, 138.2, 129.33, 129.26, 126.9, 123.0, 120.7, 119.8, 119.7, 99.5, 53.3, 52.3, 35.2, 32.5. HRMS (ESI) m/z calculated for C₂₄H₂₄N₆O₄ (M+H)⁺=461.1932. found=461.1933.

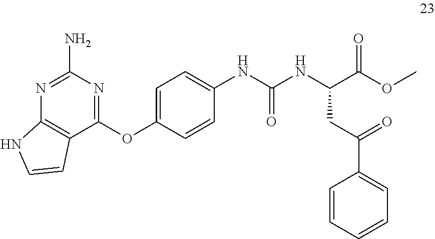 [SMILES: COC(=O)[C@H](CC(=O)c1ccccc1)NC(=O)Nc1ccc(Oc2nc(N)nc3[nH]ccc23)cc1]

Methyl(S)-2-(3-(4-((2-amino-7H-pyrrolo[2,3-d]pyrimidin-4-yl)oxy)phenyl) ureido)-4-oxo-4-phenylbutanoate (23): 26.7 mg (0.111 mmol) of 4-(4-aminophenoxy)-7H-pyrrolo[2,3-d]pyrimidin-2-amine (3), 36.5 mg (0.225 mmol, 2.03 eq.) of CDI, 0.4 mL of DMSO, 54.0 mg (0.222 mmol, 2.00 eq.) of(S)-1-methoxy-1,4-dioxo-4-phenylbutan-2-aminium chloride, and 50 μL (36.3 mg, 0.358 mmol, 3.23 eq.) of TEA. The solution was acidified with 100 μL acetic acid and purified via HPLC, yielding 14.0 mg (0.0295 mmol, 26.7% yield) of methyl(S)-2-(3-(4-((2-amino-7H-pyrrolo[2,3-d]pyrimidin-4-yl)oxy)phenyl) ureido)-4-oxo-4-phenylbutanoate (23) as a white solid. ¹H NMR (400 MHZ, DMSO-d₆) δ 11.10 (s, 1H), 8.86 (s, 1H), 7.96 (d, J=7.7 Hz, 2H), 7.64 (t, J=7.4 Hz, 1H), 7.52 (t, J=7.6 Hz, 2H), 7.35 (d, J=8.6 Hz, 2H), 7.03 (d, J=8.5 Hz, 2H), 6.82 (t, J=2.8 Hz, 1H), 6.69 (d, J=8.6 Hz, 1H), 5.95 (d, J=4.0 Hz, 3H), 4.78 (dt, J=8.9, 4.8 Hz, 1H), 3.67 (dd, J=18.3, 5.4 Hz, 1H), 3.60 (s, 3H), 3.55 (dd, J=18.5, 4.6 Hz, 3H). ¹³C NMR (101 MHz, DMSO-d₆) δ 197.8, 172.6, 162.6, 159.4, 156.1, 154.8, 147.1, 137.2, 135.9, 133.8, 128.9, 128.1, 122.2, 120.2, 118.7, 98.3, 97.0, 52.2, 48.2, 40.9. HRMS (ESI) m/z calculated for C₂₄H₂₂N₆O₅ (M+H)⁺=475.1724. found=475.1723.

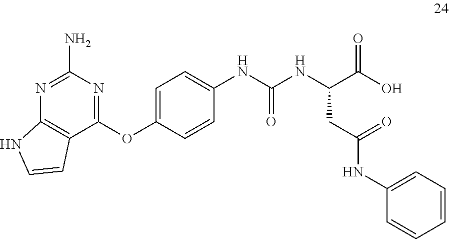 [SMILES: Nc1nc(Oc2ccc(NC(=O)N[C@@H](CC(=O)Nc3ccccc3)C(=O)O)cc2)c2cc[nH]c2n1]

N²-((4-((2-amino-7H-pyrrolo[2,3-d]pyrimidin-4-yl)oxy)phenyl) carbamoyl)-N⁴-phenyl-L-asparagine (24): 15.3 mg (0.0634 mmol) of 4-(4-aminophenoxy)-7H-pyrrolo[2,3-d]pyrimidin-2-amine (3), 12.4 mg (0.0765 mmol, 1.21 eq.) of CDI, 0.4 mL of DMSO, 13.0 mg (0.0624 mmol, 0.98 eq.) of N⁴-phenyl-L-asparagine suspended in 1.2 mL DMSO, and 35 μL (25.4 mg, 0.251 mmol, 3.96 eq.) of TEA. The solution was acidified with 100 μL acetic acid and purified via HPLC, yielding 6.1 mg (0.0128 mmol, 20.2% yield) of N²-((4-((2-amino-7H-pyrrolo[2,3-d]pyrimidin-4-yl)oxy)phenyl) carbamoyl)-N⁴-phenyl-L-asparagine (24) as a white solid. ¹H NMR (600 MHz, DMSO-d₆) δ 11.11 (s, 1H), 10.29 (s, 1H), 8.96 (s, 1H), 7.59 (d, J=7.5 Hz, 2H), 7.40 (d, J=8.9 Hz, 2H), 7.29 (dd, J=8.5, 7.4 Hz, 2H), 7.09-7.00 (m, 3H), 6.86 (dd, J=3.5, 2.2 Hz, 1H), 6.59 (d, J=8.1 Hz, 1H), 5.99 (dd, J=3.5, 2.0 Hz, 2H), 5.97 (s, 2H), 4.51 (dt, J=8.3, 5.3 Hz, 1H), 2.89 (dd, J=15.9, 5.4 Hz, 1H), 2.82 (dd, J=15.9, 5.3 Hz, 1H). ¹³C NMR (151 MHz, DMSO-d₆) δ 173.4, 168.8, 162.6, 159.4, 156.1, 154.9, 146.9, 139.1, 137.4, 128.7, 123.1, 122.1, 120.1, 119.0, 118.6, 98.2, 97.0, 49.3, 40.1. HRMS (ESI) m/z calculated for C₂₃H₂₁N₇O₅ (M+H)⁺=476.1677. found=476.1690.

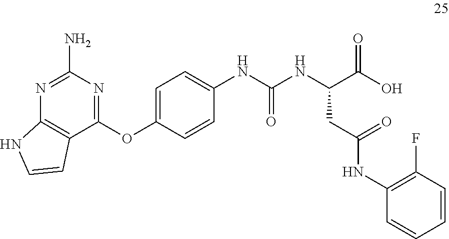 [SMILES: Nc1nc(Oc2ccc(NC(=O)N[C@@H](CC(=O)Nc3ccccc3F)C(=O)O)cc2)c2cc[nH]c2n1]

N²-((4-((2-amino-7H-pyrrolo[2,3-d]pyrimidin-4-yl)oxy)phenyl) carbamoyl)-N⁴-(2-fluorophenyl)-L-asparagine (25): 14.3 mg (0.0593 mmol) of 4-(4-aminophenoxy)-7H-pyrrolo[2,3-d]pyrimidin-2-amine (3), 19.6 mg (0.121 mmol, 2.04 eq.) of CDI, 0.5 mL of DMSO, 26.1 mg (0.115 mmol, 1.95 eq.) of N⁴-(2-fluorophenyl)-L-asparagine suspended in 0.5 mL DMSO, and 35 μL (25.4 mg, 0.251 mmol, 4.23 eq.) of TEA. The solution was acidified with 100 μL acetic acid and purified via HPLC, yielding 6.7 mg (0.0136 mmol, 22.9% yield) of N²-((4-((2-amino-7H-pyrrolo[2,3-d]pyrimidin-4-yl)oxy)phenyl) carbamoyl)-N⁴-(2-fluorophenyl)-L-asparagine (25) as a white solid. ¹H NMR (500 MHZ, DMSO-d₆) δ 11.11 (s, 1H), 10.27 (br s, 1H), 8.96 (s, 1H), 8.02-7.88 (m, 1H), 7.47-7.35 (m, 2H), 7.24 (ddd, J=11.1, 7.3, 2.1 Hz, 1H), 7.14 (dt, J=8.9, 4.0 Hz, 2H), 7.09-7.00 (m, 2H), 6.86 (dd, J=3.5, 2.2 Hz, 1H), 6.57 (d, J=7.9 Hz, 1H), 5.99 (dd, J=3.5, 1.9 Hz, 2H), 5.97 (br s, 2H), 4.49 (q, J=5.4, 5.0 Hz, 1H), 2.94 (dd, J=15.9, 5.2 Hz, 1H), 2.88 (dd, J=16.0, 5.5 Hz, 1H). ¹³C NMR (126 MHz, DMSO-d₆) δ 173.3, 169.3, 162.6, 159.4, 156.1, 154.8, 146.9, 137.4, 126.2, 125.0, 124.2, 123.8, 122.1, 120.1, 118.6, 115.5, 115.3, 98.2, 97.0, 49.3, (last peak obscured by DMSO). ¹H NMR (500 MHZ, DMF-d₇) δ 11.15 (s, 1H), 10.30 (s, 1H), 9.27 (br s, 1H), 8.16 (t, J=7.6 Hz, 1H), 7.60 (d, J=8.5 Hz, 2H), 7.26-7.11 (m, 5H), 6.98 (dd, J=3.5, 1.7 Hz, 1H), 6.90 (br s, 1H), 6.12 (dd, J=3.6, 1.5 Hz, 1H), 6.00 (s, 2H), 4.76 (dd, J=7.9, 5.3 Hz, 1H), 3.18 (dd, J=15.8, 5.4 Hz, 1H), 3.09 (dd, J=15.9, 5.4 Hz, 1H). ¹³C NMR (126 MHz, DMF-d₁) § 170.1, 163.3, (aryl peak obscured by DMF) 160.3, 157.0, 155.7, 147.7, 138.4, 127.4, 127.3, 125.0, 124.9, 124.54, 124.51, 123.7, 122.3, 120.4, 118.6, 115.6, 115.5, 98.7, 97.8, 50.3, 39.8. HRMS (ESI) m/z calculated for C₂₃H₂₀FN₇O₅ (M+H)⁺=494.1583. found=494.1579.

### Synthetic Scheme for Modified Amino Acids:

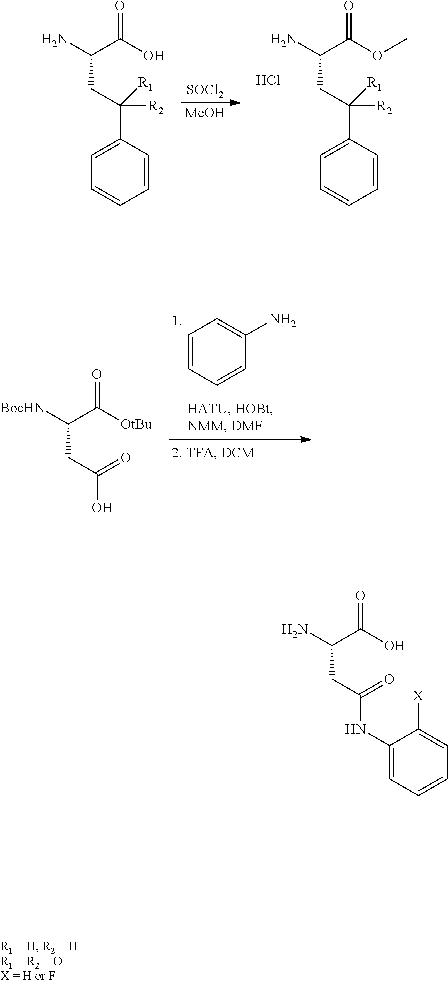

General Procedure for the Protection of Amino Acids as Methyl Esters: To a flame dried flask equipped with a stir bar was added amino acid. The flask was flushed with nitrogen, followed by methanol, and finally SOCl₂ (3.0-5.0 eq.) dropwise. The reaction was allowed to warm to room temperature and was stirred overnight. Upon completion, the solution was concentrated in vacuo, leaving methyl ester protected amino acid as an HCl salt.

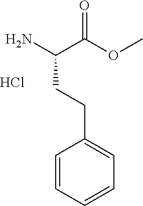

(S)-1-methoxy-1-oxo-4-phenylbutan-2-aminium chloride: 97.4 mg (0.543 mmol) of(S)-2-amino-4-phenylbutanoic acid, 120 μL (197 mg, 1.65 mmol, 3.0 eq.) of SOCl₂, and 4 mL of MeOH. 121.1 mg (0.527 mmol, 97.1% yield) of(S)-1-methoxy-1-oxo-4-phenylbutan-2-aminium chloride was isolated as a white solid. ¹H NMR (400 MHZ, Methanol-d₄) δ 7.37-7.15 (m, 5H), 4.08 (t, J=6.2 Hz, 1H), 3.82 (s, 3H), 2.82 (ddd, J=13.7, 10.0, 5.8 Hz, 1H), 2.73 (ddd, J=13.5, 9.7, 6.2 Hz, 1H), 2.22 (tq, J=15.4, 8.3, 7.8 Hz, 2H). ¹³C NMR (101 MHz, Methanol-d₄) δ 170.7, 141.0, 129.6, 129.4, 127.5, 53.7, 53.5, 33.5, 31.9. MS (ESI) m/z=194.1 [M+H]⁺, m/z=216.1 [M+Na]⁺.

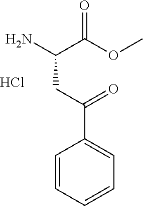

(S)-1-methoxy-1,4-dioxo-4-phenylbutan-2-aminium chloride: 76.5 mg (0.333 mmol) of (S)-2-amino-4-oxo-4-phenylbutanoic acid, 120 μL (197 mg, 1.65 mmol, 5.0 eq.) of SOCl₂, and 4 mL of MeOH. 79.8 mg (0.327 mmol, 98.3% yield) of(S)-1-methoxy-1,4-dioxo-4-phenylbutan-2-aminium chloride was isolated as a white solid. ¹H NMR (400 MHZ, Methanol-d₄) δ 8.05 (d, J=7.6 Hz, 2H), 7.68 (t, J=7.3 Hz, 1H), 7.55 (t, J=7.5 Hz, 2H), 4.54 (t, J=4.5 Hz, 1H), 3.89-3.80 (m, 5H). ¹³C NMR (101 MHz, Methanol-d₄) δ 197.5, 170.3, 136.7, 135.3, 130.0, 129.4, 53.9, 49.9, 39.4. MS (ESI) m/z=229.9 [M+Na]⁺.

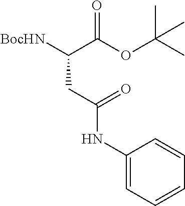 [SMILES: CC(C)(C)OC(=O)N[C@@H](CC(=O)Nc1ccccc1)C(=O)OC(C)(C)C]

Tert-butyl N²-(tert-butoxycarbonyl)-N⁴-phenyl-L-asparaginate: To an oven dried flask equipped with a stir bar was added 65.6 mg (0.227 mmol) of(S)-4-(tert-butoxy)-3-((tert-butoxycarbonyl)amino)-4-oxobutanoic acid, and 86.6 mg (0.228 mmol, 1.00 eq.) of HBTU. The flask was purged with nitrogen, at which point 2 mL of DMF were added, followed by 20 μL (20.6 mg, 0.221 mmol, 0.97 eq.) of aniline, and 250 μL (230 mg, 2.27 mmol, 10.0 eq.) of N-methylmorpholine. The solution was stirred overnight at room temperature, at which point the solution was concentrated in vacuo. The residue was partitioned between EtOAc and sat. Na₂CO₃, washed with brine, and the organic layer was dried with Na₂SO₄ and filtered. The dried organic layer was concentrated in vacuo and purified by column chromatography (10:1 DCM: MeOH), yielding 60.8 mg (0.167 mmol, yield-73.6%) of tert-butyl N²-(tert-butoxycarbonyl)-N⁴-phenyl-L-asparaginate as a white solid. ¹H NMR (400 MHz, Chloroform-d) δ 7.94 (s, 1H), 7.50 (d, J=7.9 Hz, 2H), 7.29 (t, J=7.8 Hz, 2H), 7.08 (t, J=7.4 Hz, 1H), 5.73 (d, J=5.4 Hz, 1H), 4.45 (dt, J=9.5, 5.1 Hz, 1H), 2.98 (dd, J=15.5, 5.4 Hz, 1H), 2.86 (dd, J=16.0, 4.5 Hz, 1H), 1.45 (s, 9H), 1.43 (s, 9H). ¹³C NMR (101 MHZ, Chloroform-d) δ 170.5, 168.4, 156.0, 137.9, 129.1, 124.4, 120.0, 82.6, 80.15, 39.8, 38.8, 28.4, 28.0. MS (ESI) m/z=365.1 [M+H]⁺, m/z=387.2 [M+Na]⁺.

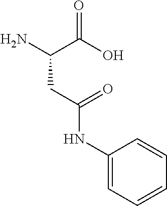 [SMILES: N[C@@H](CC(=O)Nc1ccccc1)C(=O)O]

N⁴-phenyl-L-asparagine: To a vial equipped with a stir bar and 60.8 mg (0.167 mmol) of tert-butyl N²-(tert-butoxycarbonyl)-N⁴-phenyl-L-asparaginate was added 2 mL of DCM and 2 mL of TFA. The solution was stirred at room temperature for 2 hours under ambient atmosphere, at which point the solution was concentrated in vacuo. The residue was purified via reverse phase chromatography, yielding 13.0 mg (0.0624 mmol, yield=37.4%) of N⁴-phenyl-L-asparagine as a white solid. ¹H NMR (400 MHZ, Methanol-d₄) δ 7.56 (d, J=8.0 Hz, 2H), 7.30 (t, J=7.8 Hz, 2H), 7.10 (t, J=7.5 Hz, 1H), 4.28 (dd, J=7.1, 4.1 Hz, 1H), 3.14 (dd, J=17.1, 4.0 Hz, 1H), 3.04 (dd, J=18.6, 5.9 Hz, 1H). ¹³C NMR (101 MHz, Methanol-d₄) δ 171.4, 169.4, 139.5, 129.9, 125.4, 121.1, 51.1, 36.4.

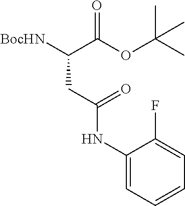 [SMILES: CC(C)(C)OC(=O)N[C@@H](CC(=O)Nc1ccccc1F)C(=O)OC(C)(C)C]

Tert-butyl N²-(tert-butoxycarbonyl)-N⁴-(2-fluorophenyl)-L-asparaginate: To an oven dried flask equipped with a stir bar was added 64.5 mg (0.223 mmol) of(S)-4-(tert-butoxy)-3-((tert-butoxycarbonyl)amino)-4-oxobutanoic acid, 85.2 mg (0.224 mmol, 1.00 eq.) of HATU, and 30.8 mg (0.228 mmol, 1.02 eq.) of HOBt. The flask was purged with nitrogen, at which point 2 mL of DMF were added, followed by 20 μL (23.0 mg, 0.207 mmol, 0.93 eq.) of 2-fluoroaniline, and 250 μL (230 mg, 2.27 mmol, 10.2 eq.) of N-methylmorpholine. The solution was stirred overnight at room temperature, at which point the solution was concentrated in vacuo. The residue was partitioned between EtOAc and sat. Na₂CO₃, washed with brine, and the organic layer was dried with Na₂SO₄ and filtered. The dried organic layer was concentrated in vacuo and purified by column chromatography (0-100% EtOAc in hexanes), yielding 53.4 mg (0.140 mmol, yield=62.6%) of tert-butyl N²-(tert-butoxycarbonyl)-N⁴-(2-fluorophenyl)-L-asparaginate as a white solid. ¹H NMR (600 MHZ, Acetone-d₆) δ 9.06 (s, 1H), 8.22 (t, J=7.6 Hz, 1H), 7.21-7.06 (m, 3H), 6.18 (d, J=8.7 Hz, 1H), 4.46 (dt, J=8.9, 5.7 Hz, 1H), 3.04 (dd, J=15.8, 6.2 Hz, 1H), 2.96 (dd, J=15.5, 5.2 Hz, 1H), 1.43 (s, 9H), 1.41 (s, 9H). ¹³C NMR (151 MHZ, Acetone-d₆) δ 171.3, 169.8, 156.3, 154.7, 153.1, 127.63, 127.56, 125.40, 125.35, 125.14, 125.11, 123.7, 115.9, 115.8, 81.7, 79.3, 52.1, 39.4, 28.5, 28.1. MS (ESI) m/z=365.1 [M+H]⁺, m/z=387.2 [M+Na]⁺.

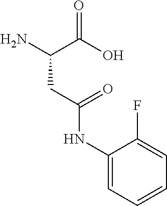 [SMILES: N[C@@H](CC(=O)Nc1ccccc1F)C(=O)O]

N⁴-(2-fluorophenyl)-L-asparagine: To a vial equipped with a stir bar and 53.4 mg (0.140 mmol) of tert-butyl N²-(tert-butoxycarbonyl)-N⁴-(2-fluorophenyl)-L-asparaginate was added 2 mL of DCM and 2 mL of TFA. The solution was stirred at room temperature for 1 hour under ambient atmosphere, at which point the solution was concentrated in vacuo, yielding 26.4 mg (0.117 mmol, yield=93.6%) of N⁴-(2-fluorophenyl)-L-asparagine as a white solid. ¹H NMR (500 MHZ, Methanol-d₄) δ 8.04-7.92 (m, 1H), 7.20-7.08 (m, 3H), 4.21 (dd, J=7.7, 3.8 Hz, 1H), 3.21 (dd, J=17.3, 3.8 Hz, 1H), 3.08 (dd, J=17.3, 7.7 Hz, 1H). ¹³C NMR (126 MHZ, Methanol-d₄) δ 171.7, 170.2, 156.4, 154.5, 127.01, 126.95, 126.9, 126.8, 125.4, 125.32, 125.29, 116.5, 116.3, 51.4, 36.5. MS (ESI) m/z=225.0 [M+H]⁺.

### Determination of Enantiopurity of Ligand Series

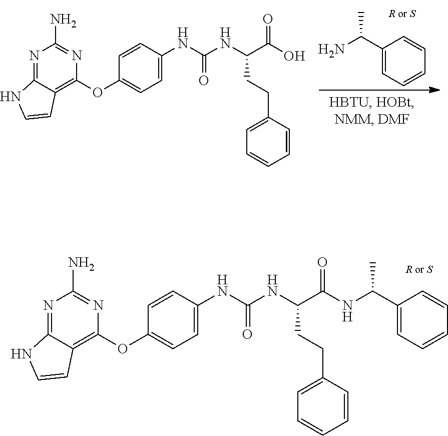

General Procedure for the Attachment of Chiral Resolving Agents: To an oven dried flask equipped with a stir bar was added(S)-2-(3-(4-((2-amino-7H-pyrrolo[2,3-d]pyrimidin-4-yl)oxy)phenyl) ureido)-4-phenylbutanoic acid (1 eq.), HBTU, (1 eq.), and HOBt (1 eq.). The vessel was purged with nitrogen, followed by 2 mL of DMF, (R)- or (S)-1-phenylethan-1-amine (1 eq.), and N-methyl morpholine (10 eq.). The solution was stirred at room temperature overnight. The solution was concentrated in vacuo and partitioned between EtOAc and sat. NaHCO₃. The mixture was extracted with EtOAc and the organic layers were combined, dried with Na₂SO₄ and filtered. The solution was concentrated in vacuo and an NMR was taken. The sample was then purified via reverse phase chromatography and analyzed by NMR again. Finally, ˜ 1 mg of each diastereomer were combined and analyzed by NMR.

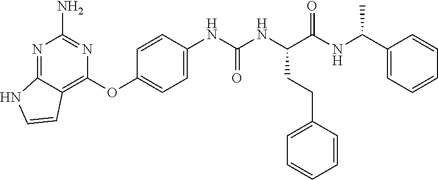 [SMILES: C[C@@H](NC(=O)[C@H](CCc1ccccc1)NC(=O)Nc1ccc(Oc2nc(N)nc3[nH]ccc23)cc1)c1ccccc1]

(S)-2-(3-(4-((2-amino-7H-pyrrolo[2,3-d]pyrimidin-4-yl)oxy)phenyl) ureido)-4-phenyl-N—((R)-1-phenylethyl) butanamide: 8.2 mg (0.0184 mmol) of(S)-2-(3-(4-((2-amino-7H-pyrrolo[2,3-d]pyrimidin-4-yl)oxy)phenyl) ureido)-4-phenylbutanoic acid, 7.7 mg (0.0203 mmol, 1.1 eq.) of HBTU, 2.3 mg (0.0170 mmol, 0.93 eq.) of HOBt, 10 μL (9.5 mg, 0.0784 mmol, 4.3 eq.) of (R)-1-phenylethan-1-amine, 20 μL (18.4 mg, 0.182 mmol, 9.9 eq.) of N-methylmorpholine, and 2 mL DMF. After purification by reverse phase HPLC, 7.0 mg (0.0127 mmol, 69% yield) of(S)-2-(3-(4-((2-amino-7H-pyrrolo[2,3-d]pyrimidin-4-yl)oxy)phenyl) ureido)-4-phenyl-N—((R)-1-phenylethyl) butanamide was isolated as a white solid. ¹H NMR (500 MHZ, DMSO-d₆) δ 11.17 (s, 1H), 8.83 (s, 1H), 8.62 (d, J=8.0 Hz, 1H), 7.42 (d, J=9.0 Hz, 2H), 7.37-7.28 (m, 4H), 7.29-7.19 (m, 3H), 7.16 (t, J=7.4 Hz, 1H), 7.11-7.05 (m, 4H), 6.87 (dd, J=3.6, 2.2 Hz, 1H), 6.52 (d, J=8.2 Hz, 1H), 6.21-5.80 (m, 3H), 4.98 (p, J=7.2 Hz, 1H), 4.37 (td, J=7.5, 5.3 Hz, 1H), 2.49-2.42 (m, 2H), 1.95-1.86 (m, 1H), 1.79 (dddd, J=18.8, 14.9, 11.1, 7.4 Hz, 1H), 1.39 (d, J=7.0 Hz, 3H); ¹³C NMR (126 MHz, DMSO-d₆) & 170.8, 162.6, 159.1, 154.8, 146.9, 144.7, 141.4, 137.4, 128.4, 128.2, 128.1, 126.7, 125.84, 125.80, 122.1, 120.2, 118.6, 98.3, 97.0, 52.4, 47.9, 35.4, 31.0, 22.4; HRMS (ESI) m/z calculated for C₃₁H₃₁N₇O₃ (M+H)⁺=550.2561. found=550.2566.

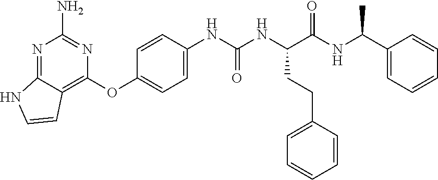 [SMILES: C[C@H](NC(=O)[C@H](CCc1ccccc1)NC(=O)Nc1ccc(Oc2nc(N)nc3[nH]ccc23)cc1)c1ccccc1]

(S)-2-(3-(4-((2-amino-7H-pyrrolo[2,3-d]pyrimidin-4-yl)oxy)phenyl) ureido)-4-phenyl-N—((S)-1-phenylethyl) butanamide: 12.2 mg (0.0273 mmol) of(S)-2-(3-(4-((2-amino-7H-pyrrolo[2,3-d]pyrimidin-4-yl)oxy)phenyl) ureido)-4-phenylbutanoic acid, 10.6 mg (0.0280 mmol, 1.0 eq.) of HBTU, 3.8 mg (0.0281 mmol, 1.0 eq.) of HOBt, 10 μL (9.5 mg, 0.0784 mmol, 2.9 eq.) of(S)-1-phenylethan-1-amine, 30 μL (27.6 mg, 0.273 mmol, 10 eq.) of N-methylmorpholine, and 2 mL DMF. After purification by reverse phase HPLC, 6.8 mg (0.0124 mmol, 45% yield) of(S)-2-(3-(4-((2-amino-7H-pyrrolo[2,3-d]pyrimidin-4-yl)oxy)phenyl) ureido)-4-phenyl-N—((S)-1-phenylethyl) butanamide was isolated as a white solid. ¹H NMR (500 MHZ, DMSO-d₆) δ 11.17 (s, 1H), 8.83 (s, 1H), 8.62 (d, J=8.0 Hz, 1H), 7.42 (d, J=9.0 Hz, 2H), 7.37-7.28 (m, 4H), 7.29-7.19 (m, 3H), 7.16 (t, J=7.4 Hz, 1H), 7.11-7.05 (m, 4H), 6.87 (dd, J=3.6, 2.2 Hz, 1H), 6.52 (d, J=8.2 Hz, 1H), 6.21-5.80 (m, 3H), 4.98 (p, J=7.2 Hz, 1H), 4.37 (td, J=7.5, 5.3 Hz, 1H), 2.49-2.42 (m, 2H), 1.95-1.86 (m, 1H), 1.79 (dddd, J=18.8, 14.9, 11.1, 7.4 Hz, 1H), 1.39 (d, J=7.0 Hz, 3H); ¹³C NMR (126 MHz, DMSO-d₆) δ 170.8, 162.6, 159.1, 154.8, 146.9, 144.7, 141.4, 137.4, 128.4, 128.2, 128.05, 126.7, 125.84, 125.80, 122.1, 120.2, 118.6, 98.3, 97.0, 52.4, 47.9, 35.4, 31.0, 22.4; HRMS (ESI) m/z calculated for C₃₁H₃₁N₇O₃ (M+H)⁺=550.2561. found=550.2565.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance.

Embodiment 1 provides a compound of Formula I, or a pharmaceutically acceptable salt, tautomer, or enantiomer thereof:

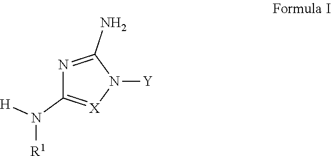

wherein,
Y is selected from the group consisting of —CH₂-R², —C(═O)NR²R³, optionally substituted C₅₋₁₀ heteroaryl, and optionally substituted C₅₋₆ heterocycloalkyl;
wherein the optional substitution is at least one substituent selected from the group consisting of C₁₋₅ alkyl, C₁₋₅ alkoxy, C₁₋₅ thioalkyl, C₁₋₅ aminoalkyl, C₅₋₁₀ aryl, C₅₋₁₀ heteroaryl, ═O (oxo), F, Cl, Br, I, C(═O)OR, NHC(═O)R, and OH;
R¹ and R² are each independently selected from the group consisting of C₂₋₆ alkenyl, C₃₋₇ cycloalkyl, C₆₋₁₀ aryl, 5-10 membered heteroaryl, 4-10 membered heterocycloalkyl, C₆₋₁₀-5-6 membered heterobiaryl, 5-6 membered-C₆₋₁₀ heterobiaryl, and C₆₋₁₀-C₆₋₁₀ biaryl,
each optionally substituted by at least one group of formula (LL)_{zz}-GG, wherein
   LL is at each occurrence independently selected from the group consisting of —O—, —OR, —S—, —S(═O)—, —S(═O)₂—, —NR², —CH═, —C═, —CH₂—, —CHR—, —CR₂—, —CH₃, —C(═O)—, —C(═NR)—;
   zz is an integer from 1 to 30;
   GG is a substituent selected from the group consisting of F, Cl, Br, I, OR,

OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, and C(═NH)N(R)₂, wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
R³ is H or C₁₋₄ hydrocarbyl;
X is N or C—R⁴, wherein R⁴ is H or C₁₋₄ hydrocarbyl optionally substituted by 1 to 3 substituents selected from the group consisting of OR′, NHR′, and NR′₂,
   wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl; or wherein two R′ groups bonded to a nitrogen atom or to adjacent nitrogen atoms can together with the nitrogen atom or atoms form a heterocyclyl.

Embodiment 2 provides the compound of embodiment 1, wherein R¹ is C₆₋₁₀ aryl. Embodiment 3 provides the compound of any one of embodiments 1, wherein R² is C₆₋₁₀-5-6 membered heterobiaryl, 5-6 membered-C₆₋₁₀ heterobiaryl, or C₆₋₁₀-C₆₋₁₀ biaryl, each of which is at least disubstituted on a terminal ring.

Embodiment 4 provides the compound of any one of embodiments 1-3, wherein X is N.

Embodiment 5 provides the compound of any one of embodiments 1-4, wherein R³ is H.

Embodiment 6 provides the compound of any one of embodiments 1-5, wherein R¹ has the structure:

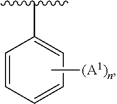

wherein:
each occurrence of A¹ is independently selected from the group consisting of SO₂R, SO₂N(R)₂, C(═O)R, and C(═O)OR, wherein each occurrence of R is independently selected from the group consisting of hydrogen, (C₁-C₆) hydrocarbyl, and (C₃-C₁₈)heterocycloalkyl; and
n is an integer from 0 to 5.

Embodiment 7 provides the compound of any one of embodiments 1-6, wherein the compound is of Formula Ia:

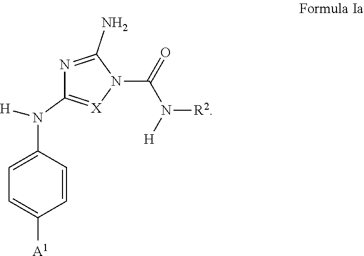

Embodiment 8 provides the compound of any one of embodiments 1-7, wherein A¹ is C(—O)NH—C₁₋₆ alkyl or C(—O)—C₆ heterocycloalkyl.

Embodiment 9 provides the compound of any one of embodiments 1-8, wherein A¹ is C(═O)NHMe or SO₂NH₂.

Embodiment 10 provides the compound of any one of embodiments 1-9, wherein the compound is of Formula Ib, Formula Ic, or Formula Id:

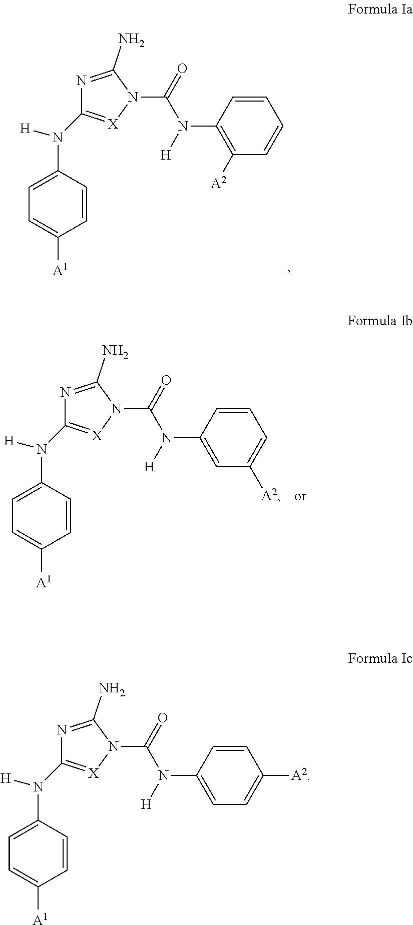

Embodiment 11 provides the compound of any one of embodiments 1-10, wherein A² is selected from the group consisting of:

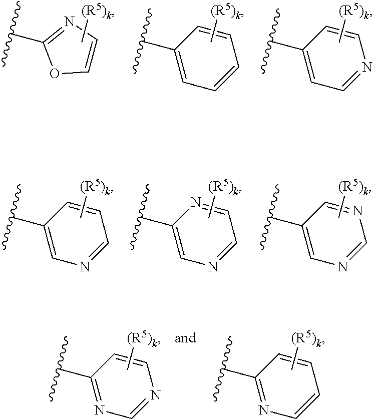

wherein k is 2 or 3.

Embodiment 12 provides the compound of any one of embodiments 1-11, wherein each R⁵ is independently (LL)_{zz}-GG,
LL is selected from group consisting of —O—, —CH₂—, and —CHR—;
zz is 0, 1, 2, or 3; and
GG is selected from the group consisting of C(═O)OH, C(═O)O—C₁₋₄ alkyl, (C₆-C₁₀)aryl, and (C₅-C₁₀)heteroaryl.

Embodiment 13 provides the compound of any one of embodiments 1-12, wherein the compound is selected from the group consisting of:

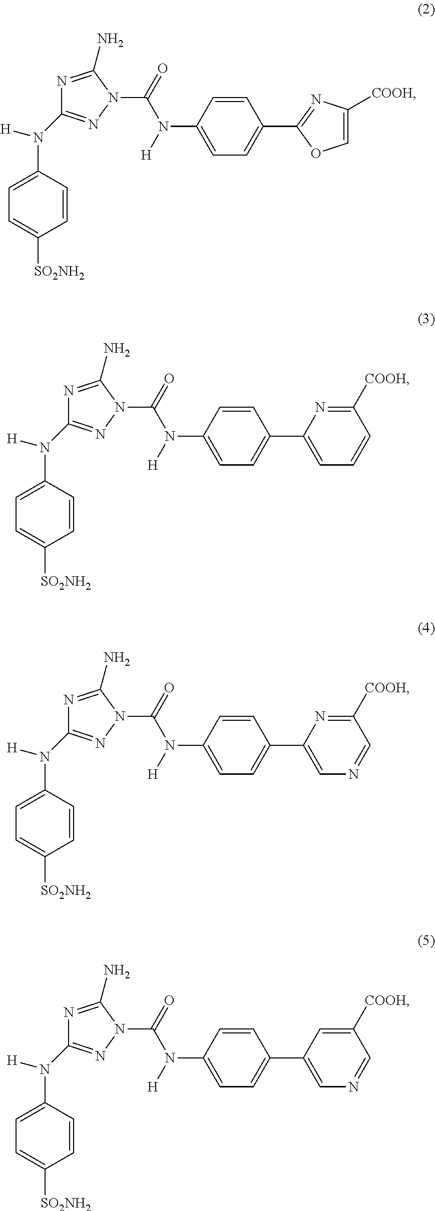 [SMILES: Nc1nc(Nc2ccc(S(N)(=O)=O)cc2)nn1C(=O)Nc1ccc(-c2nc(C(=O)O)co2)cc1 ; Nc1nc(Nc2ccc(S(N)(=O)=O)cc2)nn1C(=O)Nc1ccc(-c2cccc(C(=O)O)n2)cc1 ; Nc1nc(Nc2ccc(S(N)(=O)=O)cc2)nn1C(=O)Nc1ccc(-c2cncc(C(=O)O)n2)cc1 ; Nc1nc(Nc2ccc(S(N)(=O)=O)cc2)nn1C(=O)Nc1ccc(-c2cncc(C(=O)O)c2)cc1]

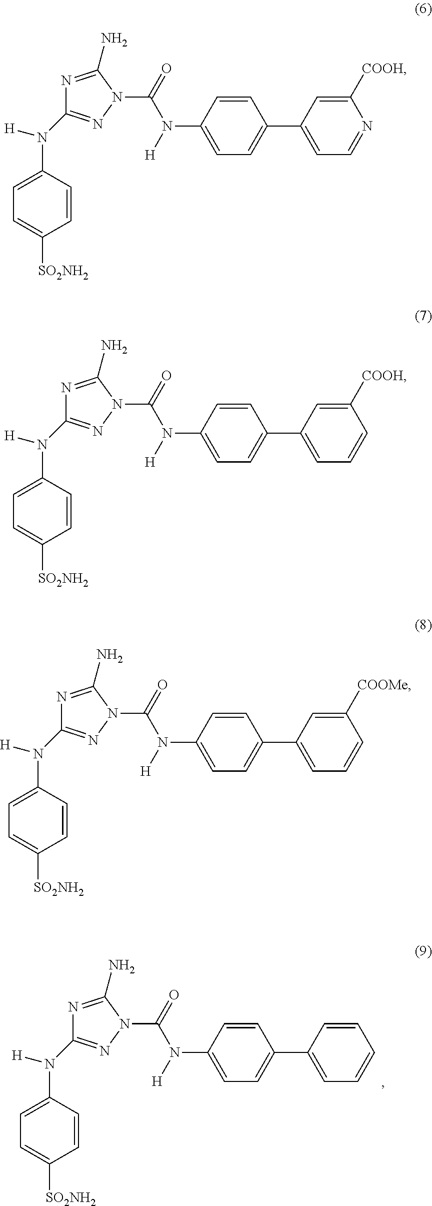 [SMILES: Nc1nc(Nc2ccc(S(N)(=O)=O)cc2)nn1C(=O)Nc1ccc(-c2ccnc(C(=O)O)c2)cc1 ; Nc1nc(Nc2ccc(S(N)(=O)=O)cc2)nn1C(=O)Nc1ccc(-c2cccc(C(=O)O)c2)cc1 ; COC(=O)c1cccc(-c2ccc(NC(=O)n3nc(Nc4ccc(S(N)(=O)=O)cc4)nc3N)cc2)c1 ; Nc1nc(Nc2ccc(S(N)(=O)=O)cc2)nn1C(=O)Nc1ccc(-c2ccccc2)cc1]

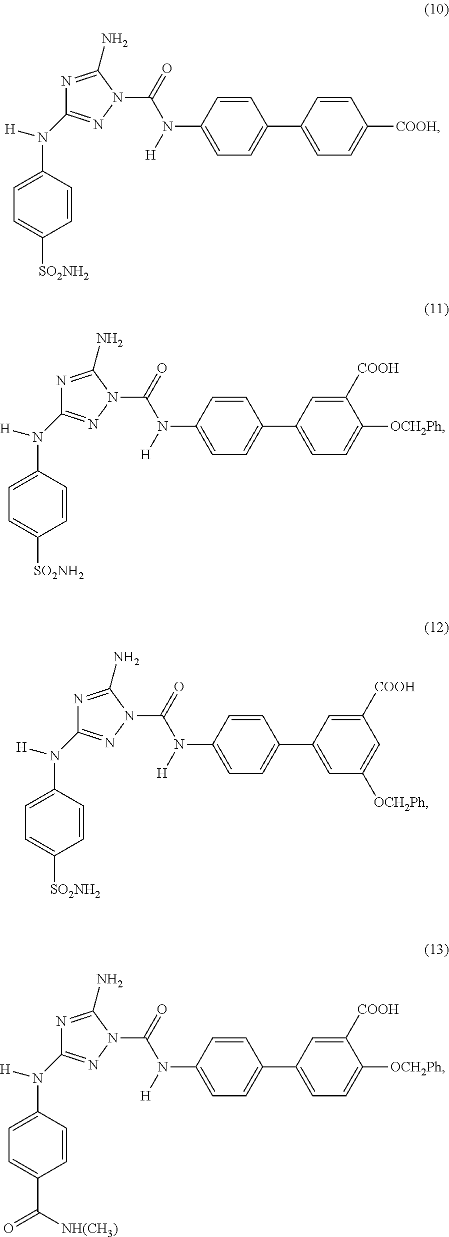

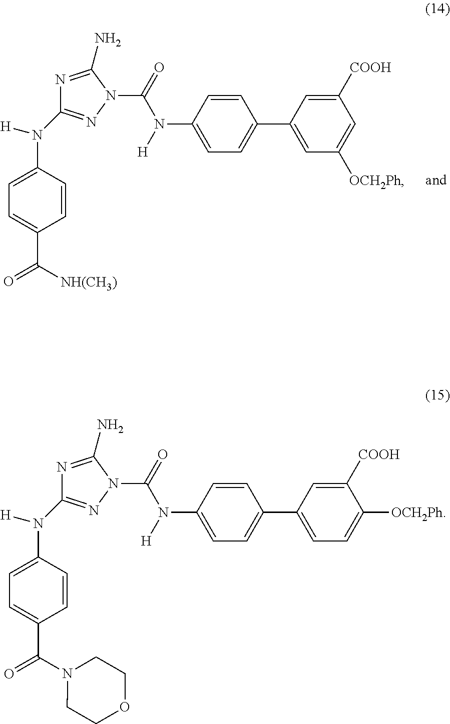

Embodiment 14 provides a compound of Formula II, or a pharmaceutically acceptable salt, tautomer, or enantiomer thereof:

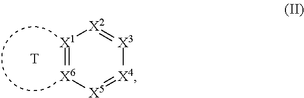

wherein
T is an optional 5 or 6 membered heterocyclic fused ring that is optionally substituted by at least one -(LL)_{zz}-GG;
each of X¹-X⁶ is independently N or C—Y;
each occurrence of Y is independently absent, H, -(Q)ₙ-(C₃-C₁₂)cycloalkyl, -(Q)ₙ-(C₃-C₁₈)heterocycloalkyl, -(Q)ₙ-(C₆-C₁₈)aryl, or -(Q)ₙ-(C₅-C₁₈)heteroaryl;
Q is absent, or independently selected at each occurrence from the group consisting of O, CH₂, NH, and N—C₁₋₄ alkyl;
n is an integer from 1 to 10;
each cycloalkyl, heterocycloalkyl, aryl, and heteroaryl in Y is optionally substituted by at least one -(LL)_{zz}-GG,
at each occurrence LL is independently selected from the group consisting of —O—, —OR, —S—, —S(—O)—, —S(—O)₂—, —NR—, —NR², —CH≡, —C≡, —CH₂—, —CHR—, —CR₂—, —CH₃, —C(═O)—, —C(═NR)—;
zz is an integer from 1 to 30;
GG is a substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂) o-2N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl;
wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl,

(C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
wherein each alkyl, alkenyl, alkynyl, cycloalkyl, heterocycloalkyl, aryl, and heteroaryl in GG is optionally substituted aby at least one substituent selected from the group consisting of H, F, Cl, Br, I, OR′, OC(═O)N(R′)₂, CN, NO₂, CF₃, OCF₃, R′, N(′R)₂, SR′, SOR′, SO₂R′, SO₂N(R′)₂, SO₃R′, C(═O) R′, C(═O)OR′, OC(═O) R′, O(CH₂)₀₋₂C(═O)OR′, C(═O)N(R′)₂, OC(═O)N(R′)₂, (CH₂)₀₋₂N(R′)C(═O) R′, N(R′)SO₂R′, N(R′)C(═O)OR′, N(R′)C(═O) R′, N(R′)C(═O)N(R′)₂, and C(═NH)N(R′)₂;
wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof;
with the proviso that at least two of X¹-X⁶ is N and at least one of X¹-X⁶ is C—NH₂.

Embodiment 15 provides the compound of embodiment 14, having the formula:

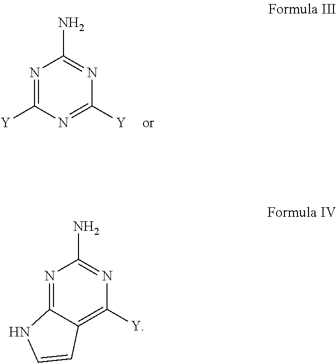

Embodiment 16 provides the compound of any one of embodiments 14-15, having the formula:

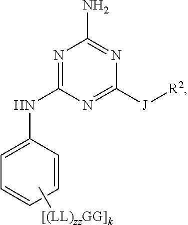

wherein
J is O or NH;
k is an integer from 1 to 5; and
R² is selected from the group consisting of C₆₋₁₀ aryl, C₆₋₁₀ heteroaryl, and combinations thereof, each of which is optionally substituted.

Embodiment 17 provides the compound of any one of embodiments 14-16, having the formula:

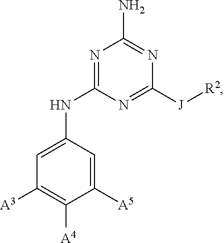

wherein A³, A⁴, A⁵ are each independently selected from the group consisting of H, F, CF₃, CN, OCF₃, SO₂R, SO₂N(R)₂, C(═O)R, and C(═O)OR, C(═O) (CH₂)ₘOCH₃, O(CH₂)ₘOCH₃, (C₅-C₁₀)heteroaryl, and (C₃-C₁₈)heterocycloalkyl, wherein the heteroaryl and heterocycloalkyl are each optionally substituted, and wherein each occurrence of R is independently selected from the group consisting of hydrogen, (C₁-C₆) hydrocarbyl, (C₅-C₁₀) heteroaryl, and (C₃-C₁₈)heterocycloalkyl.

Embodiment 18 provides the compound of any one of embodiments 14-17, wherein A⁴ is selected from the group consisting of CN, SO₂NH₂, and C(═O)NHCH₃.

Embodiment 19 provides the compound of any one of embodiments 14-18, wherein J is O.

Embodiment 20 provides the compound of any one of embodiments 14-19, wherein R² is selected from the group consisting of:

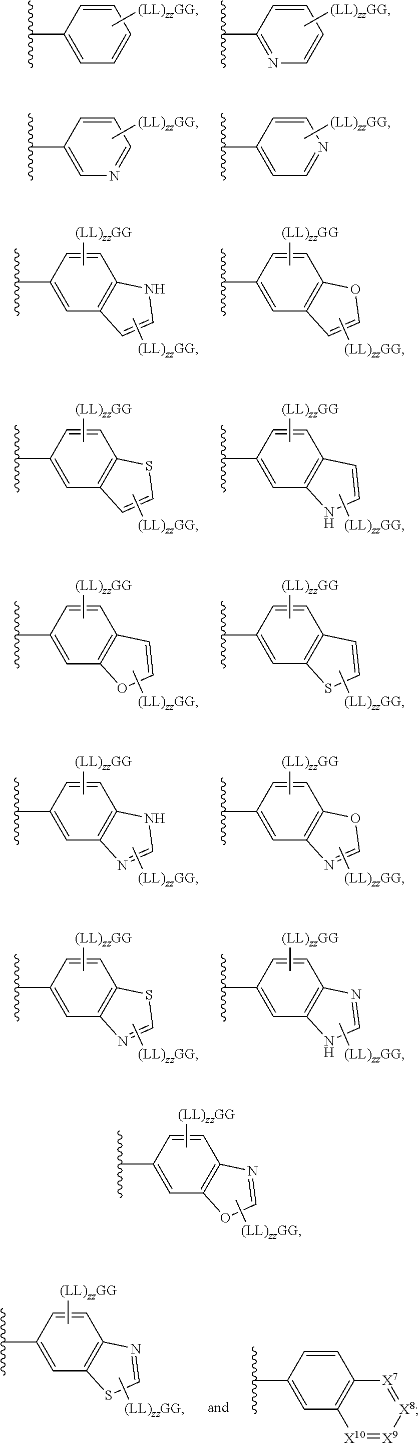

wherein X⁷, X⁸, X⁹, and X¹⁰ are each independently N, NH, or C—Y; and
wherein each -(LL)_{zz}GG moiety represents at least one substituent.

Embodiment 21 provides the compound of any one of embodiments 14-20, wherein R² is selected from the group consisting of

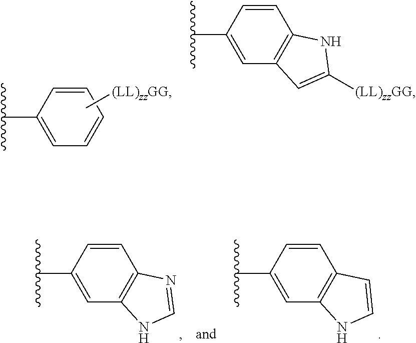

Embodiment 22 provides the compound of any one of embodiments 14-21, wherein R² is selected from the group consisting of

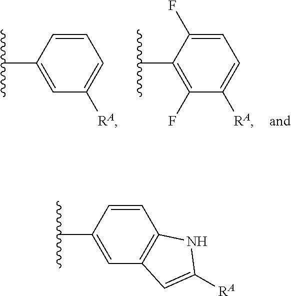

wherein R^{A} is selected from the group consisting of H, CO₂H, NH₂, OH, OCH₃, CH₂OCH₂COOH, and trans C═C(H)(COOH).

Embodiment 23 provides the compound of any one of embodiments 14-22, wherein R² is selected from the group consisting of:

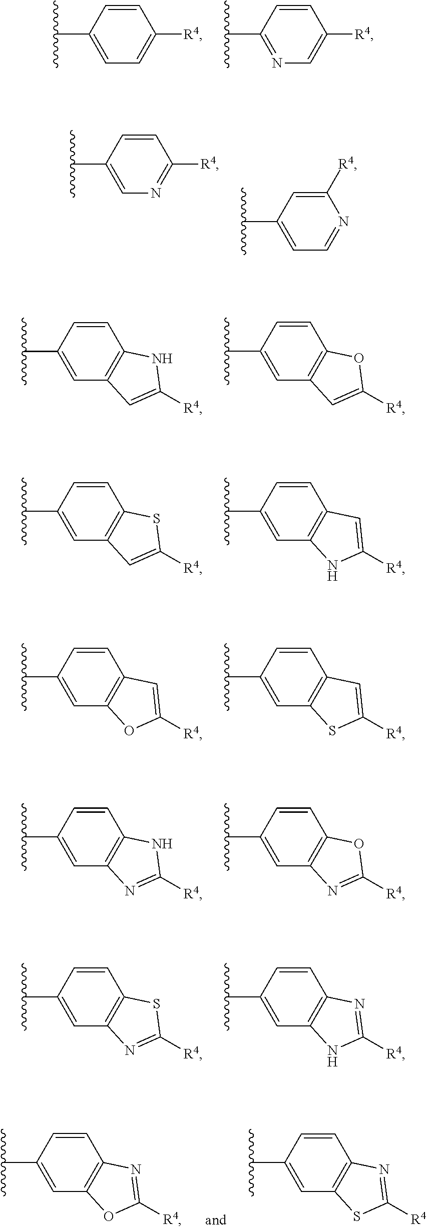

wherein
R⁴ is

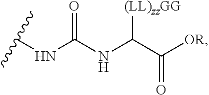

each LL is independently selected from the group consisting of —CH₂—, —NH—, —C(═O)—, and —O—;
R is C₁₄ alkyl or H;
zz is an integer from 2 to 6; and
GG is optionally substituted phenyl.

Embodiment 24 provides the compound of any one of embodiments 14-23, wherein (LL)_{zz}GG is selected from the group consisting of (CH₂)₂Ph, (CH₂) 3Ph, CH₂C(═O)NHPh, and CH₂(C—O)Ph.

Embodiment 25 provides the compound of any one of embodiments 14-15, having the formula:

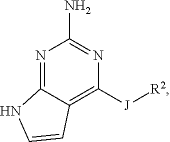

wherein
J is O or NH;
R² is selected from the group consisting of C₆₋₁₀ aryl, C₆₋₁₀ heteroaryl, and combinations thereof, each of which is optionally substituted.

Embodiment 26 provides the compound of any one of embodiments 14, 15, or 24-25, wherein J is O.

Embodiment 27 provides the compound of any one of embodiments 14, 15, or 24-26, wherein R² is selected from the group consisting of:

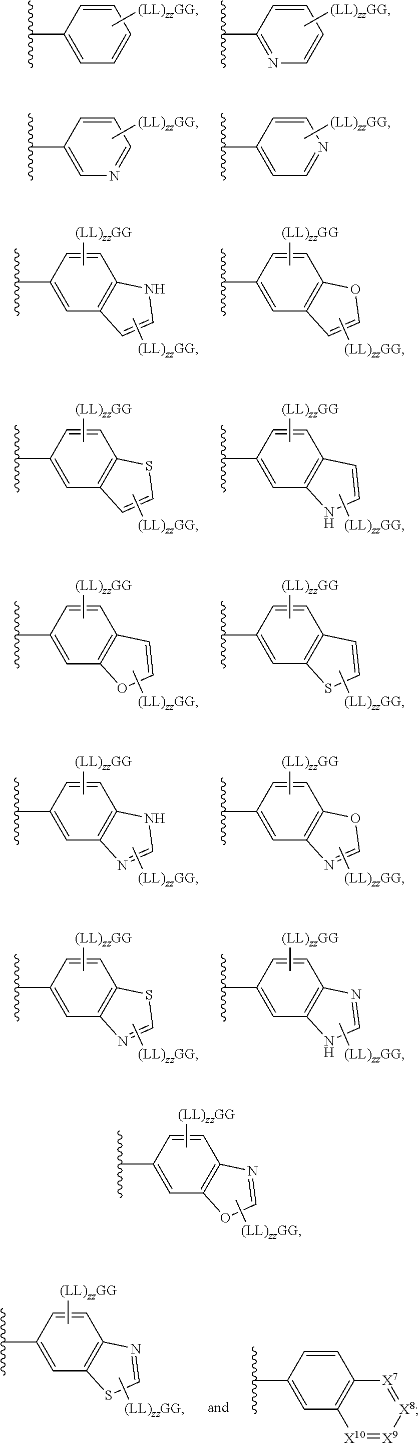

wherein X⁷, X⁸, X⁹, and X¹⁰ are each independently N, NH, or C—Y; and
wherein each -(LL)_{zz}GG moiety represents at least one substituent.

Embodiment 28 provides the compound of any one of embodiments 14, 15, or 24-27, wherein R² is selected from the group consisting of

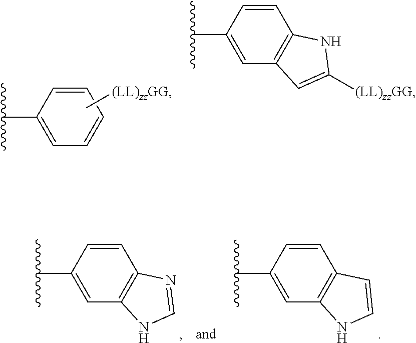

Embodiment 29 provides the compound of any one of embodiments 14, 15, or 24-28, wherein R² is selected from the group consisting of

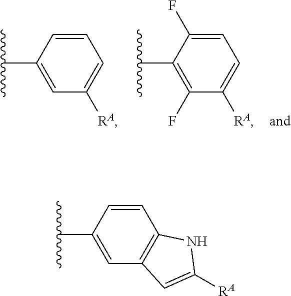

wherein R^{A} is selected from the group consisting of —H, —CO₂H, —NH₂, —OH, —OCH₃, —CH₂OCH₂COOH, and trans —C═C(H)COOH.

Embodiment consisting 30 provides the compound of any one of embodiments 14, 15, or 24-29, wherein R² is selected from the group consisting of:

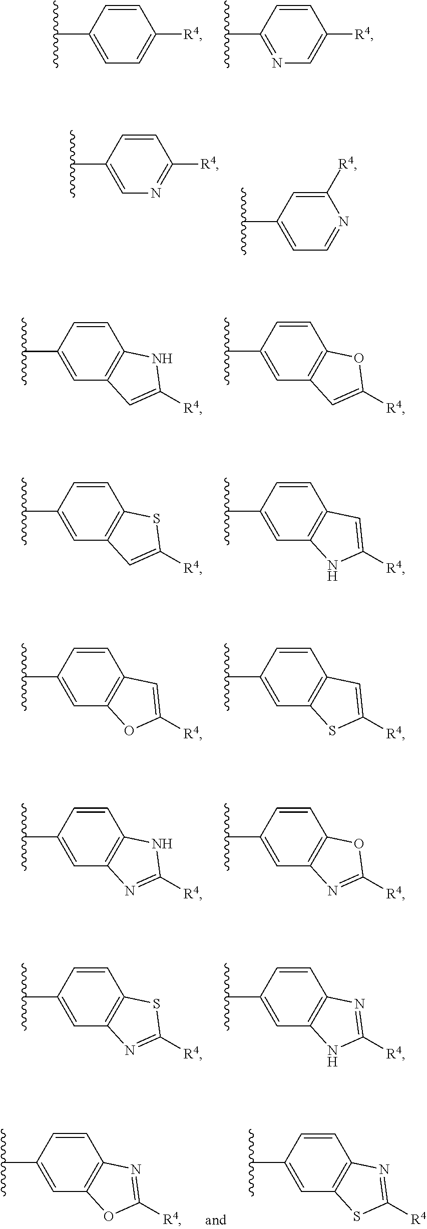

wherein
R⁴ is

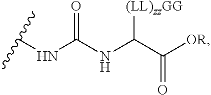

each LL is independently selected from the group consisting of —CH₂—, —NH—, —C(—O)—, and —O—;
R is C₁₋₄ alkyl or H;
zz is an integer from 2 to 6; and
GG is optionally substituted phenyl.

Embodiment 31 provides the compound of any one of embodiments 14, 15, or 24-30, wherein (LL)_{zz}GG is selected from the group consisting of

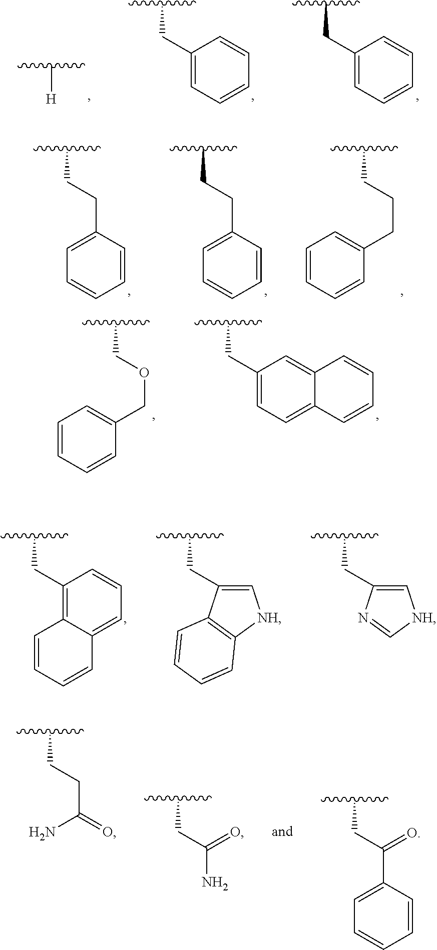

Embodiment 32 provides the compound of any one of embodiments 14, 15, or 24-31, which is selected from the group consisting of

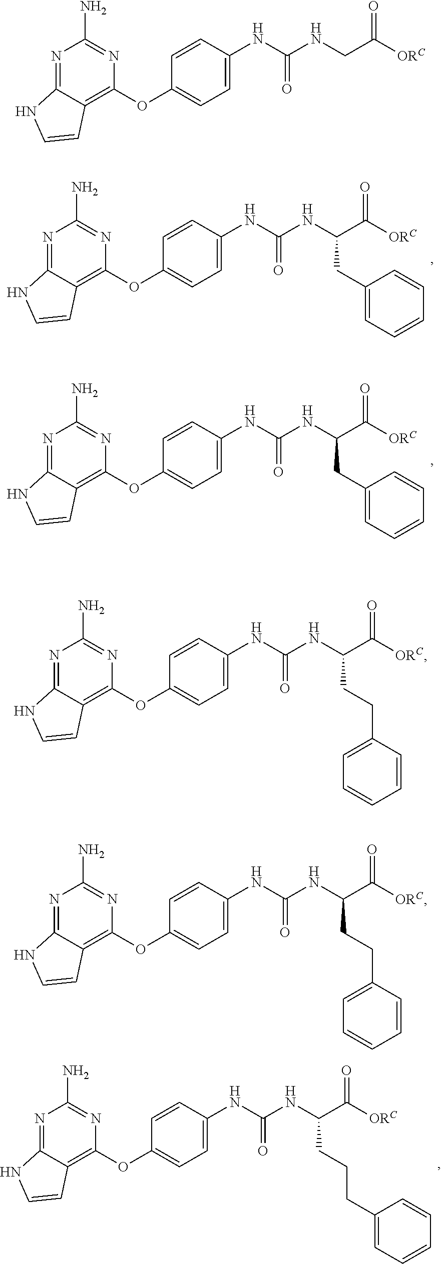

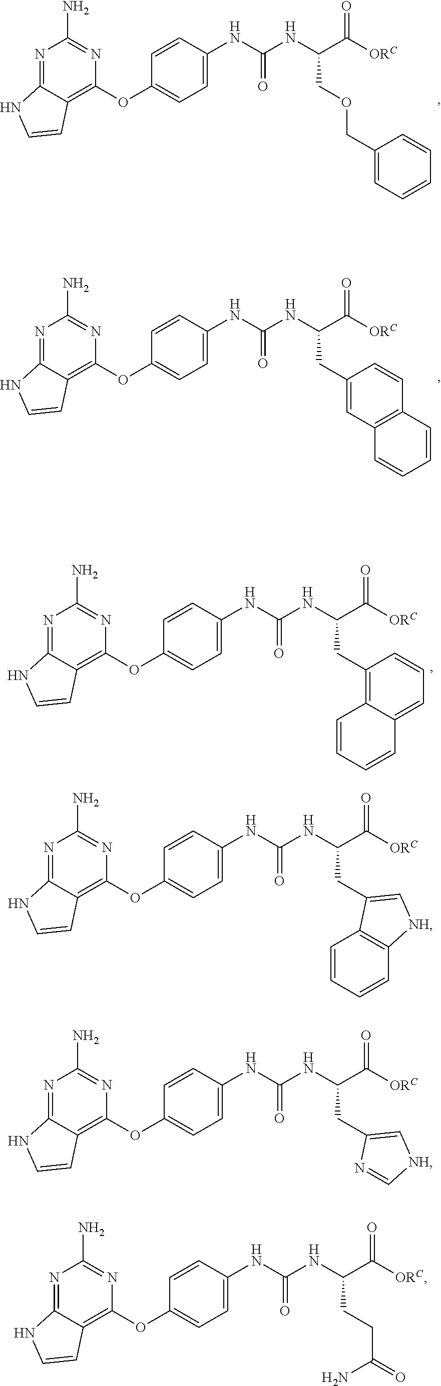

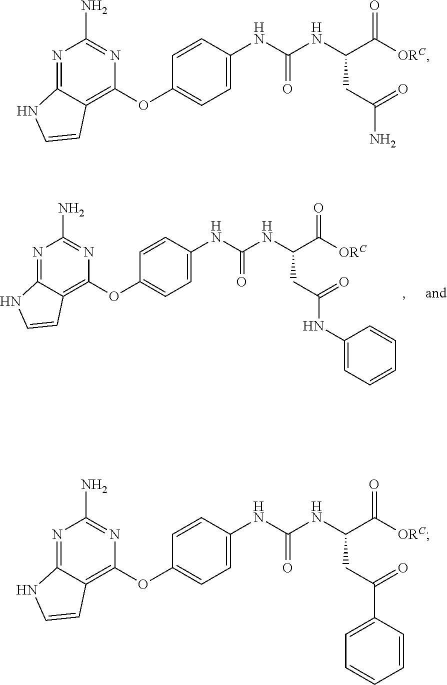

wherein RC is H or CH₃.

Embodiment 33 provides method of treating, ameliorating, and/or preventing a myeloproliferative neoplasm in a patient, the method comprising administering to the patient a therapeutically effective amount of the compound of any one of embodiments 1-32.

Embodiment 34 provides the method of embodiment 33, wherein the myeloproliferative neoplasm is selected from the group consisting of chronic myelogenous leukemia (CML), polycythemia vera, primary myelofibrosis, essential thrombocythemia, chronic neutrophilic leukemia, and chronic eosinophilic leukemia.

Embodiment 35 provides the method of any one of embodiments 33-34, wherein the composition comprises at least one pharmaceutically acceptable excipient.

Embodiment 36 provides the method of any one of embodiments 33-35, wherein the patient is a mammal.

Embodiment 37 provides the method of any one of embodiments 33-36, wherein the patient is human.

Embodiment 38 provides the method of any one of embodiments 33-37, wherein the compound is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical.

Embodiment 39 provides the method of any one of embodiments 33-38, further comprising concurrently or sequentially administering at least one additional agent.

Embodiment 40 provides the method of any one of embodiments 33-39, wherein the at least one additional agent is selected from the group consisting of Adriamycin PFS (Doxorubicin Hydrochloride), Adriamycin RDF (Doxorubicin Hydrochloride), Arsenic Trioxide, Azacitidine Cerubidine (Daunorubicin Hydrochloride), Clafen 5 (Cyclophosphamide), Cyclophosphamide, Cytarabine, Cytosar-U (Cytarabine), Cytoxan (Cyclophosphamide), Dacogen (Decitabine), Dasatinib, Daunorubicin Hydrochloride, Decitabine Doxorubicin Hydrochloride, Etoposide Phosphate, Gleevec (Imatinib Mesylate), Imatinib Mesylate, Jakafi (Ruxolitinib Phosphate), Nilotinib, Rubidomycin (Daunorubicin Hydrochloride), Ruxolitinib Phosphate, Sprycel (Dasatinib), Tarabine PFS (Cytarabine), Tasigna (Nilotinib), Trisenox (Arsenic Trioxide), and Vidaza (Azacitidine).

1. A compound of Formula I, or a pharmaceutically acceptable salt, tautomer, or enantiomer thereof: 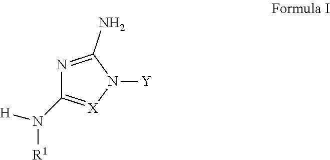
wherein:
Y is selected from the group consisting of —CH₂—R², —C(═O)NR²R³, optionally substituted C₅₋₁₀ heteroaryl, and optionally substituted C₅₋₆ heterocycloalkyl;
wherein the optional substitution is at least one substituent selected from the group consisting of C₁₋₅ alkyl, C₁₋₅ alkoxy, C₁₋₅ thioalkyl, C₁₋₅ aminoalkyl, C₅₋₁₀ aryl, C₅₋₁₀ heteroaryl, ═O (oxo), F, Cl, Br, I, C(═O)OR, NHC(═O)R, and OH;
R¹ and R² are each independently selected from the group consisting of C₂₋₆ alkenyl, C₃₋₇ cycloalkyl, C₆₋₁₀ aryl, 5-10 membered heteroaryl, 4-10 membered heterocycloalkyl, C₆₋₁₀-5-6 membered heterobiaryl, 5-6 membered-C₆₋₁₀ heterobiaryl, and C₆₋₁₀-C₆₋₁₀ biaryl,
each optionally substituted by at least one group of formula (LL)_{zz}-GG, wherein:
LL is at each occurrence independently selected from the group consisting of —O—, —OR, —S—, —S(═O)—, —S(—O)₂—, —NR², —CH═, —C═, —CH₂—, —CHR—, —CR₂—, —CH₃, —C(═O)—, and —C(═NR)—;
zz is an integer from 1 to 30;
GG is a substituent selected from the group consisting of F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, and C(═NH)N(R)₂,
each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
R³ is H or C₁₋₄ hydrocarbyl;
X is N or C—R⁴, wherein R⁴ is H or C₁₋₄ hydrocarbyl optionally substituted by 1 to 3 substituents selected from the group consisting of OR′, NHR′, and NR′2,
each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl; or wherein two R′ groups bonded to a nitrogen atom or to adjacent nitrogen atoms can together with the nitrogen atom or atoms form a heterocyclyl.

2. The compound of claim 1, wherein at least one of the following applies:
i) R¹ is C₆₋₁₀ aryl;
ii) R² is C₆₋₁₀-5-6 membered heterobiaryl, 5-6 membered-C₆₋₁₀ heterobiaryl, or C₆₋₁₀-C₆₋₁₀ biaryl, each of which is at least disubstituted on a terminal ring;
iii) X is N;
iv) R³ is H; and
v) R¹ has the structure: 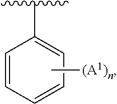
wherein:
each occurrence of A¹ is independently selected from the group consisting of SO₂R, SO₂N(R)₂, C(═O)R, and C(═O)OR, wherein each occurrence of R is independently selected from the group consisting of hydrogen, (C₁-C₆) hydrocarbyl, and (C₃-C₁₈)heterocycloalkyl; and
n is 0, 1, 2, 3, 4, or 5.

3-6. (canceled)

7. The compound of claim 2, wherein the compound is of Formula Ia:

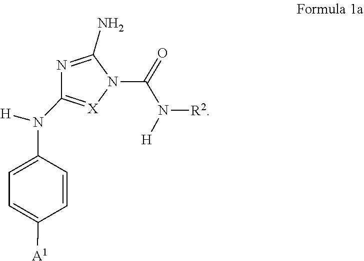

8. The compound of claim 7, wherein A¹ is C(═O)NH—C₁₋₆ alkyl or C(—O)—C₆ heterocycloalkyl.

9. The compound of claim 8, wherein A¹ is C(—O)NHMe or SO₂NH₂.

10. The compound of claim 8, wherein the compound is of Formula Ib, Formula Ic, or Formula Id:

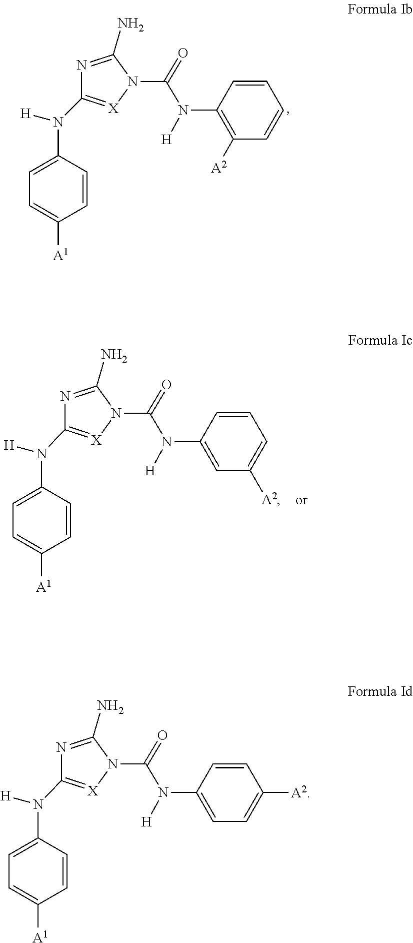

11. The compound of claim 8, wherein A² is selected from the group consisting of: 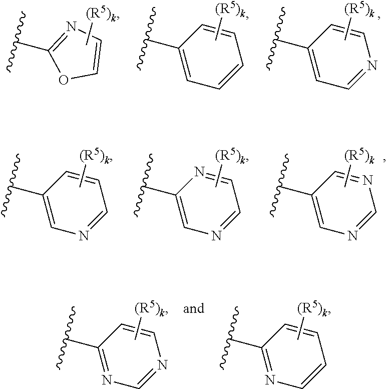
wherein k is 2 or 3.

12. The compound of claim 11, wherein
each R⁵ is independently (LL)_{zz}-GG;
LL is selected from group consisting of —O—, —CH₂—, and —CHR—;
zz is 0, 1, 2, or 3; and
GG is selected from the group consisting of C(—O)OH, C(═O)O—C₁₄ alkyl, (C₆-C₁₀)aryl, and (C₅-C₁₀)heteroaryl.

13. The compound of claim 1, wherein the compound is selected from the group consisting of:

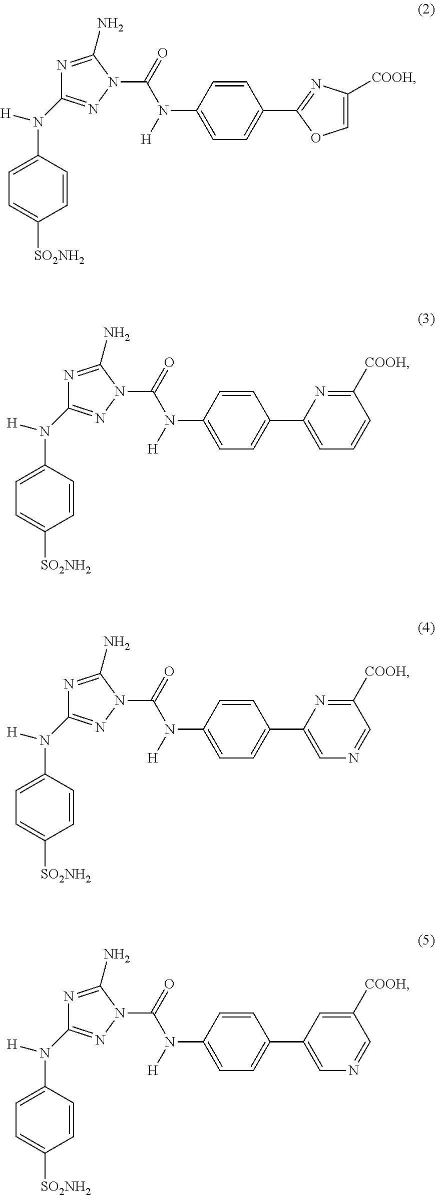 [SMILES: Nc1nc(Nc2ccc(S(N)(=O)=O)cc2)nn1C(=O)Nc1ccc(-c2nc(C(=O)O)co2)cc1 ; Nc1nc(Nc2ccc(S(N)(=O)=O)cc2)nn1C(=O)Nc1ccc(-c2cccc(C(=O)O)n2)cc1 ; Nc1nc(Nc2ccc(S(N)(=O)=O)cc2)nn1C(=O)Nc1ccc(-c2cncc(C(=O)O)n2)cc1 ; Nc1nc(Nc2ccc(S(N)(=O)=O)cc2)nn1C(=O)Nc1ccc(-c2cncc(C(=O)O)c2)cc1]

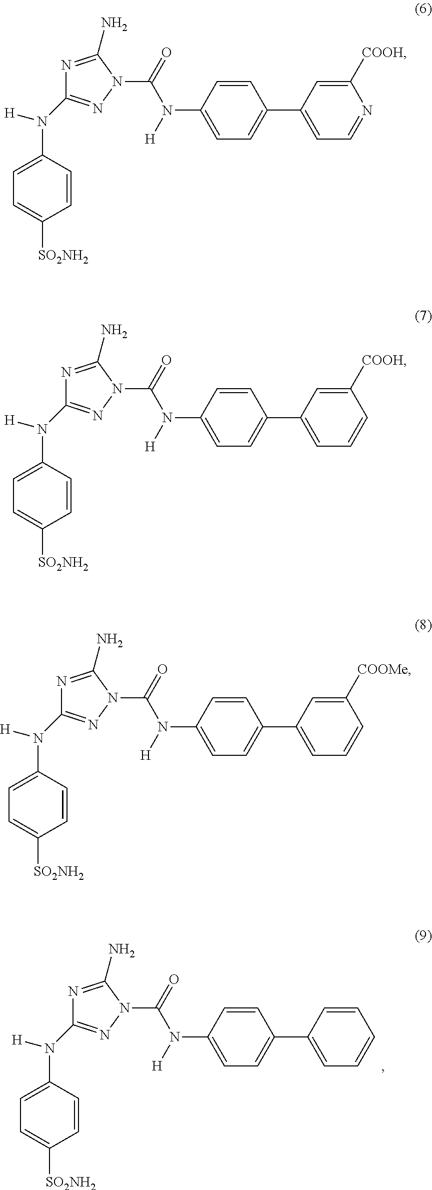 [SMILES: Nc1nc(Nc2ccc(S(N)(=O)=O)cc2)nn1C(=O)Nc1ccc(-c2ccnc(C(=O)O)c2)cc1 ; Nc1nc(Nc2ccc(S(N)(=O)=O)cc2)nn1C(=O)Nc1ccc(-c2cccc(C(=O)O)c2)cc1 ; COC(=O)c1cccc(-c2ccc(NC(=O)n3nc(Nc4ccc(S(N)(=O)=O)cc4)nc3N)cc2)c1 ; Nc1nc(Nc2ccc(S(N)(=O)=O)cc2)nn1C(=O)Nc1ccc(-c2ccccc2)cc1]

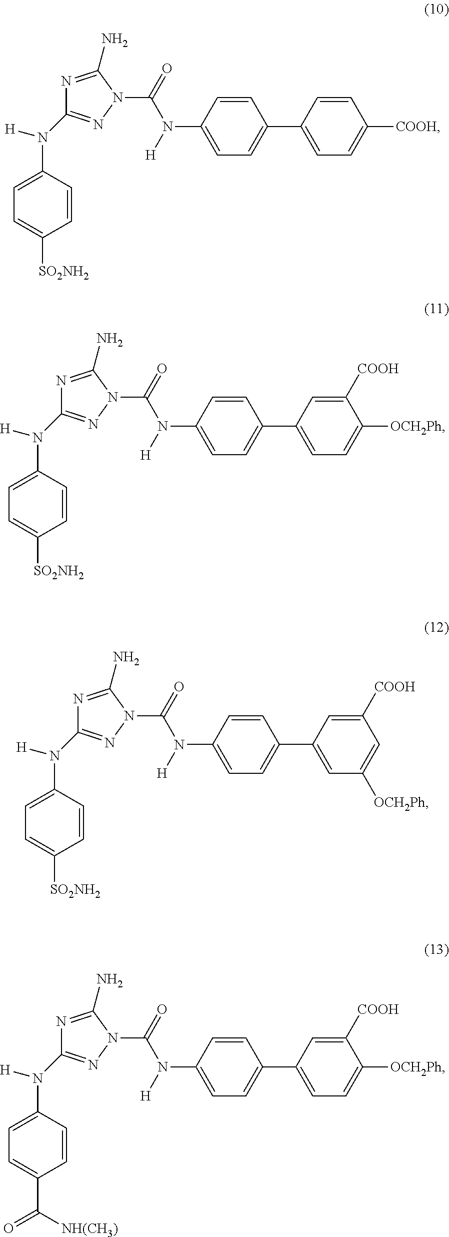

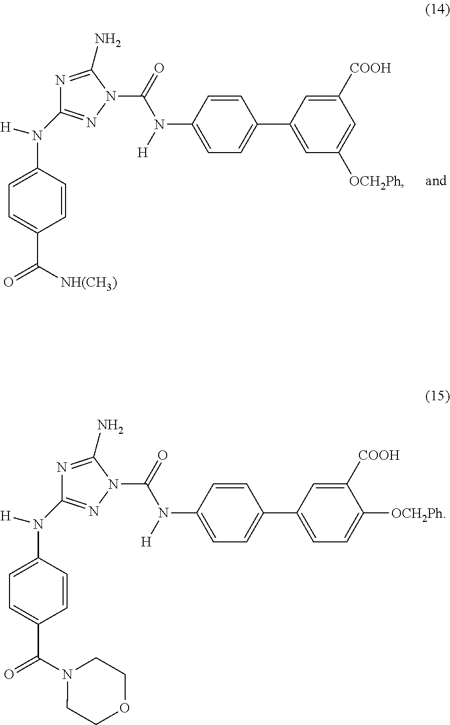

14. A compound of Formula II, or a pharmaceutically acceptable salt, tautomer, or enantiomer thereof: 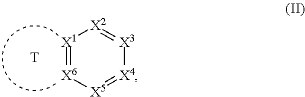
wherein:
T is an optional 5 or 6 membered heterocyclic fused ring that is optionally substituted by at least one -(LL)_{zz}-GG;
each of X¹-X⁶ is independently N or C—Y;
each occurrence of Y is independently absent, H, -(Q)ₙ-(C₃-C₁₂)cycloalkyl, -(Q)ₙ-(C₃-C₁₈)heterocycloalkyl, -(Q)ₙ-(C₆-C₁₈)aryl, or -(Q)ₙ-(C₅-C₁₈)heteroaryl;
Q is absent, or independently selected at each occurrence from the group consisting of O, CH₂, NH, and N—C₁₋₄ alkyl;
n is an integer from 1 to 10;
each cycloalkyl, heterocycloalkyl, aryl, and heteroaryl in Y is optionally substituted by at least one -(LL)_{zz}-GG,
at each occurrence LL is independently selected from the group consisting of —O—, —OR, —S—, —S(═O)—, —S(═O)₂—, —NR—, —NR², —CH═, —C═, —CH₂—, —CHR—, —CR₂—, —CH₃, —C(═O)—, —C(═NR)—;
zz is an integer from 1 to 30;
GG is a substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl;
wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
wherein each alkyl, alkenyl, alkynyl, cycloalkyl, heterocycloalkyl, aryl, and heteroaryl in GG is optionally substituted by at least one substituent selected from the group consisting of H, F, Cl, Br, I, OR′, OC(═O)N(R′)₂, CN, NO₂, CF₃, OCF₃, R′, N(′R)₂, SR′, SOR′, SO₂R′, SO₂N(R′)₂, SO₃R′, C(═O) R′, C(═O)OR′, OC(═O) R′, O(CH₂)₀₋₂C(═O)OR′, C(═O)N(R′)₂, OC(═O)N(R′)₂, (CH₂)₀₋₂N(R′)C(═O) R′, N(R′)SO₂R′, N(R′)C(═O)OR′, N(R′)C(═O) R′, N(R′)C(═O)N(R′)₂, and C(═NH)N(R′)₂;
wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof;
with the proviso that at least two of X¹-X⁶ is N and at least one of X¹-X⁶ is C—NH₂.

15. The compound of claim 14, having the formula:

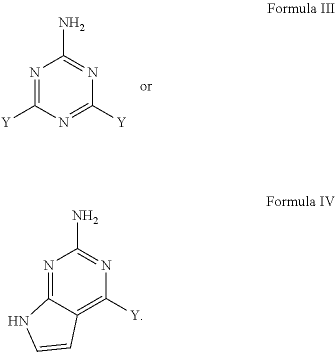

16. The compound of claim 15, having the formula: 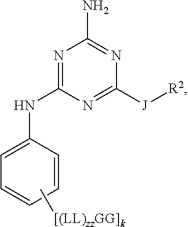
wherein
J is O or NH;
k is an integer from 1 to 5; and
R² is selected from the group consisting of C₆₋₁₀ aryl, C₆₋₁₀ heteroaryl, and combinations thereof, each of which is optionally substituted by at least one substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl.

17. The compound of claim 16, having the formula: 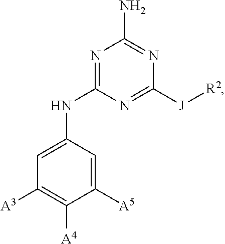
wherein A³, A⁴, A⁵ are each independently selected from the group consisting of H, F, CF₃, CN, OCF₃, SO₂R, SO₂N(R)₂, C(═O)R, and C(═O)OR, C(═O) (CH₂)ₘOCH₃, O(CH₂)ₘOCH₃, (C₅-C₁₀)heteroaryl, and (C₃-C₁₈)heterocycloalkyl,
wherein the heteroaryl and heterocycloalkyl are each optionally substituted by at least one substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl, and
wherein each occurrence of R is independently selected from the group consisting of hydrogen, (C₁-C₆) hydrocarbyl, (C₅-C₁₀)heteroaryl, and (C₃-C₁₈)heterocycloalkyl.

18. The compound of claim 17, wherein A⁴ is selected from the group consisting of CN, SO₂NH₂, and C(═O)NHCH₃.

19. The compound of claim 16, wherein J is O.

20. The compound of claim 18, wherein R² is selected from the group consisting of: 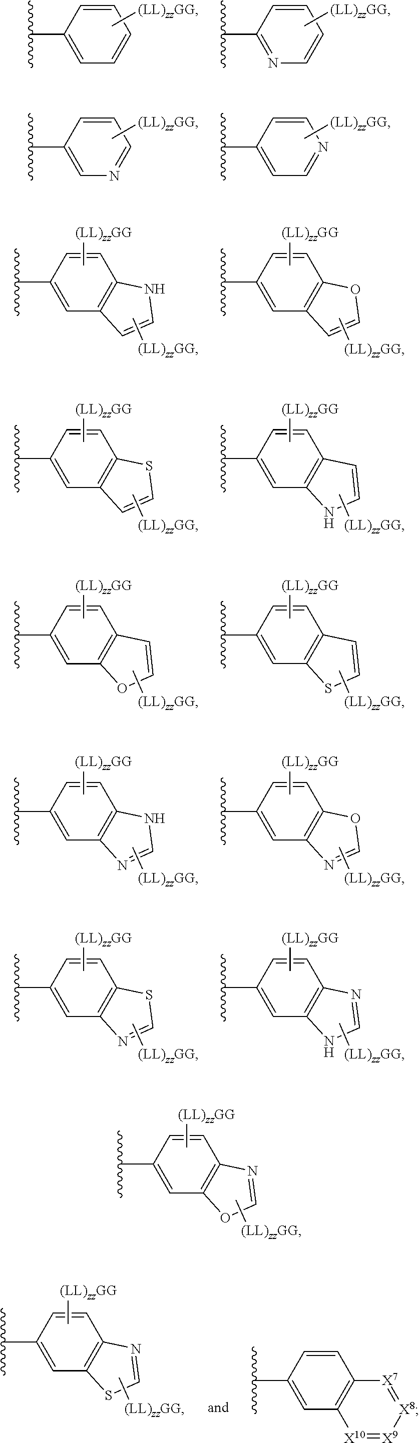
wherein X⁷, X⁸, X⁹, and X¹⁰ are each independently N, NH, or C—Y; and
wherein each -(LL)_{zz}GG moiety represents at least one substituent.

21. The compound of claim 20, wherein R² is selected from the group consisting of

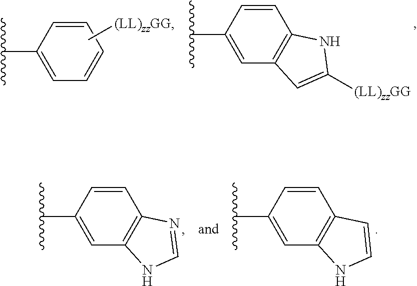

22. The compound of claim 21, wherein R² is selected from the group consisting of 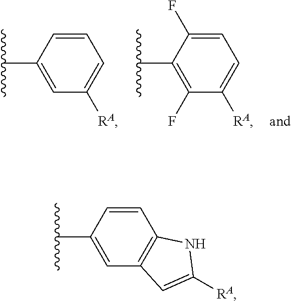
wherein R^{A} is selected from the group consisting of H, CO₂H, NH₂, OH, OCH₃, CH₂OCH₂COOH, and trans C═C(H)(COOH).

23. The compound of claim 20, wherein R² is selected from the group consisting of: 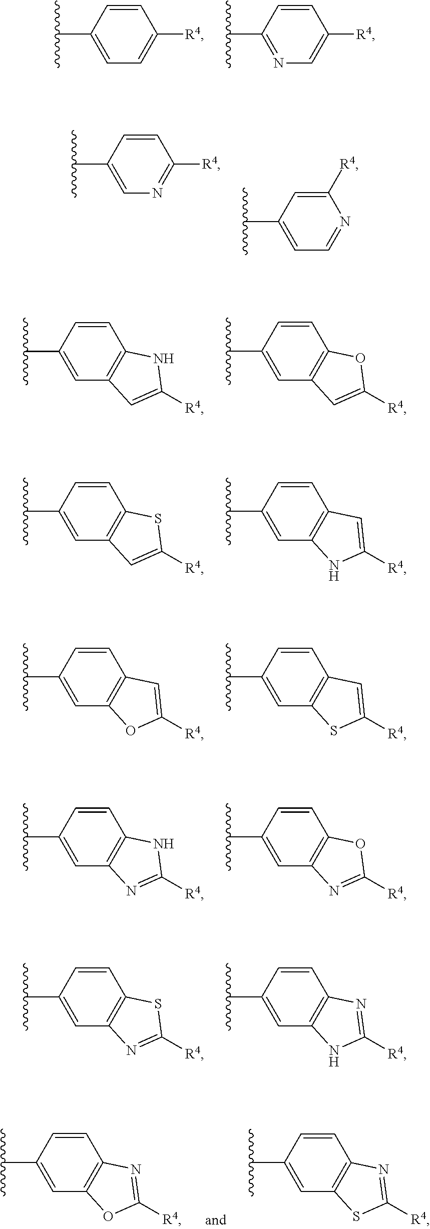
wherein
R⁴ is 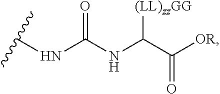
each LL is independently selected from the group consisting of —CH₂—, —NH—, —C(═O)—, and —O—;
R is C₁₋₄ alkyl or H;
zz is an integer from 2 to 6; and
GG is phenyl optionally substituted by at least one substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(—O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl.

24. The compound of claim 23, wherein (LL)_{zz}GG is selected from the group consisting of (CH₂)₂Ph, (CH₂) 3Ph, CH₂C(═O)NHPh, and CH₂ (C═O)Ph.

25. The compound of claim 15, having the formula: 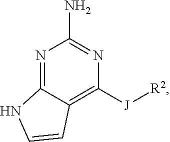
wherein:
J is O or NH;
R² is selected from the group consisting of C₆₋₁₀ aryl, C₆₋₁₀ heteroaryl, and combinations thereof, each of which is optionally substituted by at least one substituent selected from the group consisting of H, F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl.

26. The compound of claim 25, wherein J is O.

27. The compound of claim 25, wherein R² is selected from the group consisting of: 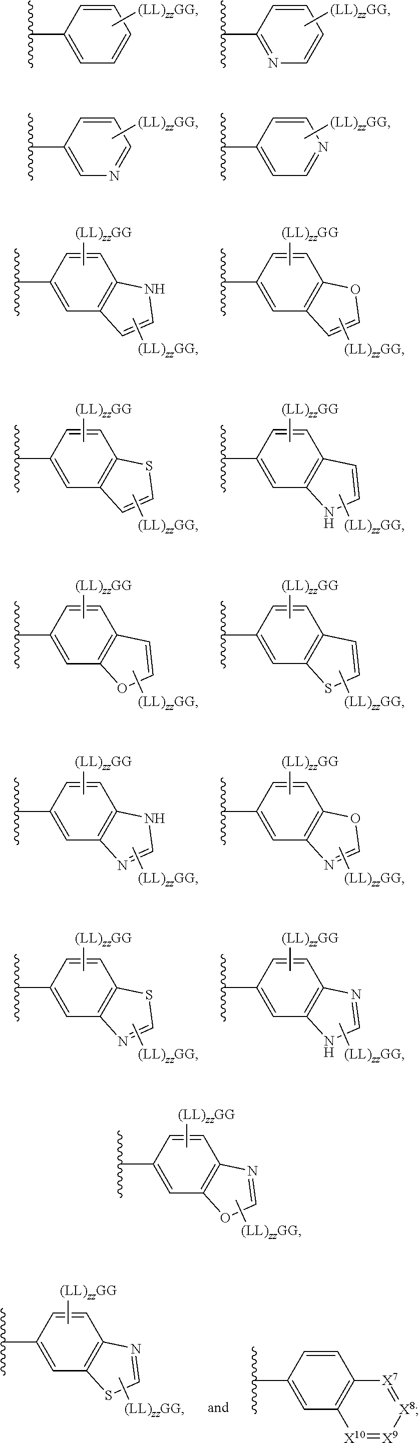
wherein X⁷, X⁸, X⁹, and X¹⁰ are each independently N, NH, or C—Y; and
wherein each -(LL)_{zz}GG moiety represents at least one substituent.

28. The compound of claim 27, wherein R² is selected from the group consisting of

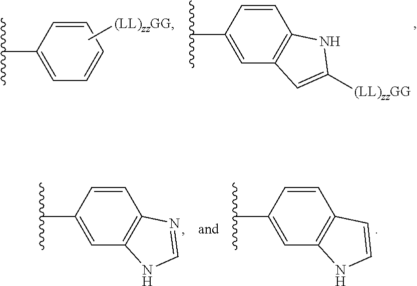

29. The compound of claim 28, wherein R² is selected from the group consisting of 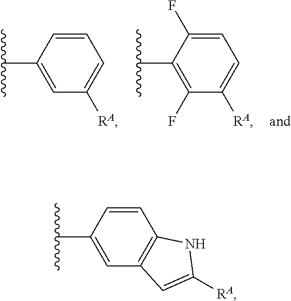
wherein R^{A} is selected from the group consisting of —H, —CO₂H, —NH₂, —OH, —OCH₃, —CH₂OCH₂COOH, and trans —C═C(H)COOH.

30. The compound of claim 27, wherein R² is selected from the group consisting of: 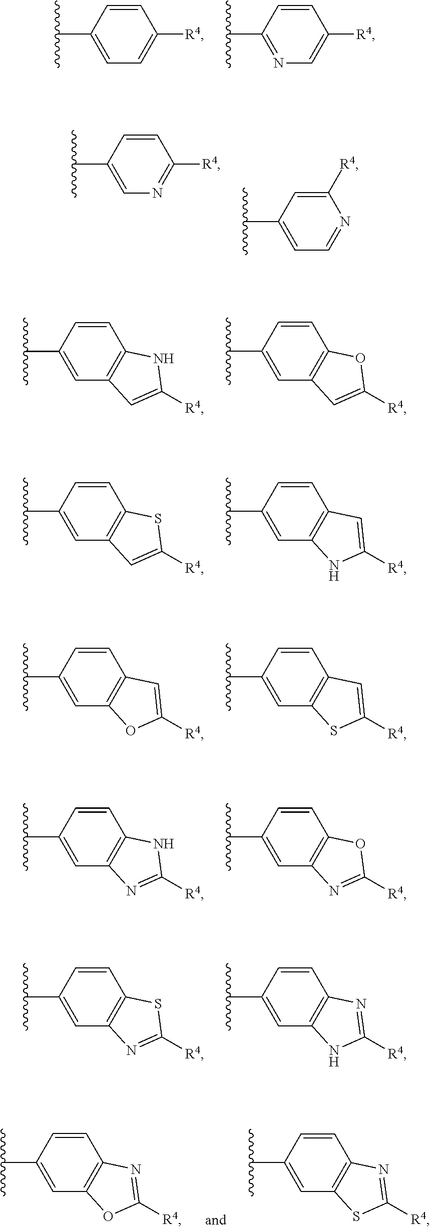
wherein
R⁴ is 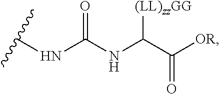
each LL is independently selected from the group consisting of —CH₂—, —NH—, —C(═O)—, and —O—;
R is C₁₋₄ alkyl or H;
zz is an integer from 2 to 6; and
GG is phenyl optionally substituted by at least one substituent selected from the group consisting of H, F, C₁, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O) R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl.

31. The compound of claim 23, wherein (LL)_{zz}GG is selected from the group consisting of

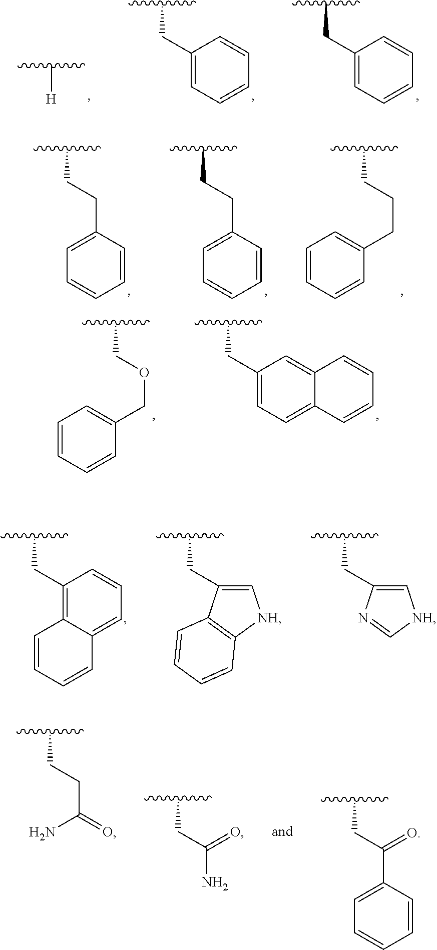

32. The compound of claim 25, which is selected from the group consisting of 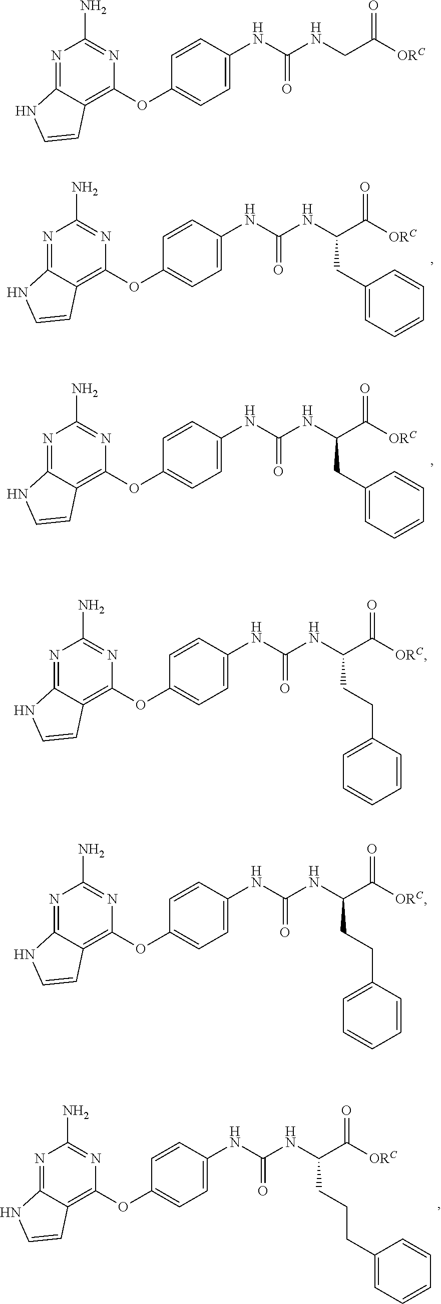 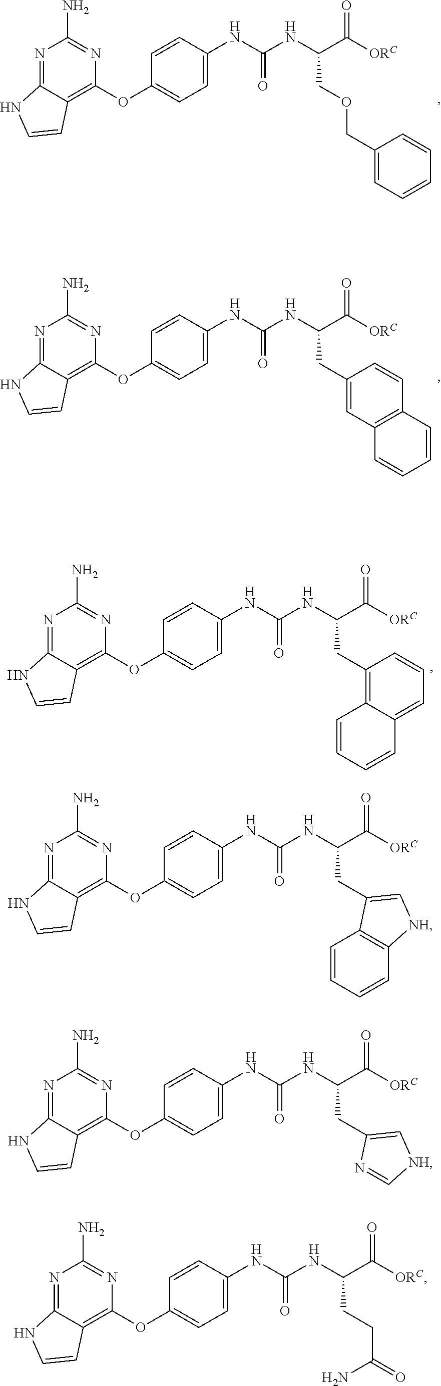 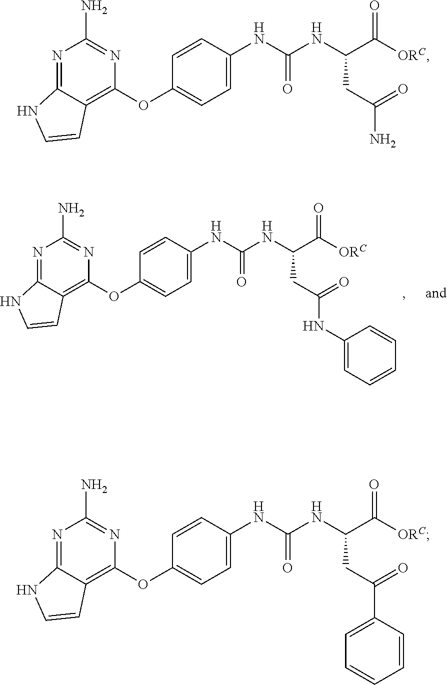
wherein RC is H or CH₃.

33. A method of treating or ameliorating a myeloproliferative neoplasm in a patient in need thereof, the method comprising:
administering to the patient a therapeutically effective amount of a compound of Formula I or Formula II, or a pharmaceutically acceptable salt, tautomer, or enantiomer thereof, optionally wherein the compound or pharmaceutically acceptable salt, tautomer, or enantiomer thereof is formulated as a pharmaceutical composition further comprising at least one pharmaceutically acceptable excipient: 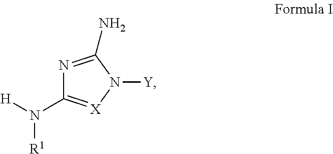
wherein:
Y is selected from the group consisting of —CH₂-R², —C(═O)NR²R³, optionally substituted C₅₋₁₀ heteroaryl, and optionally substituted C₅₋₆ heterocycloalkyl;
wherein the optional substitution is at least one substituent selected from the group consisting of C₁₋₅ alkyl, C₁₋₅ alkoxy, C₁₋₅ thioalkyl, C₁₋₅ aminoalkyl, C₅₋₁₀ aryl, C₅₋₁₀ heteroaryl, ═O (oxo), F, Cl, Br, I, C(═O)OR, NHC(—O)R, and OH;
R¹ and R² are each independently selected from the group consisting of C₂₋₆ alkenyl, C₃₋₇ cycloalkyl, C₆₋₁₀ aryl, 5-10 membered heteroaryl, 4-10 membered heterocycloalkyl, C₆₋₁₀-5-6 membered heterobiaryl, 5-6 membered-C₆₋₁₀ heterobiaryl, and C₆₋₁₀-C₆₋₁₀ biaryl, each optionally substituted by at least one group of formula (LL)_{zz}-GG, wherein:
LL is at each occurrence independently selected from the group consisting of —O—, —OR, —S—, —S(═O)—, —S(═O)₂—, —NR², —CH═, —C═, —CH₂—, —CHR—, —CR₂—, —CH₃, —C(═O)—, and —C(═NR)—;
zz is an integer from 1 to 30;
GG is a substituent selected from the group consisting of F, Cl, Br, I, OR, OC(═O)N(R)₂, CN, NO₂, CF₃, OCF₃, R, N(R)₂, SR, SOR, SO₂R, SO₂N(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, and C(═NH)N(R)₂,
each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
R³ is H or C₁₋₄ hydrocarbyl;
X is N or C—R⁴, wherein R⁴ is H or C₁₋₄ hydrocarbyl optionally substituted by 1 to 3 substituents selected from the group consisting of OR′, NHR′, and NR′₂,
each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl; or wherein two R′ groups bonded to a nitrogen atom or to adjacent nitrogen atoms can together with the nitrogen atom or atoms form a heterocyclyl;
or 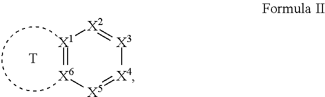
wherein:
T is an optional 5 or 6 membered heterocyclic fused ring that is optionally substituted by at least one -(LL)_{zz}-GG;
each of X¹-X⁶ is independently N or C—Y;
each occurrence of Y is independently absent, H, -(Q)ₙ-(C₃-C₁₂)cycloalkyl, -(Q)ₙ-(C₃-C₁₈)heterocycloalkyl, -(Q)ₙ-(C₆-C₁₈)aryl, or -(Q)ₙ-(C₅-C₁₈)heteroaryl;
Q is absent, or independently selected at each occurrence from the group consisting of O, CH₂, NH, and N—C₁₋₄ alkyl;
n is an integer from 1 to 10;
each cycloalkyl, heterocycloalkyl, aryl, and heteroaryl in Y is optionally substituted by at least one -(LL)_{zz}-GG,
at each occurrence LL is independently selected from the group consisting of —O—, —OR, —S—, —S(═O)—, —S(═O)₂—, —NR—, —NR², —CH═, —C═, —CH₂—, —CHR—, —CR₂—, —CH₃, —C(═O)—, —C(═NR)—;
zz is an integer from 1 to 30;
GG is a substituent selected from the group consisting of H, F, Cl, Br, I, OR, OC(═O)N(R)₂, CN, NO₂, CF₃, OCF₃, R, N(R)₂, SR, SOR, SO₂R, SON(R)₂, SO₃R, C(═O)R, C(═O)OR, OC(═O)R, O(CH₂)₀₋₂C(═O)OR, C(═O)N(R)₂, OC(═O)N(R)₂, (CH₂)₀₋₂N(R)C(═O)R, N(R)SO₂R, N(R)C(═O)OR, N(R)C(═O)R, N(R)C(═O)N(R)₂, C(═NH)N(R)₂, (C₃-C₁₂)cycloalkyl, (C₃-C₁₈)heterocycloalkyl, (C₆-C₁₈)aryl, and (C₅-C₁₈)heteroaryl;
wherein each occurrence of R is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof; or wherein two R groups bonded to a nitrogen atom or to adjacent nitrogen atoms can together with the nitrogen atom or atoms form a heterocyclyl;
wherein each alkyl, alkenyl, alkynyl, cycloalkyl, heterocycloalkyl, aryl, and heteroaryl in GG is optionally substituted by at least one substituent selected from the group consisting of H, F, Cl, Br, I, OR′, OC(═O)N(R′)₂, CN, NO₂, CF₃, OCF₃, R′, N(′R)₂, SR′, SOR′, SO₂R′, SO₂N(R′)₂, SO₃R′, C(O) R′, C(═O)OR′, OC(═O) R′, O(CH₂)₀₋₂C(═O)OR′, C(═O)N(R′)₂, OC(═O)N(R′)₂, (CH₂)₀₋₂N(R′)C(═O) R′, N(R′)SO₂R′, N(R′)C(═O)OR′, N(R′)C(═O) R′, N(R′)C(—O)N(R′)₂, and C(═NH)N(R′)₂;
wherein each occurrence of R′ is independently selected from the group consisting of hydrogen, (C₁-C₁₀)alkyl, (C₂-C₁₀)alkenyl, (C₂-C₁₀)alkynyl, (C₃-C₁₂)cycloalkyl, (C₃-C₁₈)heterocycloalkyl, (C₆-C₁₈)aryl, (C₅-C₁₈)heteroaryl, and combinations thereof;
with the proviso that at least two of X¹-X⁶ is N and at least one of X¹-X⁶ is C—NH₂.

34-36. (canceled)

37. The method of claim 33, wherein at least one of the following applies:
(a) the patient is human;
(b) the myeloproliferative neoplasm is selected from the group consisting of chronic myelogenous leukemia (CML), polycythemia vera, primary myelofibrosis, essential thrombocythemia, chronic neutrophilic leukemia, and chronic eosinophilic leukemia;
(c) the compound is administered by a route selected from the group consisting of oral, transdermal, intravesical, intrapulmonary, intraduodenal, intragastrical, intrathecal, subcutaneous, intramuscular, intradermal, intra-arterial, intravenous, intrabronchial, inhalation, and topical;
(d) the patient is concurrently or sequentially administered at least one additional agent selected from the group consisting of azacitidine, doxorubicin hydrochloride, arsenic trioxide, daunorubicin hydrochloride, cyclophosphamide, cytarabine, decitabine, dasatinib, etoposide phosphate, imatinib mesylate, ruxolitinib phosphate, and nilotinib.

38-40. (canceled)
